(19) United States
(12) Patent Application Publication
(10) Pub. No.: US 2026/0053870 A1
(43) Pub. Date: Feb. 26, 2026

(54) METHANOLYTIC AGENTS AND METHODS OF USING THE SAME

(71) Applicant: Lumen Bioscience, Inc., Seattle, WA (US)

(72) Inventors: James Michael ROBERTS, Seattle, WA (US); Nhi Yen KHUONG, Kenmore, WA (US); Kristie Miller KEENEY, Seattle, WA (US); Mesfin Mulugeta GEWE, Bothell, WA (US); Colin Matthew Watson BRADY, Seattle, WA (US); Mark Lewis HEINNICKEL, Seattle, WA (US)

(21) Appl. No.: 19/383,248

(22) Filed: Nov. 7, 2025

Related U.S. Application Data
(63) Continuation of application No. PCT/US2024/029499, filed on May 15, 2024.
(60) Provisional application No. 63/502,394, filed on May 15, 2023.

(51) Int. Cl. A61K 35/748; A23K 10/18; A23K 50/10; A61K 38/48; A61P 1/14; C12N 9/52; C12N 15/74

(52) U.S. Cl. CPC A61K 35/748; A23K 10/18; A23K 50/10; A61K 38/4873; A61P 1/14; C12N 9/52; C12N 15/74; C12N 2800/101

(57) ABSTRACT

The present disclosure is related to compositions and methods comprising genetically modified Arthrospira platensis strains that express methanogen-killing lytic enzymes (methanolysins) to inhibit methane emission produced by enteric archaea.

### CROSS-REFERENCE TO RELATED APPLICATIONS

This application is a continuation of International Application No. PCT/US2024/029499, filed on May 15, 2024, which claims priority to U.S. Application No. 63/502,394, filed on May 15, 2023, the contents of each of which is incorporated by reference herein in its entirety for all purposes.

### REFERENCE TO AN ELECTRONIC SEQUENCE LISTING

The contents of electronic sequence the listing (LUBI_036_01US_SeqList_ST26.xml; Size: 171,210 bytes; and Date of Creation: Nov. 7, 2025) are herein incorporated by reference in their entirety.

### FIELD

The present disclosure is related to compositions and methods comprising genetically modified Arthrospira platensis (e.g., Spirulina) strains that express methanogen-killing lytic enzymes (methanolysins) to inhibit methane emission produced by enteric microbes.

### BACKGROUND

Enteric fermentation by ruminants is the single largest source of greenhouse gas (GHG) emissions by the agricultural industry. Methane, a more potent greenhouse gas than CO₂, is produced by microorganisms that reside in the rumen of livestock. Existing feed supplementation approaches for mitigating enteric methane have relied on chemical inhibitors produced synthetically or in a red alga that can be grown in coastal environments. These approaches are only partially effective, not scalable, and can be toxic to the animal, product, and/or environment. Biologics-based approaches are effective and safe, but the approach suffers from the lack of a cost-effective manufacturing platform.

Thus, there is a need for alternatives to address enteric fermentation in ruminants. The present disclosure addresses these needs.

### SUMMARY

Provided herein are modified Spirulina cells, comprising: an exogenous sequence encoding a methanolysin. In aspects, the exogenous sequence encoding the methanolysin is genomically integrated into the Spirulina cell genome. In aspects, the genomic integration is within a neutral genomic region. In aspects, the neutral genomic region is selected from the group consisting of: kanamycin aminoglycoside acetyltransferase, NS1, and both kanamycin aminoglycoside acetyltransferase and NS1. In aspects, the neutral genomic region is the kanamycin aminoglycoside acetyltransferase. In aspects, the methanolysin is from an archaea organism. In aspects, the organism is from archaea selected from the group consisting of: Methanobrevibacter ruminantium, Methanobrevibacter gottschalkii and Methanosphaera cuniculi. In aspects, the archaea is Methanobrevibacter ruminantium M1. In aspects, the Methanobrevibacter ruminantium methanolysin is PeiR-MC. In aspects, the PeiR-MC methanolysin comprises an amino acid sequence with at least about 80%, 85%, 90%, 95%, 98%, or 99% identity to SEQ ID NO: 78. In aspects, the PeiR-MC methanolysin comprises SEQ ID NO: 78. In aspects, the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.], TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.]. In aspects, the methanolysin comprises an amino acid sequence with at least about 80%, 85%, 90%, 95%, 98%, or 99% identity to SEQ ID NO. 1-102. In aspects, the methanolysin comprises a sequence of SEQ ID NOs: 1-102. In aspects, the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspects, the Spirulina is the A. platensis. In aspects, provided herein is a population of Spirulina, comprising: the modified Spirulina cell disclosed herein.

Also provided herein are populations of Spirulina, comprising: two or more Spirulina cells, wherein each of the two or more Spirulina cells express different methanolysin proteins. In aspects, the two or more Spirulina cells express different methanolysin proteins that comprise an amino acid sequence with at least about 80%, 85%, 90%, 95%, 98%, or 99% identity to SEQ ID NO: 1 to SEQ ID NO: 102. In aspects, the two or more Spirulina cells comprise two or more methanolysins selected from the group consisting of: SEQ ID NO: 1 to SEQ ID NO: 102.

Provided herein are also food supplement compositions, comprising: modified Spirulina cells disclosed herein; or populations disclosed herein.

Provided herein are also nutraceutical or pharmaceutical compositions comprising the modified Spirulina cells disclosed herein; or populations disclosed herein.

Provided herein are also vectors, comprising: a sequence encoding for methanolysin, wherein the sequence comprises at least 80% identity to any one of SEQ ID NOs: 122, 123, or 124. In aspects, the sequence comprises SEQ ID NO: 122 or SEQ ID NO: 123. In aspects, the vector further comprises a native Spirulina promoter. In aspects, the native Spirulina promoter is Pcpc600. In aspects, the sequence encoding for methanolysin is flanked by a pair of homology arms. In aspects, the homology arms comprise a sequence homologous to a neutral site in the Spirulina genome. In aspects, the neutral site is selected from the group consisting of: kanamycin aminoglycoside acetyltransferase, NS1, and both kanamycin aminoglycoside acetyltransferase and NS1. In aspects, the neutral site comprises a portion of a kanamycin aminoglycoside acetyltransferase gene. In aspects, the first homology arms comprises at least 80% identity to SEQ ID NO: 103, and the second homology arm comprises at least 80% identity to SEQ ID NO: 104. In aspects, the first homology arms comprises SEQ ID NO: 103, and the second homology arm comprises SEQ ID NO: 104. In aspects, the methanolysin is in a fusion protein. In aspects, the fusion protein comprises a tag selected from the group consisting of: maltose binding protein, thioredoxin, phycocyanin, and SUMO.

Provided herein are also kits, comprising: modified Spirulina cells disclosed herein; populations disclosed herein; food supplements disclosed herein; nutraceuticals disclosed herein; and/or vectors disclosed herein. In aspects, the kit further comprising instructions for use thereof.

Provided are also methods of making a modified Spirulina cell, the methods comprising: introducing into a wild type Spirulina cell the vector disclosed herein, thereby generating a modified Spirulina cell. In aspects, a method further comprises culturing modified Spirulina cells thereby generating a population of modified Spirulina cells. In aspects, culturing comprises antibiotic selection. In aspects, the antibiotic is kanamycin, streptomycin, spectinomycin, and/or G418. In aspects, the modified Spirulina cell is dried.

In aspects, provided herein are methods, comprising: administering modified Spirulina cells disclosed herein; populations disclosed herein; food supplements disclosed herein; and/or nutraceuticals or pharmaceuticals disclosed herein.

Provided herein are also methods of reducing methane emissions, the methods comprising: administering a composition comprising Spirulina modified to express a methanolysin to a subject. In aspects, a composition comprises two or more Spirulina that each express a different methanolysin. In aspects, a methanolysin is from an archaea organism. In aspects, an organism is from archaea selected from the group consisting of: Methanobrevibacter ruminantium M1, Methanobrevibacter gottschalkii and Methanosphaera cuniculi. In aspects, the archaea is Methanobrevibacter ruminantium M1. In aspects, the Methanobrevibacter ruminantium M1 methanolysin is PeiR-MC. In aspects, the PeiR-MC methanolysin comprises an amino acid sequence with at least about 80%, 85%, 90%, 95%, 98%, or 99% identity to SEQ ID NO: 78. In aspects, the PeiR-MC methanolysin comprises SEQ ID NO: 78. In aspects, the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.]. TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.]. In aspects, the methanolysin comprises an amino acid sequence with at least about 80%, 85%, 90%, 95%, 98%, or 99% identity to SEQ ID NO: 1 to SEQ ID NO: 102. In aspects, the methanolysin comprises a sequence selected from the group consisting of: SEQ ID NO: 1 to SEQ ID NO: 102. In aspects, the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspects, the Spirulina is A. platensis. In aspects, the administration is effective at reducing methane emissions by at least about 1-fold, 5-fold, 10-fold, 20-fold, or 50-fold as compared to an otherwise comparable method lacking the administration. In aspects, the reduction in methane emissions is determined by detecting methane in metabolic gases generated by the subject. In aspects, the method further comprising re-administering the composition. In aspects, the composition is readministered within about 2 hours, 4 hours, 8 hours, 10 hours, one day, 3 days, 5 days, or a week following the administration. In aspects, the composition is readministered daily. In aspects, the subject is mammalian. In aspects, the mammalian is human. In aspects, the mammalian is non-human. In aspects, the non-human mammalian subject is a ruminant. In aspects, the ruminant is domesticated. In aspects, the ruminant is selected from the group consisting of: cow, bovine, goat, sheep, giraffe, deer, gazelle, and antelope. In aspects, the ruminant is a cow. In aspects, the cow is selected from the group consisting of: Holstein Friesian, Hereford, Simmental, Aberdeen Angus, Brown Swiss, Galloway, Red Angus, Belted Galloway, Braford, Limousin, Belgian Blue, Brangus, Beefalo, Chillingham, Ongole, Cachena, Gelbvieh, American Angus, Chianina, Aubrac, Twinner, Parda Alpina, Diary Shorthorn, Maine-Anjou, Danish Red, Retinta, Horro, Montbeliarde, Heck, White Park, British White, Hallikar, Red Poll, Senepol, Caracu, Lakenvelder, Beef Shorthorn, Corriente, Florida Cracker, Tuli, Hungarian Grey, English longhorn, Tarentaise, Australian Lowline, Boskarin, Black Baldy, French Brown, Podolica, Pustertaler Sprinzen, and any combination thereof. In aspects, the Spirulina is administered in the form of Spirulina biomass. In aspects, the Spirulina biomass is administered at a dose of at least about 0.25 grams to at most about 30 grams. In aspects, the Spirulina biomass is administered at a dose of at least about 0.25 grams to about 2 grams: 0.5 grams to about 1 gram; or 1 gram to about 2 grams.

Provided herein are also methods of treating or preventing disease or bodily dysfunction, the methods comprising: orally administering to a subject a composition comprising Spirulina modified to express a methanolysin. In aspects, the subject is a mammal. In aspects, the mammalian is human. In aspects, the mammalian is non-human. In aspects, the composition is administered in the form of Spirulina biomass. In aspects, the Spirulina biomass is administered at a dose of at least about 0.25 grams to at most about 30 grams. In aspects, the Spirulina biomass is administered at a dose of at least about 0.25 grams to about 2 grams; 0.5 grams to about 1 gram; or 1 gram to about 2 grams. In aspects, the administration is effective at reducing methane emissions as compared to an otherwise comparable method lacking the administration. In aspects, the administration is effective at reducing methanogens in the subject as compared to an otherwise comparable method lacking the administration. In aspects, the subject has a gastrointestinal disease. In aspects, the subject has a gastrointestinal disease selected from the group consisting of: Crohn's disease, ulcerative colitis, proctitis, pouchitis, cuffitis, constipation-predominant irritable bowel syndrome (IBS-C), functional constipation, small intestinal bacterial overgrowth (SIBO), halitosis, gastrointestinal cancer, colorectal cancer, anorexia, and obesity. In aspects, the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.]. TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.]. In aspects, the methanolysin comprises an amino acid sequence with at least about 80%, 85%, 90%, 95%, 98%, or 99% identity to SEQ ID NO: 1 to SEQ ID NO: 102. In aspects, the methanolysin comprises a sequence selected from the group consisting of: SEQ ID NO: 1 to SEQ ID NO: 102. In aspects, the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspects, the Spirulina is A. platensis.

### BRIEF DESCRIPTION OF THE FIGURES

The accompanying figures, which are incorporated herein and form a part of the specification, illustrate some, but not the only or exclusive, example aspects and/or features. It is intended that the aspects and figures disclosed herein are to be considered illustrative rather than limiting.

FIG. 1 shows a plasmid map of an exemplary Spirulina transformation vector. As the gene of interest, a sequence comprising a methanolysin is inserted into the region labeled “GOI.”

FIG. 2A shows an automated western blot analysis using capillary electrophoresis-based immunoassay demonstrates accumulation of a methanolysin from M. cuniculi (labeled as methanolysin) in both the soluble and total protein fractions of Spirulina extracts. Secondary analysis estimates the soluble methanolysin in this strain accumulated to over 2% of the total dry weight. FIG. 2B shows methanogen lysin activity enzyme assay using M. ruminatum ghost cells as the substrate. Cell lysis causes a reduction in turbidity that is followed by a decrease in optical density measurements, OD600 nm. The positive control, PeiR purified from Escherichia coli (PeiR, gray), rapidly lyses the methanogen cells. Spirulina strains expressing methanolysin from M. cuniculi (SP-Methanolysin, blue) also exhibits methanogen lysis activity. WT Spirulina lysate exhibits little lysis activity (WT, orange).

FIGS. 3A-3C show the effect of SEQ ID NO: 78 on the lytic activity of three major rumen methanogens, Methanobrevibacter ruminantium (FIG. 3A), Methanobrevibacter gottschalkii (FIG. 3B) and Methanosphaera cuniculi (FIG. 3C).

FIGS. 4A-4C show the effect of SEQ ID NO: 78 on the lytic activity of three major rumen methanogens, Methanobrevibacter ruminantium (FIG. 4A), Methanobrevibacter gottschalkii (FIG. 4B) and Methanosphaera cuniculi (FIG. 4C) in clarified rumen fluid.

FIGS. 5A-5C show the effect of methanolysin on different activities against different methanogens. FIG. 5A shows the maximum linear rate change (over a 5 min time frame) determine to be correlated with enzyme concentration. Using a hemocytometer, a ratio of methanogens to OD was determined (FIG. 5B). Using the methanogen/OD ratio (FIG. 5B) and rates indicated on the left (FIG. 5A), the lytic rate of methanolysins could be calculated (FIG. 5C).

FIG. 6 shows the effects of SEQ ID NO: 78MC (x-axis) on living methanogens.

FIG. 7 shows a structural analysis enabling identification of methanolysin subdomains. A structural model of PeiR from AlphaFold is shown (left). Amino acid sequences associated with the predicted pseudomurein binding domain (blue sequence), catalytic domain (red sequence), and linker (brown sequence) are highlighted (right). Cysteine residues are highlighted in teal. The complete sequence is identified as SEQ ID NO: 71.

FIG. 8 shows a structural model and amino acid sequence of PeiR catalytic domain (red sequence) and linker sequence (brown sequence). Cysteine residues are highlighted in teal. The complete sequence is identified as SEQ ID NO: 72.

FIG. 9 shows a structural model and amino acid sequence of PeiR catalytic domain (red sequence). Hydrophobic region that may be exposed by truncation of the linker sequence is shown (blue arrow). Cysteine residues are highlighted in teal. The complete sequence is identified as SEQ ID NO: 73.

FIG. 10 shows a molecular electrostatic analysis identifying two residues in the PeiR catalytic domain that could be mutated to improve solubility (mutated residues indicated in bold red). Cysteine residues are highlighted in teal. Surface electrostatics of the predicted structure of PeiR catalytic domain with (right) and without (left) proposed mutations indicate the change in surface charge of the solvent exposed region. The complete sequence is identified as SEQ ID NO: 74.

FIG. 11 shows an amino acid sequence of the predicted subdomains (N-terminal domain, brown; pseudomurein binding domain, blue; catalytic domain, red) of a methanolysin from M. cuniculi and AlphaFold structural model of the predicted catalytic domain. Lytic activity by the catalytic domain will be measured in vitro and the domain will be expressed in Spirulina. The full-length sequence is shown as SEQ ID NO: 75, the binding and lytic domains are shown as SEQ ID NO: 76, and the lytic domain only is SEQ ID NO: 77.

FIGS. 12A-12C shows the effect of SP2620 on lysis activity in buffer for three major rumen methanogens, Methanobrevibacter ruminantium (FIG. 12A), Methanobrevibacter gottschalkii (FIG. 12B) and Methanosphaera cuniculi (FIG. 12C).

FIGS. 13A-13B shows the effect of SP2620 on lysis activity in clarified rumen for two rumen methanogens, Methanobrevibacter ruminantium (FIG. 13A) and Methanosphaera cuniculi (FIG. 13B). In both figures, the dark line shows lysis caused by the addition of SP2620 (0.2 mg/mL) and the light line is a control with no added material.

FIG. 14 shows the dose dependent methane content in rumen fluid supplemented with H₂/CO₂ at different dosages of SP2620.

FIG. 15 shows the dose dependent methane content in rumen fluid supplemented with alfalfa/corn at different dosages of SP2620.

FIG. 16 shows an exemplary cloning scheme.

FIG. 17 shows an exemplary manufacturing scheme.

FIG. 18A and FIG. 18B show a prototype Spirulina strain expressing a methanogen lysin lyses Methanobrevibacter archaea.

### DETAILED DESCRIPTION

The disclosure provides compositions and methods comprising methanolysin-expressing Spirulina strains. Also provided are methods utilizing methanolysin generated by the methanolysin-expressing Spirulina strains for reducing methane emissions by mammals. In aspects, a mammal is human. In aspects, a mammal is non-human. In aspects, a mammal is a ruminant.

### Definitions

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences) whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The recitation “polynucleotide” or “nucleic acid” as used herein designates mRNA, RNA, CRNA, rRNA, cDNA, or DNA. The term typically refers to polymeric form of nucleotides of at least 10 bases in length, either ribonucleotides or deoxynucleotides or a modified form of either type of nucleotide. The term includes single and double stranded forms of DNA and RNA.

As used herein, the term “DNA” includes a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally occurring polynucleotide sequences that may be found in a given wild-type cell or organism. A vector, plasmid, or other man-made construct that includes an endogenous polynucleotide sequence combined with polynucleotide sequences of the unmodified vector etc. is, as a whole, an exogenous polynucleotide and may also be referred to as an exogenous polynucleotide including an endogenous polynucleotide sequence. Also, a particular polynucleotide sequence that is isolated from a first organism and transferred to second organism by molecular biological techniques is typically considered an “exogenous” polynucleotide with respect to the second organism.

Polynucleotides may comprise a native sequence (e.g., an endogenous sequence that encodes protein described herein) or may comprise a variant or fragment, or a biological functional equivalent of such a sequence. Polynucleotide variants may contain one or more substitutions, additions, deletions and/or insertions, as further described herein, preferably such that the enzymatic activity of the encoded polypeptide is not substantially diminished relative to the unmodified or reference polypeptide. The effect on the enzymatic activity of the encoded polypeptide may generally be assessed as described herein and known in the art.

As will be understood by those skilled in the art, the polynucleotide sequences of this disclosure can include genomic sequences, extra-genomic, and plasmid-encoded sequences and smaller engineered gene segments that express, or may be adapted to express, proteins, polypeptides, peptides and the like. Such segments may be naturally isolated or modified synthetically by the hand of man.

Polynucleotides may be single-stranded (coding or antisense) or double-stranded, and may be DNA (genomic, cDNA, or synthetic) or RNA molecules. Additional coding or non-coding sequences may, but need not, be present within a polynucleotide of the present invention, and a polynucleotide may, but need not, be linked to other molecules and/or support materials.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T.” is complementary to the sequence “T-C-A.” Complementarity may be “partial,” in which only some of the nucleic acids' bases are matched according to the base pairing rules. Or there may be “complete” or “total” complementarity between the nucleic acids. The degree of complementarity between nucleic acid strands has significant effects on the efficiency and strength of hybridization between nucleic acid strands.

“Polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions. Exemplary nucleotide sequences that encode the proteins and enzymes of the application encompass full-length reference polynucleotides, as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide.

“Transformation” refers to the stable, heritable alteration in a cell resulting from the uptake and incorporation of exogenous nucleotides into the host-cell genome; also, the transfer of an exogenous gene from one organism into the genome of another organism. Exogenous nucleotides may include gene foreign to the target organism or addition of a nucleotide sequence present in the wild-type organism.

“Competent” refers to the ability of a cell to take up extracellular nucleotides from the surrounding environment. A cell may be “naturally competent” or “artificially competent.” Naturally, competent cells are able to take up nucleotides from their surrounding environment under natural conditions. Artificially competent cells are made passively permeable to extracellular nucleotides by exposing the cell to conditions that do not normally occur naturally including incubation in a solution of divalent cations, heat shock, electroporation, and ultrasound.

The terms “wild-type” and “naturally occurring” are used interchangeably to refer to an organism, gene, or gene product that has the characteristics of that organism, gene or gene product (e.g., a polypeptide) when isolated from a naturally occurring source. A wild-type organism, gene, or gene product is that which is most frequently observed in a population and is thus arbitrarily designed the “normal” or “wild-type” form.

As used herein, a “pharmaceutically effective amount” refers to an amount sufficient to ameliorate or prevent a symptom or a sign of a medical disorder. Pharmaceutically effective amount also refers to an amount sufficient to allow or facilitate diagnosis. The effective amount for a particular patient may vary depending on factors such as the disease to be treated, the general health of the patient, the route of method, the dose of administration, and the severity of side effects. The pharmaceutically effective amount may be the maximum dose or administration regimen that avoids significant side effects or toxic effects. The effect will result in an improvement of the diagnostic measure or parameter by at least 5%, such as at least 10%, further such as at least 20%, further such as at least 30%, further such as at least 40%, further such as at least 50%, further such as at least 60%, further such as at least 70%, further such as at least 80%, and even further such as at least 90%, wherein 100% is defined as the diagnostic parameter displayed by a normal subject.

The term “pharmaceutically acceptable” refers to those compounds, materials, compositions and/or dosage forms which are suitable for being in contact with the tissues of human beings and animals without excessive toxicity, irritation, allergic response, or other problems or complications, and are commensurate with a reasonable benefit/risk ratio. For example, a pharmaceutically acceptable substance may be incorporated into a pharmaceutical composition administered to a patient without causing any significant undesirable biological effects or interacting in a deleterious manner with any of the other components of the composition in which it is contained. The pharmaceutically acceptable carrier or excipient preferably meets the requisite toxicological and manufacturing test standards and/or is included in the Inactive Ingredient Guide provided by U.S. Food & Drug Administration (FDA). FDA's Food Additives Status List, formerly called Appendix A of the Investigations Operations Manual (IOM), organizes additives found in many parts of 21 CFR into one alphabetized list. Additives included are those specified in the regulations promulgated under the FD&C Act, under Sections 401 (Food Standards), and 409 (Food Additives). The GRAS Substances (SCOGS) database allows access to opinions and conclusions from 115 SCOGS reports published between 1972-1980 on the safety of over 370 Generally Recognized As Safe (GRAS) food substances.

The term “pharmaceutically acceptable carrier” as used herein includes any and all solvents, dispersion media, coating agents, surfactants, antioxidants, preservatives (e.g., antibacterial agents or antifungal agents), isotonic agents, absorption delaying agents, salts, preservatives, drugs, drug stabilizers, binders, excipients, disintegrants, lubricants, sweeteners, flavoring agents, dyes, and the like, and combinations thereof, as known to those skilled in the art (see, e.g., Remington's Pharmaceutical Sciences, 18^{th} edition, Mack Printing Company, 1990, 1289-1329; and Remington: The Science & Practice of Pharmacy, 23^{rd} Edition, Editor: Adeboye Adejare, Academic Press, Oct. 30, 2020). The use of any conventional carrier in therapeutic or pharmaceutical compositions is contemplated herein unless it is incompatible with the active ingredient(s) of the present disclosure.

As used herein “Spirulina” is synonymous with “Arthrospira.” The genus Arthrospira includes 57 species of which 22 are currently taxonomically accepted. Thus, reference to “Spirulina” or “Arthrospira” without further designation includes reference to any of the following species: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

### Methanolysins

Provided herein are methanolysins and compositions comprising the same. Methanolysins are methanogen-killing lytic enzymes. Methanolysins of the disclosure can be used to reduce or eliminate methanogens. In aspects, a methanogen is a methane-producing single celled organism. In aspects, a methanogen is an archaeon. Methanogens can belong to the domain archaea and be members of the phylum Euryarchaeota. Methanogens are common in wetlands, where they are responsible for marsh gas, and can also occur in the digestive tracts of animals including ruminants and humans, where they are responsible for the methane content of belching and/or flatulence. In aspects, the methanolysins of the disclosure can be used to reduce or eliminate methane emissions in mammals. Exemplary mammals are provided elsewhere herein and can include human and non-human mammals.

In aspects, methanolysins are from phage that infect archaea. In aspects, a methanolysin can be from a phage that has integrated into the archaea genome. In aspects, a methanolysin is from an archaea organism or from a phage (e.g., archaeaphage) that infects an archaea organism.

In aspects, methanolysins are derived from an archaea organism. Archaea is a domain of single-celled organisms that lack cell nuclei and are therefore prokaryotic. In aspects, the major phyla in archaea comprise Crenarchaeota, Euryarchaeota, Korarchaeota, Nanoarchaeota, and Thaumarchaeota. In aspects, the Euryarchaeota phylum comprises Methanobrevibacterium. In aspects, a methanogen is Methanobrevibacterium.

Pseudomurein, the major component of the methanogen cell wall, is comprised of N-acetyl-L-talosaminuronic acid and N-acetyl-D-glucosamine connected by a β (1-3) glycosidic bond. Methanolysins can target these unique structures and have been shown to inhibit >90% of cell growth and >60% of methane production in several rumen methanogens. These lytic enzymes are derived from viruses that are the natural predators to methanogens and have evolved over millions of years to degrade the unique conserved structure of the methanogen cell wall. The first two methanolysins discovered, PeiP and PeiW, are endoisopeptidases from thermophilic archaea that act by hydrolyzing the ε-(alanine)-lysine bond of the peptide linker between adjacent pseudomurein layers. Another methanolysin, PeiR, from ruminal methanogens cleaves a threonine-glutamate bond that is also part of this peptide linker. In aspects provided are engineered methanolysins for expression in Spirulina with improved potency of the molecules. In aspects, efficacy can be improved by combining multiple Spirulina-methanolysin strains into a cocktail to target multiple cell wall bonds and/or multiple methanogen strains to prevent treatment escape.

Methanolysins target bonds that are specific to the unique cell wall of archaea and leave the commensal bacteria and host cells unperturbed. In aspects, Spirulina-methanolysin dried Spirulina product are administered orally as a feed additive and have reduced or no effects on the meat or milk products obtained from fed ruminants. Due to the small size of the protein, any methanolysin that enters systemic circulation can be quickly cleared via excretion by the kidneys.

Any methanolysin can be employed in the compositions and methods of the disclosure. Exemplary methanolysins comprise PeiP, PeiW and PeiR. Both PeiP and PeiW are capable of hydrolyzing the ε-(alanine)-lysine bond of the linker between layers of pseudomurein. Moreover, both enzymes contain four N-terminal pseudomurein binding repeat (PMBR) domains essential for cell wall binding and a C-terminal catalytic domain. PeiR cleaves a threonine-glutamate bond that is part of a peptide linker. Moreover, PeiR possesses an N-terminal cell wall binding domain, referred to as the pseudomurein binding domain, and a C-terminal catalytic domain that cleaves peptide bonds that are essential to the structural integrity of the archaeal cell wall.

Archaea can be found in the intestinal tract of mammals. In aspects, archaea in the intestinal tract of mammals is referred to as enteric archaea. In aspects, the enteric archaea can be found in the intestinal tract of humans. In aspects, the enteric archaea can be found in the intestinal tract of ruminants.

In aspects, the ruminants comprise cattle. In aspects, enteric archaea comprise methanogens. Enteric archaea produce methane emissions as a metabolic byproduct. In aspects, the methanogens are anaerobic. In aspects, the methanogens comprise Methanobrevibacter smithii, Methanobrevibacter oralis, Methanobrevibacter massiliense, Methanosphaera stadtmanae, Methanobrevibacter arboriphilus, Methanobrevibacter millerae, Methanomassiliicoccus luminyensis, Methanobrevibacter ruminantium, Methanobrevibacter gottschalkii and Methanosphaera cuniculi. In aspects, the methanogens comprise Methanobrevibacter ruminantium, Methanobrevibacter gottschalkii and Methanosphaera cuniculi. In aspects, the archaea methanogen is Methanobrevibacter ruminantium M1.

In aspects, methanolysins are derived from archaea. In aspects, archaea comprise Methanobrevibacterium. In aspects, the Methanobrevibacterium comprise species Methanothermobacter marburgensis, Methanothermobacter wolfeii, and Methanobrevibacter ruminantium M1. In aspects, PeiP is derived from Methanothermobacter marburgensis, PeiW is derived from Methanothermobacter wolfeii and PeiR is derived from Methanobrevibacter ruminantium M1. In aspects, the Methanobrevibacter ruminantium methanolysin is PeiR.

In aspects, a methanolysin comprises an amino acid sequence with at least about 80%, 85%, 90%, 95%, 98%, or 99% identity to SEQ ID NO: 78. In aspects, wherein the PeiR-MC methanolysin comprises SEQ ID NO: 78.

In aspects, the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.]. TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.]. In aspects, a methanogen is from Methanobrevibacter.

In aspects, the methanolysin comprises an amino acid sequence with at least about 80%, 85%, 90%, 95%, 98%, or 99% identity to a sequence in Table 1. In aspects, the methanolysin comprises a sequence in Table 1.

In aspects, methanolysins can be resourced from organisms of the genus Spirulina. In aspects, the genus Arthrospira comprises species A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspects, methanolysins can be derived from Spirulina. Spirulina is one of the photoautotrophic, planktonic, filamentous green-blue algae. In addition, Spirulina is a filamentous, gram-negative cyanobacterium. In aspects, Spirulina can express methanolysins. In aspects, Spirulina can be genetically modified. In aspects, genetically modified Spirulina can express methanolysins.

In an aspect, a modified Spirulina cell, comprises an exogenous sequence encoding a methanolysin. In aspects, the exogenous sequence encoding the methanolysin is genomically integrated into the Spirulina cell genome. In aspects, the genomic integration is within a neutral genomic region. In aspects, the neutral genomic region is selected from the group consisting of: kanamycin aminoglycoside acetyltransferase, NS1, and both kanamycin aminoglycoside acetyltransferase and NS1. In aspects, the neutral genomic region is the kanamycin aminoglycoside acetyltransferase.

In aspects, methanolysins can reduce or eliminate the generation of methane resulting from enteric fermentation in ruminants. In aspects, methanolysins are capable of inhibiting methane emissions produced by enteric archaea. In aspects, genetically modified Spirulina strains can express methanolysins capable of inhibiting methane emissions by enteric archaea.

Exemplary sequences of methanolysins are shown in Table 1. In aspects, a methanolysin comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 70 and SEQ ID NO: 78-SEQ ID NO: 100 shown in Table 1.

In aspects, the methanolysin comprises a sequence in Table 1. In aspects, the methanolysin comprises SEQ ID NO: 1. In aspects, the methanolysin comprises SEQ ID NO: 2. In aspects, the methanolysin comprises SEQ ID NO: 3. In aspects, the methanolysin comprise SEQ ID NO: 4. In aspects, the methanolysin comprises SEQ ID NO: 5. In aspects, the methanolysin comprises SEQ ID NO: 6. In aspects, the methanolysin comprises SEQ ID NO: 7. In aspects, the methanolysin comprises SEQ ID NO: 8. In aspects, the methanolysin comprises SEQ ID NO: 9. In aspects, the methanolysin comprises SEQ ID NO: 10. In aspects, the methanolysin comprises SEQ ID NO: 11. In aspects, the methanolysin comprises SEQ ID NO: 12. In aspects, the methanolysin comprises SEQ ID NO: 13. In aspects, the methanolysin comprise SEQ ID NO: 14. In aspects, the methanolysin comprises SEQ ID NO: 15. In aspects, the methanolysin comprises SEQ ID NO: 16. In aspects, the methanolysin comprises SEQ ID NO: 17. In aspects, the methanolysin comprises SEQ ID NO: 18. In aspects, the methanolysin comprises SEQ ID NO: 19. In aspects, the methanolysin comprises SEQ ID NO: 20. In aspects, the methanolysin comprises SEQ ID NO: 21. In aspects, the methanolysin comprises SEQ ID NO: 22. In aspects, the methanolysin comprises SEQ ID NO: 23. In aspects, the methanolysin comprise SEQ ID NO: 24. In aspects, the methanolysin comprises SEQ ID NO: 25. In aspects, the methanolysin comprises SEQ ID NO: 26. In aspects, the methanolysin comprises SEQ ID NO: 27. In aspects, the methanolysin comprises SEQ ID NO: 28. In aspects, the methanolysin comprises SEQ ID NO: 29. In aspects, the methanolysin comprises SEQ ID NO: 30. In aspects, the methanolysin comprises SEQ ID NO: 31. In aspects, the methanolysin comprises SEQ ID NO: 32. In aspects, the methanolysin comprises SEQ ID NO: 33. In aspects, the methanolysin comprise SEQ ID NO: 34. In aspects, the methanolysin comprises SEQ ID NO: 35. In aspects, the methanolysin comprises SEQ ID NO: 36. In aspects, the methanolysin comprises SEQ ID NO: 37. In aspects, the methanolysin comprises SEQ ID NO: 38. In aspects, the methanolysin comprises SEQ ID NO: 39. In aspects, the methanolysin comprises SEQ ID NO: 40. In aspects, the methanolysin comprises SEQ ID NO: 41. In aspects, the methanolysin comprises SEQ ID NO: 42. In aspects, the methanolysin comprises SEQ ID NO: 43. In aspects, the methanolysin comprise SEQ ID NO: 44. In aspects, the methanolysin comprises SEQ ID NO: 45. In aspects, the methanolysin comprises SEQ ID NO: 46. In aspects, the methanolysin comprises SEQ ID NO: 47. In aspects, the methanolysin comprises SEQ ID NO: 48. In aspects, the methanolysin comprises SEQ ID NO: 49. In aspects, the methanolysin comprises SEQ ID NO: 50. In aspects, the methanolysin comprises SEQ ID NO: 51. In aspects, the methanolysin comprises SEQ ID NO: 52. In aspects, the methanolysin comprises SEQ ID NO: 53. In aspects, the methanolysin comprise SEQ ID NO: 54. In aspects, the methanolysin comprises SEQ ID NO: 55. In aspects, the methanolysin comprises SEQ ID NO: 56. In aspects, the methanolysin comprises SEQ ID NO: 57. In aspects, the methanolysin comprises SEQ ID NO: 58. In aspects, the methanolysin comprises SEQ ID NO: 59. In aspects, the methanolysin comprises SEQ ID NO: 60. In aspects, the methanolysin comprises SEQ ID NO: 61. In aspects, the methanolysin comprises SEQ ID NO: 62. In aspects, the methanolysin comprises SEQ ID NO: 63. In aspects, the methanolysin comprise SEQ ID NO: 64. In aspects, the methanolysin comprises SEQ ID NO: 65. In aspects, the methanolysin comprises SEQ ID NO: 66. In aspects, the methanolysin comprises SEQ ID NO: 67. In aspects, the methanolysin comprises SEQ ID NO: 68. In aspects, the methanolysin comprises SEQ ID NO: 69. In aspects, the methanolysin comprises SEQ ID NO: 70. In aspects, the methanolysin comprises SEQ ID NO: 78. In aspects, the methanolysin comprises SEQ ID NO: 79. In aspects, the methanolysin comprises SEQ ID NO: 80. In aspects, the methanolysin comprises SEQ ID NO: 81. In aspects, the methanolysin comprises SEQ ID NO: 82. In aspects, the methanolysin comprises SEQ ID NO: 83. In aspects, the methanolysin comprise SEQ ID NO: 84. In aspects, the methanolysin comprises SEQ ID NO: 85. In aspects, the methanolysin comprises SEQ ID NO: 86. In aspects, the methanolysin comprises SEQ ID NO: 87. In aspects, the methanolysin comprises SEQ ID NO: 88. In aspects, the methanolysin comprises SEQ ID NO: 89. In aspects, the methanolysin comprises SEQ ID NO: 90. In aspects, the methanolysin comprises SEQ ID NO: 91. In aspects, the methanolysin comprises SEQ ID NO: 92. In aspects, the methanolysin comprises SEQ ID NO: 93. In aspects, the methanolysin comprise SEQ ID NO: 94. In aspects, the methanolysin comprises SEQ ID NO: 95. In aspects, the methanolysin comprises SEQ ID NO: 96. In aspects, the methanolysin comprises SEQ ID NO: 97. In aspects, the methanolysin comprises SEQ ID NO: 98. In aspects, the methanolysin comprises SEQ ID NO: 99. In aspects, the methanolysin comprises SEQ ID NO: 100.

In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 1-3, 1-5, 1-10, 5-20, 1-3, 1-5, 1-10, 1-20, 3-8, 3-10, 3-15, 5-8, 5-10, or 5-20 residues. In aspects, a modification can occur in up to 1, 2, 3, 4, 5, 6, 7, 8, 9, 10, 11, 12, 13, 14, 15, 20, 30, 40, 50, 60, 70, 80, 90, 100, 200, 300, 400, or 450 residues.

In aspects, provided are also modified methanolysins comprising increased killing activity and/or accumulation in a Spirulina host cell as compared to an otherwise comparable unmodified methanolysin.

In aspects, provided are also novel methanolysins identified from public sequence databases that include archaea genomic DNA.

In aspects, methanolysins may be expressed as an inactive proprotein to increase accumulation in Spirulina. Methanolysins can be designed for activation during manufacturing or in the rumen.

TABLE 1
Exemplary Methanolysins.

| SEQ ID | |
|---|---|
| NO: | Protein Sequences |
| 1 | >SEK59543.1 |
| | MVRFSKKEMQKASKIIRESIKKGKGQPLSVKMVEMDKGKTIKVSKAYYMGLF |
| | EAQNIFIRNKGRKPNYVTLNSKANNPLVMDYQDNAYTCCPTSFSMATQLLFD |
| | NISEKECAKALNTVLGSGTDPSDLIKNSGKLGYKVTPIKRDYKSVKSSLDKGF |
| | AVIAHIQTRPATCLGYSGDYGHYVLLYGAYRKLLTKYYKVADPTKGLKTCK |
| | ASILDKATNGRNIKYYSVGIL |
| 2 | >WP_042693405.1 |
| | MIKFMNDVIVKASKTIRKNIKEGKGVPTMLVMMDSNGNSHKLSKKEVAGIFE |
| | ARNVFILKKGRLPNYVTLNSTANNPLVMDYQNNKYNCCPTSLSMAIQMLYSY |
| | KSENVCAKALKTNTAGTAPLDLVSGAKNLGYEVKRISRNKASVKAQLNMGK |
| | SVIAHIQTKSATCLGYVGDYGHYVLIYGMSSDNYKIADPTKGIKTCRTGILDK |
| | ATNGREIHYYSVSPL |
| 3 | >MBR1610527.1 |
| | MTRFSKNEMNKASKTIVKSIRNGKGLPRIVEMKEMDHNKTIRVDKSHYNGLF |
| | EAQNVFIRGHGRLPNYVTMNSTANNPLVMDYQDNGYTCGPTSLSMAIQMLY |
| | GWKSEKRCAEACHTGSSGTSPDDLIAGAKSLGYKVTPIKRNFAGVKQSLNKG |
| | YPVIAHIETGGRTKPKCLGYVNNYGHYILIYGTTASGGYKVADPTKGLRVCSA |
| | SELDKATNGRSINYYSVKPL |
| 4 | >MBO7043925.1 |
| | MKDAMTDGAKRVFKFIKEHERYPKTLRMVDSTNKSHVLTKAQYMGLFEAEN |
| | IFRIKHGRQPNYTTYNSTASNPLVLDYQDGKMTCCPTSLSMCSQLLFHFKSEN |
| | QCKKALKTDPKEGTSPDKLVNNAKDLGFKVVVIGRDYKKVVKALDCGYGVI |
| | MHIETAKKTRPKCLGYTNNYGHYVCCYKAKNNKYYIADPTKGMKTCKASEI |
| | DKAKNDRDLKYYIIKPL |
| 5 | >MBQ2832900.1 |
| | MTRFLNSEMVKASKTIRNNIKNGNGLPRSIKMIDTDGKVHTVEKKYYNGLFE |
| | SRNVFTLKNGRMPNYVTLNQTANNPLVLNYQDDKYSCCVASFNMCVQMLY |
| | DWIPESKIKKLFKTNQYGTSPANMVAGAKALGYKVEQIGRNFSSVQKAISKGF |
| | PVLTHYETAGKTKPKCVGFIQNYGHYGMIYGVTKDGYYLFADPTKGLKKCTP |
| | KELDYATNGRSLGYYMVGLV |
| 6 | >MBQ6345181.1 |
| | MIRINKKEYTDARKRIRSFIKKHKRLPNYCQFMTNQKKTVDLNKEEYCGLFEG |
| | YMHFCLKNGREPNYLTLTSKANNPIVINYQDDKVSCCPASFLMGMMFLFEYK |
| | SEATVKKALGTTSNGTSPTQLINNAGKLGYVVKRIPREFREVKKALEQYKPVL |
| | MHIQTKPATCLGFVSDYGHWIICWKVKDNKYYVIDPTKGSKVCNYQILNRAT |
| | NGRNIGYYTMEIK |
| 7 | >MBP5421670.1 |
| | MVRFNESEIKKAIKTIRASLKANKGLPSTVTMKDTDGKSRKLSKAQYNGLLE |
| | ARNVFRLKNGRMPNYTTLNSTANNPIVLNYQDDSVSCACASFNMCVQMLYE |
| | WIDEKKIKQVFQTGSSGTAPSQVISGAKKLGYNVTKIDRNSKAVKKALDKGY |
| | PIMAHIDTIKAPCLGYAKNYGHWIMIHRINDTSYYVLDPTKGSKVCKFANIDK |
| | AMLDRTIYYYKVTIV |
| 8 | >MBQ8017191.1 |
| | MKYSKTEMKTAAKTIRKNIKKGNGRPEKIKMTDTNGDKHKLKLKQYLGLFK |
| | QWNIFRLKNGRYPNYVTYVGKSKEPTVINYQDNGYQCACASFNMALQSLFD |
| | WEDETTIAQAFNTGKYGTSPSDMINGAKKFGYKIQPINRNQKALQKAFDKGY |
| | GVIAHIDTIKAPCLGYKNNYGHYINIQRITKAGNYRVFDPTKGVLSVKPSCIDN |
| | AMLNRTLNYYQVVIE |
| 9 | >MCL2114967.1 |
| | MDCTKVNLKKGSKGPLVGELQKYLRDHGFYKREIDNDFGSFTEDAVKLLQR |
| | KQGNDPDGVFGPKTCSKSGLNTYTQEINSSKTIASAIDNKTTVNDDSESSKNSS |
| | SDVKGGEISPPEYNMGKQTDGYYYCSCGEKGYEYGRWKVTFVDRCAHADD |
| | ASHKYKSSKLKWSDKKDVAPLEGQWTCEGCDADYCAVTGKEKSSTSKKRLT |
| | VIKTEKMPIKKDVKEPTNVNNPPKDKDIVKTYSKEELANNAEYIINETKKNGK |
| | NPDSIQIKNRENNNLDLLNKPQYMGLYENQNIFWVNNKRMPNWTTLATTAN |
| | NPVKMNYQSNAYNCGPMSMSMASQMLFKNTFESEFVKVCE |
| | TNKNGTDPKNLSLISKLGFSIKPIDRNSKAVKEALARGSAVIAHIETKPASCLG |
| | YQNNYGHYVLIDRINNSQYHVMDPTKGIKWCDFTTLDNATNGRNISYYEMK |
| | VA |
| 10 | >WP_095608514.1 |
| | MLNCKTINLKKGSKGAEVKELQTILQNKGYYDGKIDGDYGDITVQAVKLLQR |
| | KQGNDPDGNFGPKTCSKLDQKTGTTPTGNINASNQKFLKDEIIRAGKVFRQHI |
| | KNNNNYPNYIEMKNSSGKTYQVSKALYMGLFEGMNMFAIKNGRLPNFVVAS |
| | STANNPLCIDYQNNGVNCGPTSLSMCIQMYGEYISEPILARACDTGSNGTGPSE |
| | LKMGAKKYGYKLVEIPRNINAVKESLAKCRPVIMHIHTSYSGGRSCLGYRGA |
| | YGHYIMCYGTNGNYYKLADPTKGFKTCSSTSIDNARSGEFMKYYSVCIV |
| 11 | >MBQ9026945.1 |
| | MSSFSKSEVLRGAKIFRNSIVKGKGVPKEVTLKVRETGKNKTLTRKQFAGIME |
| | ATNVFRLKQGRYPNYTTLNSTANNPLVMNYQDDSVSCGCASFNMCVQLTYG |
| | EWIDEKAIKKTFGTGSSGTAPSQIIAGAKKYGYAVEKIARNSSAVKKALEKGY |
| | AILAHIDTKPATCLGYQNNYGHWTVIYGLDDGKYKIADPTKGIKTCSTKIYDK |
| | AMLGRTIYYYQVRPL |
| 12 | >WP_013826120.1 |
| | MSIRINKAQLLDGANGISNFIKANKRFPNYATLTDSNNKQQKVLKANYLDFY |
| | KRAFQWAVNHGDIFPNYGTVIGTGTSPIPQNYQDSSTTCGPTSLSMGSCGLFK |
| | YKSEAQFKAACNTTSSGTTPENLIAGAAKLGFKLTKISRNIAGVKAALNQCKP |
| | VIAHIQTKNATCLGYKGDYGHYVLIKGLSGDDHYLINDPTKGENITCLSTILDN |
| | ATDGREIYYYSMELA |
| 13 | >MCR5027398.1 |
| | MGFLDWIKYLFINNKKMARFITKNITKTFGEKEQLEVALFEGENPLSDRNVNI |
| | SINGMTYVRKTDENGIARLNINLGVGEYKADFEFMDDDFNLANAFCKVFVNP |
| | VISTQDMNMTEKDGSQFIAIATSSTGVRLSGVPIIFNINGVDYKRTSDMVGEAK |
| | LNINLGKGTYEITTSSLKVSAKNIIHIDEKPKKVTRMEGVNISKAFSDKTAYQC |
| | AVYDDNSRVSGTVVISVNGVDYTKTPDSTGLYKLNINLQPGTYQISAQYMGD |
| | NEHLPSQIVNTIHIVADPVKTVYNPIRIKYQPNSYTCGPTSLSMCSQILGNEVSI |
| | NSFANACYTDGSGTSPSNLIAGARKLGFKVTPIGRSYAAVKKAIDAGKPVIAH |
| | VMTKTMKCLGWQGNYGHYVVIYDYDNGYYHVADPTKGISWCYKDQVDGA |
| | TIGYEGIQYYSVERI |
| 14 | >MBR0271156.1 |
| | MNFIDFIKNIFSNNKRNARIITKDLNKIFGEKDALEIALYENNSPLVDKNLNIKI |
| | NGVTYSRKTDNDGIARLNVNLGVGLYEALISFIDEDFNYTSAYCKVTVNPVLS |
| | TQDMRMQEHDGSQFIAIASSSTGVRLSGVPVIFNINGVNYERTTDMVGEAKLN |
| | INLGKGDYNISTSSFNTVKKNTIHIDEKPKKVTRMEGTNITKAFSDKTAYQCA |
| | VYDDNGRVAGSVDITINGVTYTRTPDESGLYKLNINLQPGTYQLTAKYRGDD |
| | THLASQIINTIHVVADPVKKEIVYNPIKIQYQPNSYTCGPTSLAMCSQILGKSTSI |
| | SAFANACYTTGNGTSPANLIAGAKKLGFKVTEIPRNINGVKAAIDAGKPVLGH |
| | IMTGGVTCGGWQGNFGHYIVIYDYGTVYYYVADPTKGFYRCTHSMIDNAMR |
| | GYAGIKYYSVERI |
| 15 | >WP_095608516.1 |
| | MLDCKTINLKKGSTGTKVKELQIILQKKGYYDGVIDAIYGDLTVQAVKLLQRE |
| | QGNSPDGWFGTKTCSKINQNTTLETPQGNTNASGQKFMKDEIIKAGKVFRQHI |
| | KNNNNYPNYLEMKNSNGKTYQVSRALYMGIFEGMNMFSIKKGRLPNYVVA |
| | NSTANNPLCIDYQNNGYNCGPTSLSMCIQMYGEYISESTLANVCGTTRNGTSP |
| | ASLNSGAKKIWIQIIRNST |
| 16 | >MCL4403918.1 |
| | MLKLKPFEQTSGLCGPASLKIVLEYYGVRKSEAALAKLSGASRGSGVEASGL |
| | VRAAEKLGFAAIVKDRATVDDIRRHLKRGIPVIVDWFSKDDGHYSVVAGIEN |
| | GRIHIQDPEIGRLRSLDLETFQRVWFDFPGDVLRSPKDVICQRMIVIRPRK |
| 17 | >MSR71654.1 |
| | MSKVILDVKPFQETLHADMCGPASLKIILHYYGIEKSETELAKLTHLVPGLGID |
| | DTSIVDAARTLGFKAEIKNESDFSDIEKWLKRGVPLIIDWFTRGRNDYSDNEV |
| | ADGHYSVVYGIDDIYVYIQDPETGSVRKIGKEDFMKVWFDFLGKVIKPNELVI |
| | RQIIAIYK |
| 18 | >OGZ62880.1 |
| | MKKIFLRVKPFQETLSGYCGPASLLILLSYYGVNKTERELGRLCKTNKNLGTT |
| | AKGIKEAAEKLGFKVKIKINSNFKDIERWLKRGVPVIVDWFTRGRSDYNDSEI |
| | TDGHYSVLVGLDSKFIYLQDPEIGRIRKIKKEDFMVVWFDFKGKHIKPNELVIR |
| | QIIAIYK |
| 19 | >OGZ64231.1 |
| | MQKILLKVKPFQETLHADMCGPASLKIVLDYYGIEKSEKELAKLCKIKKGLGV |
| | DDKGIKKVAEKLGFRVEIKNNSSFKDIEKWLKKGVPVIVDWFTRGRNDYPES |
| | ETADGHYSVVCGLDDKFIYLQDPEIGKIRKIKRDDFMKVWFDFSGKYIKAEEL |
| | IVRQIIAIYK |
| 20 | >MBU2081944.1 |
| | MKNKIIFVPKNIMGVYEWQDRNYRIDRVKNIKDFRARKVKFFGTTIGKKFLG |
| | YKIKKFINLNGIDVWFVNGDKLRSGVKSGDIDFTMGGHTYRYLYVPEGEIWIE |
| | SVYQRTKELMSIVWHEYLEQSLMCFGMSYLRAHSLASIFEMTLREGKYTMLP |
| | IGTYRQKTKFTCGPAALKIVLEYFRRPISEKYLAKLSKTTKIKGTNPQDLVEGA |
| | KKLGFSAYQKEYITPNEVKNLIKKGIPIIANYQYEPRFGEGHYAVIIGFSKDNFI |
| | LSDPGAMEGYQMVSIKEFMKNWFELEDKTHRQGVIISGNKKL |
| 21 | >KKU65937.1 |
| | MLNVKPFQETLHHGLCGPASLKIVLDYYGLVKSEQELAELTGTTSGMGTDDK |
| | SIARVAESLGFKTVIKNESDFSDIKEWLKKGVPVIVDWFTRGREDYPPSEMAD |
| | GHYSAVVGLDDNLIYLQDPETGGLREIARDDFKKVWFDFTGEYIRNDELIIRQ |
| | LIAIYR |
| 22 | >PIP24522.1 |
| | MWLALWPVPMLWLLLGAKINFKNLFMIKLKPFRETPGLCGPASLKIVMDYY |
| | GVSVSEADIAKIAGATQEKGVSVAGLIKAARHFGFKVLVKENSSLDDLSYYV |
| | KKEIPVIVDWFSEDDGHFSVVADIDKNNVILMDPAIRKFLIYIRRRKFPRETFLR |
| | IWFDFPGDYIKTSKDLVLRLMIAVLPKYVGKL |
| 23 | >MCR4334590.1 |
| | MKIPYRSQNNHFYCAPASLQMLFEFFGVYKTQNDIAKKLNTNPQKGTEDKSII |
| | DFVTKQGFFCYVNQDSTLEEIDDFIKKGHPVIVNFIEPSQDEGHYALVVGMDE |
| | EEIILNDPWNGKGFKMTKKDFKKRWHNSDNRSKRWMMVISRENFNLGKQFL |
| | AKEKL |
| 24 | >KKR85642.1 |
| | MLNIKPYKQTRGMCGPASLKMVLEYFGMEKSEEELGQLTHCNPDTGTKAEDI |
| | VLAAKQLGFNALIKDMADFDDIKKYLDQEIPVIVDWFSENDGHYSVAVNMD |
| | KENIYLQDPEIGSIRELPLETFKRVWFDFPDKFINSKDDLILRRIIVIQK |
| 25 | >MBS3074143.1 |
| | MKKISLIDVPYDHQDTDYTCGPSSLKMVLSFFGVRESELKLAHEAHTSRKKGT |
| | AHAELIKLARRHGFYCYIRRDSNINTIRHFIRLGLPVIINYRNIPDNESHYAVVV |
| | GYGRGRIILNDPWNGRNFKIDDKTFIRHWIGENNTKKWVFVISKEPVIRGKQY |
| | NPMKR |
| 26 | >OGZ63401.1 |
| | MEKILLKVKPFQETLQADMCGPASLKIVLDYYGIEKLEKELAKLCKIKKGLG |
| | WNDKDIKKVAEKLGFKVEIKNNSSFKDIEKWLKKGAPVIVDWFTRGRNDYPE |
| | SETADGHYSVVCGLDDKFIYLQDPEIGKIRKIKRDDFMRVWFDFSGEYIKVEE |
| | LIVRQIIAIYK |
| 27 | >WP_095608517.1 |
| | MVKYDTSKPIHLAMDYTRCRNCDRDRSMLNQIENSLKSKGFNVVGQRYSPN |
| | GLALLTKDDFAKVKGHVGISLVNGADPASLYEVAQIKDGNYLRSPSGSARSG |
| | SFKYAEQNLAPRNNIVCMAYWYGSCDPVNPGGKCEKRIRGSETTGPIYNPREF |
| | IGKHHIPAFAKSDSKDPLVIADNIAKMFTVTKKKDSNGKNLSLMDVIVQFGWS |
| | EGKANTIEHELEFYGVAGESDINNNNNNNTNTVTISPDDTVSQIIIEETYTKAYF |
| | QQIQPLKTDPNGSFKQSLLLPYNGKYTVNIHYGGSRYHAPTTASYNIDNKGAV |
| | DFTPTLIEQCITTTTKEGKTTIQKEGDASKVKHTYTVRTTITYENGAEKSRSVE |
| | TIQNDTRVKGATTTTNTNTNTNTNTNYSITGKKDPFSVDIPVITNSDGTVEPDV |
| | AHMGNSTYRYKKYSPSETVTLLKEHYTEVMHRDSVSMQLNNYKESKYTAFR |
| | TKEEPNVFRVISRTRWNAVEECILRYLVHGQGIAWPDEITINFGEQKTIINGTTV |
| | NFKSKDVVNISVKDKQNTGYTCGPTSCSACTQVLHNYYSEQYLQRLIGATSG |
| | SGSAPSAHVRALNKLNFKAELYSSRDTLISHLRSGRPAVIHVWNHYICAASISP |
| | DESKILMINSTESGNYSPTTGWADTSTVEKRRGGRSAVRVSLNWTITDTDKAK |
| | FNHFYNSMGGAWNKPYVHEKICNANSGVNVFL |
| 28 | >PIV45327.1 |
| | KPFRETPGLCGPASLKIVMDYYGVSVSEADIAKIAGATQEKGVSVAGLIKAAR |
| | HFGFKVLVKENSSLDDLSYYVKKEIPVIVDWFSEDDGHFSVVADIDKNNVILM |
| | DPAIRKFLIYIRRRKFPRETFLRIWFDFPGDYIKTSKNLVLRLMIAVLPKYAGKL |
| 29 | >OGC38145.1 |
| | MAKLYLPPDLAGLYERQENRRIERVDSLKRHHEKSSRFRRARNFSRRYRVQKI |
| | ARLSGVNVWLVDGTGIRRDVDVDFTMGGHAYRYLYIPPNEIWVESSLAEGDL |
| | WPTIWHEFTERRMMRAGLNYDRAHTYASRLEIVLRDGRTFVLPVGTFRQTEG |
| | LCGPAALKIVAEYHGYNFSEPHLARLCHTTKEKGTDPADIIRAARKLRFTAWQ |
| | REHFTVEMVKETLKRGLPIIANFQLTPDFGEGHYAVIIGFTAKEFILSDPQEDA |
| | GYRKVPIKKFMGLWYELEDKTAAQGIIVSVP |
| 30 | >TSC83912.1 |
| | MLRVLPFRQSSGFCGPASLKMVLEYFGIKKSEKELAKLSNAHPHHGTSAKNL |
| | VLTAKKLGLKGFYKDFSSIKEIKKYLDKKIPVIVDWFSHDDGHYSVVIGIDKKF |
| | IYLQDPELGKINKIDLVTFQRIWFDFEEKMLNKKQDIFIRRLIVIYR |
| 31 | >KKS79316.1 |
| | MCGPASLKMVLEYFGMEKSEEELGQLTHCNPDTGTKAEDIVLAAKQLGFNA |
| | LIKDMADFDDIKKYLDQEIPVIVDWFSENDGHYSVAVNMDKENIYLQDPEIGS |
| | IRELPLETFKRVWFDFPDKFINSKDDLILRRIIVIQK |
| 32 | >MAG26693.1 |
| | MFLLKISGDKMKIKLTKQSEGYCGPASLKMVLSIYGINKSENELAKLTKTSRK |
| | KGCDEKDIVKVAKKLGL |
| | KGYVKKNSSIFEIKKLVNKGIPVIVDWFSPEEAGHYSVVVGFEKDKILIADPHF |
| | GKVKKYRIDWFEERWF |
| | DMPFKKIIKKEIIVITK |
| 33 | >MBI4138842.1 |
| | MAITLNPFRQSPGYCGPASLKILLEYYGKSFSEDELARICGSTQEKGTDHAELV |
| | AALETLGEKPVAKERATIEDVRSYIRKGTPVIVGWWSTDEDHYSVVYDMRDE |
| | TISLMDPEVEGGSVVMPITDFEKVWHDFDGAQNIRVERWMLAIERPAPERP |
| 34 | >MBW2996858.1 |
| | MLKLKPYKQSRGYCGPACLKMVLGYYGVVKSERTLAKMTKTSRTKGCEEK |
| | NIVAAAKKLGFKAYVKQKSSIAELRRLVRKGIPVIVDWFSPEEAGHYSVVVGF |
| | EKDNIILADPHFGRIKKHKIDWFSDRWFDRPFKKTLIREIIVIQG |
| 35 | >MBI2278604.1 |
| | MTRKGVKLLNVKPFRETSGFCGPASLKMVLAYYGVRKSERALARLAGATRS |
| | QGVEAPGLVKAARRLGFIARIKDRATLADIKKYIRKCIPVIVDWFSNDDGHYS |
| | VVVGITKSTIYLQDPEIGRVRAMDTKTFKRVWFDFPGDILRSKNAVVLQRML |
| | VVRRSGKN |
| 36 | >VVB80060.1 |
| | MKLKIPFFKQRTGFYCGPASLEMVLAFFKKYKTQKELAKEARTKKDGTTHEE |
| | MIKVARREGFYCYVHNNSTIEEIKHFIKQGLPVIINYKEPSSEEGHYAVISGYK |
| | KSRLILNDPWNGNNFKIKEKEFLRRWHDYHKKHEYKNWILVLSRKNLNLGKL |
| | FLPTK |
| 37 | >PIR88484.1 |
| | MLPVKPFRQRTGYCGPASLKMVLDYFGIRKTEKELAVLSGATKKQGVQDGE |
| | GLVKAARSLGLKAALKDKATLADIRKYIKQEIPVIVNWFSVNEGHYSVVVDID |
| | KENLYIKDPEWGYTRAMRLDTFMEVWFSIRGNVPSSKEDFNIRRLIAVEPHKP |
| | RRGRAKKK |
| 38 | >QGH71204.1 |
| | MDIKLKEFKDMIRRVKHFLSTKKRPPRYVNTSMGRITYNQYIDALKRYTDFV |
| | KKNKREPKYVRIYSEAKPTHKPGGPNQELFEKTFNVQIESAQDLYAWLRDNV |
| | KYSYYYNDKYDNREVIEKIKRGEGVNCTDLAQFCRPILRDLGYECDFIRAEVK |
| | CSNNKWYGHVFLRIRGKEYNDWVYFDGTAVTHENFDRPLGKLICTGGTREIT |
| | INPKWLLNAEQNL |
| 39 | >PKL66861.1 |
| | MIISYKEYQDMSVRIMQFQMERGRKPKYVTLNGSKIGQRQYEDMMRRVDLFI |
| | KSKGRNPKSVRLDEYIEIVKPSLGRKKSASWIKLEEALDKKFNDAKDLYKAIA |
| | DQGEYKYYYNDKFDNKMALTRLRKGLGINCTDYAQLIRPVLEDMGYDVRY |
| | AHGRVKCGDKQWYGHVWLQIKGRDYGNWVNYDVVAVTHHGIKRPIGSLVC |
| | VNGVKDVEYNPKWLI |
| 40 | >MBI4812974.1 |
| | MIISYKQYQDMSIRIMQFQMERGRKPKYVTLNGTKIGQKQYEDMMKRVDVFI |
| | KAKGRNPKSVRLDEDLEIIKPSLGKKKSASWIKLEEALKNKFNNAEELYNALS |
| | DQGQYKYYYNDKFDNKMALTRLRKGLGINCTDYSQLLRPVLEDMGYDVRY |
| | AHGRVKCGDKQWYGHVWLQIKGRAYDNWINYDVVAVTHHGIKRPIGSLVC |
| | VAGVKDVEYDPNWLAQ |
| 41 | >WP_069583118.1 |
| | MTEKIKRDNYQDVIERTLLYVFKNGKLPSYASINGKKILKKDIQDALTRSNNY |
| | FKKNGKCAGNVNMVLQDSTPVSTNPIKTELLKTIEKAVNGTFKTATQFYNLV |
| | KANEKYDHYSNDIYPQGKALTRLINNQGLNCADFAQIGHASIFELNKVYRTK |
| | YQVDYVHVACKSSSGQYNIGHIVLRVKGEEFKDWTVFDVAEAASGGLPIGRT |
| | MCSLGYKVLSYNDPWLLSDDGKT |
| 42 | >WP_158005554.1 |
| | MDGLDQKHAKKMNQENTSTSKDEVIKTIEKYGGITVTGPVSLYNNFKKFKYD |
| | YYYNDIYPLSTELKRIANNQGLNCTDLAQLYYTAYKEMGFTNEIQIVRGTVTC |
| | KSGKTFGHVWCRVKDDGKWINVDPSAAAAHGYSYGTLICTNGYTITNINPN |
| | WALSDDGKT |
| 43 | >WP_069584474.1 |
| | MLFAVLLISSLSLTGITGASATDVGTAQTQAVNNSTVQEKANIQINNTCNTNK |
| | SVATQNSTDASVKTVQNTSTAISNVSTNQTTVKTQAVAQNTVSTKDNQTAVT |
| | STATSINSTSKNSTNTTSTQTQEAAAGETKVSKNFTVSQITDAAARVKAYVET |
| | NHRLPNYVTIGTTQVEMPEFLKLLTAGLLQLNNGTTTSITLKDINSAAKPSESV |
| | KSGNINKAGYLDLAKRVNAFIDANGALPNYATSSLGKLNYQSLIYTFSKIMAF |
| | YKTNERLPSYVTVKSWSTVGTSSTSTSNSSNTSTSVPASLQQYLKSTTNCQVT |
| | NSQIQALAKSITSGKTSTYDKAVAIFNWVRDNIGYSFYYNTKYGAVGTLSKKT |
| | GNCVDTAHLLIALERAAGIPARYEHVKAKFTSGNWYGHVIAQVYVNGKWYN |
| | ADATSSKNTFGVIKNWNTGTATYKGTYATLPF |
| 44 | >WP_084689123.1 |
| | MGIAGASAADAGTAQTQAVNNSTVQEKANIQINNTCNTNKSVAAQNSTDAS |
| | VKTVQNTSTATSNVSTNQTTVKTQAVAQNTVSTKDNQTAVTSTATSINSTSK |
| | NSTNTTSTQTQAAAAGETKVSKNFTVSQITDAAARVKAYVETNHKLPNYVTI |
| | GTTQVEMPEFLKLLTAGLLQLNNGTTTSITLKDINSAAKPSESVKSGNINKAG |
| | YLDLAKRVNAFIDANGALPNYATSSLGKLNYQSLIYTFSKIMAFYKTNERLPS |
| | YVTVKSWSTVGTSSTSTSNSSNTSTSVPASLQQYLKSTTNCQVTNSQIQALAK |
| | SITSGKTSTYDKAVAIFNWVRDNIGYSFYYNTKYGAVGTLSKKTGNCVDTAH |
| | LLIALERAAGIPARYEHVKAKFTSGNWYGHVIAQVYVNGKWYNADATSSKN |
| | TFGVIKNWNTGTATYKGTYATLPF |
| 45 | >WP_069584472.1 |
| | MGIGGVIIKKKLPFAVLLILGLFLASIAGASAADVGTAQSQAVNNSTVQENAGI |
| | HTNDTYNENESVATQNSTDENITEGTSTATSNTSTNQTTVKTEPVAQDIANTS |
| | ADQTATTLTVTSTNSTSKNSTVSETNTKNAVSTALNNSQNSTSTKTNTWNNT |
| | TSTQTQSTGYDTIKNNDSYQKAAAGETKVTSKSFTAKQINNAAAKVKAYVET |
| | NHRLPNYVTIGTIQVQMSDFLKLLTANILQLYNKKTSSITLKSINSAAKPSESV |
| | KSGTMTKSCYLDLAKRVNVFINTNGALPNYATSSLGKLNYKSLIYTFSKVLAF |
| | YNTNSRLPNYVTVKPWSKVGTSDTSNSSTPVPASLQQYLKSTTNCQVNNAQI |
| | QALAKSITSGKSSTYAKAAAIFNWVRDNIGYSFYYNTKYGAVGTLSSKTANC |
| | VDTSHLLIALERAAGIPARYEHVYAKFSSGTWYGHVIAQVWVNGKWYYADA |
| | SSSRNTFGVINNWNTTTATVYGTYTSISF |
| 46 | >WP_048192858.1 |
| | NVLTITLNNNLTKATQYAIILNPGSITDLAGNPINAYGIRFTTNGTTFTTTQITTA |
| | AASVKSYIETNNKLPNYVTIGTNQITMPQFLQLLVTSLLQINSGTNTAISIGNVS |
| | SPTNSTGDYIYGNINQTEFLKIAQSIKSFIDSNGTAPSYASSSLGKVQYETLIYM |
| | FSKVVNYYGTNKVLPNYVSVDSAVANPGSYSIPSDLQQYLQATTNCQVTNSQ |
| | IHALAKSITSGKSSTYDKAVAIFNWVRDNIGYSFYYNTKYGAIGVLSSKTANC |
| | VDTAHLLIALERAAGIPARYEHVYAKFSSGNWYGHVIAQVWINGKWYNADA |
| | TSSSNTFGVINSWNTATATVYGTYATLPF |
| 47 | >MTK63138.1 |
| | MVVNANTTTAASVNQTNGTNSVYKVGNNNTVSSTTLTDNNKSTNPISKVAV |
| | NSTTKPKINSTMAAGEPVKVNGLTLTQMKDGISRAEAFYKKNGRLPNYISFGN |
| | RNISITTFQKNIATQGLKINTTTTTKINGLTIAQIKEGISRAEAFYKKNGRLPNYV |
| | SYGSRNISITTFQKNVATAGLKINTSSSNKIDTSSVSALAASLKKGSSSQYETAT |
| | KIFNWVRDHISYSFYYNSKYGASGTLQKRIGNCCDTANLMVALARAAGIQAR |
| | YLHGTCKFSSGTWYGHVWAQLYVNGKWLNADGTSYKNSLGVVKNWNTAT |
| | YTLHGIYQKLPF |
| 48 | >MBO5180147.1 |
| | MTKIATINKIAKKVKSNVDKNQKLPTFDNYTQPQYAYLLAQSVITPNKDIKSI |
| | KVAKAPNASGTSISRSIDKADYIKLAKYFVKFIKDNGRLPNYVQFKTYKIKPK |
| | VFIYAMAKIVVFYEENKKLPSYCNFNSKVFSTAPKVTGNEVFDYFVKIFGNVK |
| | TIDEAFAKVKDRGYSYYYDDKYANKTVIDRIKNKWGVNCTDSCQMFWHIAK |
| | ALGYEVKCMHVQCSTGGHVRLKLKHSKHTEGNWINRDPAAILSDNGQPLTYI |
| | WCSNAPLNATNPKWFLDNLYR |
| 49 | >WP_069583985.1 |
| | NNGSSGYALFRYGLINSSFGMDSTIPTVKSIDPANNSVNVPINKVIKVTFSESIK |
| | RGTLSIYVKNSSTGKYVPITTSISGNVLTITLNSNLTKGTTYNIILNAGSITDLAG |
| | NALKAYTTKFTTDATAPTVKSIDPSNNSANIPINKTIKITFSEPVKAGTLSIYLK |
| | NLKTGKYVTVTKKISSNILTITPAISLTKATLYYVILNAGSITDLAGNAIRSYTT |
| | KFTTDSTAPTVKSIDPSNNSVNVPLGKVINVTFSESIKAGTLGVGVKDRTGKYI |
| | PITTSISGNVLTITPKSNLTNGTLYYIVLNASSVTDLAGNALKAYTTQFTTNGTI |
| | FTITQINAAAASVKSYVESNNRLPNYVTIGTSQVKMPQFLQLLVTSLLQINNKT |
| | NTFMIPGSINAPESPTGSYMYGNIVLTEYLSIAQNIRNYINSNNCAPNYMTSSL |
| | GNIQYESLVYIYSKILNYYKTNGNLPNYVLVDSSVTNTAIPSELQQYLQATTN |
| | CQVTNSQIQTLAKSLTSGKISAYDKAVAIFNWVRDKLGYSFYYNTKYGAVGT |
| | LNAMTGNCVDTAHLLIALERAAGIPARYEHVYAKFSSGNWYGHVIAQVWVN |
| | GKWYNVDATSYSNTFGVIKNWNASTATYKGTYATLPF |
| 50 | >WP_095652110.1 |
| | MLSINCISAATETGNTTNTSEQNITSNVINSSNTTIQKSMDTANTTNSSNTTIQK |
| | SMEAAGGTSYSKVKAVWIPSDYVSSLNVTALQNLGITDVFVKSNLISTPSYSS |
| | VLTSILQKLKGTNIRVHAWITCFKDANGDWIDPANKTQQDFLINSITSIVKNYN |
| | VSGINLDYIRYSGSGSNTAGESGTAAITAFVQRVYNTVKSINSSVAVSADLMP |
| | EGSANAYYYGQNYTQLAQYLDFLVPMIYKGNYGYNSSTGTSSSGKNGTSWI |
| | ASTVAYIVSQANGTPVIAGLQTYRSDKNVTAIPASELQNDIDAAINNGSSGYA |
| | LFRYGLINSSFGMDSTIPTVKSIDPANNSVNVPINKVIKVTFSESIKRGTLSIYVK |
| | NSSTGKYVPITTSISGNVLTITLNSNLTKGTTYNIILNAGSITDLAGNALKAYTT |
| | KFTTDATAPTVKSIDPSNNSANIPINKTIKITFSEPVKAGTLSIYLKNLKTGKYV |
| | TVTKKISSNILTITPAISLTKATLYYVILNAGSITDLAGNSLKSYTTKFTTDKTAP |
| | TIKSIDPSNNSANIAINKVIKVTFSEPVKAGTLSIRVKNLKTGSYISIAKKISGNT |
| | LTITPTSNLIKATQYAVVLNAGSITDLAGNSLKSYTTKFTTDKTAPTIKSIDPSN |
| | NSANIAINKVIKVTFNEPVKTGTLSIKLKNLKTGTYVSITKTISGNTLTIKPTSTL |
| | TKATQYAVVLNAGSITDLAGNAIRSYTTKFTTDSTAPTVKSIDPSNNSVNVPL |
| | GKVINVTFSESIKAGTLGVGVKDRTGKYIPITTSISGNVLTITPKSNLTNGTLYY |
| | IVLNASSVTDLAGNALKAYTTQFTTNGTIFTITQINAAAASVKSYVESNNRLPN |
| | YVTIGTSQVKMPQFLQLLVTSLLQINNKTNTFMIPGSINAPESPTGSYMYGNIV |
| | LTEYLSIAQNIRNYINSNNCAPNYMTSSLGNIQYESLVYIYSKILNYYKINGNL |
| | PNYVLVDSSVTNTAIPSELQQYLQATTNCQVTNSQIQTLAKSLTSGKISAYDK |
| | AVAIFNWVRDKLGYSFYYNTKYGAVGTLNAMTGNCVDTAHLLIALERAAGI |
| | PARYEHVYAKFSSGNWYGHVIAQVWVNGKWYNVDATSYSNTFGVIKNWNA |
| | STATYKGTYATLPF |
| 51 | >WP_013645370.1 |
| | MVVNVNTTTAASVNQTNGSSSVYKVGNNNTVSNITLTDTSKSTTPISKVTVNS |
| | TNKSKINSTMAAGEPVKVNGLTLTQMKDGISRAEAFYKKNGRLPNYLSFGTR |
| | NISITTFQKNIATQGLKINTTTTTKINGLTIAQIKDGISRAEAFYKKNGRLPNYLS |
| | FGTRNISITTFQKNVSTAGLKINTSGSGNKIDTSSVSALAASLKKGSSSQYQTA |
| | VNIFNWVRDHISYSFYYNSKYGASGTLKNRIGNCCDTANLMVALARAAGIQA |
| | RYLHGTCKFSSGTWYGHVWAQLYVNGKWLNADGTSYKNSLGVVKNWNTA |
| | TYTLHGIYQTLPF |
| 52 | >QGZ14604.1 |
| | MKDRFDIDEFLELKRRWEAFRFTHGRKPKYIRTKDGEEIPQCIFDDMILRYENF |
| | VKKYNRRPRVVSILSDKWIRTGFWKQDKQDTGYTCGPSSLQMALSALGCNV |
| | SEDVLAKAGGTGTSGTGHEGMLRAVEYASKKCKMKITGRFQSFNGYGWSKV |
| | VDGIKAGAEYVIHLKPLPALKYDYKGSQVWKSFTGGHYVYLVGVNMVDRL |
| | VLIADPTKGLRIFGMKQVEDAMAGISKESLLILRKG |
| 53 | >MBO5476297.1 |
| | MTKKENVAKIAKEVKTSVEKKYKIPSKTDAGIGYTYPEYAYILAKAVITPGKD |
| | VAHKKNIGEAPKPSGSTISRTVKKGDYVKLAKNLISFVDNNKRLPNYLTYGG |
| | YKIRTRVYIDAFARIVNYYYTNKRMPAYVNVNSKAFTKPSTAAQSKDEVFNY |
| | FVKKFGNVKTIDEAFNKVKERGYGYYYDDVYSNKTSIDRMYSKSGINCTDSC |
| | QVFWHIGKALGYSVRCLHVRCSGGDGHVRLQFKHPKNTEGNWINRDPAAILS |
| | ANGRPLTYNWCTSNYTLLATNPSWFTANLNR |
| 54 | >WP_157197549.1 |
| | METIGGGIIKRHSLLVMLIICIMAISTIGSSYATVDNQQSAETNADSALNNNTTV |
| | NQNPVETVKNNQTGIQNRVQNNSSTGTTGNSTTKTLQAQNTTNSTSNISSNSQ |
| | NSINALTNTQNAVQTAETNNQNSANTSLTNSANNTTIPQLAAAGTTYTNVHGI |
| | WLRAEDAGNITVSELKKANITDIFVKANLISAPTYSSVLSSIITKFKNSGIRVHA |
| | WITCFQDANGKWINPANTTQQTTLLNAIKKIVINYSVDGIFLDYVRYSGVGSN |
| | TAGTSGTKTITSFVQKVYNAVKSIKPKAAVSATVMPEGATNANTYGQNYTQL |
| | SKYLDFLVPMIYKGNYGQNTTWIGTTTKYIVNNAGGKPVIVGLQSYISDYDTT |
| | PLSAGELNADIKSALSNGASGYALYRYGLISKDFIAPPSENTSDIQSAAAKVKA |
| | YIETNHKLPNYVTIGTTQVTMPAFLKLMVKGLIQINSKITTPIILKTVNNPQNST |
| | GSFTKGNINKTSYMDIAQRINSFIDSNGLAPNYATTGLGKVQYEQLVYMFSKI |
| | LNYYNTNKALPSYVSMDPGVKVSPSSGSDIQTTQVFTLDQIKSAATSVKSYIES |
| | NHDLPNYVTIGSVQVKMTDFLRLLLIGTIEVNDGSNAQISLKTVSAAATPSESI |
| | KSGNITKANYMDLANRVKAFIDANGALPNHASTTLGEIGYSSLIYMYSKILAY |
| | DSTNKALPNYVSVSPWSIVSTVPIPSELQQYLKETKNCQVTNSQIQALAKSITS |
| | GKSSTYDKAVAIFNWVRDNIGYSFYYNTKYGAVGTLNAKTGNCVDTAHLLI |
| | ALERAAGIPARYEHVKAQFTSGTWYGHVIAQVWVNGKWYNADGTSSKNTF |
| | GVINNWNTATATYYGTYAELPF |
| 55 | >WP_255590822.1 |
| | MLLLFSITLLSTDYIYATTPNQQNSDIIANTNIVGEITVDVQQDVKSTANIQQTT |
| | VGTLNDVQAVSNTVTSQKATKNAVKTVSSSVTSKDSSKNSVKIQSASEAAGD |
| | VNNNKAAGTQTTVKSTTVKYTLSDIKDAASRVKIFIESKKKLPKYVQMGTRQI |
| | TMPQFLQLLTAGLTQVNKGSTGSIALKSVETSLKPTQNLKSGTIKKSEYLVMA |
| | GRITSFINSNGKTPNFASSSLGKIQYESLVYMYSRIMKYQSVNKDLPNYATMQ |
| | PWQKSADTPVTPVTPIPAELQKYLLPTNHCQSTDSRIIALASSITQGSSSAKDRA |
| | TKLFNWVRDNIGYSFYYDTNYGAVGTLNSRTANCVDTSHLLVALSRASGIPA |
| | RYVHGVCKFSSGTWYGHVWAQLYVDGTWYNADAISYRNSLGVINNWNTAS |
| | YTLKGIYAALPF |
| 56 | >WP_069583527.1 |
| | MLIICIMAMSTLGSSYAAADNQQSAETTADSALNNNTTVNQNPVETVKNNQT |
| | GIQNRVQNTSLTGTTGNRTTQDLQAQNTANSTLNISSNSQNSINTLTNTQNAL |
| | QTAGTNNQNSANASLTNSANNTTIQPAAGTTYTNVHGIWLRAEDAGNITVSE |
| | LKKANITDIFVKANLISAPTYSSVLSGIITKFKNSGIRIHAWITCFQDANGKWIN |
| | PANTTQQTTLLNAIKKIVTNYSVDGIFLDYVRYSGAGSNTAGTSGTKTITSFVQ |
| | KVYNAVKSIKPKVAVSATVMPEGATNANTYGQNYTQLSKYLDFIVPMIYKG |
| | NYGQNTTWIGTTTKYIVNNAGGKPVIVGLQSYISDYDTTPLSAGELNADIKSA |
| | LSNGASGYALYRYGLISKDFIAPPSFNTSDIQSAAAKVKAYIETNHKLPNYVTI |
| | GTTQVTMSAFLKLMVKGLLQINSKITTPITLKTVNNPQNSTGSFTKGNINKTSY |
| | IDIAQRINSFIDSNGLAPNYATTGLGKVQYEQLVYMFSKILNYYNTNKALPNY |
| | VSMDPGVKVSPPSDSDIQTTQVFTLSQIKSAATSVKSYIESNHDLPNYVTIGLV |
| | QVKMTDFLRLLIIGTIEVNDGSNAQISLKTVNAAAKPSESIKGGNITKANYVDL |
| | ANRVKAFIDANGALPNHATTTLGEIGYPSLIYMYSKILAYDSTNKALPNYVSV |
| | SPWSAVSTVPIPSELQQYLKETKNCQVTNSQIQALAKSITSGKSSTYDKAVAIF |
| | NWVRDNIGYSFYYNTKYGAVGTLNAKTGNCVDTAHLLIALERAAGIPARYEH |
| | VKAQFTSGTWYGHVIAQVWVNGKWYNADGTSSKNTFGVINNWNTATATYY |
| | GTYAELPF |
| 57 | >WP_209584099.1 |
| | MSRNFFQTIISEMYISVHGSEFQGRSGHGGGIIKRNLLLAMLLVMSLALSNVGS |
| | IYAYSLSETVSDNNTSNAQSLLENTTNYTNTTTNYTNTQTTANLTVNTTSSVN |
| | ATNTTNDSGISNTATSNTASNSSTNATAAAGGDYSKIQGIWVSSTDALKVNVT |
| | QLLSMGITDIYVKCNIYSDPTYKTLLSQVVQMENGTGIRVNAWVTCFKDANG |
| | NWIDPQGTYNYTVKTPVITSTQVAVKNYYKSWYKSWYKSWYKSWYKSWY |
| | KSWYKYRGHWKYKWKSTWKSTWKYTWKYVWKYTLKYTTSYKTVTTTTYT |
| | TKTVTSYNTTHNDEVISAISDMVKNYGVDGVNLDYVRYPGTAYKYANGTDA |
| | VTSFVEKVYTTVKNINQNAAVSADLMPEGAANAYYYGQDYAQLSQYLDYL |
| | VPMIYNGNYNEDTDWIGDMTDYIVKHSGGKPVVVGIQTYESDSNVTPLPASE |
| | LNQDIKAAIDNGASGYVLFRYGLIDDGFSGYTGNMSSGTTTSDNSTEENNTST |
| | SNNTTISLADIEDAATRVADYVEANYRLPTYVTIGSQNINMSNFLNLLVNALR |
| | NIYNNDTDSVVLGNFTSATSSSENVTAGNIYTTEYLSMANSIASYMALNGKAP |
| | NYASSTLGNMGFESIVYMYSKILRFEDTNARLPSYVGVKAWSVVTTNSSSTTT |
| | VPASLQQYLQATTNCQSTNAQIVALSKSIIASAGATTTYAKAVAIFNWVRDNI |
| | GYSFYYNTKYGAVGTLNAKTGNCVDTTHLLIALERAAGIPARYVHGYCKFSS |
| | GSWYGHVWAEIYVDGKWYSADAISYRNTFGVINNWNTATIYGRYASISF |
| 58 | >MBR3213492.1 |
| | YYFAKAIKKPNTSIQKLSFEEAKNPTGDYISRDVPKADYLDMAKRLITYVEKN |
| | KKMPNYVTYKGIKVLPRLVIYAFAKILIYYEDHNKTAPKYVTISSKAYTKPTE |
| | TGNVVYDYWVKKFGFKPTSLDEILEYVADHFEYQYYYDDQKSNREVIDSQA |
| | GNCTDLLQMLYNLTKQLGYEIEVLHVLCSGGDGHVRGRFKHPKNTDGEWVY |
| | RDIACAASSGNPTCNWCTSGYTLLAVNPSWFFESINR |
| 59 | >WP_052374296.1 |
| | MFDLDQIFILFDFVKQGGGIINRKLLLSVLLFFSLALALNASTSSAASMDQKNIS |
| | ISNTSPDNLGNNICSSNYITGNANSTTKTNIKNTTTKKAKINSTMAAGAPVKVN |
| | GLTLAQLKGGTSRVQSFYNKNNRLPKYVTFGTRKISISTFKKNIATQGLKIKTK |
| | TISTKTVTTKKVNGLTVLQLKEGTYRVQSFYNKNGRLPKYVKFGTRKITMAT |
| | FQENITTQGLKINTTRKTVKIDTSSVASLAKSLKAGSTSTYNTAVAIFNWVRD |
| | NINYSFYYNTRYGASGTLKNRSGNCVDTSHLLISLARSAGITARYVHGTCKFS |
| | SGTWYGHVWAQLYLNGKWVIADGTSYRNSLGVVKNWDTSTYKLNGIYTTL |
| | PF |
| 60 | >MCL2115004.1 |
| | MGLLTDSNMCIDKTTFKDMLKRKINFVNINKKDPSIVYLSPNAQKDWIEIKQF |
| | NDMERRWNSWIDIKKSEPNCVFIGNTFNSPTLQTGNVYVNKYINDYNIRQDTS |
| | YYCALNIIQNIWYTLFGQIIKESEIAKVAGTTNPNGTDHKGIEKALNYLAEKYK |
| | VTISVEWKHFSDIGWNGVEKIYKSQNQAIGFHVLYKLKFGHYEVGYFINKSTN |
| | TIGIVNSLGNKDKNGTFLGYFEKRSFKEMLSYINGINQPSCLIVTKK |
| 61 | >WP_202879458.1 |
| | MELKKNKNTPEYVSITQFKDMLSRYNRFKEFNGREPRVVFIYSGGGPSVSLET |
| | FKDMCKRYNQFLEENRREPRIVYVTPPEPPVPEEVREMRRVLGEFKTATQLYT |
| | LVSRRCKYKFYYNDQTPNREALKKMVTDGINCTDACQLFKPVIEGLGYSVRI |
| | EHVKVKCNDNKWYGHYFLRVAGKELASVSLPPERWTVWDYVSATKTGRPL |
| | GAPCCSRGIQHLGWGIVGPVYDK |
| 62 | >MCQ8904452.1 |
| | MPLKNGKIEIKKFREMLRRYEDFVKNEGHEPNIIYIEQGKSDHVSLETFKDML |
| | RRYDRFKKETGREPALIYLDPLGRSGDYVNLATFKDMLRRYDRFKADKGREP |
| | SYVMIESMRPLPRLDGKWTRRVIQEIGLFLDASELYRLVSEKCKYNYYYNDQ |
| | FSSEEALRRMTTTGINCTDACQLFEKVFLEMGYETRIEHCKVKCRDGKWYGH |
| | YLLNVKGFELKDWVIWDYVSATKTGRPLGKPCCLNGLKHLGWGIVSPKND |
| 63 | >WP_237778922.1 |
| | DMLRRYEDFVRINGREPNTIYIEQGKSDHVPLGTFKDMLRRYEDFVRINGREP |
| | NYISIQPQPSLKGPWTTKVIEKIGTFHDATSLYEQVKKTCKYKYYYNDQVPNH |
| | VAVMRMTTSGINCTDACQLFQRVMEEMGYMVKIEHVQVKCSDGEWYGHYL |
| | LNVKGYEFKDWVIWDYVSATKTGRPLGKPCCLNGLKHLGWGIVSPKHD |
| 64 | >MBI4812974.1 |
| | MIISYKQYQDMSIRIMQFQMERGRKPKYVTLNGTKIGQKQYEDMMKRVDVFI |
| | KAKGRNPKSVRLDEDLEIIKPSLGKKKSASWIKLEEALKNKFNNAEELYNALS |
| | DQGQYKYYYNDKFDNKMALTRLRKGLGINCTDYSQLLRPVLEDMGYDVRY |
| | AHGRVKCGDKQWYGHVWLQIKGRAYDNWINYDVVAVTHHGIKRPIGSLVC |
| | VAGVKDVEYDPNWLAQ |
| 65 | >PKL66861.1 |
| | MIISYKEYQDMSVRIMQFQMERGRKPKYVTLNGSKIGQRQYEDMMRRVDLFI |
| | KSKGRNPKSVRLDEYIEIVKPSLGRKKSASWIKLEEALDKKFNDAKDLYKAIA |
| | DQGEYKYYYNDKFDNKMALTRLRKGLGINCTDYAQLIRPVLEDMGYDVRY |
| | AHGRVKCGDKQWYGHVWLQIKGRDYGNWVNYDVVAVTHHGIKRPIGSLVC |
| | VNGVKDVEYNPKWLI |
| 66 | >QGH71204.1 |
| | MDIKLKEFKDMIRRVKHFLSTKKRPPRYVNTSMGRITYNQYIDALKRYTDFV |
| | KKNKREPKYVRIYSEAKPTHKPGGPNQELFEKTFNVQIESAQDLYAWLRDNV |
| | KYSYYYNDKYDNREVIEKIKRGEGVNCTDLAQFCRPILRDLGYECDFIRAEVK |
| | CSNNKWYGHVFLRIRGKEYNDWVYFDGTAVTHENFDRPLGKLICTGGTREIT |
| | INPKWLLNAEQNL |
| 67 | >MBC7109230.1 |
| | MEQQRAGIGDKKMIDISNTTICLKKENFKDMQNRYQNFLKTKGREPNIIYIKK |
| | NGNTYITLQQFKNMKQRWEEFIKTNNREPNCVYIRKIDPNEPATNFDLYEVVY |
| | YQVEKQDTGYTCGP |
| 68 | >MBC7129280.1 |
| | MVDISNITICLPKETFKDMKQRWEEFIQKNKREPNIIYTKKGGDTFVTLKRFKD |
| | MKQRWEEFIQKNNREPNCVYIRKIDPNEPATNFDLYEVVYYQVEKQDTGYTC |
| | GPSSLLMMLRELAQSALPQYSEAYVAKLAGTSTGGTTHDGMINAAKKIAADL |
| | GWKLTAKRVNFSDTSWEQLGKWIADPKVGVVAHVMTAGLPYWGNSDYGH |
| | YIFPIGLDLKDKTITIADPARGVPDVYKATFDQFARACRRVTWAPSLLLFIKG |
| 69 | >MTK63138.1 |
| | MVVNANTTTAASVNQTNGTNSVYKVGNNNTVSSTTLTDNNKSTNPISKVAV |
| | NSTTKPKINSTMAAGEPVKVNGLTLTQMKDGISRAEAFYKKNGRLPNYISFGN |
| | RNISITTFQKNIATQGLKINTTTTTKINGLTIAQIKEGISRAEAFYKKNGRLPNYV |
| | SYGSRNISITTFQKNVATAGLKINTSSSNKIDTSSVSALAASLKKGSSSQYETAT |
| | KIFNWVRDHISYSFYYNSKYGASGTLQKRIGNCCDTANLMVALARAAGIQAR |
| | YLHGTCKFSSGTWYGHVWAQLYVNGKWLNADGTSYKNSLGVVKNWNTAT |
| | YTLHGIYQKLPF |
| 70 | >WP_013645370.1 |
| | MVVNVNTTTAASVNQTNGSSSVYKVGNNNTVSNITLTDTSKSTTPISKVTVNS |
| | TNKSKINSTMAAGEPVKVNGLTLTQMKDGISRAEAFYKKNGRLPNYLSFGTR |
| | NISITTFQKNIATQGLKINTTTTTKINGLTIAQIKDGISRAEAFYKKNGRLPNYLS |
| | FGTRNISITTFQKNVSTAGLKINTSGSGNKIDTSSVSALAASLKKGSSSQYQTA |
| | VNIFNWVRDHISYSFYYNSKYGASGTLKNRIGNCCDTANLMVALARAAGIQA |
| | RYLHGTCKFSSGTWYGHVWAQLYVNGKWLNADGTSYKNSLGVVKNWNTA |
| | TYTLHGIYQTLPF |
| 78 | >WP_095608514.1 |
| | MGGLNCKTINLKKGSKGAEVKELQTILQNKGYYDGKIDGDYGDITVQAVKLL |
| | QRKQGNDPDGNFGPKTCSKLDQKTGTTPTGNINASNQKFLKDEIIRAGKVFRQ |
| | HIKNNNNYPNYIEMKNSSGKTYQVSKALYMGLFEGMNMFAIKNGRLPNFVV |
| | ASSTANNPLCIDYQNNGVNCGPTSLSMCIQMYGEYISEPILARACDTGSNGTG |
| | PSELKMGAKKYGYKLVEIPRNINAVKESLAKCRPVIMHIHTSYSGGRSCLGYR |
| | GAYGHYIMCYGTNGNYYKLADPTKGFKTCSSTSIDNARSGEFMKYYSVCIVS |
| | SGGS |
| 79 | >Pei002 |
| | MGSLSSTTLSTFNSDKTAYLKLFSESVGVKFNTFTQGFNLIANRVYGHYANDV |
| | YSQLNALNRLKKKLGLNCSDITQLLYQMGKDLGYNVAYEHVMCKVSGGHIR |
| | LKISGGEFGNLGTQIKNWKTVDGAAQLKSGQGINNIWCPDGAIVSYNDKWLQ |
| | SDDGKTGSG |
| 80 | >Pei005 |
| | MGLISYDIYKDMQIRFDEYVEKNNTDPERIFILPGKSESTILTHDLKALAYLNP |
| | DKFPIEYNTGENPIESKPDTVKSDLDKLRDKFGYFDTICNWYDNVVRKCDYA |
| | YYYNQSHSVRQIIDTLLNDGFIALNCTDWSLITSWIAKELGYPFRVYNVVCSS |
| | GAGHVRPAIQDRDNLGDWMSTDPAAIADKSEIYYACLFDVWCDTKNGATIV |
| | DTSDENQLLGWMHWGSG |
| 81 | >Pei008 |
| | MGTKFSKKEMKKAGKTIRENIKKGKGLIRTINMTDMSGKTHRLNKADYAGLF |
| | EAQNVFILKNNRYPNYVTLNSEANNPLVLNYQDDKYSCCVASFNMAVQMLY |
| | GWINEDKIKKTFKTDINGTDPSNMIIGAKNLGYKVSRISRNFAGVKQSLNKGY |
| | PVIAHIQTKSAKCLGYQNDYGHYVLIYGTTPSGAYKVADPTKGLKVCKANEL |
| | DKATNGRSINYYSVGILGSG |
| 82 | >Pei009 |
| | MGSSFSKSEVLRGAKIFRNSIVKGKGVPKEVTLKVRETGKNKTLTRKQFAGIM |
| | EATNVFRLKQGRYPNYTTLNSTANNPLVMNYQDDSVSCGCASFNMCVQLTY |
| | GEWIDEKAIKKTFGTGSSGTAPSQIIAGAKKYGYAVEKIARNSSAVKKALEKG |
| | YAILAHIDTKPATCLGYQNNYGHWTVIYGLDDGKYKIADPTKGIKTCSTKIYD |
| | KAMLGRTIYYYQVRPLGSG |
| 83 | >Pei010 |
| | MGTRFMNDVIVKASKTIQKSIREGKGLPDKISMKDSNSKTHNLSKKEYAGLFE |
| | SRNVFILKHGRVPNYVTKNSTANNPLVMDYQDSSVTCGPTSLSMAAQMLYG |
| | NTSESAFKKACNTDSNGTSPDELVAGAKKLGYKVTKIGRNSSAVKNSLKLGK |
| | PVVAHIETGGGTKPKCLGFLNNYGHYILIYAVSGDYYLVADPTKGIKKCAFSQ |
| | INKATNGRSIHYYSVSPLGSG |
| 84 | >Pei011 |
| | MGTRESKNEMNKASKTIVKSIRNGKGLPRIVEMKEMDHNKTIRVDKSHYNGL |
| | FEAQNVFIRGHGRLPNYVTMNSTANNPLVMDYQDNGYTCGPTSLSMAIQML |
| | YGWKSEKRCAEACHTGSSGTSPDDLIAGAKSLGYKVTPIKRNFAGVKQSLNK |
| | GYPVIAHIETGGRTKPKCLGYVNNYGHYILIYGTTASGGYKVADPTKGLRVCS |
| | ASELDKATNGRSINYYSVKPLGSG |
| 85 | >Pei012 |
| | MGLDCKTINLKKGSTGTKVKELQIILQKKGYYDGVIDAIYGDLTVQAVKLLQ |
| | REQGNSPDGWFGTKTCSKINQNTTLETPQGNTNASGQKFMKDEIIKAGKVFR |
| | QHIKNNNNYPNYLEMKNSNGKTYQVSRALYMGIFEGMNMFSIKKGRLPNYV |
| | VANSTANNPLCIDYQNNGYNCGPTSLSMCIQMYGEYISESTLANVCGTTRNG |
| | TSPASLNSGAKKIWIQIIRNSTGSG |
| 86 | >Pei013 |
| | MGMVRFSKKEMQKASKIIRESIKKGKGQPLSVKMVEMDKGKTIKVSKAYYM |
| | GLFEAQNIFIRNKGRKPNYVTLNSKANNPLVMDYQDNAYTCCPTSFSMATQL |
| | LFDNISEKECAKALNTVLGSGTDPSDLIKNSGKLGYKVTPIKRDYKSVKSSLD |
| | KGFAVIAHIQTRPATCLGYSGDYGHYVLLYGAYRKLLTKYYKVADPTKGLKT |
| | CKASILDKATNGRNIKYYSVGILGSG |
| 87 | >Pei014 |
| | MGSNPIRFSKAEMKKAAKTIQNNVKKGKGQPKTIKMTDLNGKLHSLKKKEY |
| | CGLFDNQAGYFNEHGRLPNYTTLLYETPTAFRGQKQPTSVECGDTGLHNAMT |
| | QLMIYHTVAECKKACGTNQNGTTPSQLIAGGKKLGVEVKKIKRTFQAVKEAI |
| | DKGYSVIAHIQTAGSTRPKCLGYVYDYGHWISIYNISGKDKFKVYDPTKLYKV |
| | CTAIEIIKATGGRSINFYSVSPLGSG |
| 88 | >Pei024 |
| | MGDRDHISFTEYTYLMCRIAYWLINNAKKFNTRNVYGYMNRSADYGTIIRVI |
| | KERSTNYQPDSLIAEFVECAIYDNTDLSFLPNYVTGRNGTKYFKQTYVNMAQ |
| | RVSAYEVLNGVSPAIVYLKKEELTSNNKSSILNEARKIFGDFNTCDGWLGKIQ |
| | GRGYSFYYNNKYSNRNTLQRIKNKQGANCTDSTELSYQILVGLGYTVQVIHV |
| | MCKSGTGHVRLRCKHPKYTDNQWIYRDPAAVLKGNGIRSNWCANGRVVAY |
| | DPKWIMDTINWGSG |
| 89 | >Pei033 |
| | MGLHCNTINLTKGSKGETVKELQTILKNKGYYTGKIDGYYGDLTIQAVKLLQ |
| | RQQGNSEDGQFGPKTCRKLNQNINNNTTHTTPTNNTNPVGKKFLLDDLITAR |
| | KTFRKHIKNNNNYPNYITIPDNTGKKYDVLKADYMGIFEAMHMFAIRNGRLP |
| | NWVIHNTTANNPLVIDYQNNGYNCGPTSLSMCLQMFADYISEPTLAKYAGTN |
| | RNGTGPADLKRTAKHYGYGMTEIGRNSKAVQDSLSKCRPVLMHIHTSYAGG |
| | RSCLGYRGAYGHYIMCYGMRNGRYLIADPTKGFKVCNPVAIDNARSSSNMK |
| | YYSISILGSG |
| 90 | >Pei034 |
| | MGLNCNTINLTKGSKGETVKELQTILKNKGYYTGKIDGYYGDLTIQAVKLLQ |
| | RQQGNSEDGQFGPKTCRKLNQNINNNTTHTTPTNNTNPVGKKFLLDDLITAR |
| | KTFRKHIKNNNNYPNYITIPDNTGKKYDVLKADYMGIFEAMHMFAIRNGRLP |
| | NWVIHNTTANNPLVIDYQNNGYNCGPASLSMCLQMFADYISEPTLAKYAGTN |
| | RNGTGPADLKRTAKHYGYGMTEIGRNSKAVQDSLSKCRPVLMHIHTSYAGG |
| | RSCLGYRGAYGHYIMCYGMRNGRYLIADPTKGFKVCNPVAIDNARSSSNMK |
| | YYSISILGSG |
| 91 | >Pei035 |
| | MGVVNANTTTAASVNQTNGSISANKVGNNNTVSNITLTDTSKSTTPISKVAVN |
| | STTKSKINSTMAAGEPVKVNGLTLTQMKDGISRAEAFYKKNGRLPNYLSFGN |
| | RNISITTFQKNIATQGLKINTSTATKINGLTIAQIKEGISRAEAFYKKNGRLPNYL |
| | SFGNRNISITTFQKNVSTAGLKINTSSSNKVDTSSVSALAASLKKGSSSQYETA |
| | VKIFNWVRDHINYSFYYNSKYGASGTLRNRTGNCCDTSNLMVALARAAGIQ |
| | ARYLHGTCKFSSGTWYGHVWAQLYVNGKWLNADGTSYKNSLGVVKNWNT |
| | ATYTLHGIYQTLPFGSG |
| 92 | >Pei036 |
| | MGVVNVNTTTAASVNQTNGSSSVYKVGNNNTVSNITLTDTSKSTTPISKVTV |
| | NSTNKSKINSTMAAGEPVKVNGLTLTQMKDGISRAEAFYKKNGRLPNYLSFG |
| | TRNISITTFQKNIATQGLKINTTTTTKINGLTIAQIKDGISRAEAFYKKNGRLPNY |
| | LSFGTRNISITTFQKNVSTAGLKINTSGSGNKIDTSSVSALAASLKKGSSSQYQT |
| | AVNIFNWVRDHISYSFYYNSKYGASGTLKNRIGNCCDTANLMVALARAAGIQ |
| | ARYLHGTCKFSSGTWYGHVWAQLYVNGKWLNADGTSYKNSLGVVKNWNT |
| | ATYTLHGIYQTLPFGSG |
| 93 | >Pei043 |
| | MGVGTAQSQAVNNSTVQENAGIHTNDTYNENESVATQNSTDENITEGTSTAT |
| | SNTSTNQTTVKTEPVAQDIANTSADQTATTLTVTSTNSTSKNSTVSETNTKNA |
| | VSTALNNSQNSTSTKTNTWNNTTSTQTQSTGYDTIKNNDSYQKAAAGETKVT |
| | SKSFTAKQINNAAAKVKAYVETNHRLPNYVTIGTIQVQMSDFLKLLTANILQL |
| | YNKKTSSITLKSINSAAKPSESVKSGTMTKSCYLDLAKRVNVFINTNGALPNY |
| | ATSSLGKLNYKSLIYTFSKVLAFYNTNSRLPNYVTVKPWSKVGTSDTSNSSTP |
| | VPASLQQYLKSTTNCQVNNAQIQALAKSITSGKSSTYAKAAAIFNWVRDNIGY |
| | SFYYNTKYGAVGTLSSKTANCVDTSHLLIALERAAGIPARYEHVYAKFSSGT |
| | WYGHVIAQVWVNGKWYYADASSSRNTFGVINNWNTTTATVYGTYTSISFGS |
| | G |
| 94 | >Pei054 |
| | MGDKESINYTNYSNICNRIAYWLVNNNKKFNTRNVYGYMNRSADYGTIIRVI |
| | KERGTNYQSDSLITEFVECAIHDNKDLSFLPNYVIDKNGKKYMKDTYVDMCR |
| | RVSAYEVLNGVSPAIVYLTGNTPVQNTTNNNKLHNYLTNKGCSGMGQCTPY |
| | NCACNSLQQGFYRLTGIHVSESTIASVAGTTTAGTGHQGINTTVVWFNRKYG |
| | KNIKIAWKNFNDLGSSDSARWNKLNEYASNGAVFCHILYRNKYGHYEVLKS |
| | VNGNNVTVLNSLGSRCSKPAYCGYIETRSKSNQLSYMRGISQPSVAILTKGGS |
| | G |
| 95 | >Pei058 |
| | MGVNEHISFTKYTYLMNRIAYWLVNNNKKFNTRQVYSYMNRVADYGTIIKAI |
| | KERGTNYQQDSLIAEFVECAIFDNKDLSFLPNYVSDTDGTKYYKNCYVSMAN |
| | RVSAYEVLNGVSPAIVYLEDPHGNGTTSDTTDITLKRFTDKFGGVTDIDSCLN |
| | KIRGRGYGYYYNSKYNTQETINKIYNKQGVNCTDSSQLFYRLGLALGYNVQF |
| | IHVRCRSGTGHVRLRLKHSKHTGGSWIYRDPAAVLNGNSVSSNWCMNGTILA |
| | YDPAWIFSDLYQGSG |
| 96 | >Pei059 |
| | MGVNEHISFTKYSYLMNRIAYWLINNNKKENTRPVYGYMNRTADYGTIIKVI |
| | KERGTNYQSDGLITEFVECAIHDNKDLSFLPNYVSDENGVKYYKNCFVSMAN |
| | RVSAYEVLNGVSPAIVYIEDPHGNGTTSDTTDTTLKRFTDKFGGVTDIDSCLN |
| | KIRGRGYGYYYNSKYNTQETINKIYNKQGVNCTDSSQLFYRLGLALGYNVQF |
| | IHVRCRSGTGHVRLRLKHSKHTGGSWIYRDPAAVLNGNSVSSNWCMNGTILA |
| | GSG |
| 97 | >Pei060 |
| | MGSKERRMYELYGNDMNRIAYWLVNNNKKFNTRPKYGYQGREADYATIIR |
| | VIKERGTNYQSDGLITEFVECAIVDNKDHSFLPNYVTGKDGKRFYKSTYVDM |
| | AKRCSLWEVEHNGQSPNFIWYETSTNTSASSSVVGNVTKGISNATNTKISDYK |
| | SLYNAFYKGRYGLYYNDVYTQSQSLSRLKSRLPLNCTDLNQLAYYSLKEQGY |
| | NVRIVRGLIYCNKTYGHVWCQVFLNGKWVNFDASAAAKGKGLGSLICGSRF |
| | SVTNINPAWAVSDDGRTGSG |
| 98 | >Pei063 |
| | MGSSNSISWTRFCQGMNSICYWLIQNKKKYKKRDYYQILKLKGSCADVEKK |
| | AKKLGNNKLVAMYTMAVIKDNKSLDFLPNYVTLKNGLQIDKAEYIDMAIRTE |
| | AYITANKRLPAIVYRMSKLPDYKDTTMNFFIKTFNYKGNTIDEALAIIANKKL |
| | YSKYFDSQKTDKQTINDASKGKGSNCVDWGQVYYRIAKSLGYDVQFIHVKC |
| | RVSGTGHIRLRLRHKKHTSGNWINRDPAAVADTTSGNVRSLWCEDGYVIAYD |
| | PSWIFTDLYSSGSG |
| 99 | >Pei065 |
| | MGTREYINYTNYSYMCNRIAYWLVNNNKKFNTRNVYGYMNRSADYGTIIRV |
| | IKERGTNYPSDSLITEFVECAIHDNKDLSFLPNYVVDKNGKKYMKDTYVDMC |
| | RRVSAYEVLHGVSPAIVYLTGNTSVQNTTNNNKLYNYLTTSGCAGMGQCTP |
| | YNCACNSLQQCFYRLTGIQVSESTIASVAGTTTSGTGHPGINTAVAWFNRKYG |
| | QNIKITWKNFKDLGGSDSARWNKLNQYASNGAVFCHILYRNKWGHYEVLKS |
| | VNGNNVTVLNSLGNRCHKPAYCGYIETRSKSNQLSYMRGISQPSVAILTKGGS |
| | G |
| 100 | >Pei067 |
| | MGKYSKTEMKTAAKTIRKNIKKGNGRPEKIKMTDTNGDKHKLKLKQYLGLF |
| | KQWNIFRLKNGRYPNYVTYVGKSKEPTVINYQDNGYQCACASFNMALQSLF |
| | DWEDETTIAQAFNTGKYGTSPSDMINGAKKFGYKIQPINRNQKALQKAFDKG |
| | YGVIAHIDTIKAPCLGYKNNYGHYINIQRITKAGNYRVFDPTKGVLSVKPSCID |
| | NAMLNRTLNYYQVVIEGSG |

### Engineered Methanolysins

Provided are also engineered methanolysins. In aspects, a methanolysin of the disclosure, such as any of the sequences of Table 1 can be modified. Multiple strategies can be used to modify methanolysins. In aspects, a methanolysin is modified to increase killing activity. In aspects, a methanolysin is modified to have increased accumulation in Spirulina. A methanolysin can be expressed as a fusion protein. Described herein are fusion proteins comprising at least first protein and a second protein. In aspects, at least one of the first protein and the second protein is a methanolysin. In aspects, a fusion protein comprises at least two proteins, such as three, four, five, six, seven, eight, nine, or ten proteins, or more. In aspects, the proteins in the fusion proteins are linked via a linker. In aspects, the fusion proteins comprise one or more protease cleavage sites, such as one or more chymosin cleavage sites.

In aspects, a fusion protein may comprise, from N-terminus to C-terminus, the first protein and the second protein. In aspects, a fusion protein may comprise, from N-terminus to C-terminus, the second protein and the first protein. In aspects, a fusion protein comprises, in order from N-terminus to C-terminus, a first protein and a second protein, wherein the first protein and/or the second protein is a methanolysin of Table 1 or Table 4. In aspects, a fusion protein comprises, in order from N-terminus to C-terminus, a second protein and a first protein, wherein the first protein and/or the second protein is a methanolysin of Table 1 or Table 4.

In aspects, it may be desirable to cleave the fusion protein to separate its constituent proteins. For example, it may be desirable to cleave the fusion protein to separate its constituent proteins so that the proteins may individually be used. In aspects, a protease cleavage site may be located between the first protein and the second protein. In aspects, a protease cleavage site may be located between a methanolysin and a tag.

For example, a methanolysin of the disclosure can be expressed as a fusion protein comprising a tag. In aspects, a fusion comprises one or more of: maltose binding protein, thioredoxin, phycocyanin, and SUMO.

In aspects, a methanolysin is expressed in a fusion protein with a marker. Proteins typically used as a marker may include, for example, fluorescent proteins (e.g., green fluorescent protein (GFP)). Other examples include yellow fluorescent protein (YFP), orange fluorescent protein, blue fluorescent protein (BFP), cyan fluorescent protein (CFP), or red fluorescent protein (RFP). Other examples of marker proteins include, but are not limited to, bacterial or other enzymes (e.g., β-glucuronidase (GUS), β-galactosidase, luciferase, chloramphenicol acetyltransferase).

In aspects, a methanolysin can be modified to increase accumulation in Spirulina. For example, domains that can slow enzymatic activity and/or lower expression can be at least partially deleted. In aspects, domains that can slow enzymatic activity and/or lower expression can be deleted. For example, PeiR possesses an N-terminal cell wall binding domain (further referred to as the pseudomurein binding domain) and a C-terminal catalytic domain that cleaves peptide bonds that are essential to the structural integrity of the archaeal cell wall. Removal of the binding domain from the lysin PlyCD10 can increase both Spirulina accumulation and specific activity without affecting specificity.

In aspects, a methanolysin can be modified via molecular electrostatic analysis to design mutations to complement solvent exposed regions with charged residues to aid protein folding and solubility. Accordingly, any of the methanolysins can be modified with one or more amino acid modifications selected from substitutions, deletions, insertions, and combinations thereof.

In aspects, a methanolysin can be subject to structural analysis. For example, an N-terminal domain upstream of a pseudomurein binding domain with structural similarity to domains found in gram-negative lysins is identified. Based on structural models, novel methanolysin constructs can be designed to remove the N-terminal domain and the pseudomurein binding domain to facilitate expression of the catalytic domain in Spirulina.

### Mammals

Provided are also various mammals which can benefit from any of the compositions and methods of the disclosure. Mammals of the disclosure can be human or non-human. In aspects, a mammal is human. In aspects, a mammal is female or male. In aspects, a mammal is a non-human mammal. Non-human mammals can be domesticated. In aspects, a non-human mammal is a pet, livestock, or beast of burden.

Exemplary pets include dogs, cats, ferrets, hamsters, monkeys, rabbit, mouse, rat, guinea pig, and the like. Exemplary livestock include cattle, sheep, pigs, goats, duck, water buffalo, yak, llama, and the like. Exemplary beasts of burden include horses, camels, donkeys, elephant, and the like. In aspects, a mammal is a ruminant.

### Ruminants

Provided are also ruminants which may be utilized in any methods of the disclosure. Ruminants are herbivorous grazing or browsing artiodactyls belonging to the suborder Ruminantia that are able to acquire nutrients from plant-based food by fermenting it in a specialized stomach prior to digestion, principally through microbial actions. In aspects, ruminants of the disclosure comprise one or more of the methanogens described herein. In methods of the disclosure upon ingestion by a ruminant, the pH of the rumen (from about pH 5.5 to about 7.0) facilitates release of methanolysins from Spirulina. The released methanolysin become active upon solubilization and target the cell wall of archaea which includes methanogens.

A ruminant can comprise any mammal of the suborder Ruminantia (order Artiodactyla). Exemplary ruminants include but are not limited to: pronghorns, giraffes, okapis, deer, chevrotains, cattle, antelopes, sheep, and goats.

In aspects, a ruminant comprises cattle. In aspects, a cow of the disclosure is of a species selected from the group consisting of: Holstein Friesian, Hereford, Simmental, Aberdeen Angus, Brown Swiss, Galloway, Red Angus, Belted Galloway, Braford, Limousin, Belgian Blue, Brangus, Beefalo, Chillingham, Ongole, Cachena, Gelbvieh, American Angus, Chianina, Aubrac, Twinner, Parda Alpina, Diary Shorthorn, Maine-Anjou, Danish Red, Retinta, Horro, Montbeliarde, Heck, White Park, British White, Hallikar, Red Poll, Senepol, Caracu, Lakenvelder, Beef Shorthorn, Corriente, Florida Cracker, Tuli, Hungarian Grey, English longhorn, Tarentaise, Australian Lowline, Boskarin, Black Baldy, French Brown, Podolica, Pustertaler Sprinzen, and any combination thereof. In aspects, ruminants of the disclosure are cattle.

In aspects, cow or cattle of the disclosure is used to source milk. In aspects, the milk cow is selected from the group consisting of: Holstein, Jersey, Brown Swiss, Guernsey, Ayshire, Red and White Holstein, and Milking shorthorn. In aspects, the cow is a Holstein cow.

In aspects, cow or cattle of the disclosure is used to course beef or steak. In aspects, the cow is selected from the group comprising of: Angus, Hereford, Brahman, Holstein, Gelbvieh, Limousin, Simmental, Belted Galloway, Charolais, Dexter, Piedmontese, Red Angus, Scottish Highlandm, Shorthorn, Simmental, Texas Longhorn, and Watusi. In aspects, the cow is Angus.

### Ruminant Feed

Provided herein are also compositions comprising ruminant feed. In aspects, the ruminant feed may be for use in feeding any of the described ruminants. Ruminant feed can also be mixed with any of the described compositions. In aspects, ruminant feed is mixed with a modified Spirulina.

Ruminant feed can contain many ingredients. For example, ruminant feed can contain one or more of grains, brans, protein meals/cakes, chunies, agro-industrial by-products and minerals and vitamins. In aspects, a feed may comprise two or more of the aforementioned feed components.

In aspects, the feed is cow feed. In aspects, cow feed comprises one or more grains. Exemplary grains are selected from the group comprising of: corn, barley, oats, wheat, rye, maize, sorghum, wheat, rice, ragi, or millets. In aspects, cattle feed comprises one or more brans. Exemplary brans are selected from the group comprising of: de-oiled rice bran, rice polish, wheat bran, and maize bran. In aspects, cattle feed comprises one or more protein meals/cakes. Exemplary protein meals/cakes are selected from the group comprising of: rapeseed, soybean, cottonseed, groundnut, coconut, palm kernel, sesame, linseed, maize germ oil, maze gluten, sunflower, kardi and guar. In aspects, cattle feed comprises one or more chunies. Exemplary chunies are selected from the group comprising of: guar, tur, urd, moong, and gram. In aspects, cattle feed comprises one or more agro-industrial by-products. Exemplary agro-industrial by-products are selected from the group comprising of: molasses, babul chunni, tamarind seed powder, mango kernel extraction, tapioca and waste. In aspects, cattle feed comprises one or more minerals and vitamins. Exemplary minerals and vitamins are selected from the group comprising of: mineral mixture, calcite powder, common salt, di-calcium phosphate, and Vitamins A, D3, and E.

In aspects, any of the compositions of the disclosure can comprise cattle feed. Feed supplementation approaches for mitigating enteric methane have relied on chemical inhibitors produced synthetically or in a red alga that can be grown in coastal environments. These approaches are only partially effective, not scalable, and can be toxic to the animal, product, and/or environment. Biologics-based approaches are effective and safe, but the approach suffers from the lack of a cost-effective manufacturing platform. The provided methanolysin-expressing Spirulina strains solve the efficacy, safety, and scalability problems faced by existing solutions.

In aspects, a composition of the disclosure comprises one or more methanolysins and cattle feed. In aspects, cattle feed comprises a grain. In aspects, cattle feed comprises one or more grains. Exemplary grains are selected from the group consisting of: corn, barley, oats, wheat, and rye. In aspects, cattle feed can also comprise grass.

In aspects, cattle feed can comprise a single Spirulina strain or a cocktail of Spirulina strains to target the diversity of methanogens in the rumen. In aspects, a method of the disclosure comprises determining a methanogen in a ruminant and feeding the ruminant with a cattle feed comprising one or more Spirulina strains that target the methanogen.

In aspects, any of the compositions of the disclosure can comprise sheep feed. In aspects, a composition of the disclosure comprises one or more methanolysins and sheep feed. In aspects, sheep feed comprises one or more grains. Exemplary grains are selected from the group consisting of: corn, barley, oats, wheat, and rye. In aspects, cattle feed can also comprise grass.

In aspects, any of the compositions of the disclosure can comprise goat feed. In aspects, a composition of the disclosure comprises one or more methanolysins and goat feed. In aspects, goat feed comprises one or more grains. Exemplary grains are selected from the group consisting of: corn, barley, oats, wheat, and rye. In aspects, cattle feed can also comprise grass.

In aspects, the provided compositions and methods can be utilized in conjunction with existing methane-reducing options. For example, alternative approaches for inhibiting methanogenesis include feed supplementation with the small-molecule 3-nitrooxypropanol, chlorinated solvents or toxic chemicals, e.g., bromoform, and natural products like red seaweed that contain bromoform. In aspects, a compound, 3-nitroxypropanol (3-NOP), inhibits methyl-COM reductase (Mcr), an enzyme that reduces methyl-coenzyme M (methyl-COM) to CH4 and is present in all methanogens can be utilized in combination with the present compositions and methods. In aspects, the provided compositions and methods can be utilized in conjunction with halogenated hydrocarbons such as chloroform, chlorobromoform, and bromoform inhibit the corrinoid enzymes, preventing the transfer of cobamide-dependent methyl groups.

In aspects, the described cattle feed may comprise modified Spirulina expressing a methanolysin selected from Table 1 or Table 4. In aspects, feed of the disclosure comprises Spirulina expressing a methanolysin. In aspects, feed of the disclosure comprises either a single strain of Spirulina or a cocktail of strains Spirulina strains. In aspects, the cocktail of strains could target a diversity of methanogens in the rumen. In aspects, cattle feed may comprise modified Spirulina expressing SEQ ID NO: 78.

In aspects, the feed comprises the methanolysin comprising a sequence in Table 1. In aspects, the feed comprises the methanolysin comprising SEQ ID NO: 1. In aspects, the feed comprises the methanolysin comprising SEQ ID NO: 2. In aspects, the feed comprises the methanolysin comprising SEQ ID NO: 3. In aspects, the methanolysin comprise SEQ ID NO: 4. In aspects, the feed comprises the methanolysin comprising SEQ ID NO: 5. In aspects, the feed comprises the methanolysin comprising SEQ ID NO: 6. In aspects, the feed comprises the methanolysin comprising SEQ ID NO: 7. In aspects, the feed comprises the methanolysin comprising SEQ ID NO: 8. In aspects, the feed comprises the methanolysin comprising SEQ ID NO: 9. In aspects, the feed comprises the methanolysin comprising SEQ ID NO: 10. In aspects, the feed comprises the methanolysin comprising SEQ ID NO: 11. In aspects, the feed comprises the methanolysin comprising SEQ ID NO: 12. In aspects, the feed comprises the methanolysin comprising SEQ ID NO: 13. In aspects, the methanolysin comprise SEQ ID NO: 14. In aspects, the feed comprises the methanolysin comprising SEQ ID NO: 15. In aspects, the feed comprises the methanolysin comprising SEQ ID NO: 16. In aspects, the feed comprises the methanolysin comprising SEQ ID NO: 17. In aspects, the feed comprises the methanolysin comprising SEQ ID NO: 18. In aspects, the feed comprises the methanolysin comprising SEQ ID NO: 19. In aspects, the feed comprises the methanolysin comprising SEQ ID NO: 20. In aspects, the feed comprises the methanolysin comprising SEQ ID NO: 21. In aspects, the feed comprises the methanolysin comprising SEQ ID NO: 22. In aspects, the feed comprises the methanolysin comprising SEQ ID NO: 23. In aspects, the methanolysin comprise SEQ ID NO: 24. In aspects, the feed comprises the methanolysin comprising SEQ ID NO: 25. In aspects, the feed comprises the methanolysin comprising SEQ ID NO: 26. In aspects, the feed comprises the methanolysin comprising SEQ ID NO: 27. In aspects, the feed comprises the methanolysin comprising SEQ ID NO: 28. In aspects, the feed comprises the methanolysin comprising SEQ ID NO: 29. In aspects, the feed comprises the methanolysin comprising SEQ ID NO: 30. In aspects, the feed comprises the methanolysin comprising SEQ ID NO: 31. In aspects, the feed comprises the methanolysin comprising SEQ ID NO: 32. In aspects, the feed comprises the methanolysin comprising SEQ ID NO: 33. In aspects, the methanolysin comprise SEQ ID NO: 34. In aspects, the feed comprises the methanolysin comprising SEQ ID NO: 35. In aspects, the feed comprises the methanolysin comprising SEQ ID NO: 36. In aspects, the feed comprises the methanolysin comprising SEQ ID NO: 37. In aspects, the feed comprises the methanolysin comprising SEQ ID NO: 38. In aspects, the feed comprises the methanolysin comprising SEQ ID NO: 39. In aspects, the feed comprises the methanolysin comprising SEQ ID NO: 40. In aspects, the feed comprises the methanolysin comprising SEQ ID NO: 41. In aspects, the feed comprises the methanolysin comprising SEQ ID NO: 42. In aspects, the feed comprises the methanolysin comprising SEQ ID NO: 43. In aspects, the methanolysin comprise SEQ ID NO: 44. In aspects, the feed comprises the methanolysin comprising SEQ ID NO: 45. In aspects, the feed comprises the methanolysin comprising SEQ ID NO: 46. In aspects, the feed comprises the methanolysin comprising SEQ ID NO: 47. In aspects, the feed comprises the methanolysin comprising SEQ ID NO: 48. In aspects, the feed comprises the methanolysin comprising SEQ ID NO: 49. In aspects, the feed comprises the methanolysin comprising SEQ ID NO: 50. In aspects, the feed comprises the methanolysin comprising SEQ ID NO: 51. In aspects, the feed comprises the methanolysin comprising SEQ ID NO: 52. In aspects, the feed comprises the methanolysin comprising SEQ ID NO: 53. In aspects, the methanolysin comprise SEQ ID NO: 54. In aspects, the feed comprises the methanolysin comprising SEQ ID NO: 55. In aspects, the feed comprises the methanolysin comprising SEQ ID NO: 56. In aspects, the feed comprises the methanolysin comprising SEQ ID NO: 57. In aspects, the feed comprises the methanolysin comprising SEQ ID NO: 58. In aspects, the feed comprises the methanolysin comprising SEQ ID NO: 59. In aspects, the feed comprises the methanolysin comprising SEQ ID NO: 60. In aspects, the feed comprises the methanolysin comprising SEQ ID NO: 61. In aspects, the feed comprises the methanolysin comprising SEQ ID NO: 62. In aspects, the feed comprises the methanolysin comprising SEQ ID NO: 63. In aspects, the methanolysin comprise SEQ ID NO: 64. In aspects, the feed comprises the methanolysin comprising SEQ ID NO: 65. In aspects, the feed comprises the methanolysin comprising SEQ ID NO: 66. In aspects, the feed comprises the methanolysin comprising SEQ ID NO: 67. In aspects, the feed comprises the methanolysin comprising SEQ ID NO: 68. In aspects, the feed comprises the methanolysin comprising SEQ ID NO: 69. In aspects, the feed comprises the methanolysin comprising SEQ ID NO: 70. In aspects, the feed comprises the methanolysin comprising SEQ ID NO: 78. In aspects, the feed comprises the methanolysin comprising SEQ ID NO: 79. In aspects, the feed comprises the methanolysin comprising SEQ ID NO: 80. In aspects, the feed comprises the methanolysin comprising SEQ ID NO: 81. In aspects, the feed comprises the methanolysin comprising SEQ ID NO: 82. In aspects, the feed comprises the methanolysin comprising SEQ ID NO: 83. In aspects, the methanolysin comprise SEQ ID NO: 84. In aspects, the feed comprises the methanolysin comprising SEQ ID NO: 85. In aspects, the feed comprises the methanolysin comprising SEQ ID NO: 86. In aspects, the feed comprises the methanolysin comprising SEQ ID NO: 87. In aspects, the feed comprises the methanolysin comprising SEQ ID NO: 88. In aspects, the feed comprises the methanolysin comprising SEQ ID NO: 89. In aspects, the feed comprises the methanolysin comprising SEQ ID NO: 90. In aspects, the feed comprises the methanolysin comprising SEQ ID NO: 91. In aspects, the feed comprises the methanolysin comprising SEQ ID NO: 92. In aspects, the feed comprises the methanolysin comprising SEQ ID NO: 93. In aspects, the methanolysin comprise SEQ ID NO: 94. In aspects, the feed comprises the methanolysin comprising SEQ ID NO: 95. In aspects, the feed comprises the methanolysin comprising SEQ ID NO: 96. In aspects, the feed comprises the methanolysin comprising SEQ ID NO: 97. In aspects, the feed comprises the methanolysin comprising SEQ ID NO: 98. In aspects, the feed comprises the methanolysin comprising SEQ ID NO: 99. In aspects, the feed comprises the methanolysin comprising SEQ ID NO: 100.

In aspects, the feed comprises a methanolysin comprising a sequence in Table 4. In aspects, the feed comprises the methanolysin comprising SEQ ID NO:101. In aspects, the feed comprises the methanolysin comprising SEQ ID NO: 102.

### Modified Spirulina

Provided is also modified Spirulina and methods of making the same. An exemplary method of modifying Spirulina is disclosed in U.S. Pat. No. 10,415,012 which is hereby incorporated by reference in its entirety.

In aspects, prior to transformation, Spirulina may be cultured and washed with an osmotic stabilizer. In aspects, an osmotic stabilizer may be a polyether including (e.g., w/v 0.1-20%) polyethylene glycol (PEG), poly(oxyethylene), or poly(ethylene oxide) (PEO). The PEG or PEO may have any molecular weight from 200 to 10,000, from 1000 to 6000, or from 2000 to 4000. In aspects, the pH balancer or buffer may be used instead of or in addition to the osmotic stabilizer.

Spirulina may be cultured in any suitable media for growth of cyanobacteria such as SOT medium. SOT medium includes NaHCO3 1.68 g, K₂HPO₄ 50 mg, NaNO₃ 250 mg, K₂SO₄ 100 mg, NaCl 100 mg, MgSO4·7H₂O, 20 mg, CaCl2·2H2O 4 mg, FeSO4·7H2O 1 mg, Na₂EDTA·2H₂O 8 mg, A5 solution 0.1 mL, and distilled water 99.9 mL. A5 solution includes H₃BO₃ 286 mg, MnSO4·5H₂O) 217 mg, ZnSO4·7H₂O 22.2 mg, CuSO4·5H₂O 7.9 mg, Na₂MoO4·2H₂O 2.1 mg, and distilled water 100 mL. Cultivation may occur with shaking (e.g., 100-300 rpm) at a temperature higher than room temperature (e.g., 25-37° C.) and under continuous illumination (e.g., 20-2,000, 50-500, or 100-200 μmol photon m-2 s-1). The growing cells may be harvested when the optical density at 750 nm reaches a predetermined threshold (e.g., OD750 of 0.3-2.0, 0.5-1.0, or 0.6-0.8). A volume of the harvested cells may be concentrated by centrifugation then resuspended in a solution of pH balancer and salt. The pH balancer may be any suitable buffer that maintains viability of Spirulina while keeping pH of the media between 6 and 9 pH, between 6.5 and 8.5 pH, or between 7 and 8 pH. Suitable pH balancers include HEPES, HEPES-NaOH, sodium or potassium phosphate buffer, and TES. The salt solution may be NaCl at a concentration of between 50 mM and 500 mM, between 100 mM and 400 mM, or between 200 mM and 300 mM. In aspects, between 1-50 mL of 1-100 mM pH balance may be used to neutralize the pH. Cells collected by centrifugation may be washed with an osmotic stabilizer and optionally a salt solution (e.g., 1-50 mL of 0.1-100 mM NaCl).

The present disclosure also describes creation of targeted mutations in Spirulina through homologous recombination by introduction of a vector to competent cells. In aspects, a vector of the disclosure comprises one or more methanolysins from Table 1 or a sequence having at least about 85%, 90%, 95%, 96%, 97%, 98%, 99%, or 100% identity to a sequence thereof. In aspects, a vector of the disclosure comprises a sequence from Table 1. In aspects, a vector of the disclosure comprises one or more methanolysins from Table 4 or a sequence having at least about 85%, 90%, 95%, 96%, 97%, 98%, 99%, or 100% identity to a sequence thereof. In aspects, a vector of the disclosure comprises a sequence from Table 4.

In aspects, the vector comprises a methanolysin comprising a sequence in Table 1. In aspects, the vector comprises the methanolysin comprising SEQ ID NO: 1. In aspects, the vector comprises the methanolysin comprising SEQ ID NO: 2. In aspects, the vector comprises the methanolysin comprising SEQ ID NO: 3. In aspects, the methanolysin comprise SEQ ID NO: 4. In aspects, the vector comprises the methanolysin comprising SEQ ID NO: 5. In aspects, the vector comprises the methanolysin comprising SEQ ID NO: 6. In aspects, the vector comprises the methanolysin comprising SEQ ID NO: 7. In aspects, the vector comprises the methanolysin comprising SEQ ID NO: 8. In aspects, the vector comprises the methanolysin comprising SEQ ID NO: 9. In aspects, the vector comprises the methanolysin comprising SEQ ID NO: 10. In aspects, the vector comprises the methanolysin comprising SEQ ID NO: 11. In aspects, the vector comprises the methanolysin comprising SEQ ID NO: 12. In aspects, the vector comprises the methanolysin comprising SEQ ID NO: 13. In aspects, the methanolysin comprise SEQ ID NO: 14. In aspects, the vector comprises the methanolysin comprising SEQ ID NO: 15. In aspects, the vector comprises the methanolysin comprising SEQ ID NO: 16. In aspects, the vector comprises the methanolysin comprising SEQ ID NO: 17. In aspects, the vector comprises the methanolysin comprising SEQ ID NO: 18. In aspects, the vector comprises the methanolysin comprising SEQ ID NO: 19. In aspects, the vector comprises the methanolysin comprising SEQ ID NO: 20. In aspects, the vector comprises the methanolysin comprising SEQ ID NO: 21. In aspects, the vector comprises the methanolysin comprising SEQ ID NO: 22. In aspects, the vector comprises the methanolysin comprising SEQ ID NO: 23. In aspects, the methanolysin comprise SEQ ID NO: 24. In aspects, the vector comprises the methanolysin comprising SEQ ID NO: 25. In aspects, the vector comprises the methanolysin comprising SEQ ID NO: 26. In aspects, the vector comprises the methanolysin comprising SEQ ID NO: 27. In aspects, the vector comprises the methanolysin comprising SEQ ID NO: 28. In aspects, the vector comprises the methanolysin comprising SEQ ID NO: 29. In aspects, the vector comprises the methanolysin comprising SEQ ID NO: 30. In aspects, the vector comprises the methanolysin comprising SEQ ID NO: 31. In aspects, the vector comprises the methanolysin comprising SEQ ID NO: 32. In aspects, the vector comprises the methanolysin comprising SEQ ID NO: 33. In aspects, the methanolysin comprise SEQ ID NO: 34. In aspects, the vector comprises the methanolysin comprising SEQ ID NO: 35. In aspects, the vector comprises the methanolysin comprising SEQ ID NO: 36. In aspects, the vector comprises the methanolysin comprising SEQ ID NO: 37. In aspects, the vector comprises the methanolysin comprising SEQ ID NO: 38. In aspects, the vector comprises the methanolysin comprising SEQ ID NO: 39. In aspects, the vector comprises the methanolysin comprising SEQ ID NO: 40. In aspects, the vector comprises the methanolysin comprising SEQ ID NO: 41. In aspects, the vector comprises the methanolysin comprising SEQ ID NO: 42. In aspects, the vector comprises the methanolysin comprising SEQ ID NO: 43. In aspects, the methanolysin comprise SEQ ID NO: 44. In aspects, the vector comprises the methanolysin comprising SEQ ID NO: 45. In aspects, the vector comprises the methanolysin comprising SEQ ID NO: 46. In aspects, the vector comprises the methanolysin comprising SEQ ID NO: 47. In aspects, the vector comprises the methanolysin comprising SEQ ID NO: 48. In aspects, the vector comprises the methanolysin comprising SEQ ID NO: 49. In aspects, the vector comprises the methanolysin comprising SEQ ID NO: 50. In aspects, the vector comprises the methanolysin comprising SEQ ID NO: 51. In aspects, the vector comprises the methanolysin comprising SEQ ID NO: 52. In aspects, the vector comprises the methanolysin comprising SEQ ID NO: 53. In aspects, the methanolysin comprise SEQ ID NO: 54. In aspects, the vector comprises the methanolysin comprising SEQ ID NO: 55. In aspects, the vector comprises the methanolysin comprising SEQ ID NO: 56. In aspects, the vector comprises the methanolysin comprising SEQ ID NO: 57. In aspects, the vector comprises the methanolysin comprising SEQ ID NO: 58. In aspects, the vector comprises the methanolysin comprising SEQ ID NO: 59. In aspects, the vector comprises the methanolysin comprising SEQ ID NO: 60. In aspects, the vector comprises the methanolysin comprising SEQ ID NO: 61. In aspects, the vector comprises the methanolysin comprising SEQ ID NO: 62. In aspects, the vector comprises the methanolysin comprising SEQ ID NO: 63. In aspects, the methanolysin comprise SEQ ID NO: 64. In aspects, the vector comprises the methanolysin comprising SEQ ID NO: 65. In aspects, the vector comprises the methanolysin comprising SEQ ID NO: 66. In aspects, the vector comprises the methanolysin comprising SEQ ID NO: 67. In aspects, the vector comprises the methanolysin comprising SEQ ID NO: 68. In aspects, the vector comprises the methanolysin comprising SEQ ID NO: 69. In aspects, the vector comprises the methanolysin comprising SEQ ID NO: 70. In aspects, the vector comprises the methanolysin comprising SEQ ID NO: 78. In aspects, the vector comprises the methanolysin comprising SEQ ID NO: 79. In aspects, the vector comprises the methanolysin comprising SEQ ID NO: 80. In aspects, the vector comprises the methanolysin comprising SEQ ID NO: 81. In aspects, the vector comprises the methanolysin comprising SEQ ID NO: 82. In aspects, the vector comprises the methanolysin comprising SEQ ID NO: 83. In aspects, the methanolysin comprise SEQ ID NO: 84. In aspects, the vector comprises the methanolysin comprising SEQ ID NO: 85. In aspects, the vector comprises the methanolysin comprising SEQ ID NO: 86. In aspects, the vector comprises the methanolysin comprising SEQ ID NO: 87. In aspects, the vector comprises the methanolysin comprising SEQ ID NO: 88. In aspects, the vector comprises the methanolysin comprising SEQ ID NO: 89. In aspects, the vector comprises the methanolysin comprising SEQ ID NO: 90. In aspects, the vector comprises the methanolysin comprising SEQ ID NO: 91. In aspects, the vector comprises the methanolysin comprising SEQ ID NO: 92. In aspects, the vector comprises the methanolysin comprising SEQ ID NO: 93. In aspects, the methanolysin comprise SEQ ID NO: 94. In aspects, the vector comprises the methanolysin comprising SEQ ID NO: 95. In aspects, the vector comprises the methanolysin comprising SEQ ID NO: 96. In aspects, the vector comprises the methanolysin comprising SEQ ID NO: 97. In aspects, the vector comprises the methanolysin comprising SEQ ID NO: 98. In aspects, the vector comprises the methanolysin comprising SEQ ID NO: 99. In aspects, the vector comprises the methanolysin comprising SEQ ID NO: 100.

In aspects, the vector comprises a methanolysin comprising a sequence in Table 4. In aspects, the vector comprises the methanolysin comprising SEQ ID NO: 101. In aspects, the vector comprises the methanolysin comprising SEQ ID NO: 102. I

In aspects, the strains comprising the Spirulina-methanolysin cocktail methane mitigation product may vary regionally to specifically target the species in a given locality.

In aspects, the Spirulina-methanolysin strains can also be applied to other material containing methanogens to kill the organism and prevent methane production. For example, ruminant manure also contains methanogens that produce and emit methane, a potent GHG. The Spirulina-methanolysin strains can be applied directly to the manure to inhibit methane production.

In aspects, during the manufacturing process, the Spirulina-methanolysin biomass can be lysed, filtered, and the resulting soluble extract spray dried to produce a dried powder product containing the methanolysin. This product would allow the methanolysin to be immediately activated and available after resuspension. In contrast, the feed additive invention described above using spray dried whole cell biomass would require that the biomass be exposed to the appropriate pH to release the lysin from the cells. The whole cell biomass may be the superior feed additive by providing some protection from proteases to the methanolysin until it reaches the desired site of action, but the spray dried extract may be superior for other use cases.

In aspects, other biologics that kill methanogens may be expressed in Spirulina to inhibit methane production.

In aspects, multiple methanolysins could be expressed within a composition comprising Spirulina cells to generate a cocktail. In aspects, two or more methanolysins are expressed by a single Spirulina cell. In aspects, two or more methanolysins are expressed by two or more different Spirulina cells.

Spirulina is grown at commercial scale in outdoor ponds to produce nutritional supplements sold for about $10/kg. Harvested biomass is spray dried to produce a stable powder product that does not require a cold chain for distribution or storage. The high potential potency of our feed additive-engineered Spirulina can accumulate up to 15% recombinant protein as a proportion of dry weight-could result in a product that eliminates methanogenesis in cattle with modest administration of biomass (<0.1% dry matter intake). The provided Spirulina-methanolysin strains, manufactured using current industry practices achieves a price point that makes livestock administration feasible. Accordingly, the provided compositions result in increased manufacturing scalability as compared to otherwise comparable compositions lacking modified Spirulina.

In aspects, the removal of methanogens from the rumen also reduces dry matter intake without a corresponding reduction in daily weight gain. Accordingly, the provided compositions result in increased economic benefit as compared to otherwise comparable compositions lacking modified Spirulina.

### Populations of Spirulina

In aspects, provided herein are also populations of Spirulina that comprises a modified Spirulina cell. In aspects, a population comprises two or more Spirulina cells. In aspects, the two or more Spirulina cells comprise different methanolysin proteins. In aspects, the two or more Spirulina cells each comprise the different methanolysin proteins. In aspects, the two or more Spirulina cells comprise two or more different methanolysin proteins from Table 1 or Table 4, or engineered or modified versions thereof.

In aspects, the population of Spirulina cells comprises three or more different methanolysin proteins from Table 1. In aspects, the population of Spirulina cells comprises four or more different methanolysin proteins from Table 1. In aspects, the population of Spirulina cells comprises five or more different methanolysin proteins from Table 1. In aspects, the population of Spirulina cells comprises six or more different methanolysin proteins from Table 1. In aspects, the population of Spirulina cells comprises seven or more different methanolysin proteins from Table 1. In aspects, the population of Spirulina cells comprises eight or more different methanolysin proteins from Table 1. In aspects, the population of Spirulina cells comprises nine or more different methanolysin proteins from Table 1. In aspects, the population of Spirulina cells comprises ten or more different methanolysin proteins from Table 1.

In aspects, the population of modified Spirulina is formulated for oral administration. In aspects, the two or more Spirulina cells express different methanolysin proteins that comprise an amino acid sequence with at least about 80%, 85%, 90%, 95%, 98%, or 99% identity to SEQ ID NOs: 101 and 102. In aspects, the two or more Spirulina cells comprise SEQ ID NOs: 101 and 102 methanolysins. In aspects, the two or more Spirulina cells comprise SEQ ID NOs: 103 and 104 methanolysins.

### Supplements and Nutraceuticals

Provided herein are also supplements and nutraceuticals. In aspects, a food supplement composition comprises a modified Spirulina cell. In aspects, the food supplement is selected from a group comprising of minerals, vitamins, protein, fiber, probiotics, growth promoters, natural additives, and hydration supplements. In aspects, the food supplement is selected from the group comprising of salt block, calcium supplements, phosphorus supplements, selenium supplements, magnesium supplements, Vitamin A, Vitamin D, Vitamin E, Vitamin K, Vitamin B complex, soybean meal, cottonseed meal, fish meal, blood meal, alfalfa meal, corn, barley, oats, wheat, molasses, hay, straw, alfalfa, beet pulp, silage, probiotic supplements, yeast supplements, enzyme supplements, ionophores (e.g., monensin, lasalocid), hormonal supplements, apple cider vinegar, garlic, oregano oil, diatomaceous earth, electrolyte powders and water additives.

In aspects, a nutraceutical composition comprises a modified Spirulina cell. In aspects, the nutraceutical is selected from the group comprising of prebiotics, probiotics, essential fatty acids, antioxidants, herbal extracts, mycotoxin binders, immune modulators, gut health enhancers, joint supplements, natural growth promoters, stress alleviators, bone health supplements, organic acids, enzymes. In aspects, the nutraceutical is selected from a group comprising of: fructooligosaccharides (fos), mannan oligosaccharides (mos), inulin, Lactobacillus spp., Bifidobacterium spp., Enterococcus spp., Saccharomyces cerevisiae, omega-3 fatty acids, omega-6 fatty acids, Vitamin C, Vitamin E, selenium, zinc, echinacea, ginseng, licorice root, milk thistle, bentonite, activated charcoal, yeast cell walls, beta-glucans, mannoproteins, yeast-derived products, butyric acid, short-chain fatty acids, glutamine, glucosamine, chondroitin sulfate, MSM (methylsulfonylmethane), essential oils (e.g., thyme, oregano), plant extracts (e.g., garlic, turmeric), adaptogens (e.g., ashwagandha, rhodiola), L-tryptophan, calcium, phosphorus, Vitamin D3, formic acid, propionic acid, acetic acid, phytase, amylase, and protease.

In aspects, the nutraceutical composition is administered to a ruminant. In aspects, the nutraceutical composition is administered to a human.

The supplements and nutraceuticals of the disclosure can comprise one or more of the modified Spirulina expressing one or more methanolysins of the disclosure.

### Pharmaceutical and Non-Pharmaceutical Compositions and their Administration

Administration of the compositions of the present disclosure can be via any method which delivers a compound of this disclosure systemically and/or locally, including oral routes, sublingual routes, transdermal routes, etc. In accordance with this formulation, the compound of the present disclosure may be in a solid, semi-solid, or liquid form. Thus, the compositions of the present disclosure can be administered by any of the following administration methods where necessary and/or appropriate: orally, sublingually, parenteral administration (e.g., intravenous (IV), intramuscular, subcutaneous (SQ or Sub-Q) injections, or intramedullary), transdermal, via inhalation, topically, via suppository, sublingual administration, and buccal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions, or solutions.

A pharmaceutical composition can be in the form of a hard or soft gelatin capsule. Capsules may contain mixtures of a compound of the present disclosure with inert fillers and/or diluents such as the pharmaceutically acceptable starches (e.g., corn, potato, or tapioca starch), sugars, artificial sweetening agents, powdered celluloses, such as crystalline and microcrystalline celluloses, flours, gelatins, gums, etc. For oral administration in a capsule form, useful diluents include lactose and dried corn starch.

If desired, certain sweetening and/or flavoring and/or coloring agents may be added. In aspects, the initial amount of flavoring and/or flavoring oil utilized in the methods and compositions of the present disclosure is zero (i.e., no flavoring or flavoring oil is added).

In aspects, the tablet can comprise a unit dosage of a compound of the present disclosure together with an inert diluent or carrier such as a sugar or sugar alcohol, for example lactose, sucrose, sorbitol, or mannitol. In the case of tablets for oral use, carriers which are commonly used include lactose and corn starch. Lubricating agents, such as magnesium stearate, can also be added. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g., swellable crosslinked polymers such as crosslinked carboxymethylcellulose), lubricating agents (e.g., stearates), preservatives (e.g., parabens), antioxidants (e.g., BHT), buffering agents (for example phosphate or citrate buffers), and effervescent agents such as citrate/bicarbonate mixtures.

The tablet can be a coated tablet. The coating can be a protective film coating (e.g., a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

When aqueous suspensions and/or emulsions are administered orally, the compound of the present disclosure may be suspended or dissolved in an oily phase is combined with emulsifying and/or suspending agents.

The pharmaceutical compositions for use in the methods of the present disclosure can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

Among the surfactants for use in the compositions of the disclosure are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present disclosure also provides packaging and kits comprising pharmaceutical compositions for use in the methods of the present disclosure. The kit can comprise one or more containers selected from the group consisting of a bottle, a vial, an ampoule, a blister pack, and a syringe. The kit can further include one or more of instructions for use in treating and/or preventing a disease, condition, or disorder of the present disclosure, one or more syringes, one or more applicators, or a sterile solution suitable for reconstituting a pharmaceutical composition of the present disclosure.

The following references, provide detailed, background information on methods of preparing pharmaceutical compositions, methods of formulating pharmaceutical compositions, and methods of their administration: U.S. Pat. Nos. 8,278,277; 8,776,802; 9,789,104; 10,463,736; 9,034,376; 10,052,286; 10,292,966; 11,878,025; 9,072,663; 9,408,835; 9,457,086; 9,918,982; 9,274,109; 9,480,661; 10,500,282; 10,226,423; 11,541,021; 11,975,104; 11,957,691; 11,877,999; 11,865,372; 11,826,325; 11,813,314; 11,773,394; 11,701,402; 11,654,183; and 11,654,114. Some of the detailed description in this section are derived from one or more of these references.

In aspects, the compositions, including pharmaceutical compositions, disclosed herein may be prepared by any suitable method including but not limited to spray-drying, exposure to hot air, refractory window belt drying, drying in an oven, vacuum drying, vacuum belt drying, continuous vacuum belt drying, conveyor belt with vacuum suction, evaporation, fluidized bed drying, lyophilization, and combinations thereof. The following references provide detailed, background information on methods of preparing compositions: U.S. Pat. Nos. 6,470,597; 8,586,094; 9,427,604; 10,512,654; 10,639,283; 10,973,759; 11,191,726; 11,197,843; 11,433,027; 11,660,272; 11,752,105; 11,964,002; 11,801,222; and 11,938,133. See also, e.g., Kozanoglu et al., (2012), Influence of particle size on vacuum-fluidized bed drying, Drying Technology, 30 (2), 138-145; Haron et al., (2017), Recent advances in fluidized bed drying, IOP Conf. Mater. Sci. Eng. 243, 012038; Majumder et al., (2022), A comprehensive review of fluidized bed drying: sustainable design approaches, hydrodynamic and thermodynamic performance characteristics, and product quality, Sustainable Energy Technologies and Assessments, Vol. 53, Part C; Lakat et al., (2021), Lyophilization and homogenization of biological samples improves reproducibility and reduces standard deviation in molecular biology techniques, Amino Acids, 53 (6): 917-928; P. Manohar and N. Ramesh, (2019), Improved lyophilization conditions for long-term storage of bacteriophages. Sci Rep 9, 15242; Kasper et al., (2013), Recent advances and further challenges in lyophilization, European Journal of Pharmaceutics and Biopharmaceutics, Vol. 85, Issue 2:162-169; Sinha, et al., (2022). Manipulation of spray-drying conditions, Pharmaceutics, 14 (7): 1432; F. Gaspar, (2014), Spray drying in the pharmaceutical industry, European Pharmaceutical Review, Issue 5; and Pinto et al., (2021) Progress in spray-drying of protein pharmaceuticals: literature analysis of trends in formulation and process attributes, Drying Technology, 39 (11): 1415-1446.

In aspects, the compositions, including pharmaceutical compositions, disclosed herein may also comprise other conventional pharmaceutically acceptable ingredients, commonly referred to as carriers, excipients, or adjuvants. Excipients or adjuvants include, but are not limited to: disintegrants, binders, lubricants, glidants, stabilizers, fillers, diluents, colorants, sweeteners, flavoring agents, and preservatives. For example, useful additives include materials such as agents for retarding dissolution (e.g., paraffin), resorption accelerators (e.g., quaternary ammonium compounds), surface active agents (e.g., cetyl alcohol, glycerol monostearate, and sodium lauryl sulfate), adsorptive carriers (e.g., kaolin and bentonite), preservatives, sweeteners, coloring agents, flavoring agents (e.g., chocolate mint, citric acid, menthol, glycine or orange powder), stabilizers (e.g., citric acid or sodium citrate), binders (e.g., hydroxypropylmethylcellulose), and mixtures thereof. Those of ordinary skill in the art may, by conventional experimentation, select one or more of the above carriers based on the desired properties of the dosage form without undue burden. The amount of each carrier used is within the conventional range in the art.

In aspects, the compositions of the present disclosure may be a powdered extract which may optionally be combined with one or more inactive, neutral compounds/ingredients which can be pharmaceutically acceptable excipients or carriers, including, but not limited to, binders, antioxidants, adjuvants, synergists and/or preservatives.

Some aspects of the present disclosure are directed to dosage forms that are formulated as solid articles suitable for sublingual or oral administration, such as troches, lozenges, pills, oral dissolving strips, caps, pouches, or boluses. These solid dosage forms may comprise additional excipients. Both hard and chewable lozenges and troches are within the scope of the present disclosure. In aspects, the oral dosage forms are formulated as capsules in which the compositions of the present disclosure are encapsulated in soft or hard gelatin capsules.

Some aspects of the present disclosure are directed to dosage forms that are formulated as topical formulations, including but not limited to creams, ointments, and gel, using formulation methods as are known in the art. In aspects, the topical formulation is a transdermal patch, using formulation methods and technologies as are known in the art.

Some aspects of the present disclosure are directed to dosage forms that are formulated as solid articles suitable for administration as vaginal ovules or rectal suppositories, using formulation methods as are known in the art.

Some aspects of the present disclosure are directed to dosage forms that are formulated as liquids, including but not limited to emulsions, liposomes, dispersions, oils, and tinctures, and beverages using formulation methods as are known in the art. A dispersible or liquid format is probably required for pediatric medical delivery.

In aspects, the dosage forms of the present disclosure may optionally be formulated to further comprise one or several adjuvants or synergists. If adjuvants or synergists are used, non-limiting examples of those that can be used include citric acid, EDTA (ethylenediaminetetraacetate) and salts, hydroxyquinoline sulfate, phosphoric acid, and/or tartaric acid.

In aspects, the dosage forms of the present disclosure may optionally further comprise one or several preservatives. If preservatives are used, non-limiting examples of those that can be used include benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sodium nitrate, potassium sorbate, sorbic acids and salts, o-phenylethyl alcohol, and/or thimerosal.

Examples of pharmaceutically acceptable surfactants for use in the present disclosure include but are not limited to polyvinylpyrrolidone, polyethylene glycol surfactants, oleic acid, and/or lecithin.

Examples of pharmaceutically acceptable lubricants and pharmaceutically acceptable glidants include, but are not limited to: silica gel, magnesium trisilicate, starch, talc, tricalcium phosphate, magnesium stearate, aluminum stearate, calcium stearate, magnesium carbonate, magnesium oxide, polyethylene glycol, powdered cellulose, and/or microcrystalline cellulose.

Examples of pharmaceutically acceptable fillers and pharmaceutically acceptable diluents include, but are not limited to: powdered sugar, compressible sugar, glucose binding agents, dextrin, dextrose, lactose, mannitol, maltitol, xylitol, microcrystalline cellulose, powdered cellulose, sorbitol, sucrose, and/or talc.

The amount of each composition or formulation of the present disclosure that may be combined with the carrier materials to produce a single dosage form will vary depending upon the individual treated and the mode of administration. Individual characteristics to be considered for proper dosage include but are not limited to weight, height, body mass index, age, health status, existing conditions, etc. In aspects, when the composition or formulation comprises another active agent in addition to the active ingredient, the unit dosage forms as described herein will contain a certain amount of each agent of the combination that is typically administered when the agent is administered alone.

In aspects, the drug-carrier complex disclosed herein is administered to a patient in need thereof in the form of a pharmaceutical composition. In aspects, the drug-carrier complex disclosed herein is present in a pharmaceutically effective amount.

The pharmaceuticals of the disclosure can comprise one or more of the modified Spirulina expressing one or more methanolysins of the disclosure.

### Vectors

Provided are also vectors that encode for methanolysins. Vectors of the disclosure can be polynucleotide molecules, preferably a DNA molecule derived, for example, from a plasmid, bacteriophage, yeast, or virus, into which a polynucleotide can be inserted or cloned. A vector may contain one or more synthetic nucleotides or nucleic acid analogues. A vector may contain one or more unique restriction sites. The vector can be one which, when introduced into the host cell, is integrated into the genome and replicated together with the chromosome(s) into which it has been integrated. Such a vector may comprise one or more specific sequences that allow recombination into a particular, desired site of the host chromosome. These specific sequences may be homologous to sequences present in the wild-type genome. A vector system can comprise a single vector or plasmid, two or more vectors or plasmids, some of which increase the efficiency of targeted mutagenesis, or a transposition. The choice of the vector will typically depend on the compatibility of the vector with the host cell into which the vector is to be introduced. The vector can include a reporter gene, such as a green fluorescent protein (GFP), which can be either fused in frame to one or more of the encoded polypeptides or expressed separately. The vector can also include a positive selection marker such as an antibiotic resistance gene that can be used for selection of suitable transformants. The vector can also include a negative selection marker such as the type II thioesterase (tesA) gene or the Bacillus subtilis structural gene (sacB). Use of a reporter or marker allows for identification of those cells that have been successfully transformed with the vector.

An exemplary vector of the disclosure can comprise a sequence with at least about 50%, 60%, 70%, 80%, 90%, 95%, 97%, 98%, 99%, or up to 100% identity to:

(SEQ ID NO: 124)
CGAGCGGTATCAGCTCACTCAAAGGCGGTAATACGGTTATCCACAGAATCA
GGGGATAACGCAGGAAAGAACATGTGAGCAAAAGGCCAGCAAAAGGCCAG
GAACCGTAAAAATAGCGGAGTGTATACTGGCTTACTATGTTGGCACTGAT
GAGGGTGTCAGTGAAGTGCTTCATGTGGCAGGAGAAAAAAGGCTGCACCG
GTGCGTCAGCAGAATATGTGATACAGGATATATTCCGCTTCCTCGCTCAC
TGACTCGCTACGCTCGGTCGTTCGACTGCGGCGAGCGGAAATGGCTTACG
AACGGGGCGGAGATTTCCTGGAAGATGCCAGGAAGATACTTAACAGGGAA
GTGAGAGGGCCGCGGCAAAGCCGTTTTTCCATAGGCTCCGCCCCCCTGAC
AAGCATCACGAAATCTGACGCTCAAATCAGTGGTGGCGAAACCCGACAGG
ACTATAAAGATACCAGGCGTTTCCCCCTGGCGGCTCCCTCGTGCGCTCTC
CTGTTCCTGCCTTTCGGTTTACCGGTGTCATTCCGCTGTTATGGCCGCGT
TTGTCTCATTCCACGCCTGACACTCAGTTCCGGGTAGGCAGTTCGCTCCA
AGCTGGACTGTATGCACGAACCCCCCATTCAGTCCGACCGCTGCGCCTTA
TCCGGTAACTATCGTCTTGAGTCCAACCCGGAAAGACATGCAAAAGCACC
ACTGGCAGCAGCCACTGGTAATTGATTTAGAGGAGTTAGTCTTGAAGTCA
TGCGCCGGTTAAGGCTAAACTGAAAGGACAAGTTTTGGTGACTGCGCTCC
TCCAAGCCAGTTACCTCGGTTCAAAGAGTTGGTAGCTCAGAGAACCTTCG
AAAAACCGCCCTGCAAGGCGGTTTTTTCGTTTTCAGAGCAAGAGATTACG
CGCAGACCAAAACGATCTCAAGAAGATCATCTTATTAAGATTATCAAAAA
GGATCTTCACCTAGATCCTTTTAAATTAAAAATGAAGTTTTAAATCAATC
TAAAGTATATATGAGTAAACTTGGTCTGACAGTTACCAATGCTTAATCAG
TGAGGCACCTATCTCAGCGATCTGTCTATTTCGTTCATCCATAGTTGCCT
GACTCCCCGTCGTGTAGATAACTACGATACGGGAGGGCTTACCATCTGGC
CCCAGTGCTGCAATGATACCGCGGGACCCACGCTCACCGGCTCCAGATTT
ATCAGCAATAAACCAGCCAGCCGGAAGGGCCGAGCGCAGAAGTGGTCCTG
CAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCG
GTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGC
AGTGTTATCACTCATGGTTATGGCAGCACTGCATAATTCTCTTACTGTCA
TGCCATCCGTAAGATGCTTTTCTGTGACTGGTGAGTACTCAACCAAGTCA
TTCTGAGAATAGTGTATGCGGCGACCGAGTTGCTCTTGCCCGGCGTCAAT
ACGGGATAATACCGCGCCACATAGCAGAACTTTAAAAGTGCTCATCATTG
GAAAACGTTCTTCGGGGCGAAAACTCTCAAGGATCTTACCGCTGTTGAGA
TCCAGTTCGATGTAACCCACTCGTGCACCCAACTGATCTTCAGCATCTTT
TACTTTCACCAGCGTTTCTGGGTGAGCAAAAACAGGAAGGCAAAATGCCG
CAAAAAAGGGAATAAGGGCGACACGGAAATGTTGAATACTCATACTCTTC
CTTTTTCAATATTATTGAAGCATTTATCAGGGTTATTGTCTCATGAGCGG
ATACATATTTGAATGTATTTAGAAAAATAAACAAATAGGGGTTCCGCGCA
CATTTCCCCGAAAAGTGCCACCTGACGTCTAAGAAACCATTATTATCATG
ACATTAACCTATAAAAATAGGCGTATCACGAGGCCCTTTCGTCTCGCGCG
TTTCGGTGATGACGGTGAAAACCTCTGACACATGCAGCTCCCGGAGACGG
TCACAGCTTGTCTGTAAGCGGATGCCAAGCTTGCATGCCTGCAGGTGGCG
CGCCTATGGGGGAGGATTTTATGATCGGATGTTGAGTCAGTCTGAGTGGT
CTAAAATTCCTACGGTGGGAATTGTGTTTGCGGCCGCCAGGGTGGATACT
CTCCCTAGGGATAGTTGGGACCAGCCTTTGGCGGCGGTTTGTACAGAGGA
GGGTATATGGGAGTTTCCAAAATAACTTCTGGTGGGGGGGCAAATTATCG
GGCGATCGCCACTTATATGGTATTAATTGCTATAGCGATCGCTATGCTAT
TTCCCCTATTTTGGTTGGTGGGAACTGCCTTTAAATCCCCTACCGAAAAC
ATCTTTCAGGTTCCCCCCCAGTTTATTCCAAGGGAACCTACTTTCACTAA
TTTTGTCAGAGTTTGGCAAACCAATCCCTTTGGTCGCTATTTATTTAATA
GTACCCTCGTCGCCCTTTTAACCGTCGGATTAAATCTGCTATTTTGTTCC
TTGGCGGCCTATCCTTTGGCGCGCTTAAACTTTTGGGGTCGCGATTTTCT
CCTAAGTGCGATCGTTTCCACCATCATGATACCCTTTCAGATTGTCATGA
TTCCCCTGTATATTCTAGCAGTACAGTTAGGATTAAGAAATACTTATTTA
GGCATTATTTTTCCATCTTTAGCCTCCGCTTTTGGCATCTTTCTATTGCG
ACAGGCATTTATGGGAGTTCCCAAAGAGTTAGAAGAAGCCGCCAGAATTG
ATGGCTGTTCAGAATTAGGGATTTGGTGGCATATTATGATCCCCTCAGTC
CGTCCCGCCTTAATGACCTTAGCCATTTTTGTATTTATCGGGTCTTGGAG
TGATTTTCTCTGGCCCTTAATTGTAGTCGATCGCCCAGAATATTATACCA
TCCCTTTAGGCGTGGCAAATCTAGCCGGAACTTTTTCTCTCGATTGGCGG
ATAGTCGCCGCCGGTTCCGTCATTTCTATCGCTCCTGTGTTACTACTATT
TTTAATCGTACAACGGTATATTGTGCCTACAGATACAGCTACGGGGGTTA
AAGGTTGAATGAGGATTATTAATCTCAGTCCTAATAATCAAAATCATATC
CACCAGGCGGCGACTTTATTAGTCGCAGAATTTCGGGAAAATTGGCCGAA
TGCTTGGCCGACTTATGAACGTGGTTTAGCCGAGGTTATGGAGTCTTTTG
GCGACGATAGAGTTAATCTAGTCGCCGTCGATGAAAATGATAATCTTCTA
GGCTGGATTGGTGGTATTAGCCAATATCAAGGCCATGTTTGGGAATTACA
TCCTTTGGTAGTCAAAAGCGATTATCAAGGGTTAGGAATAGGGCGAAAAT
TGGTCGCCAATTTAGAGGATTATGTCCGCTCACAAGGAGGGTTAACCTTA
TGGTTGGGTACCGATGACGAAAATAATTTAACCTCCCTATCTGGTGTCGA
ATTATACCCCCATTTTTTGGAAAATATTGCTAACATAAAAAACCATGGTC
GTCATCCCTATGAATTTTATCAGAAATGCGGTTTTGTGATTATGGGAGTA
GTCCCCGATGCTAACGGTATCGGGAAACCTGATATTTTGATGGCTAAATC
TCTGAGAATTGACAATCATCCTAAATCCAACTGAATGTTGTCAGTTAAGT
TTATAGGGCTGCTATGGTATGATAAAAAAATCCCCCCAGCAGTTGGGGGG
GAATGATTAATTAGGTATTGAATTGGTTAAATTGGAGGTGTGTTGGGGCT
GACTTTTGCTGCCTTGTTGGGGGGGGCGATTTTAACTGAGGTCACTTTTT
CTTGGCCGGGATTGGCTAATCGCTTGTATGAGGCTATCCGTCTGCGGGAT
TATCCCACGGTTCAAGGGGTGGTGGTATTTTTGGCCGTTATTGTGGTGGC
GGCTAGTTTGGCGATCGATATTTTGAATGCTTGTATTGACCCACGGATTC
GCTATTAATTCCCTGTCAGAAAATATACTTAGTTATTAATACTTAGTTAT
TTTTCTTAATAAATTTTAACAAACCAAAGGGCGTTGGCAGTGTATAAAGG
ATATTTGAAGGGGTGAGAAGGAAGGCGAATGTTGATTTATGAAGTTTGAT
TAACATTTGTATCAAAATATAAAATTCTTCTCATAAACCCTGTACAATCT
TTTAAGATTTCGGAAAGTGTTCTAGGATACTGAAGAAATGAACCACGGGG
CAATTGTTAAAAGCCTTTGTCGATGGTTCGCCCCGGAAGGGGTCTTAGGA
GGTGACACCGATGGATTGATTGTCGTGATCATTCATGGTGTGTCCAATCC
CAACTCAACTCTAAGCAAGTCAACAAGTAGGAGATAAATCTATGTGAGAC
CACTAGTGGTCTCTCAGCCAAATAAAACGAAAGGCTCAGTCGAAAGACTG
GGCCTTTCGTTTTATCTGTTGTTTGTCGGTGAACGCTCTCCTGAGTAGGA
CAAATCCGCCGGGAGCGGATTTGAACGTTGCGAAGCAACGGCCCGGAGGG
TGGCGGGCAGGACGCCCGCCATAAACTGCCAGGCATCAAATTAAGCAGAA
GGCCATCCTGACGGATGGCCTTTTTGCGTTTCTACAAACTCCGGATCCGG
CCGGCTTGAAGACAGNNNNNNNNNNNNNNNNNNNAATGCGGAATCATGAA
TCATCCCACAACTAAGCATCAAAAAATTACATCCTGTTACTGGGGAATTT
CTGAACTACCTGGGTTAGATACAGGTAATATTAAACTCCTGAATCAATGT
GGTATTGAAAACACCCAGCAACTGCTCACGCGCGGGAGTAATAATGCTAA
TAAAATCGCCTTATCTAACCAACTAGGTATCAACATCAGAGATATCAGTA
AATGGGTCGCAATGTCCGACTTAGCCAGGGTTCCCAGTATTGGCTATCAA
TATTGTGGTGTCCTTTTGCATAGTGGCGTAAGTTCAGTTAACCAACTATC
TCAGATGTCTATTCAACAACTGCATAAACAGATTTTACGGCTCTATGTAG
GAACCTTGCAATCTCGCCAACTCTGTCCATCTGTTGATGTCATCCAAACC
TGGATTAAACAAGCTAAAATTTTGCATAAAATTAATTAATAATTACTAGA
TGAAATCATGAATTTTTCCGAAGATTTGCGACAAAATGCACCAGCCACCG
AAAGAAACCGCGAACCTATTTTAGAGGTCTTAAAAAGGGTGTTGCCCGCC
ACGGGAACCGTTTTAGAAGTAGCCAGCGGCACAGGACAACACGCGGTTTT
TTTTGCTCCCCGTCTCTCCCCTAGGCGGTGGCTACCTTCCGACCCGCAAC
CTTTGTTACAACAGAGTATTAAAGCATGGCAAATTCATTCACCTTCAGAT
AATCTTTACCCTCCCCTACAGTTAAATGTAGAAGCTAATCCTTGGCCAGT
GGAAGGGAATGATTTACCCTGGGAATTATCGGAATTTCCCATCACTGCTA
TTGTAGCAATTAACCTGATTCATATTTCGCCTTGGTCGGCTTGTGAAGGG
TTAATGGCGGGAAGCGATCGCATTTTGAAACCGGGGGGAATTTTATATCT
CTATGGACCCTATAAAATAGCTGGAAAACACACCGCACCTAGTAACGCTT
CCTTTGATGAATATTTGCGAACTTCTAACCCTAAGTGGGGTGTCAGAAAC
CTTGATGATTCACATACGCGGCCGCCTGGGCCTTGAGCTCGAATTTCCTG
CATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGC
GG.

In aspects, any of the methanolysins of the disclosure can be expressed via the aforementioned vector. For example, any of the methanolysins of Table 1 or Table 4 can be encoded by the vector of SEQ ID NO: 124. In an exemplary cloning scheme, the vector of SEQ ID NO: 124 is digested with the BsaI restriction enzyme to generate two overhangs that facilitate insertion of a coding sequence of a methanolysin of the disclosure, such as any one of Table 1 or Table 4.

To ease cloning of transgenes into Spirulina, a vector with markerless integration can be utilized. In aspects, a vector comprises integrating homology arms for the kmR locus flanking an ORF for the native kmR gene and a terminator. In aspects, a vector further comprises an antibiotic marker followed by a recombinant promoter-terminator pair for transgene expression. In aspects, a promoter-terminator pair comprises a constitutively active, native A. platensis promoter (e.g., 600 bp upstream of the cpcB gene, P_{cpc600}) and the terminator of the E. coli ribosomal RNA gene rrnB (named TrrnB). In aspects, a vector further comprises a pair of Batrachochytrium salamandrivorans restriction endonuclease sites between the promoter-terminator pair.

In aspects, vectors include one or two homology arms that are homologous to DNA sequences of the Spirulina genome which are adjacent to a targeted locus. The sequence of the homology arms may be identical or similar to the regions of the Spirulina genome to which the homology arms are complementary. “Homology” or “homologous” as used herein includes both homologous, identical sequences and homologous, non-identical sequences. Homologous non-identical sequences refer to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. As used herein, the degree of homology between the two homologous, non-identical sequences is sufficient to allow homologous recombination there between, utilizing normal cellular mechanisms. Two homologous non-identical sequences can be any length and their degree of non-homology can be as small as a single nucleotide (e.g., for a genomic point mutation introduced targeted homologous recombination) or as large as 10 or more kilobases (e.g., for insertion of a gene at a predetermined locus in a chromosome). Two polynucleotides comprising homologous non-identical sequences need not be the same length. For example, an exogenous polynucleotide (i.e., vector polynucleotide) of between 20 and 10,000 nucleotides or nucleotide pairs can be used.

The characterization of two sequences as homologous, identical sequences or homologous, non-identical sequences may be determined by comparing the percent identity between the two sequences (polynucleotide or amino acid). Homologous, identical sequences have 100% sequence identity. Homologous, non-identical sequences may have sequence identity greater than 80%, greater than 85%, greater than 90%, greater than 91%, greater than 92%, greater than 93%, greater than 94%, greater than 95%, greater than 96%, greater than 97%, greater than 98%, or greater than 99% to a sequence of: kanamycin aminoglycoside acetyltransferase, NS1, and both kanamycin aminoglycoside acetyltransferase and NS1.

In aspects, homology arms target a kanamycin aminoglycoside acetyltransferase gene. In aspects, a left homology arms comprises the nucleotide sequence:

(SEQ ID NO: 103)
TATGGGGGAGGATTTTATGATCGGATGTTGAGTCAGTCTGAGTGG
TCTAAAATTCCTACGGTGGGAATTGTGTTTGCGGCCGCCAGGGTG
GATACTCTCCCTAGGGATAGTTGGGACCAGCCTTTGGCGGCGGTT
TGTACAGAGGAGGGTATATGGGAGTTTCCAAAATAACTTCTGGTG
GGGGGGCAAATTATCGGGCGATCGCCACTTATATGGTATTAATTG
CTATAGCGATCGCTATGCTATTTCCCCTATTTTGGTTGGTGGGAA
CTGCCTTTAAATCCCCTACCGAAAACATCTTTCAGGTTCCCCCCC
AGTTTATTCCAAGGGAACCTACTTTCACTAATTTTGTCAGAGTTT
GGCAAACCAATCCCTTTGGTCGCTATTTATTTAATAGTACCCTCG
TCGCCCTTTTAACCGTCGGATTAAATCTGCTATTTTGTTCCTTGG
CGGCCTATCCTTTGGCGCGCTTAAACTTTTGGGGTCGCGATTTTC
TCCTAAGTGCGATCGTTTCCACCATCATGATACCCTTTCAGATTG
TCATGATTCCCCTGTATATTCTAGCAGTACAGTTAGGATTAAGAA
ATACTTATTTAGGCATTATTTTTCCATCTTTAGCCTCCGCTTTTG
GCATCTTTCTATTGCGACAGGCATTTATGGGAGTTCCCAAAGAGT
TAGAAGAAGCCGCCAGAATTGATGGCTGTTCAGAATTAGGGATTT
GGTGGCATATTATGATCCCCTCAGTCCGTCCCGCCTTAATGACCT
TAGCCATTTTTGTATTTATCGGGTCTTGGAGTGATTTTCTCTGGC
CCTTAATTGTAGTCGATCGCCCAGAATATTATACCATCCCTTTAG
GCGTGGCAAATCTAGCCGGAACTTTTTCTCTCGATTGGCGGATAG
TCGCCGCCGGTTCCGTCATTTCTATCGCTCCTGTGTTACTACTAT
TTTTAATCGTACAACGGTATATTGTGCCTACAGATACAGCTACGG
GGGTTAAAGGTTGA.

In aspects, a right homology arm comprises the nucleotide sequence

(SEQ ID NO: 104)
GGAATCATGAATCATCCCACAACTAAGCATCAAAAAATTACATCC
TGTTACTGGGGAATTTCTGAACTACCTGGGTTAGATACAGGTAAT
ATTAAACTCCTGAATCAATGTGGTATTGAAAACACCCAGCAACTG
CTCACGCGCGGGAGTAATAATGCTAATAAAATCGCCTTATCTAAC
CAACTAGGTATCAACATCAGAGATATCAGTAAATGGGTCGCAATG
TCCGACTTAGCCAGGGTTCCCAGTATTGGCTATCAATATTGTGGT
GTCCTTTTGCATAGTGGCGTAAGTTCAGTTAACCAACTATCTCAG
ATGTCTATTCAACAACTGCATAAACAGATTTTACGGCTCTATGTA
GGAACCTTGCAATCTCGCCAACTCTGTCCATCTGTTGATGTCATC
CAAACCTGGATTAAACAAGCTAAAATTTTGCATAAAATTAATTAA
TAATTACTAGATGAAATCATGAATTTTTCCGAAGATTTGCGACAA
AATGCACCAGCCACCGAAAGAAACCGCGAACCTATTTTAGAGGTC
TTAAAAAGGGTGTTGCCCGCCACGGGAACCGTTTTAGAAGTAGCC
AGCGGCACAGGACAACACGCGGTTTTTTTTGCTCCCCGTCTCTCC
CCTAGGCGGTGGCTACCTTCCGACCCGCAACCTTTGTTACAACAG
AGTATTAAAGCATGGCAAATTCATTCACCTTCAGATAATCTTTAC
CCTCCCCTACAGTTAAATGTAGAAGCTAATCCTTGGCCAGTGGAA
GGGAATGATTTACCCTGGGAATTATCGGAATTTCCCATCACTGCT
ATTGTAGCAATTAACCTGATTCATATTTCGCCTTGGTCGGCTTGT
GAAGGGTTAATGGCGGGAAGCGATCGCATTTTGAAACCGGGGGGA
ATTTTATATCTCTATGGACCCTATAAAATAGCTGGAAAACACACC
GCACCTAGTAACGCTTCCTTTGATGAATATTTGCGAACTTCTAAC
CCTAAGTGGGGTGTCAGAAACCTTGATGAT.

The homology arms may be any length that allows for site-specific homologous recombination. A homology arm may be any length between about 2000 bp and 500 bp including all integer values between. For example, a homology arm may be about 2000 bp, about 1500 bp, about 1000 bp, or about 500 bp. In aspects, homology arms may be the same or different length. In aspects, one or more homology arms may be any length between about 2000 bp and 500 bp including all integer values between. For example, each of the two homology arms may be about 2000 bp, about 1500 bp, about 1000 bp, or about 500 bp.

A portion of the vector adjacent to one or both (i.e., between) homology arms modifies the targeted locus in the Spirulina genome by homologous recombination. Techniques for homologous recombination in other organisms are generally known (see, e.g., Kriegler, 1990, Gene transfer and expression: a laboratory manual, Stockton Press). The modification may change a length of the targeted locus including a deletion of nucleotides or addition of nucleotides. The addition or deletion may be of any length. The modification may also change a sequence of the nucleotides in the targeted locus without changing the length. The targeted locus may be any portion of the Spirulina genome including coding regions, non-coding regions, and regulatory sequences. In aspects, the mutation may delete a gene thereby creating a knock-out organism. In aspects, the mutation may add a gene that functions as a reporter or marker (e.g., GFP or antibiotic resistance). In aspects, a mutation may add an exogenous gene. In aspects, a mutation may add an endogenous gene under control of an exogenous promoter (e.g., strong promoter, inducible promoter, etc.).

A vector for use in the targeted mutagenesis described above may be produced by assembling a vector backbone with an insert sequence. The vector may be created by any known or later developed technique including restriction enzyme digest followed by ligation or Gibson assembly. Gibson assembly may be performed by combining DNA sequences that will become portions of vector backbone with an exonuclease, DNA polymerase, and DNA ligase then incubating at 50° C. for one hour. The vector backbone may be selected for compatibility with the target organism. For Spirulina, suitable vector backbones include, but are not limited to DNA plasmids. The vector backbone may be converted from a continuous loop to a linear form by treatment with an appropriate restriction endonuclease. The ends thereby formed are treated with alkaline phosphatase to remove 5′-phosphate end groups so that the vector may not reform a continuous loop in a DNA ligase reaction without first incorporating an insert segment.

The insert sequence includes one or two homology arms and a nucleotide sequence that, due to differences between the nucleotide sequences of the insert and the wild-type genome sequence of the Spirulina, modify a locus of the Spirulina. The insert sequence includes the one or two flanking regions adjacent to the locus of interest that correspond to the homology arms which are homologous to regions of the Spirulina genome and a portion that is different from the Spirulina genome. The portion of the insert sequence that differs from the wild-type nucleotide sequence of the Spirulina leads to modification of the Spirulina genome due to those differences. The modification may include, but is not limited to, a point mutation, addition of a gene, addition of a regulatory element, addition of a coding region, addition of a non-coding region, deletion of a gene, deletion of a portion of a gene, or deletion of a regulatory element. Generally, it is well-known that strong E. coli promoters work well in Cyanobacteria. If the sequence of the target organism is known, a published sequence may be used to design the insert sequence with homology arm(s). Several strains of Spirulina have been sequenced such as Arthrospira plantensis NIES-39. If the sequence is not known a portion of the genome containing the loci of interest may be amplified using PCR and suitable primers. This region may be subsequently sequenced using techniques known to one skilled in the art. In aspects, an amplified region of the genome may be used to carry out overlap extension PCR to create a deletion. In aspects, an amplified region of the genome may be digested with a restriction endonuclease that cuts the nucleotide sequence in one place and ligated to an insert nucleotide sequence that has be prepared with compatible ends.

The insert sequence is digested with one or more restriction endonucleases to create ends that are compatible with the ends of the vector backbone. Alternatively, short sequences (e.g., 16-25 bp) that are identical to the terminal regions of the vector backbone are added to the ends of the insert sequence, and these two fragments are circularized by the Gibson assembly method. The length of the insert sequence will be the length of the modifications to the loci of interest and the length of the flanking regions. For example, if the length of the modified sequence is 500 bp and two homology arms each of 2000 bp are desired, then the entire length of the sequence will be 4500 bp. The polynucleotides sequences used as vectors in the present invention, regardless of the length of the coding sequence itself, may be combined with other sequences, such as promoters, transcriptional terminators, additional restriction enzyme sites, multiple cloning sites, other coding segments, and the like, such that their overall length may vary considerably. It is therefore contemplated that a polynucleotide fragment of almost any length may be employed, with the total length preferably being limited only by the ease of preparation and use in the intended recombinant nucleotide protocol.

In aspects, a vector comprises a sequence encoding for methanolysin. In aspects, the sequence comprises at least 80%, 85%, 90%, 95%, 98% or 99% identity to a sequence of Table 1 or Table 4. In aspects, the sequence comprises a sequence selected from Table 1 or Table 4.

In aspects, the vector further comprises a native Spirulina promoter. In aspects, the vector comprises a native Spirulina promoter is P_{cpc600}. In aspects, the vector comprises, the sequence encoding for methanolysin is flanked by a pair of homology arms. In aspects, the homology arms comprise a sequence homologous to a neutral site in the Spirulina genome. In aspects, the neutral site is selected from the group consisting of: kanamycin aminoglycoside acetyltransferase, NS1, and both kanamycin aminoglycoside acetyltransferase and NS1.

In aspects, the neutral site comprises at least a portion of a kanamycin aminoglycoside acetyltransferase gene. In aspects, the neutral site is a kanamycin aminoglycoside acetyltransferase gene.

In aspects, the first and second homology arms comprise about 60% to about 100%, about 70% to about 100%, about 80% to about 100%, about 90% to about 100%, about 50% to about 90%, about 60% to about 90%, about 70% to about 90%, about 80 to about 90%, about 50% to about 80%, about 60% to about 80%, about 70% to about 80%, about 50% to about 70%, about 60% to about 70%, or about 50% to about 60% of any one of SEQ ID NO: 103-104. In aspects, the first homology arms comprises at least 80% identity to SEQ ID NO: 103, and the second homology arm comprises at least 80% identity to SEQ ID NO: 104. In aspects, the first homology arms comprises SEQ ID NO: 103, and wherein the second homology arm comprises SEQ ID NO: 104.

In aspects, the methanolysin is in a fusion protein. In aspects, the fusion protein comprises a tag selected from the group consisting of: maltose binding protein, thioredoxin, phycocyanin, and SUMO. In aspects, the methanolysin is in a fusion protein with maltose binding protein. In aspects, the methanolysin is in a fusion protein with thioredoxin. In aspects, the methanolysin is in a fusion protein with phycocyanin. In aspects, the methanolysin is in a fusion protein with SUMO.

Exemplary fusion proteins are provided below at Table 2.

TABLE 2
Exemplary fusion proteins of the disclosure.

| Fusion | | | | |
|---|---|---|---|---|
| protein | First | Second | Third | Fourth |
| No. | Protein | Protein | Protein | Protein |
| 1 | Lysin | SUMO | | |
| | SEQ ID NO: 78 | | | |
| 2 | Lysin | MBP | | |
| | SEQ ID NO: 78 | | | |
| 3 | Lysin | Thioredoxin | | |
| | SEQ ID NO: 78 | | | |
| 4 | Lysin | Phycocyanin | | |
| | SEQ ID NO: 78 | | | |
| 5 | Lysin | SUMO | MBP | |
| | SEQ ID NO: 78 | | | |
| 6 | Lysin | SUMO | Thioredoxin | |
| | SEQ ID NO: 78 | | | |
| 7 | Lysin | SUMO | Phycocyanin | |
| | SEQ ID NO: 78 | | | |
| 8 | Lysin | MBP | Thioredoxin | |
| | SEQ ID NO: 78 | | | |
| 9 | Lysin | MBP | Phycocyanin | |
| | SEQ ID NO: 78 | | | |
| 10 | Lysin | Thioredoxin | Phycocyanin | |
| | SEQ ID NO: 78 | | | |
| 11 | Lysin | SUMO | MBP | Thioredoxin |
| | SEQ ID NO: 78 | | | |
| 12 | Lysin | SUMO | MBP | Phycocyanin |
| | SEQ ID NO: 78 | | | |
| 13 | Lysin | SUMO | Thioredoxin | Phycocyanin |
| | SEQ ID NO: 78 | | | |
| 14 | Lysin | MBP | Thioredoxin | Phycocyanin |
| | SEQ ID NO: 78 | | | |
| 15 | Lysin1 | Lysin2: | | |
| | SEQ ID NO: 101 | SEQ ID NO: 102 | | |
| 16 | Lysin1 | Lysin2: | SUMO | |
| | SEQ ID NO: 101 | SEQ ID NO: 102 | | |
| 17 | Lysin1 | Lysin2: | MBP | |
| | SEQ ID NO: 101 | SEQ ID NO: 102 | | |
| 18 | Lysin1 | Lysin2: | Thioredoxin | |
| | SEQ ID NO: 101 | SEQ ID NO: 102 | | |
| 19 | Lysin1 | Lysin2: | Phycocyanin | |
| | SEQ ID NO: 101 | SEQ ID NO: 102 | | |
| 20 | Lysin1 | Lysin2: | SUMO | MBP |
| | SEQ ID NO: 101 | SEQ ID NO: 102 | | |
| 21 | Lysin1 | Lysin2: | SUMO | Thioredoxin |
| | SEQ ID NO: 101 | SEQ ID NO: 102 | | |
| 22 | Lysin1 | Lysin2: | SUMO | Phycocyanin |
| | SEQ ID NO: 101 | SEQ ID NO: 102 | | |
| 23 | Lysin1 | Lysin2: | MBP | Thioredoxin |
| | SEQ ID NO: 101 | SEQ ID NO: 102 | | |
| 24 | Lysin1 | Lysin2: | MBP | Phycocyanin |
| | SEQ ID NO: 101 | SEQ ID NO: 102 | | |
| 25 | Lysin1 | Lysin2: | Thioredoxin | Phycocyanin |
| | SEQ ID NO: 101 | SEQ ID NO: 102 | | |

### Methods of Making

Provided are methods of making engineered methanolysins. Provided are also methods of making modified Spirulina.

In aspects, Spirulina can be made competent prior to modification. For example, transformation competence can be achieved by cocultivation of Spirulina with one or more microorganisms. Suitable microorganisms can comprise one or more of: Sphingomonas and/or Microcella.

Competency may also be created by the electroporation technique or by any other known or future technique for creating competency in Spirulina. Known techniques for introducing artificial competence in Spirulina include electroporation (with or without an osmotic stabilizer and with or without a pH balancer), incubation in a solution containing divalent cations, and ultrasound. The Spirulina cells are contacted by a vector when artificial competency is induced.

Competent Spirulina can subsequently be exposed to an integrating vector containing a selectable marker and a gene of interest, flanked on both sides by sequences homologous to the Spirulina chromosome. Targeted integration of the transforming DNA by homologous recombination can be demonstrated by sequencing of chromosomal DNA using primers flanking the insertion site. In aspects, an insertion site is a neutral site.

The present disclosure also describes creation of targeted mutations in Spirulina through homologous recombination by introduction of a vector to competent cells. For example, a vector may be mixed with a solution of Spirulina cells prior to electroporation.

A portion of the vector adjacent to one or both (i.e., between) homology arms modifies the targeted locus in the Spirulina genome by homologous recombination. In aspects, a method of making a modified Spirulina cell comprising introducing into a wild type Spirulina cell a vector of SEQ ID NO: 122 to SEQ ID NO: 123, thereby generating a modified Spirulina cell. In aspects, a method of making a modified Spirulina cell comprising introducing into a wild type Spirulina cell a vector having the backbone of SEQ ID NO: 124 and modified to express a methanolysin of Table 1, thereby generating a modified Spirulina cell. Indeed, methods of the disclosure can comprise expressing one or more of the methanolysins of Table 1 in the vector of SEQ ID NO: 124. In aspects, the method further comprises culturing the modified Spirulina cell thereby generating a population of modified Spirulina cells. In aspects, the culturing comprises antibiotic selection.

Following electroporation, the population of Spirulina cells is expanded. The cells may be grown in the presence of one or more antibiotics selected based on resistance conferred through successful transformation with the plasmid.

In aspects, the antibiotic is selected from a group comprising of: aminoglycosides, ansamycins, cabacephem carbapenems, cephalosporins, glycopeptides, lincosamides, lipopeptide, macrolides, monobactams, nitrofurans, oxazolidinones, penicillins, polypeptides, quinolones/fluoroquinolones, sulfonamides, tetracyclines.

In aspects the antibiotic is selected from a group comprising of: amikacin, gentamicin, kanamycin, neomycin, netilmicin, tobramycin, paromomysin, streptomycin, spectinomysin, geldanamycin, herbimycin, rifaximin, loracarbef, ertapenem, doripenem, imipenem, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefoperazone, cefotaxime, cefpodoxime, ceftazidime, ceftibuten, ceftizoxime, moxalactam, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, erythromycin, roxithromycin, telithromycin, spiramycin, fidaxomicin, aztreonam, furazolidone, nitrofurantoin (bs), oxazolidinones (bs), linezolid, posizolid, radezolid, tedizolid, penicillins, amoxicillin, ampicillin, azlocillin, dicloxacillin, flucloxacillin, mezlocillin, methicillin, nafcillin, oxacillin, penicillin g, penicillin v, piperacillin, penicillin g, temocillin, ticarcillin, bacitracin, colistin, polymyxin b, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, sulfonamides (bs), mafenide, sulfacetamide, sulfadiazine, silver sulfadiazine, sulfadimethoxine, sulfamethizole, sulfamethoxazole, sulfanilimide (archaic), sulfasalazine, sulfisoxazole, trimethoprim-sulfamethoxazole (co-trimoxazole) (tmp-smx), sulfonamidochrysoidine (archaic), tetracyclines (bs), demeclocycline, doxycyclinem, metacycline, minocycline, oxytetracycline, tetracycline, clofazimine, dapsone, capreomycin, cycloserine, ethambutol (bs), ethionamide, isoniazid, pyrazinamide, rifampicin (rifampin in us), rifabutin, rifapentine, streptomycin, arsphenamine, chloramphenicol (bs), fosfomycin, fusidic acid, metronidazole, mupirocin, platensimycin, quinupristin/dalfopristin, thiamphenicol, tigecycline (bs), tinidazole and trimethoprim (bs).

In aspects, the antibiotic comprises an antibiotic in the class aminoglycosides. In aspects, the antibiotic is selected from a group comprising of: amikacin, gentamicin, kanamycin, neomycin, netilmicin, tobramycin, paromomysin, streptomycin, and spectinomysin. In aspects, the antibiotic comprises kanamycin. In aspects, an antibiotic is selected from the group consisting of: kanamycin, streptomycin, spectinomycin, and G418. In aspects, an antibiotic is streptomycin. In aspects, an antibiotic is spectinomycin. In aspects, an antibiotic is G418.

Modified Spirulina cultures can be initially propagated in shake flasks in medium based on the standard cyanobacterial SOT medium under Multitron conditions. Shake flask cultures can be used to inoculate airlift reactors, with medium modified by partial replacement of sodium bicarbonate with sodium carbonate such that initial culture pH is about 9.8. Cells can be grown at light levels of about 500-2,500 μmol m⁻² s⁻¹, with temperature maintained at about 35° C. As the culture utilizes CO₂ and grows, pH rises and thus CO₂ is added to the airlift stream to maintain pH between about 9.8 and 10. Cultures can be inoculated at a concentration of about 0.1-0.5 g 11 biomass by ash-free dry weight and harvested by filtration at about 2-4 g 11.

Modified Spirulina cultures can be grown at large scale (e.g., 2501 or 2000 1). In aspects, modified Spirulina is cultured in airlift reactors

In aspects, modified Spirulina cell is dried. For example, harvested biomass can be rinsed with a dilute (0.1%) trehalose solution to remove excess media salts, concentrated again by filtration and then spray-dried in a centrifugal nozzle spray-dryer. Feed rate, air flow and inlet air temperature can be controlled to maintain an outlet air temperature of about 68-72° C. at the powder-separation hydrocyclone. Once collected from the hydrocyclone, the powder can be sealed and stored in airtight, opaque mylar bags to prevent exposure to moisture and/or light. The powder can be stored at room temperature.

### Methods of the Disclosure

Provided are various methods employing modified Spirulina. In aspects, provided are methods of administering a modified Spirulina cell. In aspects, provided are methods of making and using modified Spirulina cells. Also provided are methods of reducing or eliminating methanogens in ruminants and mammals of the disclosure. Also provided are methods of reducing or eliminating methane in any of the ruminants of the disclosure. Also provided are methods of engineering methanolysins. Provided are also methods of sourcing novel methanolysins.

In aspects, a method of the disclosure comprises administering feed comprising modified Spirulina. The feed can be provided at least once a day, twice a day, three times a day, daily, weekly, biweekly, monthly, bimonthly, and on an as needed basis. In aspects, an as needed basis comprises administering feed comprising modified Spirulina to reduce or eliminate methanogens in a ruminant.

In aspects, a method of the disclosure comprises administering a supplement comprising modified Spirulina. In aspects, a food supplement is selected from a group consisting of: minerals, vitamins, protein, fiber, probiotics, growth promoters, natural additives and hydration supplements. In aspects, a food supplement is selected from a group consisting of salt block, calcium supplements, phosphorus supplements, selenium supplements, magnesium supplements, Vitamin A, Vitamin D, Vitamin E, Vitamin K, Vitamin B complex, soybean meal, cottonseed meal, fish meal, blood meal, alfalfa meal, corn, barley, oats, wheat, molasses, hay, straw, alfalfa, beet pulp, silage, probiotic supplements, yeast supplements, enzyme supplements, ionophores (e.g., monensin, lasalocid), hormonal supplements, apple cider vinegar, garlic, oregano oil, diatomaceous earth, electrolyte powders, and water additives.

In aspects, the composition administered comprises a nutraceutical. In aspects, the nutraceutical is selected from a group comprising of prebiotics, probiotics, essential fatty acids, antioxidants, herbal extracts, mycotoxin binders, immune modulators, gut health enhancers, joint supplements, natural growth promoters, stress alleviators, bone health supplements, organic acids, enzymes. In aspects, the nutraceutical is selected from a group comprising of Fructooligosaccharides (FOS), Mannan oligosaccharides (MOS), Inulin, Lactobacillus spp., Bifidobacterium spp., Enterococcus spp., Saccharomyces cerevisiae, Omega-3 fatty acids, Omega-6 fatty acids, Vitamin C, Vitamin E, Selenium, Zinc, Echinacea, Ginseng, Licorice root, Milk thistle, Bentonite, Activated charcoal, Yeast cell walls, Beta-glucans, Mannoproteins, Yeast-derived products, Butyric acid, Short-chain fatty acids, Glutamine, Glucosamine, Chondroitin sulfate, MSM (Methylsulfonylmethane), Essential oils (e.g., thyme, oregano), Plant extracts (e.g., garlic, turmeric), Adaptogens (e.g., ashwagandha, rhodiola), L-tryptophan, Calcium, Phosphorus, Vitamin D3, Formic acid, Propionic acid, Acetic acid, Phytase, Amylase, and Protease.

Also provided are methods of preventing, treating, stabilizing, or ameliorating disease. Enteric methanogens have been associated with a variety of human diseases (Hoegenauer, Christoph, et al. “Methanogenic archaea in the human gastrointestinal tract.” (Nature Reviews Gastroenterology & Hepatology 19.12 (2022): 805-813); Mozsik, Gyula, ed. “The Gut Microbiome: Implications for Human Disease.” (2016); Cisek, Agata Anna, et al. “The Role of Methanogenic Archaea in Inflammatory Bowel Disease-A Review.” Journal of Personalized Medicine 14.2 (2024): 196; Waqar, Syed Hamza Bin, and Aiman Rehan. “Methane and constipation-predominant irritable bowel syndrome: Entwining pillars of emerging neurogastroenterology.” Cureus 11.5 (2019)). Accordingly, in aspects also provided are methods of treatment that comprise administering a composition of the disclosure in an amount sufficient to reduce or eliminate an enteric methanogen. In aspects, a method comprises administering modified Spirulina of the disclosure expressing one or more methanolysins. In aspects, a method comprises administering two or more modified Spirulina each expressing a different methanolysin. In aspects, a cocktail of modified Spirulina may provide superior methanogen reducing effects as compared to administration of a single strain of modified Spirulina.

Methods of the disclosure include the prevention and/or treatment of disease. Exemplary diseases of the disclosure include those associated with methanogens. In aspects, a subject comprises one or more methanogens in an amount sufficient to cause disease or a symptom thereof. For example, the human body harbors methanogen species represented by Methanobrevibacter smithii, Methanobrevibacter oralis, Methanosphaera stadtmanae, Methanomassiliicoccus luminyensis, Candidatus Methanomassiliicoccus intestinalis, and/or Candidatus Methanomethylophilus alvus. Thus, methods of the disclosure also comprise detection of a methanogen in a subject. Detection can be performed before, during, or after administration of a subject composition. In aspects, a composition of the disclosure is administered following detection of a methanogen.

Exemplary methods of detection of methanogens include but are not limited to microscopic examination, culturing, molecular techniques, 16S ribosomal RNA (rRNA) gene amplicon sequencing and metagenomic analysis. An exemplary microscopic examination method comprises staining a fecal sample or intestinal content and analyzing the stained sample with microscopy to visualize methanogenic cells. An exemplary culturing examination comprises culturing the methanogens in specialized media under anaerobic conditions and then confirming methane production by using biochemical tests. An exemplary molecular technique method comprises using polymerase chain reaction (PCR) and quantitative PCR (qPCR) to detect and quantify methanogen DNA in samples. An exemplary 16S rRNA gene amplicon sequencing method comprises identifying and characterizing methanogens in a microbial culture or intestinal sample based on their 16S rRNA gene sequences. An exemplary metagenomic analysis method comprises sequencing the entire DNA content of a microbial culture or intestinal sample to detect the methanogens present in the sample.

As discussed above, the present disclosure additionally refers to disease which can be prevented and treated with any of the compositions provided. In humans, methanogens have been identified in the gastrointestinal tract, mouth, and vagina. Methanogens can play a role in the progression of disease conditions in humans. Thus, in aspects, a disease of the disclosure is one of the gastrointestinal tract, skin, mouth, and/or vagina. In aspects, a disease of the disclosure is gastrointestinal.

Exemplary gastrointestinal diseases comprise: inflammatory bowel diseases (including Crohn's disease, ulcerative colitis, proctitis, pouchitis, and cuffitis), constipation-predominant irritable bowel syndrome (IBS-C), functional constipation, small intestinal bacterial overgrowth (SIBO), halitosis, gastrointestinal cancer (including colorectal cancer), anorexia, and obesity. In aspects, Spirulina-expressed methanolysins could be administered to human subjects to treat or prevent disease or disfunction associated with methanogen colonization of the gastrointestinal tract.

In aspects, a disease is Crohn's disease or a symptom thereof. Associated symptoms of Crohn's comprise: abdominal pain and cramping, diarrhea, fatigue, weight loss, fever, rectal bleeding, perianal disease, reduced appetite, nausea and vomiting, joint pain, mouth sores, and delayed growth in children. Accordingly, a composition of the disclosure can be administered in an amount sufficient to reduce any of the aforementioned symptoms of Crohn's.

In aspects, a disease is ulcerative colitis or a symptom thereof. Associated symptoms of ulcerative colitis comprise: diarrhea, rectal bleeding, abdominal pain and cramping, urgency to defecate, tenesmus, fatigue, weight loss, fever, anemia, joint pain, skin and eye problems, liver and bile problems and loss of appetite. Accordingly, a composition of the disclosure can be administered in an amount sufficient to reduce any of the aforementioned symptoms of ulcerative colitis.

In aspects, a disease is proctitis or a symptom thereof. Associated symptoms of proctitis comprise: rectal pain, rectal bleeding, rectal discharge, tenesmus, increased urgency to defecate, diarrhea or constipation, painful bowel movements, anal itching or irritation, discomfort during sexual intercourse, fatigue and fever. Accordingly, a composition of the disclosure can be administered in an amount sufficient to reduce any of the aforementioned symptoms of proctitis.

In aspects, a disease is pouchitis or a symptom thereof. Associated symptoms of pouchitis comprise: increased frequency of bowel movements, urgency to defecate, loose stools or diarrhea, rectal bleeding, abdominal cramping or pain, fever, fatigue, malaise, nausea or vomiting, loss of appetite, joint pain, and skin problems. Accordingly, a composition of the disclosure can be administered in an amount sufficient to reduce any of the aforementioned symptoms of pouchitis.

In aspects, a disease is cuffitis or a symptom thereof. Associated symptoms of cuffitis comprise: rectal bleeding, increased frequency of bowel movements, urgency to defecate, diarrhea or loose stools, rectal pain or discomfort, tenesmus, abdominal cramping, mucous discharge, fatigue, fever and loss of appetite. Accordingly, a composition of the disclosure can be administered in an amount sufficient to reduce any of the aforementioned symptoms of cuffitis.

In aspects, a disease is constipation-predominant irritable bowel syndrome (IBS-C) or a symptom thereof. Associated symptoms of IBS-C comprise: constipation, abdominal discomfort or pain, straining during bowl movements, feelings of incomplete evacuation, hardy or lumpy stools, bloating and gas, abdominal distension, rectal discomfort, mucus in stool, fatigue and anxiety or depression. Accordingly, a composition of the disclosure can be administered in an amount sufficient to reduce any of the aforementioned symptoms of IBS-C.

In aspects, a disease is functional constipation or a symptom thereof. Associated symptoms of functional constipation comprise: infrequent bowel movements, difficulty passing stool, hard or lumpy stools, feeling of incomplete evacuation, abdominal discomfort or pain, rectal discomfort, bloating and gas, straining during bowl movements, anal fissures, hemorrhoids and fatigue. Accordingly, a composition of the disclosure can be administered in an amount sufficient to reduce any of the aforementioned symptoms of functional constipation.

In aspects, a disease is small intestinal bacterial overgrowth (SIBO) or a symptom thereof. Associated symptoms of SIBO comprise: abdominal bloating, abdominal pain or cramping, flatulence, diarrhea, constipation, abdominal distension, nausea or vomiting, fatigue, unintended weight loss, malnutrition, nutritional deficiencies and bloating and discomfort after eating certain foods. Accordingly, a composition of the disclosure can be administered in an amount sufficient to reduce any of the aforementioned symptoms of SIBO.

In aspects, a disease is halitosis or a symptom thereof. Associated symptoms of halitosis comprise: unpleasant odor, dry mouth, white or yellow coating of the tongue, bad taste in mouth, postnasal drip, gum disease, poor oral hygiene and dry cracked lips. Accordingly, a composition of the disclosure can be administered in an amount sufficient to reduce any of the aforementioned symptoms of halitosis.

In aspects, a disease is gastrointestinal cancer or a symptom thereof. Associated symptoms of gastrointestinal cancer comprise: weight loss, abdominal pain and discomfort, change in bowel habits, blood in stool, fatigue, loss of appetite, nausea and vomiting, difficulty swallowing, indigestion or heartburn, jaundice, changes in bowel habits, abdominal swelling or lump, feeling of incomplete evacuation and anemia. Accordingly, a composition of the disclosure can be administered in an amount sufficient to reduce any of the aforementioned symptoms of gastrointestinal cancer.

In aspects, a disease is anorexia or a symptom thereof. Associated symptoms of anorexia comprise: significant weight loss, intense fear of gaining weight, distorted body image, restricted eating, preoccupation with food, calories and weight, denial of hunger, mood changes, fatigue, dizziness, fainting, irregular menstrual periods, dry skin, brittle nails, thinning hard, reduced cold tolerance, constipation, abdominal pain, bloating and gas, nausea and vomiting, gastroparesis, acid reflux and difficulty swallowing. Accordingly, a composition of the disclosure can be administered in an amount sufficient to reduce any of the aforementioned symptoms of anorexia.

In aspects, a disease is obesity or a symptom thereof. Associated symptoms of obesity comprise: increased body weight, breathlessness, increased visceral fat, fatigue and low energy, joint pain, sleep disturbances, heartburn, acid reflux, gastroesophageal reflux disease, high cholesterol, dyspepsia, constipation, diverticular disease, abdominal pain, bloating, diarrhea, depression and anxiety. Accordingly, a composition of the disclosure can be administered in an amount sufficient to reduce any of the aforementioned symptoms of obesity.

The present disclosure provides methods of reducing methane emission comprising administering a composition comprising Spirulina modified to express a methanolysin to a subject. In aspects, the composition comprises two or more Spirulina that each express a different methanolysin. In aspects, the methanolysin is from an archaea organism. In aspects, the organism is from archaea selected from the group consisting of: Methanobrevibacter ruminantium M1, Methanobrevibacter gottschalkii and Methanosphaera cuniculi. In aspects, the archaea is Methanobrevibacter ruminantium M1. In aspects, the Methanobrevibacter ruminantium M1 methanolysin is PeiR or PeiR-MC. In aspects, a PeiR-MC methanolysin comprises an amino acid sequence with at least about 80%, 85%, 90%, 95%, 98%, or 99% identity to SEQ ID NO: 78. In aspects the PeiR-MC methanolysin comprises SEQ ID NO: 78.

In aspects, any of the described methods employ a methanolysin selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding g protein [Methanosphaera cuniculi], C39 family peptidase [Methanobrevibacter gottschalkii], cysteine peptidase family C39 domain-containing protein [Methanobrevibacter sp.], TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.].

In aspects, the methanolysin comprises an amino acid sequence with at least about 80%, 85%, 90%, 95%, 98%, or 99% identity to SEQ ID NOs: 1-102. In aspects, the methanolysin comprises SEQ ID NO: 101 and/or 102. In aspects, the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor. In aspects, the Spirulina is the A. platensis.

In aspects, a method of the disclosure is effective at reducing methane emissions by at least about 1-fold, 5-fold, 10-fold, 20-fold, or 50-fold as compared to an otherwise comparable method lacking administration of a modified Spirulina and/or methanolysin. In aspects, the reduction in methane emissions is determined by detecting methane in metabolic gases generated by the subject. In aspects, a method of the disclosure is effective at reducing methane emissions by at least about 5%, 10%, 15%, 20%, 25%, 30%, 40%, 45%, 50%, 55%, 60%, 70%, 80%, 90%, 95%, or up to about 100% as compared to an otherwise comparable method lacking administration of a modified Spirulina and/or methanolysin. In aspects, the reduction in methane emissions is determined by detecting methane in metabolic gases generated by the subject. In aspects, the reduction in methane emissions is observed within about 1 hrs., 3 hrs., 5 hrs., 7 hrs., 9 hrs., 11 hrs., 13 hrs., 15 hrs., 17 hrs., 19 hrs., 21 hrs., 23 hrs., 1-day, 5-days, 9-days, 13-days, 17-days, 21-days, 25-days, 1 month, 6 months, or 18 months.

In aspects, a method further comprises re-administering a composition of the disclosure. In aspects, a composition is readministered within one day, 3 days, 5 days, or a week following the administration. A composition of the disclosure can be re-administered until methane is reduced by at least 1-fold, 5-fold, 10-fold, 20-fold, or 50-fold as compared to a baseline level. A composition of the disclosure can be re-administered until methane is reduced by at least 5%, 10%, 15%, 20%, 25%, 30%, 40%, 45%, or 50% as compared to a baseline level. In aspects, a composition is readministered daily.

In aspects, a subject of the disclosure is administered Spirulina biomass. Exemplary concentrations of Spirulina biomass can be from at least about or at most about: 0.25 grams, 0.5 grams, 1 gram, 1.5 grams, 2 grams, 2.5 grams, 3 grams, 3.5 grams, 4 grams, 4.5 grams, 5 grams, 5.5 grams, 6 grams, 6.5 grams, 7 grams, 7.5 grams, 8 grams, 8.5 grams, 9 grams, 9.5 grams, 10 grams, 10.5 grams, 11 grams, 11.5 grams, 12 grams, 12.5 grams, 13 grams, 13.5 grams, 14 grams, 14.5 grams, 15 grams, 15.5 grams, 16 grams, 16.5 grams, 17 grams, 17.5 grams, 18 grams, 18.5 grams, 19 grams, 19.5 grams, 20 grams, 20.5 grams, 21 grams, 21.5 grams, 22 grams, 22.5 grams, 23 grams, 23.5 grams, 24 grams, 24.5 grams, 25 grams, 25.5 grams, 26 grams, 26.5 grams, 27 grams, 27.5 grams, 28 grams, 28.5 grams, 29 grams, 29.5 grams, 30 grams, or up to about 40 grams.

In aspects, exemplary concentrations of Spirulina biomass can be from at least about or at most about: 0.1 grams, 0.2 grams, 0.3 grams, 0.4 grams, 0.5 grams, 0.6 grams, 0.7 grams, 0.8 grams, 0.9 grams, 1 gram, 1.1 grams, 1.2 grams, 1.3 grams, 1.4 grams, 1.5 grams, 1.6 grams, 1.7 grams, 1.8 grams, 1.9 grams, 2 grams, or up to about 2.5 grams.

Any of the aforementioned dosages of Spirulina biomass can be utilized in a method of the disclosure. For example, a subject of the disclosure can be administered any of the aforementioned dosages of Spirulina biomass at a frequency of about once a day, twice a day, three times a day, every other day, every third day, twice a week, or weekly. In aspects, a subject is administered Spirulina biomass every 2, 4, 6, 8, 10, or 12 hours.

In aspects, a subject of the disclosure is a ruminant that is administered Spirulina biomass at a dosage of about 0.25 g to 30 g one to three times daily. In aspects, a subject of the disclosure is a ruminant that is administered Spirulina biomass at a dosage of about 5 g to 10 g, 10 g to 15 g, 15 g to 20 g, 20 g to 30 g, one to three times daily. In aspects, a subject of the disclosure is a cow that is administered Spirulina biomass at a dosage of about 5 g to 10 g, 10 g to 15 g, 15 g to 20 g, 20 g to 30 g, one to three times daily. In aspects, a subject of the disclosure is a cow that is administered Spirulina biomass at a dosage of about 5 g to 10 g, 10 g to 15 g, 15 g to 20 g, 20 g to 30 g, once daily. In aspects, a subject of the disclosure is a cow that is administered Spirulina biomass at a dosage of about 5 g to 10 g, 10 g to 15 g, 15 g to 20 g, 20 g to 30 g, twice daily. In aspects, a subject of the disclosure is a cow that is administered Spirulina biomass at a dosage of about 5 g to 10 g, 10 g to 15 g, 15 g to 20 g, 20 g to 30 g, three times a day.

In aspects, a subject of the disclosure is a mammal that is administered Spirulina biomass at a dosage of about 0.25 g to 30 g one to three times daily. In aspects, a subject of the disclosure is a mammal that is administered Spirulina biomass at a dosage of about 5 g to 10 g, 10 g to 15 g, 15 g to 20 g, 20 g to 30 g, one to three times daily. In aspects, a subject of the disclosure is a non-human mammal that is administered Spirulina biomass at a dosage of about 5 g to 10 g, 10 g to 15 g, 15 g to 20 g, 20 g to 30 g, one to three times daily. In aspects, a subject of the disclosure is a non-human mammal that is administered Spirulina biomass at a dosage of about 5 g to 10 g, 10 g to 15 g, 15 g to 20 g, 20 g to 30 g, once daily. In aspects, a subject of the disclosure is a non-human mammal that is administered Spirulina biomass at a dosage of about 5 g to 10 g, 10 g to 15 g, 15 g to 20 g, 20 g to 30 g, twice daily. In aspects, a subject of the disclosure is a non-human mammal that is administered Spirulina biomass at a dosage of about 5 g to 10 g, 10 g to 15 g, 15 g to 20 g, 20 g to 30 g, three times a day.

In aspects, a subject of the disclosure is a human that is administered Spirulina biomass at a dosage of about 0.25 g to 5 g one to three times daily. In aspects, a subject of the disclosure is a human that is administered Spirulina biomass at a dosage of about 0.1 g to 0.5 g, 0.3 g to 0.8 g, 0.5 g to 1 g, 1 g to 1.5 g, or 1.5 g to 2 g, one to three times daily. In aspects, a subject of the disclosure is a human that is administered Spirulina biomass at a dosage of about 0.1 g to 0.5 g, 0.3 g to 0.8 g, 0.5 g to 1 g, 1 g to 1.5 g, or 1.5 g to 2 g, one to three times daily. In aspects, a subject of the disclosure is a human that is administered Spirulina biomass at a dosage of about 0.1 g to 0.5 g, 0.3 g to 0.8 g, 0.5 g to 1 g, 1 g to 1.5 g, or 1.5 g to 2 g, once daily. In aspects, a subject of the disclosure is a human that is administered Spirulina biomass at a dosage of about 0.1 g to 0.5 g, 0.3 g to 0.8 g, 0.5 g to 1 g, 1 g to 1.5 g, or 1.5 g to 2 g, twice daily. In aspects, a subject of the disclosure is a human that is administered Spirulina biomass at a dosage of about 0.1 g to 0.5 g, 0.3 g to 0.8 g, 0.5 g to 1 g, 1 g to 1.5 g, or 1.5 g to 2 g, three times a day.

In aspects, a method comprises administering modified Spirulina expressing a methanolysin of Table 1 in an amount sufficient to reduce methane emissions in a ruminant. In aspects, a method comprises administering modified Spirulina expressing a methanolysin of Table 1 in an amount sufficient to reduce methane emissions in a ruminant within about 5 days following the administration. In aspects, a method comprises administering modified Spirulina expressing a methanolysin of Table 1 in an amount sufficient to reduce methane emissions in a ruminant within about 5 days following the administration as determined by detecting reduced belching in the subject.

In aspects, a method comprises administering two or more modified Spirulina expressing two or more methanolysins of Table 1 in an amount sufficient to reduce methane emissions in a ruminant. In aspects, a method comprises administering two or more modified Spirulina expressing two or more methanolysins of Table 1 in an amount sufficient to reduce methane emissions in a ruminant within about 5 days following the administration.

In aspects, a method comprises administering modified Spirulina expressing a methanolysin of Table 4 in an amount sufficient to reduce methane emissions in a ruminant. In aspects, a method comprises administering modified Spirulina expressing a methanolysin of Table 4 in an amount sufficient to reduce methane emissions in a ruminant within about 5 days following the administration. In aspects, a method comprises administering two or more modified Spirulina expressing two or more methanolysins of Table 4 in an amount sufficient to reduce methane emissions in a ruminant. In aspects, a method comprises administering two or more modified Spirulina expressing two or more methanolysins of Table 4 in an amount sufficient to reduce methane emissions in a ruminant within about 5 days following the administration.

In aspects, a method comprises administering modified Spirulina expressing a methanolysin of Table 1 in an amount sufficient to reduce methane emissions in a ruminant. In aspects, a method comprises administering modified Spirulina expressing a methanolysin of Table 1 in an amount sufficient to reduce methane emissions in a ruminant within about 5 days following the administration. In aspects, a method comprises administering two or more modified Spirulina expressing two or more methanolysins of Table 1 in an amount sufficient to reduce methane emissions in a ruminant. In aspects, a method comprises administering two or more modified Spirulina expressing two or more methanolysins of Table 1 in an amount sufficient to reduce methane emissions in a ruminant within about 5 days following the administration. In aspects, a methanolysin in within a fusion protein comprising one or more of: maltose binding protein, thioredoxin, phycocyanin, and SUMO tags.

### Kits of the Disclosure

Disclosed herein can be kits comprising compositions of the disclosure. In aspects, a kit can include an effective amount of a composition of Spirulina cells in unit dosage form. In aspects, a kit comprises a sterile container which can contain a therapeutic composition of Spirulina cells; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In aspects, modified Spirulina cells can be provided together with instructions for administering the cells to a subject. Instructions can generally include information about the use of the composition for any of the disclosed indications and uses.

### EMBODIMENTS

### Embodiment Set 1

1. A modified Spirulina that expresses a methanolysin.
2. The modified Spirulina of embodiment 1, wherein the methanolysin is selected from the group consisting of: SEQ ID NO: 1-70.
3. The modified Spirulina of embodiment 1, wherein the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.
4. An isolated polynucleotide DNA sequence encoding a methanolysin selected from the group consisting of: SEQ ID NO: 1 to 70.
5. A vector comprising the polynucleotide of embodiment 4.
6. An isolated host cell comprising the vector of embodiment 5.
7. A kit comprising a ruminant food and a methanolysin.
8. A method of making methanolysin comprising isolating methanolysin from the modified Spirulina of embodiment 1.
9. A method of making a ruminant food comprising combining a grain and one or more methanolysins generated from the method of embodiment 8.
10. A method of reducing methane emission from a ruminant comprising feeding a ruminant the food generated by the method of embodiment 9 thereby reducing methane emission as determined by detecting reduced methane in metabolic gases generated by the ruminant.
11. A method of reducing feed consumption by a ruminant comprising feeding a ruminant the food generated by the method of embodiment 9.
12. A modified methanolysin comprising increased killing activity and/or accumulation in a Spirulina host cell as compared to an otherwise comparable unmodified methanolysin.

### Embodiment Set 2

1. A modified Spirulina cell, comprising: an exogenous sequence encoding a methanolysin.
2. The modified Spirulina cell of embodiment 1, wherein the exogenous sequence encoding the methanolysin is genomically integrated into the Spirulina cell genome.
3. The modified Spirulina cell of embodiment 2, wherein the genomic integration is within a neutral genomic region.
4. The modified Spirulina cell of embodiment 3, wherein the neutral genomic region is selected from the group consisting of: kanamycin aminoglycoside acetyltransferase, NS1, and both kanamycin aminoglycoside acetyltransferase and NS1.
5. The modified Spirulina cell of embodiment 4, wherein the neutral genomic region is the kanamycin aminoglycoside acetyltransferase.
6. The modified Spirulina cell of any one of embodiments 1-5, wherein the methanolysin is from an archaea organism.
7. The modified Spirulina cell of embodiment 6, wherein the organism is from archaea selected from the group consisting of: Methanobrevibacter ruminantium, Methanobrevibacter gottschalkii and Methanosphaera cuniculi.
8. The modified Spirulina cell of embodiment 7, wherein the archaea is Methanobrevibacter ruminantium M1.
9. The modified Spirulina cell of embodiment 8, wherein the Methanobrevibacter ruminantium methanolysin is PeiR-MC.
10. The modified Spirulina cell of embodiment 9, wherein the PeiR-MC methanolysin comprises an amino acid sequence with at least about 80%, 85%, 90%, 95%, 98%, or 99% identity to SEQ ID NO: 78.
11. The modified Spirulina cell of embodiment 9, wherein the PeiR-MC methanolysin comprises SEQ ID NO: 78.
12. The modified Spirulina cell of embodiment 6, wherein the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.], TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.].
13. The modified Spirulina cell of embodiment 6, wherein the methanolysin comprises an amino acid sequence with at least about 80%, 85%, 90%, 95%, 98%, or 99% identity to SEQ ID NO. 1-102.
14. The modified Spirulina cell of embodiment 13, wherein the methanolysin comprises a sequence of SEQ ID NOs: 1-102.
15. The modified Spirulina cell of any one of embodiments 1-14, wherein the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.
16. The modified Spirulina cell of embodiment 15, wherein the Spirulina is the A. platensis.
17. A population of Spirulina, comprising: the modified Spirulina cell of any one of embodiments 1-16.
18. A population of Spirulina, comprising: two or more Spirulina cells, wherein each of the two or more Spirulina cells express different methanolysin proteins.
19. The population of embodiment 18, wherein the two or more Spirulina cells express different methanolysin proteins that comprise an amino acid sequence with at least about 80%, 85%, 90%, 95%, 98%, or 99% identity to SEQ ID NO: 1 to SEQ ID NO: 102.
20. The population of any one of embodiments 18-19, wherein the two or more Spirulina cells comprise two or more methanolysins selected from the group consisting of: SEQ ID NO: 1 to SEQ ID NO: 102.
21. A food supplement composition, comprising: the modified Spirulina cell of any one of embodiments 1-16; or the population of any one of embodiments 17-20.
22. A nutraceutical or pharmaceutical composition comprising the modified Spirulina cell of any one of embodiments 1-16; or the population of any one of embodiments 17-20.
23. A vector, comprising: a sequence encoding for methanolysin, wherein the sequence comprises at least 80% identity to any one of SEQ ID NOs: 122, 123, or 124.
24. The vector of embodiment 23, wherein the sequence comprises SEQ ID NO: 122 or SEQ ID NO: 123.
25. The vector of any one of embodiments 23-24, wherein the vector further comprises a native Spirulina promoter.
26. The vector of embodiment 25, wherein the native Spirulina promoter is P_{cpc600}.
27. The vector of any one of embodiments 23-26, wherein the sequence encoding for methanolysin is flanked by a pair of homology arms.
28. The vector of embodiment 27, wherein the homology arms comprise a sequence homologous to a neutral site in the Spirulina genome.
29. The vector of embodiment 28, wherein the neutral site is selected from the group consisting of: kanamycin aminoglycoside acetyltransferase, NS1, and both kanamycin aminoglycoside acetyltransferase and NS1.
30. The vector of embodiment 29, wherein the neutral site comprises a portion of a kanamycin aminoglycoside acetyltransferase gene.
31. The vector of embodiment 30, wherein the first homology arms comprises at least 80% identity to SEQ ID NO: 103, and wherein the second homology arm comprises at least 80% identity to SEQ ID NO: 104.
32. The vector of embodiment 31, wherein the first homology arms comprises SEQ ID NO: 103, and wherein the second homology arm comprises SEQ ID NO: 104.
33. The vector of any one of embodiments 23-32, wherein the methanolysin is in a fusion protein.
34. The vector of embodiment 33, wherein the fusion protein comprises a tag selected from the group consisting of: maltose binding protein, thioredoxin, phycocyanin, and SUMO.
35. A kit, comprising: the modified Spirulina cell of any one of embodiments 1-16; the population of any one of embodiments 17-20; the food supplement of embodiment 21; the nutraceutical of embodiment 22; and/or the vector of any one of embodiments 23-34.
36. The kit of embodiment 35, further comprising instructions for use thereof.
37. A method of making a modified Spirulina cell, the method comprising: introducing into a wild type Spirulina cell the vector of any one of embodiments 23-34, thereby generating a modified Spirulina cell.
38. The method of embodiment 37, further comprising culturing the modified Spirulina cell thereby generating a population of modified Spirulina cells.
39. The method of embodiment 38, wherein the culturing comprises antibiotic selection.
40. The method of embodiment 39, wherein the antibiotic is kanamycin, streptomycin, spectinomycin, and/or G418.
41. The method of any one of embodiments 37-40, wherein the modified Spirulina cell is dried.
42. A method, comprising: administering: the modified Spirulina cell of any one of embodiments 1-16; the population of any one of embodiments 17-20; the food supplement of embodiment 21; and/or the nutraceutical or pharmaceutical of embodiment 22.
43. A method of reducing methane emissions, the method comprising: administering a composition comprising Spirulina modified to express a methanolysin to a subject.
44. The method of embodiment 43, wherein the composition comprises two or more Spirulina that each express a different methanolysin.
45. The method of any one of embodiments 43-44, wherein the methanolysin is from an archaea organism.
46. The method of embodiment 45, wherein the organism is from archaea selected from the group consisting of: Methanobrevibacter ruminantium M1, Methanobrevibacter gottschalkii and Methanosphaera cuniculi.
47. The method of embodiment 46, wherein the archaea is Methanobrevibacter ruminantium M1.
48. The method of embodiment 47, wherein the Methanobrevibacter ruminantium M1 methanolysin is PeiR-MC.
49. The method of embodiment 48, wherein the PeiR-MC methanolysin comprises an amino acid sequence with at least about 80%, 85%, 90%, 95%, 98%, or 99% identity to SEQ ID NO: 78.
50. The method of embodiment 49, wherein the PeiR-MC methanolysin comprises SEQ ID NO: 78.
51. The method of embodiment 45, wherein the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.], TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.].
52. The method of embodiment 45, wherein the methanolysin comprises an amino acid sequence with at least about 80%, 85%, 90%, 95%, 98%, or 99% identity to SEQ ID NO: 1 to SEQ ID NO: 102.
53. The method of embodiment 52, wherein the methanolysin comprises a sequence selected from the group consisting of: SEQ ID NO: 1 to SEQ ID NO: 102.
54. The method of any one of embodiments 43-53, wherein the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.
55. The method of embodiment 54, wherein the Spirulina is A. platensis.
56. The method of any one of embodiments 43-55, wherein the administration is effective at reducing methane emissions by at least about 1-fold, 5-fold, 10-fold, 20-fold, or 50-fold as compared to an otherwise comparable method lacking the administration.
57. The method of embodiment 56, wherein the reduction in methane emissions is determined by detecting methane in metabolic gases generated by the subject.
58. The method of any one of embodiments 43-57, further comprising re-administering the composition.
59. The method of embodiment 58, wherein the composition is readministered within about 2 hours, 4 hours, 8 hours, 10 hours, one day, 3 days, 5 days, or a week following the administration.
60. The method of embodiment 59, wherein the composition is readministered daily.
61. The method of any one of embodiments 43-60, wherein the subject is mammalian.
62. The method of embodiment 61, wherein the mammalian is human.
63. The method of embodiment 61, wherein the mammalian is non-human.
64. The method of embodiment 63, wherein the non-human mammalian subject is a ruminant.
65. The method of embodiment 64, wherein the ruminant is domesticated.
66. The method of anyone of embodiments 64-65, wherein the ruminant is selected from the group consisting of: cow, bovine, goat, sheep, giraffe, deer, gazelle, and antelope.
67. The method of embodiment 66, wherein the ruminant is a cow.
68. The method of embodiment 67, wherein the cow is selected from the group consisting of: Holstein Friesian, Hereford, Simmental, Aberdeen Angus, Brown Swiss, Galloway, Red Angus, Belted Galloway, Braford, Limousin, Belgian Blue, Brangus, Beefalo, Chillingham, Ongole, Cachena, Gelbvieh, American Angus, Chianina, Aubrac, Twinner, Parda Alpina, Diary Shorthorn, Maine-Anjou, Danish Red, Retinta, Horro, Montbeliarde, Heck, White Park, British White, Hallikar, Red Poll, Senepol, Caracu, Lakenvelder, Beef Shorthorn, Corriente, Florida Cracker, Tuli, Hungarian Grey, English longhorn, Tarentaise, Australian Lowline, Boskarin, Black Baldy, French Brown, Podolica, Pustertaler Sprinzen, and any combination thereof.
69. The method of any one of embodiments 42-68, wherein the Spirulina is administered in the form of Spirulina biomass.
70. The method of embodiment 69, wherein the Spirulina biomass is administered at a dose of at least about 0.25 grams to at most about 30 grams.
71. The method of embodiment 70, wherein the Spirulina biomass is administered at a dose of at least about 0.25 grams to about 2 grams; 0.5 grams to about 1 gram; or 1 gram to about 2 grams.
72. A method of treating or preventing disease or bodily dysfunction, the method comprising: orally administering to a subject a composition comprising Spirulina modified to express a methanolysin.
73. The method of embodiment 72, wherein the subject is a mammal.
74. The method of embodiment 73, wherein the mammalian is human.
75. The method of embodiment 73, wherein the mammalian is non-human.
76. The method of any one of embodiments 72-75, wherein the composition is administered in the form of Spirulina biomass.
77. The method of embodiment 76, wherein the Spirulina biomass is administered at a dose of at least about 0.25 grams to at most about 30 grams.
78. The method of embodiment 76, wherein the Spirulina biomass is administered at a dose of at least about 0.25 grams to about 2 grams; 0.5 grams to about 1 gram; or 1 gram to about 2 grams.
79. The method of any one of embodiments 72-78, wherein the administration is effective at reducing methane emissions as compared to an otherwise comparable method lacking the administration.
80. The method of any one of embodiments 72-78, wherein the administration is effective at reducing methanogens in the subject as compared to an otherwise comparable method lacking the administration.
81. The method of any one of embodiments 72-80, wherein the subject has a gastrointestinal disease.
82. The method of embodiment 81, wherein the subject has a gastrointestinal disease selected from the group consisting of: Crohn's disease, ulcerative colitis, proctitis, pouchitis, cuffitis, constipation-predominant irritable bowel syndrome (IBS-C), functional constipation, small intestinal bacterial overgrowth (SIBO), halitosis, gastrointestinal cancer, colorectal cancer, anorexia, and obesity.
83. The method of any one of embodiments 72-82, wherein the methanolysin is selected from the group consisting of: Methanosphaera cuniculi peptidoglycan-binding protein, hypothetical protein [Methanobacterium paludis], hypothetical protein [Methanobrevibacter sp.], cysteine peptidase family C39 domain-containing protein [Methanobrevibacter sp. V14], C39 family peptidase [Methanobrevibacter sp.], cysteine peptidase family C39 domain-containing [uncultured Methanobrevibacter sp.], C39 family peptidase [Methanobrevibacter sp.], peptidoglycan-binding protein [Methanosphaera cuniculi], C39 family peptidase [Methanobrevibacter gottschalkii], cysteine peptidase family C39 domain-containing protein [Methanobrevibacter sp.], TPA:Pseudomurein endo-isopeptidase Pei [Caudoviricetes sp.], cysteine peptidase family C39 domain-containing protein [Methanosphaera stadtmanae], cysteine peptidase family C39 domain-containing protein [Methanosphaera sp.], transglutaminase domain-containing protein [Methanobacterium sp. ERen5], transglutaminase domain-containing protein [Methanobacterium lacus], transglutaminase domain-containing protein [Methanobacterium], hypothetical protein [Caudoviricetes sp.], hypothetical protein [Methanobrevibacter smithii], Pseudomurein endo-isopeptidase Pei, partial [Caudoviricetes sp.], transglutaminase domain-containing protein [Methanobrevibacter smithii], hypothetical protein [Methanobrevibacter sp.], hypothetical protein [Caudoviricetes sp.], and cysteine peptidase family C39 domain-containing protein [Methanobrevibacter sp.].
84. The method of embodiment 83, wherein the methanolysin comprises an amino acid sequence with at least about 80%, 85%, 90%, 95%, 98%, or 99% identity to SEQ ID NO: 1 to SEQ ID NO: 102.
85. The method of embodiment 84, wherein the methanolysin comprises a sequence selected from the group consisting of: SEQ ID NO: 1 to SEQ ID NO: 102
86. The method of any one of embodiments 72-85, wherein the Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.
87. The method of embodiment 86, wherein the Spirulina is A. platensis.

### EXAMPLES

### Example 1: Genetically Engineered Spirulina-Methanolysin Strains

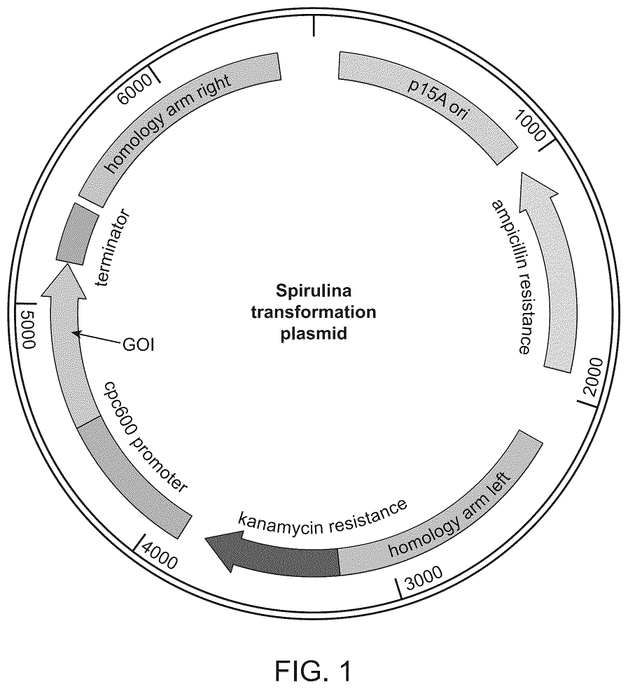

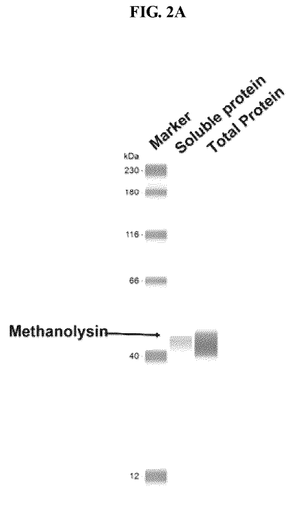

Genetically engineered methanolysin-expressing Spirulina strains were generated by transforming Spirulina with a plasmid that facilitates integration of the methanolysin gene into the Spirulina chromosome (FIG. 1) (Jester et al.). Using kanamycin as the selection agent, stable cell lines were generated by driving this chromosomal allele to fixation in the polyploid Spirulina genome. Homozygosity was assessed by polymerase chain reaction (PCR) and confirmed by sequencing and growth assays that select for the parental allele.

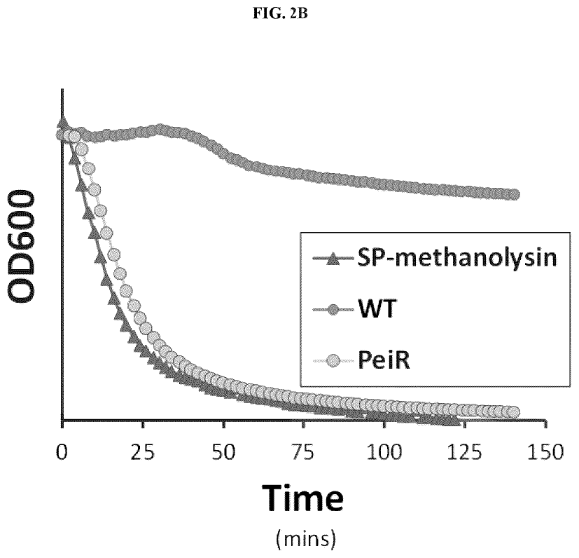

Spirulina-methanolysin cell lines were generated that each express enzymes: PeiR from the genome of Methanobrevibacter ruminantium M1, and methanolysins identified from the genomes of Methanobrevibacter gottschalkii and Methanosphaera cuniculi. Both proteins could be expressed with a hexahistidine tag at the amino or carboxy-terminus of the protein. When expressed constitutively in Spirulina, the protein from M. gottschalkii accumulated to <0.1% of dry weight whereas the protein from M. cuniculi accumulated to >2% of dry weight (FIG. 2A). The complete sequence for this methanolysin is identified as SEQ ID NO:78 in Table 1.

Spirulina can produce active methanolysin enzymes. A methanogen lysis assay was used to measure the enzymatic activity of methanolysin candidates and formulations. M. ruminantium “ghost” cells, anaerobically grown and rendered metabolically inactive, provide a structurally intact methanogen cell as the substrate. Both purified PeiR, an established methanolysin, and Spirulina lysate containing a candidate methanolysin from M. cuniculi exhibits activity in the assay (FIG. 2B). Lytic activity is not observed in wild-type Spirulina lysate (FIG. 2B).

### A. Lysis Assay in Buffer

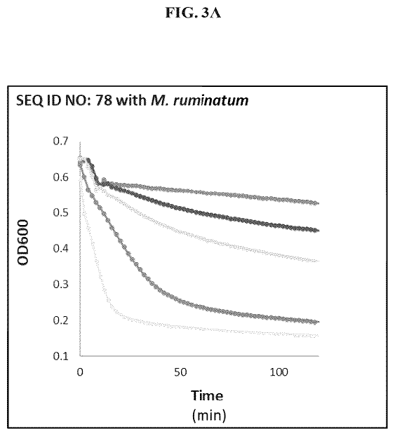

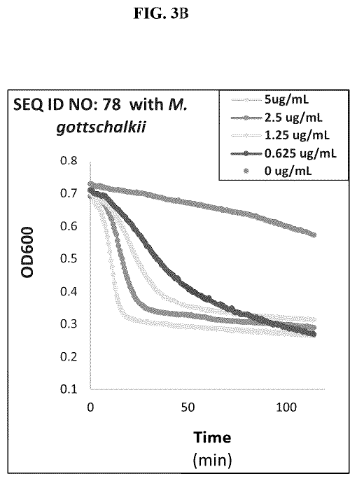

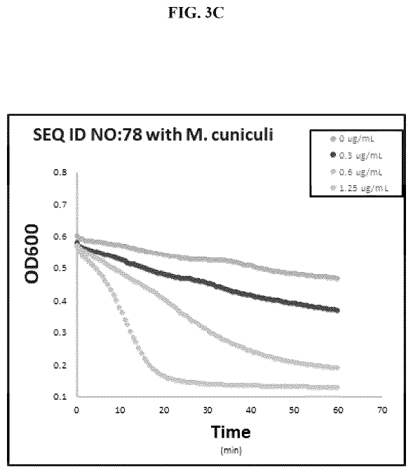

An initially study utilized lytic assays in buffers to assess the effect of SEQ ID NO: 78 on methanogens. For this study, three major rumen methanogens were grown in anaerobic conditions. The cells were washed aerobically after centrifugation. The resulting ghost cells were incubated with the concentrations of SEQ ID NO: 78 in 10 mM BisTris propane (pH 7.5) and 1 mM TCEP. Enzyme concentrations are indicated with the results in FIGS. 3A-3C. Lysis of these methanogens was indicated by a decrease in OD600.

Results show methanogen lysis within a buffer solution occurred in a dose-dependent manner with increasing concentration of methanolysin enzymes.

### B. Lysis Assay in Clarified Rumen Fluid

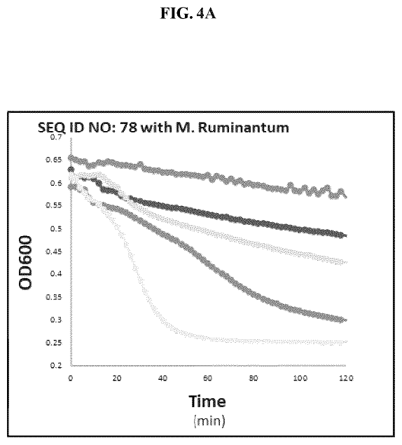

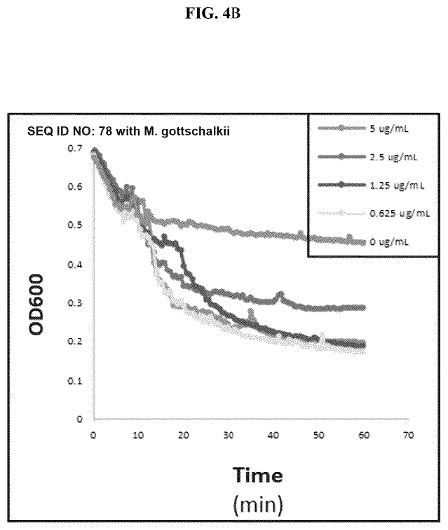

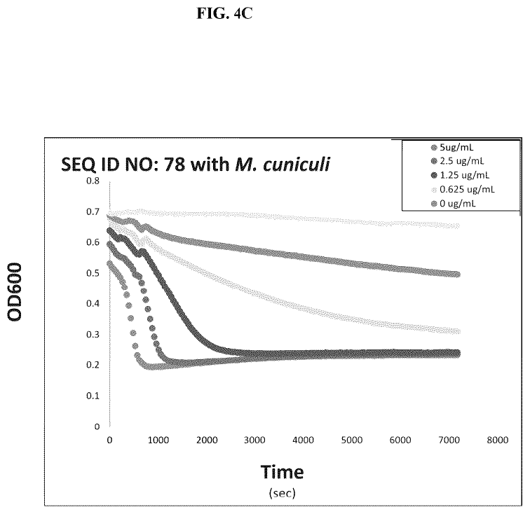

Another study used lytic assays in clarified rumen fluid to assess the effect of SEQ ID NO: 78 on methanogens. For this study, three major rumen methanogens were grown in anaerobic conditions. The cells were washed aerobically after centrifugation. The resulting ghost cells were incubated with the concentrations of SEQ ID NO: 78 in clarified rumen fluid. Rumen fluid was clarified by two round of centrifugation (5 min. at 21000×g) and filtration through a 0.2 μM filter. Enzyme concentrations are indicated with the results in FIGS. 4A-4C. Lysis of these methanogens was indicated by a decrease in OD600.

Results show methanogen lysis within clarified rumen fluid occurred in a dose-dependent manner with increasing concentration of methanolysin enzymes.

### C. Lytic Rate of Methanolysins

Additional lytic activity studies were conducted to assess the effect of methanolysin on different methanogens. For this analysis, ghost cells of three major methanogens were incubated with SEQ ID NO: 78 in 10 mM BisTris-propane (pH 7.5) and 1 mM TCEP at concentrations of 1-5 μg/mL.

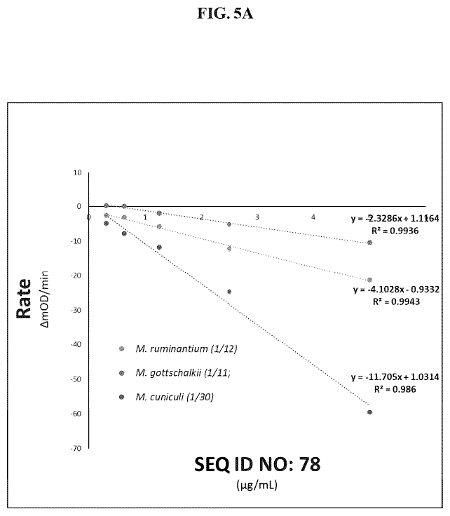

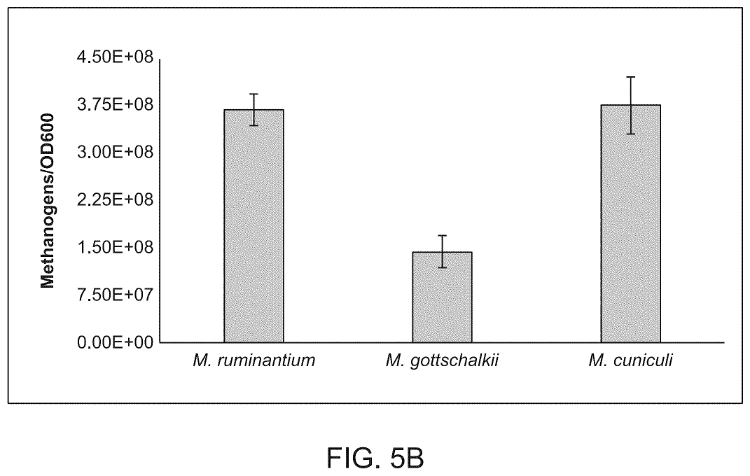

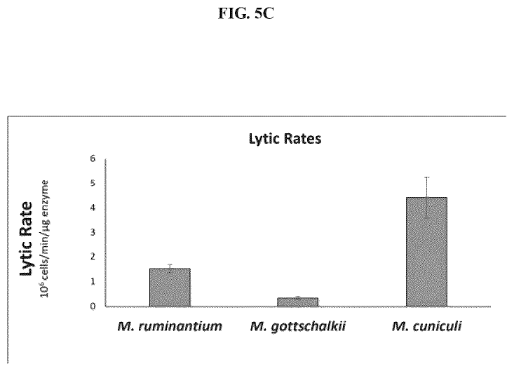

The results are in FIGS. 5A-5C. As shown, the maximum linear rate change (over a 5 min time frame) was recorded and determine to be correlated with enzyme concentration (FIG. 5A). Using a hemocytometer, a ratio of methanogens to OD was determined (FIG. 5B). Using the methanogen/OD ratio (FIG. 5B) and indicated rates (FIG. 5A), the lytic rate of methanolysins was calculated as seen in FIG. 5C.

### Example 2: Methane Emission Inhibition by Spirulina Methanolysin-Expressing Strains In Vitro

The effects of SEQ ID NO: 78 were assessed on living methanogens. For this study, Methanobrevibacter gottschalkii was grown in anaerobic conditions in hungate tubes. In an anaerobic chamber, the enzyme SEQ ID NO: 78 was added to these cultures. SEQ ID NO: 78 was incubated with 1 mM TCEP before addition to ensure all cysteines were reduced. After addition of SEQ ID NO: 78, Methanobrevibacter gottschalkii was pressurized with a 4:1 mixture of H₂/CO₂ at 6 PSI. Methane amounts were measured 24 hours after pressurization.

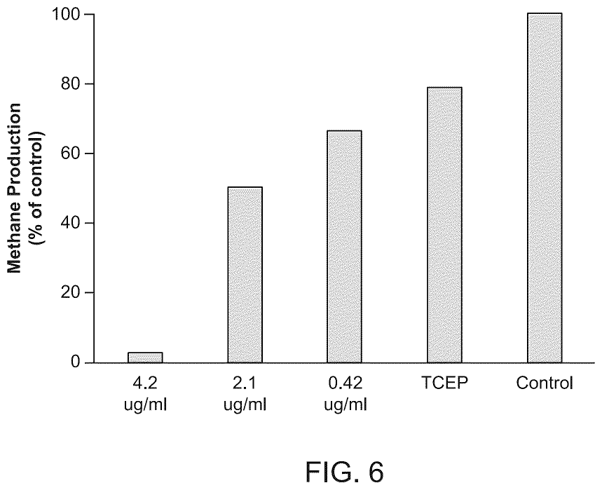

Results are shown in FIG. 6. SEQ ID NO: 78 concentrations are shown on the x-axis. The “control” sample did not have any enzyme added, while the “TCEP” control only had this molecule (no enzyme) added to the culture.

### Example 3: Identification of Novel Lysins

Candidate methanolysins were identified by sequence similarity to three proteins that had previously been shown to be active against methanogen targets, PeiR, PeiW, and PeiP. The Basic Local Alignment Search Tool (BLAST) from the National Center for Biotechnology Information (NCBI) was used to search GenBank databases for proteins with a similar sequence to the previously studied enzymes. Unique sequences with an E-value of <1E-4 were identified (Table 1). Initial studies focused on sequences with an E-value <1E-9.

For this study, Methanolysins were tested against Methanobrevibacter ruminantium, Methanosphaera cuniculi and Methanobrevibacter ruminantium. BLAST queries were performed with PeiR from Methanobrevibacter ruminantium (Altermann et al. (2018) Frontiers in Microbiology 9 10.3389/fmicb.2018.02378). Top hits were overexpressed by cell free protein expression and analyzed against three rumen methanogens (M. ruminantium, M. gottschalkii and M. cuniculi) in Table 3. Lytic rates were determined in the way displayed in FIGS. 5A-5C.

TABLE 3
Novel lysins identified with BLAST.

| | | | Measured Lytic Rate | | |
|---|---|---|---|---|---|
| | NCBI | | M. | M. | M. |
| Candidate | sequence ID | NCBI annotation | ruminantium | gottschalkii | cuniculi |
| SEQ ID | WP_095608514.1 | Methanosphaera cuniculi | 1.5 | 0.3 | 4.4 |
| NO: 78 | | peptidoglycan-binding protein | | | |
| Pei002_H6 | MCE7700070.1 | hypothetical protein | 2.3 | 0.0 | 0.0 |
| | | [Methanobacterium paludis] | | | |
| Pei005_H6 | MDR1819006.1 | hypothetical protein | 1.3 | 7.7 | 0.0 |
| | | [Methanobrevibacter sp.] | | | |
| Pei008_H6 | WP_305513774.1 | cysteine peptidase family C39 | 2.0 | 1.3 | 5.7 |
| | | domain-containing protein | | | |
| | | [Methanobrevibacter sp. V14] | | | |
| Pei009_H6 | MBQ9026945.1 | C39 family peptidase | 2.3 | 12.9 | 14.6 |
| | | [Methanobrevibacter sp.] | | | |
| Pei010_H6 | WP_296875535.1 | cysteine peptidase family C39 | 6.5 | 0.2 | 22.9 |
| | | domain-containing [uncultured | | | |
| | | Methanobrevibacter sp.] | | | |
| Pei011_H6 | MBR1610527.1 | C39 family peptidase | 18.2 | 0.6 | 25.3 |
| | | [Methanobrevibacter sp.] | | | |
| Pei012_H6 | WP_095608516.1 | peptidoglycan-binding protein | 1.0 | 14.0 | 0.0 |
| | | [Methanosphaera cuniculi] | | | |
| Pei013_H6 | WP_091699050.1 | C39 family peptidase | 0.2 | 13.7 | 0.0 |
| | | [Methanobrevibacter | | | |
| | | gottschalkii] | | | |
| Pei014_H6 | MDO5820711.1 | cysteine peptidase family C39 | 2.4 | 0.1 | 5.3 |
| | | domain-containing protein | | | |
| | | [Methanobrevibacter sp.] | | | |
| Pei024_H6 | DAF03330.1 | TPA: Pseudomurein endo- | 1.9 | 2.3 | 0.0 |
| | | isopeptidase Pei [Caudoviricetes | | | |
| | | sp.] | | | |
| Pei033_H6 | WP_303222269.1 | cysteine peptidase family C39 | 18.8 | 0.0 | 4.7 |
| | | domain-containing protein | | | |
| | | [Methanosphaera stadtmanae] | | | |
| Pei034_H6 | WP_303347937.1 | cysteine peptidase family C39 | 15.7 | 17.5 | 2.1 |
| | | domain-containing protein | | | |
| | | [Methanosphaera sp.] | | | |
| Pei035_H6 | UTB32069.1 | transglutaminase domain- | 0.0 | 11.4 | 6.9 |
| | | containing protein | | | |
| | | [Methanobacterium sp. ERen5] | | | |
| Pei036_H6 | WP_013645370.1 | transglutaminase domain- | 0.9 | 4.6 | 3.7 |
| | | containing protein | | | |
| | | [Methanobacterium lacus] | | | |
| Pei043_H6 | WP_069584472.1 | transglutaminase domain- | 1.1 | 1.8 | 2.6 |
| | | containing protein | | | |
| | | [Methanobacterium] | | | |
| Pei054_H6 | DAO11645.1 | hypothetical protein | 0.0 | 6.1 | 0.0 |
| | | [Caudoviricetes sp.] | | | |
| Pei058_H6 | WP_019262093.1 | hypothetical protein | 1.2 | 24.1 | 4.3 |
| | | [Methanobrevibacter smithii] | | | |
| Pei059_H6 | DAR45108.1 | Pseudomurein endo-isopeptidase | 0.1 | 1.6 | 1.3 |
| | | Pei, partial [Caudoviricetes sp.] | | | |
| Pei060_H6 | WP_303210353.1 | transglutaminase domain- | 0.1 | 17.8 | 0.0 |
| | | containing protein | | | |
| | | [Methanobrevibacter smithii] | | | |
| Pei063_H6 | WP_292878891.1 | hypothetical protein | 1.0 | 9.0 | 4.1 |
| | | [Methanobrevibacter sp.] | | | |
| Pei065_H6 | DAY53184.1 | hypothetical protein | 2.0 | 9.8 | 1.7 |
| | | [Caudoviricetes sp.] | | | |
| Pei067_H6 | WP_292803112.1 | cysteine peptidase family C39 | 5.2 | 3.7 | 8.8 |
| | | domain-containing protein | | | |
| | | [Methanobrevibacter sp.] | | | |

### Example 4: Engineered Methanolysins

Multiple strategies were used to engineer improved methanolysins with improved killing activity and accumulation in Spirulina. These strategies are described below.

First, the candidates were expressed as fusions to protein tags that included but were not limited to, maltose binding protein, thioredoxin, phycocyanin, and SUMO tags.

Second, to further improve activity and/or expression, domains that were hypothesized to slow enzymatic activity and lower expression were deleted. PeiR possesses an N-terminal cell wall binding domain (further referred to as the pseudomurein binding domain) and a C-terminal catalytic domain that cleaves peptide bonds that are essential to the structural integrity of the archaeal cell wall. Removal of the binding domain from the lysin PlyCD10 can increase both Spirulina accumulation and specific activity without affecting specificity (Wang Q, Euler C W, Delaune A, Fischetti V A. Using a Novel Lysin to Help Control Clostridium difficile Infections. Antimicrob Agents Chemother. 2015 December; 59 (12): 7447-57. doi: 10.1128/AAC.01357-15. Epub 2015 Sep. 21. PMID: 26392484; PMCID: PMC4649177).

Third, molecular electrostatic analysis was used to design mutations to complement solvent exposed regions with charged residues to aid protein folding and solubility.

Overall, these strategies were pursued to develop designs for candidate methanolysins and were demonstrated with the analysis of PeiR below.

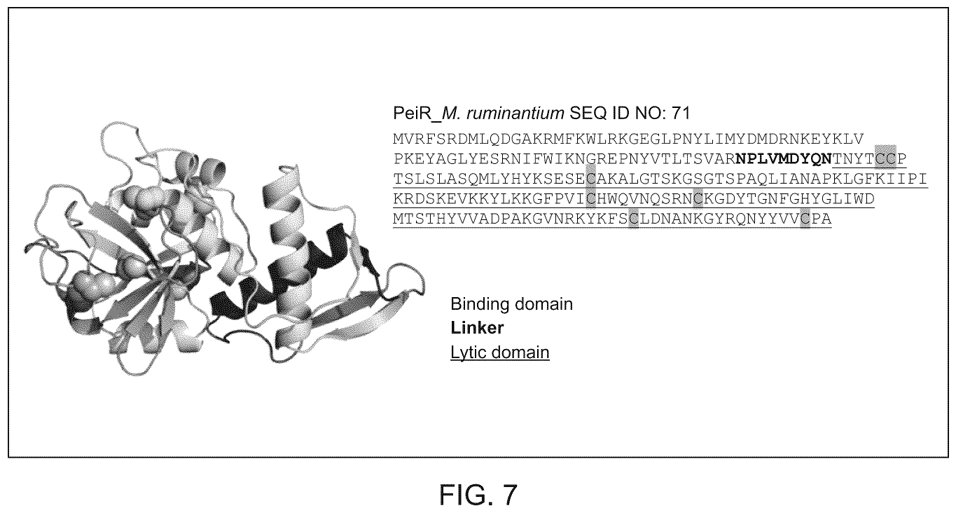

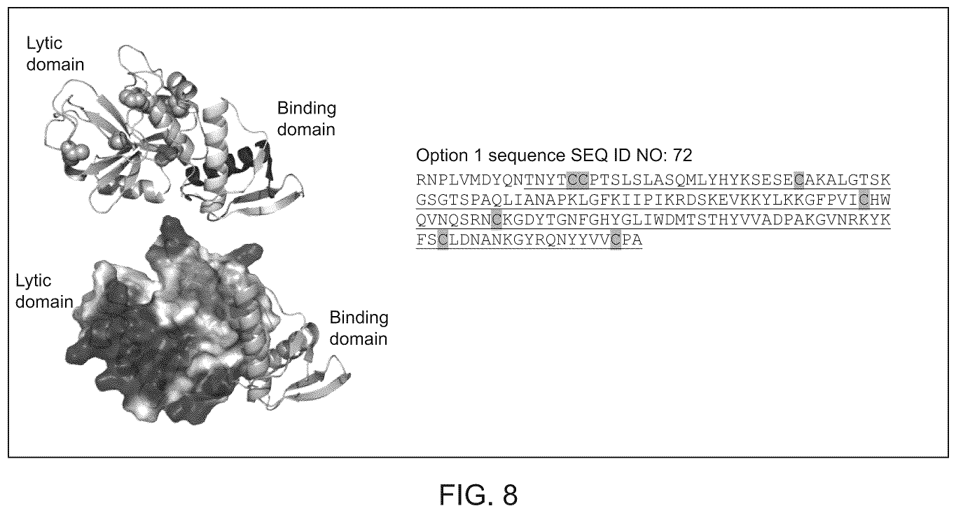

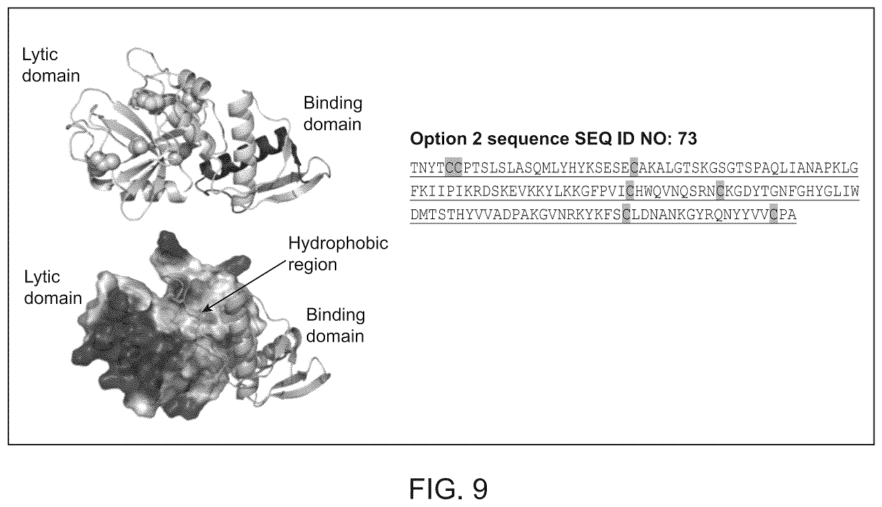

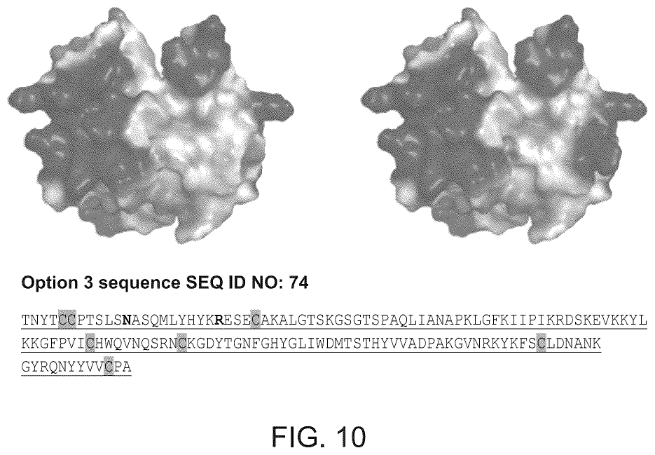

Structural analysis was initiated on the candidate molecules to delineate the protein sub-domains for in vitro activity analysis. A structural model of PeiR was constructed in AlphaFold to develop multiple expression design strategies for removal of the pseudomurein binding domain without causing negative side-effects (FIG. 7). The linker sequence connecting sub-domains could be a source of complication. Models demonstrating the removal of the N-terminal pseudomurein binding domain while preserving (FIG. 8) or deleting (FIG. 9) the linker region were developed to assess the potential impact to protein solubility. The models of PeiR suggest that removal of the linker may expose a hydrophobic region on the catalytic domain which could decrease solubility. Electrostatic static analysis of PeiR identified two residues that can be mutated to promote solubility and accumulation in Spirulina (FIG. 10).

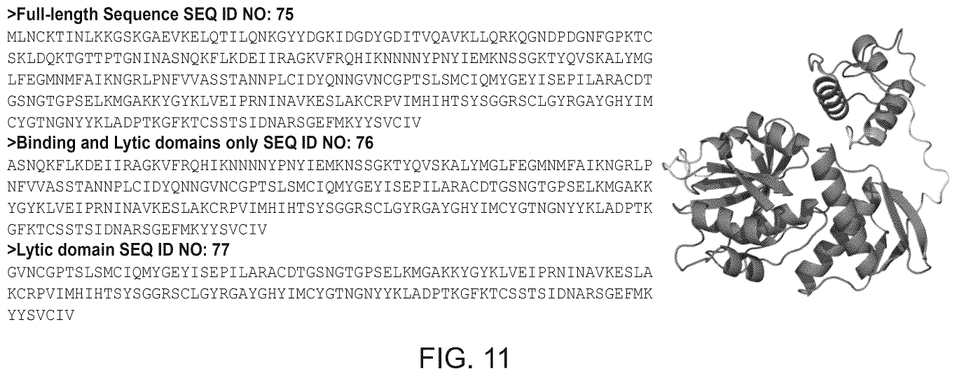

The candidate methanolysin from M. cuniculi was also subject to structural analysis. An N-terminal domain upstream of the pseudomurein binding domain with structural similarity to domains found in gram-negative lysins was identified (FIG. 11). Based on the structural models, novel constructs were designed to remove the N-terminal domain and the pseudomurein binding domain to facilitate expression of the catalytic domain (FIG. 11). The sequences are shown below in Table 4.

TABLE 4
Amino acid sequences of proteins expressed
in Spirulina.

| SEQ | Engineered | |
|---|---|---|
| ID NO: | Methanolysins | Amino Acid Sequences |
| 101 | SP2619 | MGHHHHHHGSGGLNCKTINL |
| | | KKGSKGAEVKELQTILQNKG |
| | | YYDGKIDGDYGDITVQAVKL |
| | | LQRKQGNDPDGNFGPKTCSK |
| | | LDQKTGTTPTGNINASNQKF |
| | | LKDEIIRAGKVFRQHIKNNN |
| | | NYPNYIEMKNSSGKTYQVSK |
| | | ALYMGLFEGMNMFAIKNGRL |
| | | PNFVVASSTANNPLCIDYQN |
| | | NGVNCGPTSLSMCIQMYGEY |
| | | ISEPILARACDTGSNGTGPS |
| | | ELKMGAKKYGYKLVEIPRNI |
| | | NAVKESLAKCRPVIMHIHTS |
| | | YSGGRSCLGYRGAYGHYIMC |
| | | YGTNGNYYKLADPTKGFKTC |
| | | SSTSIDNARSGEFMKYYSVC |
| | | IVSS |
| 102 | SP2620 | MGGLNCKTINLKKGSKGAEV |
| | | KELQTILQNKGYYDGKIDGD |
| | | YGDITVQAVKLLQRKQGNDP |
| | | DGNFGPKTCSKLDQKTGTTP |
| | | TGNINASNQKFLKDEIIRAG |
| | | KVFRQHIKNNNNYPNYIEMK |
| | | NSSGKTYQVSKALYMGLFEG |
| | | MNMFAIKNGRLPNFVVASST |
| | | ANNPLCIDYQNNGVNCGPTS |
| | | LSMCIQMYGEYISEPILARA |
| | | CDTGSNGTGPSELKMGAKKY |
| | | GYKLVEIPRNINAVKESLAK |
| | | CRPVIMHIHTSYSGGRSCLG |
| | | YRGAYGHYIMCYGTNGNYYK |
| | | LADPTKGFKTCSSTSIDNAR |
| | | SGEFMKYYSVCIVSSGGSHH |
| | | HHHH |

TABLE 5
Complete vector sequences expressing
the engineered proteins of Table 4.

| SEQ | Engineered | |
|---|---|---|
| ID NO: | Methanolysins | Nucleic Acid Sequences |
| 122 | SP2619 | GGAATCATGAATCATCCCACAACTAAGCATCAAAAAATTA |
| | | CATCCTGTTACTGGGGAATTTCTGAACTACCTGGGTTAGA |
| | | TACAGGTAATATTAAACTCCTGAATCAATGTGGTATTGAA |
| | | AACACCCAGCAACTGCTCACGCGCGGGAGTAATAATGCTA |
| | | ATAAAATCGCCTTATCTAACCAACTAGGTATCAACATCAG |
| | | AGATATCAGTAAATGGGTCGCAATGTCCGACTTAGCCAGG |
| | | GTTCCCAGTATTGGCTATCAATATTGTGGTGTCCTTTTGC |
| | | ATAGTGGCGTAAGTTCAGTTAACCAACTATCTCAGATGTC |
| | | TATTCAACAACTGCATAAACAGATTTTACGGCTCTATGTA |
| | | GGAACCTTGCAATCTCGCCAACTCTGTCCATCTGTTGATG |
| | | TCATCCAAACCTGGATTAAACAAGCTAAAATTTTGCATAA |
| | | AATTAATTAATAATTACTAGATGAAATCATGAATTTTTCC |
| | | GAAGATTTGCGACAAAATGCACCAGCCACCGAAAGAAACC |
| | | GCGAACCTATTTTAGAGGTCTTAAAAAGGGTGTTGCCCGC |
| | | CACGGGAACCGTTTTAGAAGTAGCCAGCGGCACAGGACAA |
| | | CACGCGGTTTTTTTTGCTCCCCGTCTCTCCCCTAGGCGGT |
| | | GGCTACCTTCCGACCCGCAACCTTTGTTACAACAGAGTAT |
| | | TAAAGCATGGCAAATTCATTCACCTTCAGATAATCTTTAC |
| | | CCTCCCCTACAGTTAAATGTAGAAGCTAATCCTTGGCCAG |
| | | TGGAAGGGAATGATTTACCCTGGGAATTATCGGAATTTCC |
| | | CATCACTGCTATTGTAGCAATTAACCTGATTCATATTTCG |
| | | CCTTGGTCGGCTTGTGAAGGGTTAATGGCGGGAAGCGATC |
| | | GCATTTTGAAACCGGGGGGAATTTTATATCTCTATGGACC |
| | | CTATAAAATAGCTGGAAAACACACCGCACCTAGTAACGCT |
| | | TCCTTTGATGAATATTTGCGAACTTCTAACCCTAAGTGGG |
| | | GTGTCAGAAACCTTGATGATTCACATACGCGGCCGCCTGG |
| | | GCCTTGAGCTCGAATTTCCTGCATTAATGAATCGGCCAAC |
| | | GCGCGGGGAGAGGCGGTTTGCGTATTGGGCGCTCTTCCGC |
| | | TTCCTCGCTCACTGACTCGCTGCGCTCGGTCGTTCGGCTG |
| | | CGGCGAGCGGTATCAGCTCACTCAAAGGCGGTAATACGGT |
| | | TATCCACAGAATCAGGGGATAACGCAGGAAAGAACATGTG |
| | | AGCAAAAGGCCAGCAAAAGGCCAGGAACCGTAAAAATAGC |
| | | GGAGTGTATACTGGCTTACTATGTTGGCACTGATGAGGGT |
| | | GTCAGTGAAGTGCTTCATGTGGCAGGAGAAAAAAGGCTGC |
| | | ACCGGTGCGTCAGCAGAATATGTGATACAGGATATATTCC |
| | | GCTTCCTCGCTCACTGACTCGCTACGCTCGGTCGTTCGAC |
| | | TGCGGCGAGCGGAAATGGCTTACGAACGGGGCGGAGATTT |
| | | CCTGGAAGATGCCAGGAAGATACTTAACAGGGAAGTGAGA |
| | | GGGCCGCGGCAAAGCCGTTTTTCCATAGGCTCCGCCCCCC |
| | | TGACAAGCATCACGAAATCTGACGCTCAAATCAGTGGTGG |
| | | CGAAACCCGACAGGACTATAAAGATACCAGGCGTTTCCCC |
| | | CTGGCGGCTCCCTCGTGCGCTCTCCTGTTCCTGCCTTTCG |
| | | GTTTACCGGTGTCATTCCGCTGTTATGGCCGCGTTTGTCT |
| | | CATTCCACGCCTGACACTCAGTTCCGGGTAGGCAGTTCGC |
| | | TCCAAGCTGGACTGTATGCACGAACCCCCCATTCAGTCCG |
| | | ACCGCTGCGCCTTATCCGGTAACTATCGTCTTGAGTCCAA |
| | | CCCGGAAAGACATGCAAAAGCACCACTGGCAGCAGCCACT |
| | | GGTAATTGATTTAGAGGAGTTAGTCTTGAAGTCATGCGCC |
| | | GGTTAAGGCTAAACTGAAAGGACAAGTTTTGGTGACTGCG |
| | | CTCCTCCAAGCCAGTTACCTCGGTTCAAAGAGTTGGTAGC |
| | | TCAGAGAACCTTCGAAAAACCGCCCTGCAAGGCGGTTTTT |
| | | TCGTTTTCAGAGCAAGAGATTACGCGCAGACCAAAACGAT |
| | | CTCAAGAAGATCATCTTATTAAGATTATCAAAAAGGATCT |
| | | TCACCTAGATCCTTTTAAATTAAAAATGAAGTTTTAAATC |
| | | AATCTAAAGTATATATGAGTAAACTTGGTCTGACAGTTAC |
| | | CAATGCTTAATCAGTGAGGCACCTATCTCAGCGATCTGTC |
| | | TATTTCGTTCATCCATAGTTGCCTGACTCCCCGTCGTGTA |
| | | GATAACTACGATACGGGAGGGCTTACCATCTGGCCCCAGT |
| | | GCTGCAATGATACCGCGGGACCCACGCTCACCGGCTCCAG |
| | | ATTTATCAGCAATAAACCAGCCAGCCGGAAGGGCCGAGCG |
| | | CAGAAGTGGTCCTGCAACTTTATCCGCCTCCATCCAGTCT |
| | | ATTAATTGTTGCCGGGAAGCTAGAGTAAGTAGTTCGCCAG |
| | | TTAATAGTTTGCGCAACGTTGTTGCCATTGCTACAGGCAT |
| | | CGTGGTGTCACGCTCGTCGTTTGGTATGGCTTCATTCAGC |
| | | TCCGGTTCCCAACGATCAAGGCGAGTTACATGATCCCCCA |
| | | TGTTGTGCAAAAAAGCGGTTAGCTCCTTCGGTCCTCCGAT |
| | | CGTTGTCAGAAGTAAGTTGGCCGCAGTGTTATCACTCATG |
| | | GTTATGGCAGCACTGCATAATTCTCTTACTGTCATGCCAT |
| | | CCGTAAGATGCTTTTCTGTGACTGGTGAGTACTCAACCAA |
| | | GTCATTCTGAGAATAGTGTATGCGGCGACCGAGTTGCTCT |
| | | TGCCCGGCGTCAATACGGGATAATACCGCGCCACATAGCA |
| | | GAACTTTAAAAGTGCTCATCATTGGAAAACGTTCTTCGGG |
| | | GCGAAAACTCTCAAGGATCTTACCGCTGTTGAGATCCAGT |
| | | TCGATGTAACCCACTCGTGCACCCAACTGATCTTCAGCAT |
| | | CTTTTACTTTCACCAGCGTTTCTGGGTGAGCAAAAACAGG |
| | | AAGGCAAAATGCCGCAAAAAAGGGAATAAGGGCGACACGG |
| | | AAATGTTGAATACTCATACTCTTCCTTTTTCAATATTATT |
| | | GAAGCATTTATCAGGGTTATTGTCTCATGAGCGGATACAT |
| | | ATTTGAATGTATTTAGAAAAATAAACAAATAGGGGTTCCG |
| | | CGCACATTTCCCCGAAAAGTGCCACCTGACGTCTAAGAAA |
| | | CCATTATTATCATGACATTAACCTATAAAAATAGGCGTAT |
| | | CACGAGGCCCTTTCGTCTCGCGCGTTTCGGTGATGACGGT |
| | | GAAAACCTCTGACACATGCAGCTCCCGGAGACGGTCACAG |
| | | CTTGTCTGTAAGCGGATGCCAAGCTTGCATGCCTGCAGGT |
| | | GGCGCGCCTATGGGGGAGGATTTTATGATCGGATGTTGAG |
| | | TCAGTCTGAGTGGTCTAAAATTCCTACGGTGGGAATTGTG |
| | | TTTGCGGCCGCCAGGGTGGATACTCTCCCTAGGGATAGTT |
| | | GGGACCAGCCTTTGGCGGCGGTTTGTACAGAGGAGGGTAT |
| | | ATGGGAGTTTCCAAAATAACTTCTGGTGGGGGGGCAAATT |
| | | ATCGGGCGATCGCCACTTATATGGTATTAATTGCTATAGC |
| | | GATCGCTATGCTATTTCCCCTATTTTGGTTGGTGGGAACT |
| | | GCCTTTAAATCCCCTACCGAAAACATCTTTCAGGTTCCCC |
| | | CCCAGTTTATTCCAAGGGAACCTACTTTCACTAATTTTGT |
| | | CAGAGTTTGGCAAACCAATCCCTTTGGTCGCTATTTATTT |
| | | AATAGTACCCTCGTCGCCCTTTTAACCGTCGGATTAAATC |
| | | TGCTATTTTGTTCCTTGGCGGCCTATCCTTTGGCGCGCTT |
| | | AAACTTTTGGGGTCGCGATTTTCTCCTAAGTGCGATCGTT |
| | | TCCACCATCATGATACCCTTTCAGATTGTCATGATTCCCC |
| | | TGTATATTCTAGCAGTACAGTTAGGATTAAGAAATACTTA |
| | | TTTAGGCATTATTTTTCCATCTTTAGCCTCCGCTTTTGGC |
| | | ATCTTTCTATTGCGACAGGCATTTATGGGAGTTCCCAAAG |
| | | AGTTAGAAGAAGCCGCCAGAATTGATGGCTGTTCAGAATT |
| | | AGGGATTTGGTGGCATATTATGATCCCCTCAGTCCGTCCC |
| | | GCCTTAATGACCTTAGCCATTTTTGTATTTATCGGGTCTT |
| | | GGAGTGATTTTCTCTGGCCCTTAATTGTAGTCGATCGCCC |
| | | AGAATATTATACCATCCCTTTAGGCGTGGCAAATCTAGCC |
| | | GGAACTTTTTCTCTCGATTGGCGGATAGTCGCCGCCGGTT |
| | | CCGTCATTTCTATCGCTCCTGTGTTACTACTATTTTTAAT |
| | | CGTACAACGGTATATTGTGCCTACAGATACAGCTACGGGG |
| | | GTTAAAGGTTGAATGAGGATTATTAATCTCAGTCCTAATA |
| | | ATCAAAATCATATCCACCAGGCGGCGACTTTATTAGTCGC |
| | | AGAATTTCGGGAAAATTGGCCGAATGCTTGGCCGACTTAT |
| | | GAACGTGGTTTAGCCGAGGTTATGGAGTCTTTTGGCGACG |
| | | ATAGAGTTAATCTAGTCGCCGTCGATGAAAATGATAATCT |
| | | TCTAGGCTGGATTGGTGGTATTAGCCAATATCAAGGCCAT |
| | | GTTTGGGAATTACATCCTTTGGTAGTCAAAAGCGATTATC |
| | | AAGGGTTAGGAATAGGGCGAAAATTGGTCGCCAATTTAGA |
| | | GGATTATGTCCGCTCACAAGGAGGGTTAACCTTATGGTTG |
| | | GGTACCGATGACGAAAATAATTTAACCTCCCTATCTGGTG |
| | | TCGAATTATACCCCCATTTTTTGGAAAATATTGCTAACAT |
| | | AAAAAACCATGGTCGTCATCCCTATGAATTTTATCAGAAA |
| | | TGCGGTTTTGTGATTATGGGAGTAGTCCCCGATGCTAACG |
| | | GTATCGGGAAACCTGATATTTTGATGGCTAAATCTCTGAG |
| | | AATTGACAATCATCCTAAATCCAACTGAATGTTGTCAGTT |
| | | AAGTTTATAGGGCTGCTATGGTATGATAAAAAAATCCCCC |
| | | CAGCAGTTGGGGGGGAATGATTAATTAGGTATTGAATTGG |
| | | TTAAATTGGAGGTGTGTTGGGGCTGACTTTTGCTGCCTTG |
| | | TTGGGGGGGGCGATTTTAACTGAGGTCACTTTTTCTTGGC |
| | | CGGGATTGGCTAATCGCTTGTATGAGGCTATCCGTCTGCG |
| | | GGATTATCCCACGGTTCAAGGGGTGGTGGTATTTTTGGCC |
| | | GTTATTGTGGTGGCGGCTAGTTTGGCGATCGATATTTTGA |
| | | ATGCTTGTATTGACCCACGGATTCGCTATTAATTCCCTGT |
| | | CAGAAAATATACTTAGTTATTAATACTTAGTTATTTTTCT |
| | | TAATAAATTTTAACAAACCAAAGGGCGTTGGCAGTGTATA |
| | | AAGGATATTTGAAGGGGTGAGAAGGAAGGCGAATGTTGAT |
| | | TTATGAAGTTTGATTAACATTTGTATCAAAATATAAAATT |
| | | CTTCTCATAAACCCTGTACAATCTTTTAAGATTTCGGAAA |
| | | GTGTTCTAGGATACTGAAGAAATGAACCACGGGGCAATTG |
| | | TTAAAAGCCTTTGTCGATGGTTCGCCCCGGAAGGGGTCTT |
| | | AGGAGGTGACACCGATGGATTGATTGTCGTGATCATTCAT |
| | | GGTGTGTCCAATCCCAACTCAACTCTAAGCAAGTCAACAA |
| | | GTAGGAGATAAATCTATGGGACATCATCATCACCACCACG |
| | | GTTCTGGTGGATTGAACTGCAAAACCATTAACTTAAAAAA |
| | | GGGAAGTAAGGGCGCGGAAGTCAAGGAACTGCAGACCATT |
| | | CTCCAGAACAAAGGGTACTATGATGGTAAAATAGACGGTG |
| | | ATTACGGCGATATCACCGTACAAGCAGTGAAATTACTTCA |
| | | GCGCAAACAAGGTAACGATCCGGACGGTAACTTCGGACCG |
| | | AAAACCTGCTCCAAACTGGATCAGAAAACTGGCACGACCC |
| | | CAACTGGCAATATTAACGCGAGCAATCAGAAGTTTCTGAA |
| | | GGATGAGATTATTCGTGCTGGCAAAGTATTTCGTCAACAC |
| | | ATTAAAAACAATAATAACTATCCCAATTACATTGAAATGA |
| | | AAAATTCCAGCGGCAAAACCTACCAGGTGTCAAAAGCGCT |
| | | GTATATGGGCTTGTTTGAAGGCATGAACATGTTCGCCATT |
| | | AAAAACGGTCGCCTGCCGAATTTTGTGGTTGCCTCGAGCA |
| | | CGGCAAACAATCCGCTTTGTATTGACTATCAGAATAACGG |
| | | TGTCAATTGTGGCCCGACGAGTTTATCAATGTGCATCCAG |
| | | ATGTACGGGGAATACATCTCAGAACCGATCCTCGCGCGTG |
| | | CGTGCGACACAGGAAGCAATGGTACAGGCCCTAGCGAACT |
| | | GAAAATGGGCGCCAAGAAATATGGCTATAAACTGGTTGAA |
| | | ATTCCGCGAAACATAAACGCGGTGAAAGAGTCGCTAGCCA |
| | | AATGTCGCCCTGTCATTATGCATATCCATACTAGTTATAG |
| | | CGGGGGTCGGTCTTGTCTGGGCTACCGTGGCGCCTATGGC |
| | | CACTACATCATGTGCTATGGTACCAACGGAAATTACTATA |
| | | AGCTGGCAGATCCAACCAAGGGGTTTAAAACCTGTTCGTC |
| | | GACGTCTATCGATAATGCTCGCTCTGGTGAGTTCATGAAA |
| | | TACTATAGCGTTTGCATTGTGTCAAGCTAACAGCCAAATA |
| | | AAACGAAAGGCTCAGTCGAAAGACTGGGCCTTTCGTTTTA |
| | | TCTGTTGTTTGTCGGTGAACGCTCTCCTGAGTAGGACAAA |
| | | TCCGCCGGGAGCGGATTTGAACGTTGCGAAGCAACGGCCC |
| | | GGAGGGTGGCGGGCAGGACGCCCGCCATAAACTGCCAGGC |
| | | ATCAAATTAAGCAGAAGGCCATCCTGACGGATGGCCTTTT |
| | | TGCGTTTCTACAAACTCCGGATCCGGCCGGCTTGAAGACA |
| | | GGTGGGACAGGAGGGAGGTAAATGC |
| 123 | SP2620 | GGAATCATGAATCATCCCACAACTAAGCATCAAAAAATTA |
| | | CATCCTGTTACTGGGGAATTTCTGAACTACCTGGGTTAGA |
| | | TACAGGTAATATTAAACTCCTGAATCAATGTGGTATTGAA |
| | | AACACCCAGCAACTGCTCACGCGCGGGAGTAATAATGCTA |
| | | ATAAAATCGCCTTATCTAACCAACTAGGTATCAACATCAG |
| | | AGATATCAGTAAATGGGTCGCAATGTCCGACTTAGCCAGG |
| | | GTTCCCAGTATTGGCTATCAATATTGTGGTGTCCTTTTGC |
| | | ATAGTGGCGTAAGTTCAGTTAACCAACTATCTCAGATGTC |
| | | TATTCAACAACTGCATAAACAGATTTTACGGCTCTATGTA |
| | | GGAACCTTGCAATCTCGCCAACTCTGTCCATCTGTTGATG |
| | | TCATCCAAACCTGGATTAAACAAGCTAAAATTTTGCATAA |
| | | AATTAATTAATAATTACTAGATGAAATCATGAATTTTTCC |
| | | GAAGATTTGCGACAAAATGCACCAGCCACCGAAAGAAACC |
| | | GCGAACCTATTTTAGAGGTCTTAAAAAGGGTGTTGCCCGC |
| | | CACGGGAACCGTTTTAGAAGTAGCCAGCGGCACAGGACAA |
| | | CACGCGGTTTTTTTTGCTCCCCGTCTCTCCCCTAGGCGGT |
| | | GGCTACCTTCCGACCCGCAACCTTTGTTACAACAGAGTAT |
| | | TAAAGCATGGCAAATTCATTCACCTTCAGATAATCTTTAC |
| | | CCTCCCCTACAGTTAAATGTAGAAGCTAATCCTTGGCCAG |
| | | TGGAAGGGAATGATTTACCCTGGGAATTATCGGAATTTCC |
| | | CATCACTGCTATTGTAGCAATTAACCTGATTCATATTTCG |
| | | CCTTGGTCGGCTTGTGAAGGGTTAATGGCGGGAAGCGATC |
| | | GCATTTTGAAACCGGGGGGAATTTTATATCTCTATGGACC |
| | | CTATAAAATAGCTGGAAAACACACCGCACCTAGTAACGCT |
| | | TCCTTTGATGAATATTTGCGAACTTCTAACCCTAAGTGGG |
| | | GTGTCAGAAACCTTGATGATTCACATACGCGGCCGCCTGG |
| | | GCCTTGAGCTCGAATTTCCTGCATTAATGAATCGGCCAAC |
| | | GCGCGGGGAGAGGCGGTTTGCGTATTGGGCGCTCTTCCGC |
| | | TTCCTCGCTCACTGACTCGCTGCGCTCGGTCGTTCGGCTG |
| | | CGGCGAGCGGTATCAGCTCACTCAAAGGCGGTAATACGGT |
| | | TATCCACAGAATCAGGGGATAACGCAGGAAAGAACATGTG |
| | | AGCAAAAGGCCAGCAAAAGGCCAGGAACCGTAAAAATAGC |
| | | GGAGTGTATACTGGCTTACTATGTTGGCACTGATGAGGGT |
| | | GTCAGTGAAGTGCTTCATGTGGCAGGAGAAAAAAGGCTGC |
| | | ACCGGTGCGTCAGCAGAATATGTGATACAGGATATATTCC |
| | | GCTTCCTCGCTCACTGACTCGCTACGCTCGGTCGTTCGAC |
| | | TGCGGCGAGCGGAAATGGCTTACGAACGGGGCGGAGATTT |
| | | CCTGGAAGATGCCAGGAAGATACTTAACAGGGAAGTGAGA |
| | | GGGCCGCGGCAAAGCCGTTTTTCCATAGGCTCCGCCCCCC |
| | | TGACAAGCATCACGAAATCTGACGCTCAAATCAGTGGTGG |
| | | CGAAACCCGACAGGACTATAAAGATACCAGGCGTTTCCCC |
| | | CTGGCGGCTCCCTCGTGCGCTCTCCTGTTCCTGCCTTTCG |
| | | GTTTACCGGTGTCATTCCGCTGTTATGGCCGCGTTTGTCT |
| | | CATTCCACGCCTGACACTCAGTTCCGGGTAGGCAGTTCGC |
| | | TCCAAGCTGGACTGTATGCACGAACCCCCCATTCAGTCCG |
| | | ACCGCTGCGCCTTATCCGGTAACTATCGTCTTGAGTCCAA |
| | | CCCGGAAAGACATGCAAAAGCACCACTGGCAGCAGCCACT |
| | | GGTAATTGATTTAGAGGAGTTAGTCTTGAAGTCATGCGCC |
| | | GGTTAAGGCTAAACTGAAAGGACAAGTTTTGGTGACTGCG |
| | | CTCCTCCAAGCCAGTTACCTCGGTTCAAAGAGTTGGTAGC |
| | | TCAGAGAACCTTCGAAAAACCGCCCTGCAAGGCGGTTTTT |
| | | TCGTTTTCAGAGCAAGAGATTACGCGCAGACCAAAACGAT |
| | | CTCAAGAAGATCATCTTATTAAGATTATCAAAAAGGATCT |
| | | TCACCTAGATCCTTTTAAATTAAAAATGAAGTTTTAAATC |
| | | AATCTAAAGTATATATGAGTAAACTTGGTCTGACAGTTAC |
| | | CAATGCTTAATCAGTGAGGCACCTATCTCAGCGATCTGTC |
| | | TATTTCGTTCATCCATAGTTGCCTGACTCCCCGTCGTGTA |
| | | GATAACTACGATACGGGAGGGCTTACCATCTGGCCCCAGT |
| | | GCTGCAATGATACCGCGGGACCCACGCTCACCGGCTCCAG |
| | | ATTTATCAGCAATAAACCAGCCAGCCGGAAGGGCCGAGCG |
| | | CAGAAGTGGTCCTGCAACTTTATCCGCCTCCATCCAGTCT |
| | | ATTAATTGTTGCCGGGAAGCTAGAGTAAGTAGTTCGCCAG |
| | | TTAATAGTTTGCGCAACGTTGTTGCCATTGCTACAGGCAT |
| | | CGTGGTGTCACGCTCGTCGTTTGGTATGGCTTCATTCAGC |
| | | TCCGGTTCCCAACGATCAAGGCGAGTTACATGATCCCCCA |
| | | TGTTGTGCAAAAAAGCGGTTAGCTCCTTCGGTCCTCCGAT |
| | | CGTTGTCAGAAGTAAGTTGGCCGCAGTGTTATCACTCATG |
| | | GTTATGGCAGCACTGCATAATTCTCTTACTGTCATGCCAT |
| | | CCGTAAGATGCTTTTCTGTGACTGGTGAGTACTCAACCAA |
| | | GTCATTCTGAGAATAGTGTATGCGGCGACCGAGTTGCTCT |
| | | TGCCCGGCGTCAATACGGGATAATACCGCGCCACATAGCA |
| | | GAACTTTAAAAGTGCTCATCATTGGAAAACGTTCTTCGGG |
| | | GCGAAAACTCTCAAGGATCTTACCGCTGTTGAGATCCAGT |
| | | TCGATGTAACCCACTCGTGCACCCAACTGATCTTCAGCAT |
| | | CTTTTACTTTCACCAGCGTTTCTGGGTGAGCAAAAACAGG |
| | | AAGGCAAAATGCCGCAAAAAAGGGAATAAGGGCGACACGG |
| | | AAATGTTGAATACTCATACTCTTCCTTTTTCAATATTATT |
| | | GAAGCATTTATCAGGGTTATTGTCTCATGAGCGGATACAT |
| | | ATTTGAATGTATTTAGAAAAATAAACAAATAGGGGTTCCG |
| | | CGCACATTTCCCCGAAAAGTGCCACCTGACGTCTAAGAAA |
| | | CCATTATTATCATGACATTAACCTATAAAAATAGGCGTAT |
| | | CACGAGGCCCTTTCGTCTCGCGCGTTTCGGTGATGACGGT |
| | | GAAAACCTCTGACACATGCAGCTCCCGGAGACGGTCACAG |
| | | CTTGTCTGTAAGCGGATGCCAAGCTTGCATGCCTGCAGGT |
| | | GGCGCGCCTATGGGGGAGGATTTTATGATCGGATGTTGAG |
| | | TCAGTCTGAGTGGTCTAAAATTCCTACGGTGGGAATTGTG |
| | | TTTGCGGCCGCCAGGGTGGATACTCTCCCTAGGGATAGTT |
| | | GGGACCAGCCTTTGGCGGCGGTTTGTACAGAGGAGGGTAT |
| | | ATGGGAGTTTCCAAAATAACTTCTGGTGGGGGGGCAAATT |
| | | ATCGGGCGATCGCCACTTATATGGTATTAATTGCTATAGC |
| | | GATCGCTATGCTATTTCCCCTATTTTGGTTGGTGGGAACT |
| | | GCCTTTAAATCCCCTACCGAAAACATCTTTCAGGTTCCCC |
| | | CCCAGTTTATTCCAAGGGAACCTACTTTCACTAATTTTGT |
| | | CAGAGTTTGGCAAACCAATCCCTTTGGTCGCTATTTATTT |
| | | AATAGTACCCTCGTCGCCCTTTTAACCGTCGGATTAAATC |
| | | TGCTATTTTGTTCCTTGGCGGCCTATCCTTTGGCGCGCTT |
| | | AAACTTTTGGGGTCGCGATTTTCTCCTAAGTGCGATCGTT |
| | | TCCACCATCATGATACCCTTTCAGATTGTCATGATTCCCC |
| | | TGTATATTCTAGCAGTACAGTTAGGATTAAGAAATACTTA |
| | | TTTAGGCATTATTTTTCCATCTTTAGCCTCCGCTTTTGGC |
| | | ATCTTTCTATTGCGACAGGCATTTATGGGAGTTCCCAAAG |
| | | AGTTAGAAGAAGCCGCCAGAATTGATGGCTGTTCAGAATT |
| | | AGGGATTTGGTGGCATATTATGATCCCCTCAGTCCGTCCC |
| | | GCCTTAATGACCTTAGCCATTTTTGTATTTATCGGGTCTT |
| | | GGAGTGATTTTCTCTGGCCCTTAATTGTAGTCGATCGCCC |
| | | AGAATATTATACCATCCCTTTAGGCGTGGCAAATCTAGCC |
| | | GGAACTTTTTCTCTCGATTGGCGGATAGTCGCCGCCGGTT |
| | | CCGTCATTTCTATCGCTCCTGTGTTACTACTATTTTTAAT |
| | | CGTACAACGGTATATTGTGCCTACAGATACAGCTACGGGG |
| | | GTTAAAGGTTGAATGAGGATTATTAATCTCAGTCCTAATA |
| | | ATCAAAATCATATCCACCAGGCGGCGACTTTATTAGTCGC |
| | | AGAATTTCGGGAAAATTGGCCGAATGCTTGGCCGACTTAT |
| | | GAACGTGGTTTAGCCGAGGTTATGGAGTCTTTTGGCGACG |
| | | ATAGAGTTAATCTAGTCGCCGTCGATGAAAATGATAATCT |
| | | TCTAGGCTGGATTGGTGGTATTAGCCAATATCAAGGCCAT |
| | | GTTTGGGAATTACATCCTTTGGTAGTCAAAAGCGATTATC |
| | | AAGGGTTAGGAATAGGGCGAAAATTGGTCGCCAATTTAGA |
| | | GGATTATGTCCGCTCACAAGGAGGGTTAACCTTATGGTTG |
| | | GGTACCGATGACGAAAATAATTTAACCTCCCTATCTGGTG |
| | | TCGAATTATACCCCCATTTTTTGGAAAATATTGCTAACAT |
| | | AAAAAACCATGGTCGTCATCCCTATGAATTTTATCAGAAA |
| | | TGCGGTTTTGTGATTATGGGAGTAGTCCCCGATGCTAACG |
| | | GTATCGGGAAACCTGATATTTTGATGGCTAAATCTCTGAG |
| | | AATTGACAATCATCCTAAATCCAACTGAATGTTGTCAGTT |
| | | AAGTTTATAGGGCTGCTATGGTATGATAAAAAAATCCCCC |
| | | CAGCAGTTGGGGGGGAATGATTAATTAGGTATTGAATTGG |
| | | TTAAATTGGAGGTGTGTTGGGGCTGACTTTTGCTGCCTTG |
| | | TTGGGGGGGGCGATTTTAACTGAGGTCACTTTTTCTTGGC |
| | | CGGGATTGGCTAATCGCTTGTATGAGGCTATCCGTCTGCG |
| | | GGATTATCCCACGGTTCAAGGGGTGGTGGTATTTTTGGCC |
| | | GTTATTGTGGTGGCGGCTAGTTTGGCGATCGATATTTTGA |
| | | ATGCTTGTATTGACCCACGGATTCGCTATTAATTCCCTGT |
| | | CAGAAAATATACTTAGTTATTAATACTTAGTTATTTTTCT |
| | | TAATAAATTTTAACAAACCAAAGGGCGTTGGCAGTGTATA |
| | | AAGGATATTTGAAGGGGTGAGAAGGAAGGCGAATGTTGAT |
| | | TTATGAAGTTTGATTAACATTTGTATCAAAATATAAAATT |
| | | CTTCTCATAAACCCTGTACAATCTTTTAAGATTTCGGAAA |
| | | GTGTTCTAGGATACTGAAGAAATGAACCACGGGGCAATTG |
| | | TTAAAAGCCTTTGTCGATGGTTCGCCCCGGAAGGGGTCTT |
| | | AGGAGGTGACACCGATGGATTGATTGTCGTGATCATTCAT |
| | | GGTGTGTCCAATCCCAACTCAACTCTAAGCAAGTCAACAA |
| | | GTAGGAGATAAATCTATGGGTGGATTGAACTGCAAAACCA |
| | | TTAACTTAAAAAAGGGAAGTAAGGGCGCGGAAGTCAAGGA |
| | | ACTGCAGACCATTCTCCAGAACAAAGGGTACTATGATGGT |
| | | AAAATAGACGGTGATTACGGCGATATCACCGTACAAGCAG |
| | | TGAAATTACTTCAGCGCAAACAAGGTAACGATCCGGACGG |
| | | TAACTTCGGACCGAAAACCTGCTCCAAACTGGATCAGAAA |
| | | ACTGGCACGACCCCAACTGGCAATATTAACGCGAGCAATC |
| | | AGAAGTTTCTGAAGGATGAGATTATTCGTGCTGGCAAAGT |
| | | ATTTCGTCAACACATTAAAAACAATAATAACTATCCCAAT |
| | | TACATTGAAATGAAAAATTCCAGCGGCAAAACCTACCAGG |
| | | TGTCAAAAGCGCTGTATATGGGCTTGTTTGAAGGCATGAA |
| | | CATGTTCGCCATTAAAAACGGTCGCCTGCCGAATTTTGTG |
| | | GTTGCCTCGAGCACGGCAAACAATCCGCTTTGTATTGACT |
| | | ATCAGAATAACGGTGTCAATTGTGGCCCGACGAGTTTATC |
| | | AATGTGCATCCAGATGTACGGGGAATACATCTCAGAACCG |
| | | ATCCTCGCGCGTGCGTGCGACACAGGAAGCAATGGTACAG |
| | | GCCCTAGCGAACTGAAAATGGGCGCCAAGAAATATGGCTA |
| | | TAAACTGGTTGAAATTCCGCGAAACATAAACGCGGTGAAA |
| | | GAGTCGCTAGCCAAATGTCGCCCTGTCATTATGCATATCC |
| | | ATACTAGTTATAGCGGGGGTCGGTCTTGTCTGGGCTACCG |
| | | TGGCGCCTATGGCCACTACATCATGTGCTATGGTACCAAC |
| | | GGAAATTACTATAAGCTGGCAGATCCAACCAAGGGGTTTA |
| | | AAACCTGTTCGTCGACGTCTATCGATAATGCTCGCTCTGG |
| | | TGAGTTCATGAAATACTATAGCGTTTGCATTGTGTCAAGC |
| | | GGCGGTAGTCACCATCACCACCATCACTAACAGCCAAATA |
| | | AAACGAAAGGCTCAGTCGAAAGACTGGGCCTTTCGTTTTA |
| | | TCTGTTGTTTGTCGGTGAACGCTCTCCTGAGTAGGACAAA |
| | | TCCGCCGGGAGCGGATTTGAACGTTGCGAAGCAACGGCCC |
| | | GGAGGGTGGCGGGCAGGACGCCCGCCATAAACTGCCAGGC |
| | | ATCAAATTAAGCAGAAGGCCATCCTGACGGATGGCCTTTT |
| | | TGCGTTTCTACAAACTCCGGATCCGGCCGGCTTGAAGACA |
| | | GGTAGGCTAAGGGCCCGGCTAATGC |

In vitro lytic activity assays and biophysical characterization of the purified proteins was conducted to inform the molecule selection and aid in the design of Spirulina transformation vectors.

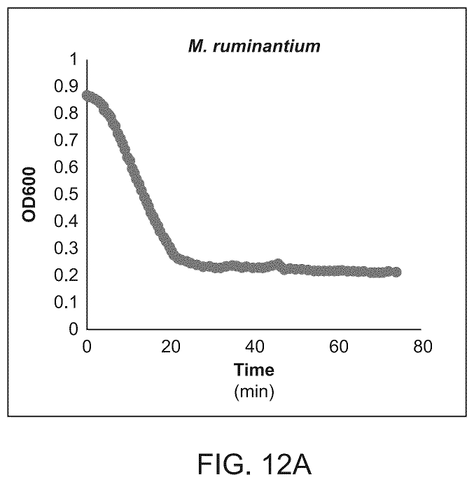

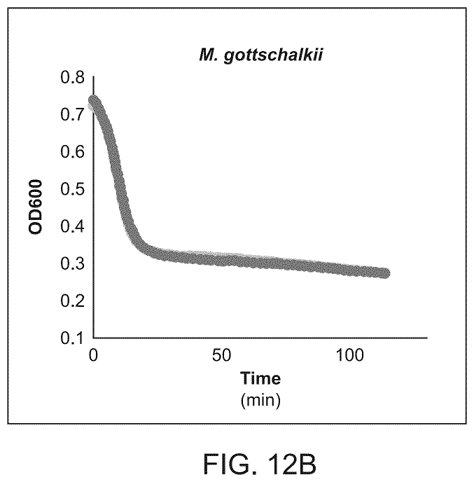

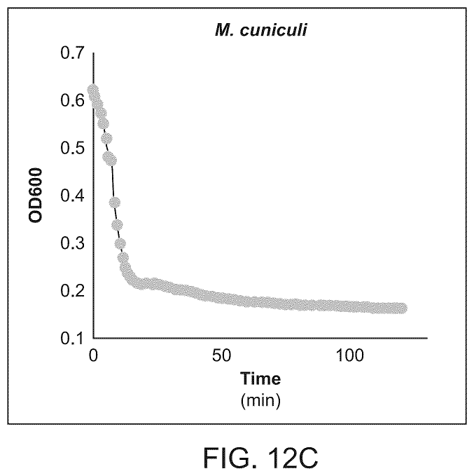

To conduct the lytic activity assays, three major rumen methanogens were grown in anaerobic conditions. The cells were washed aerobically after centrifugation. The resulting ghost cells were incubated with the Spirulina strain SP2620 at 0.2 mg/mL. SP2620 was grown in a photobioreactor and shown by immunoblotting to express SEQ ID NO: 78. Results are in FIGS. 12A-12C. Lysis of these methanogens was indicated by a decrease in OD600.

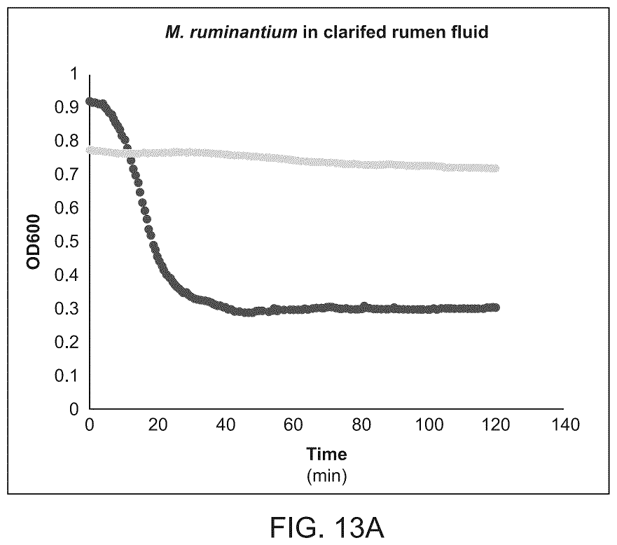

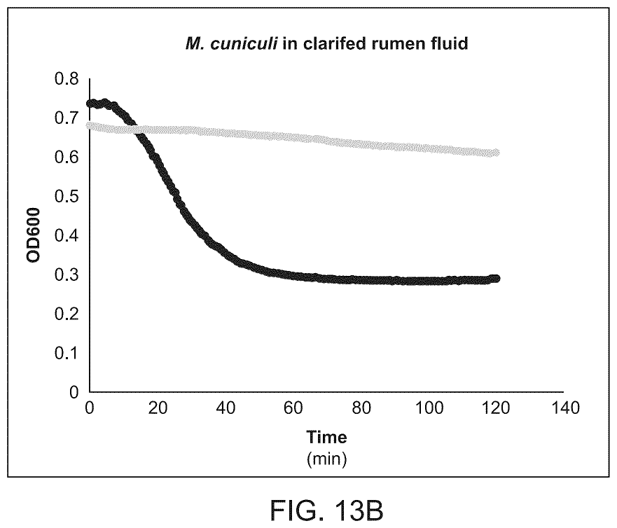

To assess the effect of SP2620 on lytic activity, lysis assays were also conducted in clarified rumen fluid. For this study, Methanobrevibacter ruminantium (FIG. 13A) and Methanosphaera cuniculi (FIG. 13B) were grown in anaerobic conditions. The cells were washed aerobically after centrifugation. The resulting ghost cells were incubated with the Spirulina strain SP2620 at 0.2 mg/mL in clarified rumen fluid. Rumen fluid was clarified by two round of centrifugation (5 min. at 21000×g) and filtration through a 0.2 μM filter. SP2620 was grown in a photobioreactor and shown by immunoblotting to express SEQ ID NO: 78. Results are in FIGS. 13A and 13B. Lysis of these methanogens was indicated by a decrease in OD600.

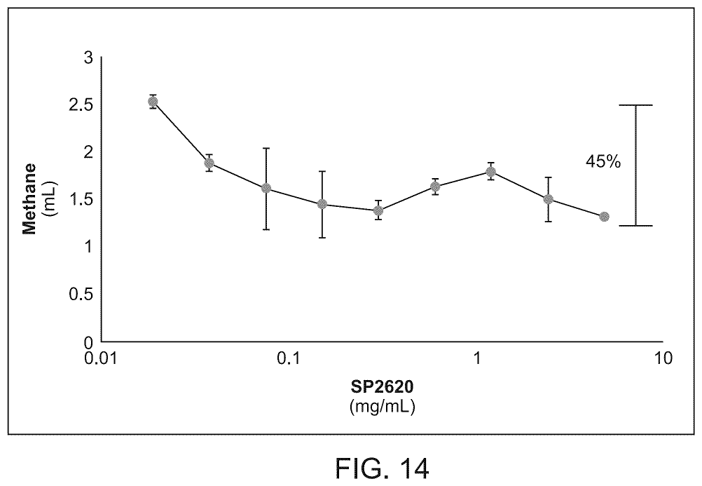

In addition, a dose dependence study was conducted to assess the methane content of rumen fluid supplemented with H₂/CO₂ in response to different SP2620 dosages. For this study, rumen fluid was taken from canulated cattle using a device that filters out large particles. The resulting fluid was stored in the dark at room temperature for about 24 hours. After this period, the material was thawed in anaerobic conditions and diluted 1:1 in saliva buffer (100 mM NaHCO₃; 100 mM NH4 (H2PO4). SP2620 was brought to the concentrations shown on the x-axis. The resulting cultures were sealed and pressurized with a 4:1 mixture of H₂/CO₂ at 6 PSI. Methane was measured using a Shimazu gas chromatograph 24 hours after inoculation and compared to a standard curve generated with pure methane gas. Results are in FIG. 14. As shown, an increase in SP2620 dose results in a decrease in methane.

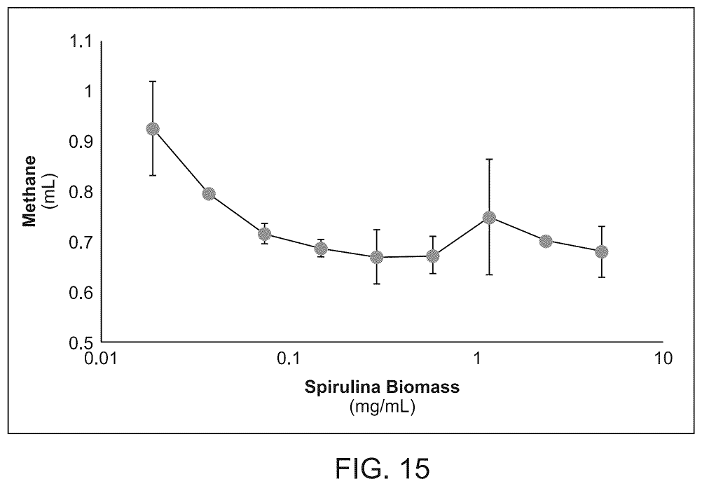

Moreover, another dose dependence study was conducted to assess the methane content in rumen fluid supplemented with alfalfa/corn in response to different SP2620 dosages. For this study, rumen fluid was taken from canulated cattle using a device that filters out large particles. The resulting fluid was stored in the dark at room temperature for about 24 hours. After this period, the material was thawed in anaerobic conditions and diluted 1:1 in saliva buffer (100 mM NaHCO₃; 100 mM NH₄(H₂PO₄). SP2620 was brought to the concentrations shown on the x-axis. Alfalfa was brought to final concentration of 18 mg/mL and rolled corn was brought to a final concentration of 2 mg/mL. Methane was measured using a Shimadzu gas chromatograph 24 hours after inoculation and compared to a standard curve generated with pure methane gas. Results are in FIG. 15. As shown, an increase in SP2620 dose results in a decrease in methane.

### Example 5: Inhibition of Methane Emission Produced by Enteric Archaea In Vivo

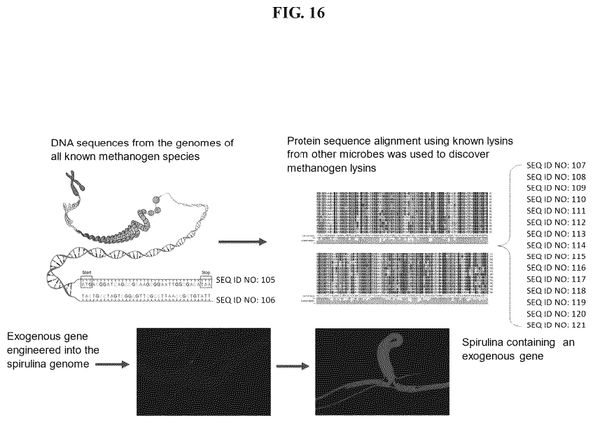

Genetically modified Arthrospira platensis (Spirulina) strains that express methanogen-killing lytic enzymes (methanolysins), cloned by a method exemplified in FIG. 16, are used to inhibit methane emission produced by enteric archaea. The Spirulina-methanolysin product is administered as a feed additive and is comprised of a single strain or cocktail of Spirulina strains to target the diversity of methanogens in a rumen. By killing the methanogens, the source of methane, emissions are expected to diminish drastically by the treated animal.

Unchecked methanogenesis may consume about 10% of the energy content of animal feed, so a substantial reduction in methanogen numbers may lead to improved livestock health and economic benefit, by allowing decreased feedstock consumption for the same amount of animal productivity.

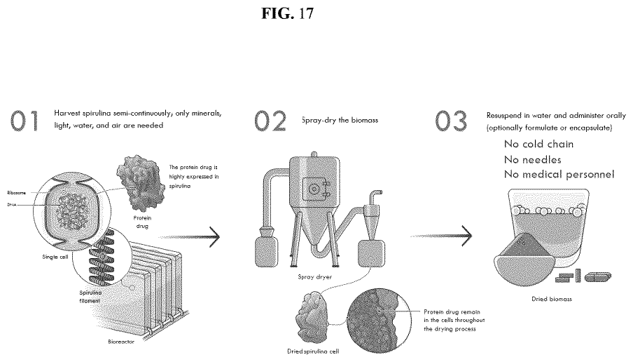

In vivo studies assess efficacy in dairy cows and beef cattle, a Spirulina-methanolysin feed additive could further be used in all ruminants, as seen in FIG. 17. The Spirulina-methanolysin strains could also be utilized to inhibit ex vivo methane emissions from sources like ruminant manure, where methanogens are present.

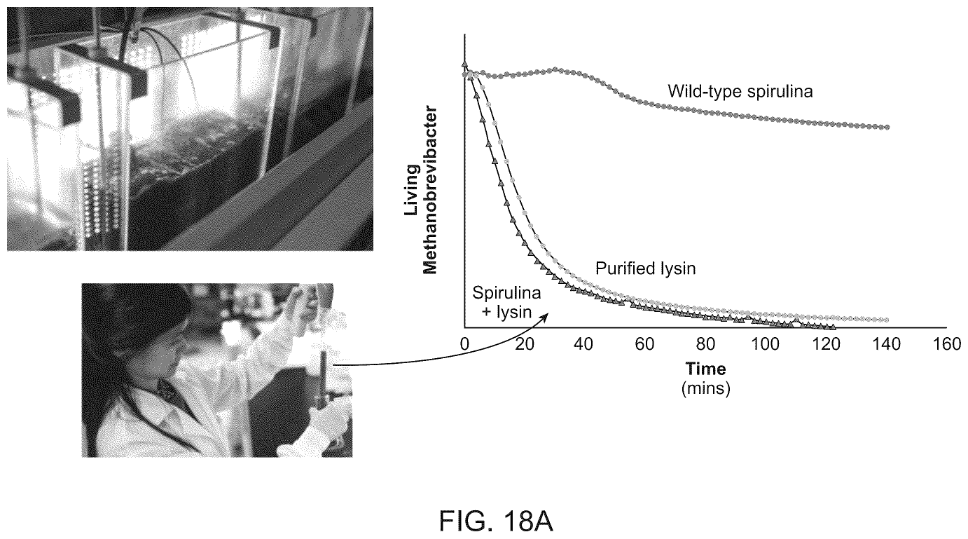

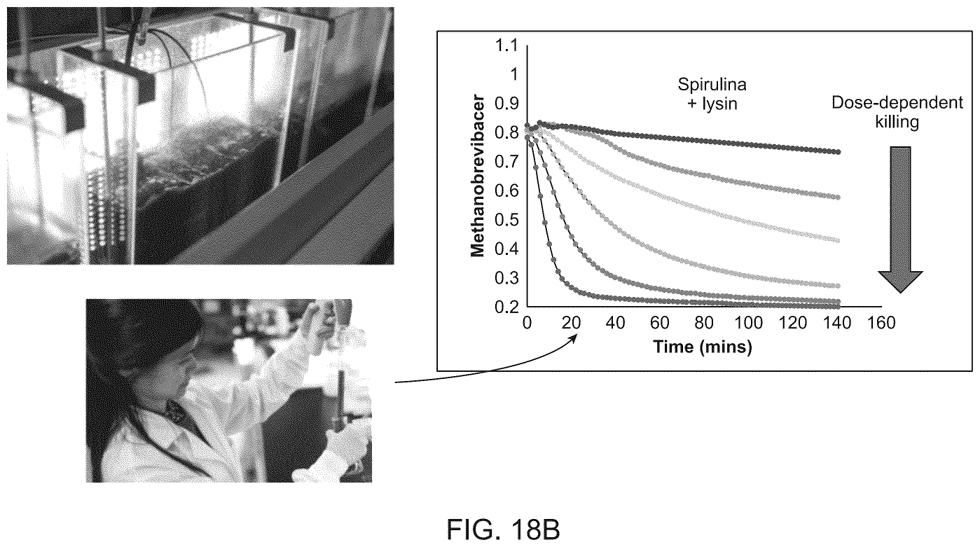

A cocktail of dried Spirulina strains, each containing a different methanolysin, is added to ruminant feed to kill the diversity of methanogens in the rumen and inhibit methane emissions. Upon ingestion, the pH of the rumen (pH 5.5-7.0) facilitates release of methanolysins from the dried killed Spirulina. The released methanolysin become active upon solubilization and specifically target the cell wall of archaea which includes methanogens. Bacteria and animal cells do not possess cellular structures that are recognized by methanolysins, so these cells are expected to remain unaffected by treatment. Dead methanogen debris and digested Spirulina and methanolysins are expected to be absorbed by the animal during digestion. The Spirulina-methanolysin feed additive is administered regularly to prevent rumen recolonization by methanogens. An exemplary result is described in FIGS. 18A and 18B. Dose and administration frequency will need to be determined empirically for each ruminant species.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

What is claimed is:

1. A modified Spirulina cell comprising an exogenous methanolysin comprising an amino acid sequence with at least 90% identity to SEQ ID NO: 78.

2. The modified Spirulina cell of claim 1, wherein the exogenous methanolysin comprises SEQ ID NO: 78.

3. The modified Spirulina cell of claim 1, wherein the exogeneous methanolysin comprises a catalytic domain as encoded by SEQ ID NO: 101 or 102.

4. The modified Spirulina cell of claim 1, wherein the modified Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

5. The modified Spirulina cell of claim 4, wherein the modified Spirulina is the A. platensis.

6. A modified Spirulina cell comprising an exogenous methanolysin, said exogenous methanolysin comprising a catalytic domain comprising at least 90% identity to SEQ ID NO: 101 or 102.

7. The modified Spirulina cell of claim 6, wherein the exogenous methanolysin comprises SEQ ID NO: 101 or 102.

8. The modified Spirulina cell of claim 6, wherein the modified Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

9. The modified Spirulina cell of claim 6, wherein the modified Spirulina is of the species A. platensis.

10. A food supplement composition, comprising: the modified Spirulina cell of claim 1.

11. A nutraceutical or pharmaceutical composition comprising the modified Spirulina cell of claim 1.

12. A method of reducing methane emissions, the method comprising: administering a composition comprising a modified Spirulina comprising an exogenous methanolysin to a subject; wherein said exogeneous methanolysin comprises an amino acid sequence with at least 90% identity to SEQ ID NO: 78.

13. The method of claim 12, wherein the exogenous methanolysin comprises SEQ ID NO: 78.

14. The method of claim 12, wherein the exogenous methanolysin comprises a catalytic domain as encoded by SEQ ID NO: 1 or SEQ ID NO: 102.

15. The method of claim 12, wherein the modified Spirulina is of a species selected from the group consisting of: A. amethystine, A. ardissonei, A. argentina, A. balkrishnanii, A. baryana, A. boryana, A. braunii, A. breviarticulata, A. brevis, A. curta, A. desikacharyiensis, A. funiformis, A. fusiformis, A. ghannae, A. gigantean, A. gomontiana, A. gomontiana var. crassa, A. indica, A. jenneri var. platensis, A. jenneri Stizenberger, A. jenneri f. purpurea, A. joshii, A. khannae, A. laxa, A. laxissima, A. leopoliensis, A. major, A. margaritae, A. massartii, A. massartii var. indica, A. maxima, A. meneghiniana, A. miniata var. constricta, A. miniata, A. miniata f. acutissima, A. neapolitana, A. nordstedtii, A. oceanica, A. okensis, A. pellucida, A. platensis, A. platensis var. non-constricta, A. platensis f. granulate, A. platensis f. minor, A. platensis var. tenuis, A. santannae, A. setchellii, A. skujae, A. spirulinoides f. tenuis, A. spirulinoides, A. subsalsa, A. subtilissima, A. tenuis, A. tenuissima, and A. versicolor.

16. The method of claim 12, wherein the modified Spirulina is of the species A. platensis.

17. The method of claim 12, wherein the administration is effective at reducing methane emissions by at least about 5-fold as compared to an otherwise comparable method lacking the administration.

18. The method of claim 17, wherein the reduction in methane emissions is determined by detecting methane in metabolic gases generated by the subject.

19. The method of claim 12, wherein the subject is a ruminant.

20. The method of claim 12, wherein the ruminant is selected from the group consisting of: cow, bovine, goat, sheep, giraffe, deer, gazelle, and antelope.


SEQUENCE LISTING (124 sequences)

SEQ ID NO: 1 - protein, 229 aa, Methanobrevibacter gottschalkii
MVRFSKKEMQKASKIIRESIKKGKGQPLSVKMVEMDKGKTIKVSKAYYMGLFEAQNIFIRNKGRKPNYVTLNSKANNPLVMDYQDNAYTCCPTSFSMATQLLFDNISEKECAKALNTVLGSGTDPSDLIKNSGKLGYKVTPIKRDYKSVKSSLDKGFAVIAHIQTRPATCLGYSGDYGHYVLLYGAYRKLLTKYYKVADPTKGLKTCKASILDKATNGRNIKYYSVGIL

SEQ ID NO: 2 - protein, 223 aa, Methanobrevibacter oralis
MIKFMNDVIVKASKTIRKNIKEGKGVPTMLVMMDSNGNSHKLSKKEVAGIFEARNVFILKKGRLPNYVTLNSTANNPLVMDYQNNKYNCCPTSLSMAIQMLYSYKSENVCAKALKTNTAGTAPLDLVSGAKNLGYEVKRISRNKASVKAQLNMGKSVIAHIQTKSATCLGYVGDYGHYVLIYGMSSDNYKIADPTKGIKTCRTGILDKATNGREIHYYSVSPL

SEQ ID NO: 3 - protein, 228 aa, Methanobrevibacter sp.
MTRFSKNEMNKASKTIVKSIRNGKGLPRIVEMKEMDHNKTIRVDKSHYNGLFEAQNVFIRGHGRLPNYVTMNSTANNPLVMDYQDNGYTCGPTSLSMAIQMLYGWKSEKRCAEACHTGSSGTSPDDLIAGAKSLGYKVTPIKRNFAGVKQSLNKGYPVIAHIETGGRTKPKCLGYVNNYGHYILIYGTTASGGYKVADPTKGLRVCSASELDKATNGRSINYYSVKPL

SEQ ID NO: 4 - protein, 223 aa, unidentified
  source 1..223: note = bacterium (gut metagenome)
MKDAMTDGAKRVFKFIKEHERYPKTLRMVDSTNKSHVLTKAQYMGLFEAENIFRIKHGRQPNYTTYNSTASNPLVLDYQDGKMTCCPTSLSMCSQLLFHFKSENQCKKALKTDPKEGTSPDKLVNNAKDLGFKVVVIGRDYKKVVKALDCGYGVIMHIETAKKTRPKCLGYTNNYGHYVCCYKAKNNKYYIADPTKGMKTCKASEIDKAKNDRDLKYYIIKPL

SEQ ID NO: 5 - protein, 227 aa, Methanobrevibacter sp.
MTRFLNSEMVKASKTIRNNIKNGNGLPRSIKMIDTDGKVHTVEKKYYNGLFESRNVFTLKNGRMPNYVTLNQTANNPLVLNYQDDKYSCCVASFNMCVQMLYDWIPESKIKKLFKTNQYGTSPANMVAGAKALGYKVEQIGRNFSSVQKAISKGFPVLTHYETAGKTKPKCVGFIQNYGHYGMIYGVTKDGYYLFADPTKGLKKCTPKELDYATNGRSLGYYMVGLV

SEQ ID NO: 6 - protein, 223 aa, Methanobrevibacter sp.
MIRINKKEYTDARKRIRSFIKKHKRLPNYCQFMTNQKKTVDLNKEEYCGLFEGYMHFCLKNGREPNYLTLTSKANNPIVINYQDDKVSCCPASFLMGMMFLFEYKSEATVKKALGTTSNGTSPTQLINNAGKLGYVVKRIPREFREVKKALEQYKPVLMHIQTKPATCLGFVSDYGHWIICWKVKDNKYYVIDPTKGSKVCNYQILNRATNGRNIGYYTMEIK

SEQ ID NO: 7 - protein, 223 aa, unidentified
  source 1..223: note = Paludibacteraceae bacterium
MVRFNESEIKKAIKTIRASLKANKGLPSTVTMKDTDGKSRKLSKAQYNGLLEARNVFRLKNGRMPNYTTLNSTANNPIVLNYQDDSVSCACASFNMCVQMLYEWIDEKKIKQVFQTGSSGTAPSQVISGAKKLGYNVTKIDRNSKAVKKALDKGYPIMAHIDTIKAPCLGYAKNYGHWIMIHRINDTSYYVLDPTKGSKVCKFANIDKAMLDRTIYYYKVTIV

SEQ ID NO: 8 - protein, 223 aa, Methanobrevibacter sp.
MKYSKTEMKTAAKTIRKNIKKGNGRPEKIKMTDTNGDKHKLKLKQYLGLFKQWNIFRLKNGRYPNYVTYVGKSKEPTVINYQDNGYQCACASFNMALQSLFDWEDETTIAQAFNTGKYGTSPSDMINGAKKFGYKIQPINRNQKALQKAFDKGYGVIAHIDTIKAPCLGYKNNYGHYINIQRITKAGNYRVFDPTKGVLSVKPSCIDNAMLNRTLNYYQVVIE

SEQ ID NO: 9 - protein, 457 aa, Methanobrevibacter sp.
MDCTKVNLKKGSKGPLVGELQKYLRDHGFYKREIDNDFGSFTEDAVKLLQRKQGNDPDGVFGPKTCSKSGLNTYTQEINSSKTIASAIDNKTTVNDDSESSKNSSSDVKGGEISPPEYNMGKQTDGYYYCSCGEKGYEYGRWKVTFVDRCAHADDASHKYKSSKLKWSDKKDVAPLEGQWTCEGCDADYCAVTGKEKSSTSKKRLTVIKTEKMPIKKDVKEPTNVNNPPKDKDIVKTYSKEELANNAEYIINETKKNGKNPDSIQIKNRENNNLDLLNKPQYMGLYENQNIFWVNNKRMPNWTTLATTANNPVKMNYQSNAYNCGPMSMSMASQMLFKNTFESEFVKVCETNKNGTDPKNLSLISKLGFSIKPIDRNSKAVKEALARGSAVIAHIETKPASCLGYQNNYGHYVLIDRINNSQYHVMDPTKGIKWCDFTTLDNATNGRNISYYEMKVA

SEQ ID NO: 10 - protein, 311 aa, Methanosphaera cuniculi
MLNCKTINLKKGSKGAEVKELQTILQNKGYYDGKIDGDYGDITVQAVKLLQRKQGNDPDGNFGPKTCSKLDQKTGTTPTGNINASNQKFLKDEIIRAGKVFRQHIKNNNNYPNYIEMKNSSGKTYQVSKALYMGLFEGMNMFAIKNGRLPNFVVASSTANNPLCIDYQNNGVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIV

SEQ ID NO: 11 - protein, 225 aa, Methanobrevibacter sp.
MSSFSKSEVLRGAKIFRNSIVKGKGVPKEVTLKVRETGKNKTLTRKQFAGIMEATNVFRLKQGRYPNYTTLNSTANNPLVMNYQDDSVSCGCASFNMCVQLTYGEWIDEKAIKKTFGTGSSGTAPSQIIAGAKKYGYAVEKIARNSSAVKKALEKGYAILAHIDTKPATCLGYQNNYGHWTVIYGLDDGKYKIADPTKGIKTCSTKIYDKAMLGRTIYYYQVRPL

SEQ ID NO: 12 - protein, 227 aa, Methanobrevibacter sp.
MSIRINKAQLLDGANGISNFIKANKRFPNYATLTDSNNKQQKVLKANYLDFYKRAFQWAVNHGDIFPNYGTVIGTGTSPIPQNYQDSSTTCGPTSLSMGSCGLFKYKSEAQFKAACNTTSSGTTPENLIAGAAKLGFKLTKISRNIAGVKAALNQCKPVIAHIQTKNATCLGYKGDYGHYVLIKGLSGDDHYLINDPTKGENITCLSTILDNATDGREIYYYSMELA

SEQ ID NO: 13 - protein, 437 aa, Methanobrevibacter sp.
MGFLDWIKYLFTNNKKMARFITKNITKTFGEKEQLEVALFEGENPLSDRNVNISINGMTYVRKTDENGIARLNINLGVGEYKADFEFMDDDFNLANAFCKVFVNPVISTQDMNMTEKDGSQFIAIATSSTGVRLSGVPIIFNINGVDYKRTSDMVGEAKLNINLGKGTYEITTSSLKVSAKNIIHIDEKPKKVTRMEGVNISKAFSDKTAYQCAVYDDNSRVSGTVVISVNGVDYTKTPDSTGLYKLNINLQPGTYQISAQYMGDNEHLPSQIVNTIHIVADPVKTVYNPIRIKYQPNSYTCGPTSLSMCSQILGNEVSINSFANACYTDGSGTSPSNLIAGARKLGFKVTPIGRSYAAVKKAIDAGKPVIAHVMTKTMKCLGWQGNYGHYVVIYDYDNGYYHVADPTKGISWCYKDQVDGATIGYEGIQYYSVERI

SEQ ID NO: 14 - protein, 439 aa, Methanobrevibacter sp.
MNFIDFIKNIFSNNKRNARIITKDLNKIFGEKDALEIALYENNSPLVDKNLNIKINGVTYSRKTDNDGIARLNVNLGVGLYEALISFIDEDFNYTSAYCKVTVNPVLSTQDMRMQEHDGSQFIAIASSSTGVRLSGVPVIFNINGVNYERTTDMVGEAKLNINLGKGDYNISTSSFNTVKKNTIHIDEKPKKVTRMEGTNITKAFSDKTAYQCAVYDDNGRVAGSVDITINGVTYTRTPDESGLYKLNINLQPGTYQLTAKYRGDDTHLASQIINTIHVVADPVKKEIVYNPIKIQYQPNSYTCGPTSLAMCSQILGKSTSISAFANACYTTGNGTSPANLIAGAKKLGFKVTEIPRNINGVKAAIDAGKPVLGHIMTGGVTCGGWQGNFGHYIVIYDYGTVYYYVADPTKGFYRCTHSMIDNAMRGYAGIKYYSVERI

SEQ ID NO: 15 - protein, 228 aa, Methanosphaera cuniculi
MLDCKTINLKKGSTGTKVKELQIILQKKGYYDGVIDAIYGDLTVQAVKLLQREQGNSPDGWFGTKTCSKINQNTTLETPQGNTNASGQKFMKDEIIKAGKVFRQHIKNNNNYPNYLEMKNSNGKTYQVSRALYMGIFEGMNMFSIKKGRLPNYVVANSTANNPLCIDYQNNGYNCGPTSLSMCIQMYGEYISESTLANVCGTTRNGTSPASLNSGAKKIWIQIIRNST

SEQ ID NO: 16 - protein, 155 aa, unidentified
  source 1..155: note = Patescibacteria group bacterium
MLKLKPFEQTSGLCGPASLKIVLEYYGVRKSEAALAKLSGASRGSGVEASGLVRAAEKLGFAAIVKDRATVDDIRRHLKRGIPVIVDWFSKDDGHYSVVAGIENGRIHIQDPEIGRLRSLDLETFQRVWFDFPGDVLRSPKDVICQRMIVIRPRK

SEQ ID NO: 17 - protein, 168 aa, unidentified
  source 1..168: note = Candidatus Taylorbacteria bacterium
MSKVILDVKPFQETLHADMCGPASLKIILHYYGIEKSETELAKLTHLVPGLGIDDTSIVDAARTLGFKAEIKNESDFSDIEKWLKRGVPLIIDWFTRGRNDYSDNEVADGHYSVVYGIDDIYVYIQDPETGSVRKIGKEDFMKVWFDFLGKVIKPNELVIRQIIAIYK

SEQ ID NO: 18 - protein, 167 aa, unidentified
  source 1..167: note = Candidatus Staskawiczbacteria bacterium RIFCSPHIGHO2_01_FULL_34_27
MKKIFLRVKPFQETLSGYCGPASLLILLSYYGVNKTERELGRLCKTNKNLGTTAKGIKEAAEKLGFKVKIKINSNFKDIERWLKRGVPVIVDWFTRGRSDYNDSEITDGHYSVLVGLDSKFIYLQDPEIGRIRKIKKEDFMVVWFDFKGKHIKPNELVIRQIIAIYK

SEQ ID NO: 19 - protein, 168 aa, unidentified
  source 1..168: note = Candidatus Staskawiczbacteria bacterium RIFCSPHIGHO2_01_FULL_36_16
MQKILLKVKPFQETLHADMCGPASLKIVLDYYGIEKSEKELAKLCKIKKGLGVDDKGIKKVAEKLGFRVEIKNNSSFKDIEKWLKKGVPVIVDWFTRGRNDYPESETADGHYSVVCGLDDKFIYLQDPEIGKIRKIKRDDFMKVWFDFSGKYIKAEELIVRQIIAIYK

SEQ ID NO: 20 - protein, 308 aa, unidentified
  source 1..308: note = Patescibacteria group bacterium
MKNKIIFVPKNIMGVYEWQDRNYRIDRVKNIKDFRARKVKFFGTTIGKKFLGYKIKKFINLNGIDVWFVNGDKLRSGVKSGDIDFTMGGHTYRYLYVPEGEIWIESVYQRTKELMSIVWHEYLEQSLMCFGMSYLRAHSLASIFEMTLREGKYTMLPIGTYRQKTKFTCGPAALKIVLEYFRRPISEKYLAKLSKTTKIKGTNPQDLVEGAKKLGFSAYQKEYITPNEVKNLIKKGIPIIANYQYEPRFGEGHYAVIIGFSKDNFILSDPGAMEGYQMVSIKEFMKNWFELEDKTHRQGVIISGNKKL

SEQ ID NO: 21 - protein, 164 aa, unidentified
  source 1..164: note = Parcubacteria group bacterium GW2011_GWA2_47_16
MLNVKPFQETLHHGLCGPASLKIVLDYYGLVKSEQELAELTGTTSGMGTDDKSIARVAESLGFKTVIKNESDFSDIKEWLKKGVPVIVDWFTRGREDYPPSEMADGHYSAVVGLDDNLIYLQDPETGGLREIARDDFKKVWFDFTGEYIRNDELIIRQLIAIYR

SEQ ID NO: 22 - protein, 190 aa, unidentified
  source 1..190: note = Candidatus Nealsonbacteria bacterium
MWLALWPVPMLWLLLGAKINFKNLFMIKLKPFRETPGLCGPASLKIVMDYYGVSVSEADIAKIAGATQEKGVSVAGLIKAARHFGFKVLVKENSSLDDLSYYVKKEIPVIVDWFSEDDGHFSVVADIDKNNVILMDPAIRKFLIYIRRRKFPRETFLRIWFDFPGDYIKTSKDLVLRLMIAVLPKYVGKL

SEQ ID NO: 23 - protein, 162 aa, unidentified
  source 1..162: note = Patescibacteria group bacterium
MKIPYRSQNNHFYCAPASLQMLFEFFGVYKTQNDIAKKLNTNPQKGTEDKSIIDFVTKQGFFCYVNQDSTLEEIDDFIKKGHPVIVNFIEPSQDEGHYALVVGMDEEEIILNDPWNGKGFKMTKKDFKKRWHNSDNRSKRWMMVISRENFNLGKQFLAKEKL

SEQ ID NO: 24 - protein, 153 aa, unidentified
  source 1..153: note = Candidatus Azambacteria bacterium GW2011_GWF1_41_10
MLNIKPYKQTRGMCGPASLKMVLEYFGMEKSEEELGQLTHCNPDTGTKAEDIVLAAKQLGFNALIKDMADFDDIKKYLDQEIPVIVDWFSENDGHYSVAVNMDKENIYLQDPEIGSIRELPLETFKRVWFDFPDKFINSKDDLILRRIIVIQK

SEQ ID NO: 25 - protein, 166 aa, unidentified
  source 1..166: note = Candidatus Pacearchaeota archaeon
MKKISLIDVPYDHQDTDYTCGPSSLKMVLSFFGVRESELKLAHEAHTSRKKGTAHAELIKLARRHGFYCYIRRDSNINTIRHFIRLGLPVIINYRNIPDNESHYAVVVGYGRGRIILNDPWNGRNFKIDDKTFIRHWIGENNTKKWVFVISKEPVIRGKQYNPMKR

SEQ ID NO: 26 - protein, 168 aa, unidentified
  source 1..168: note = Candidatus Staskawiczbacteria bacterium RIFCSPHIGHO2_01_FULL_37_17
MEKILLKVKPFQETLQADMCGPASLKIVLDYYGIEKLEKELAKLCKIKKGLGWNDKDIKKVAEKLGFKVEIKNNSSFKDIEKWLKKGAPVIVDWFTRGRNDYPESETADGHYSVVCGLDDKFIYLQDPEIGKIRKIKRDDFMRVWFDFSGEYIKVEELIVRQIIAIYK

SEQ ID NO: 27 - protein, 722 aa, Methanosphaera cuniculi
MVKYDTSKPIHLAMDYTRCRNCDRDRSMLNQIENSLKSKGFNVVGQRYSPNGLALLTKDDFAKVKGHVGISLVNGADPASLYEVAQIKDGNYLRSPSGSARSGSFKYAEQNLAPRNNIVCMAYWYGSCDPVNPGGKCEKRIRGSETTGPIYNPREFIGKHHIPAFAKSDSKDPLVIADNIAKMFTVTKKKDSNGKNLSLMDVIVQFGWSEGKANTIEHELEFYGVAGESDINNNNNNNTNTVTISPDDTVSQIIIEETYTKAYFQQIQPLKTDPNGSFKQSLLLPYNGKYTVNIHYGGSRYHAPTTASYNIDNKGAVDFTPTLIEQCITTTTKEGKTTIQKEGDASKVKHTYTVRTTITYENGAEKSRSVETIQNDTRVKGATTTTNTNTNTNTNTNYSITGKKDPFSVDIPVITNSDGTVEPDVAHMGNSTYRYKKYSPSETVTLLKEHYTEVMHRDSVSMQLNNYKESKYTAFRTKEEPNVFRVISRTRWNAVEECILRYLVHGQGIAWPDEITINFGEQKTIINGTTVNFKSKDVVNISVKDKQNTGYTCGPTSCSACTQVLHNYYSEQYLQRLIGATSGSGSAPSAHVRALNKLNFKAELYSSRDTLISHLRSGRPAVIHVWNHYICAASISPDESKILMINSTESGNYSPTTGWADTSTVEKRRGGRSAVRVSLNWTITDTDKAKFNHFYNSMGGAWNKPYVHEKICNANSGVNVFL

SEQ ID NO: 28 - protein, 161 aa, unidentified
  source 1..161: note = Candidatus Nealsonbacteria bacterium CG02_land_8_20_14_3_00_37_10
KPFRETPGLCGPASLKIVMDYYGVSVSEADIAKIAGATQEKGVSVAGLIKAARHFGFKVLVKENSSLDDLSYYVKKEIPVIVDWFSEDDGHFSVVADIDKNNVILMDPAIRKFLIYIRRRKFPRETFLRIWFDFPGDYIKTSKNLVLRLMIAVLPKYAGKL

SEQ ID NO: 29 - protein, 295 aa, unidentified
  source 1..295: note = candidate division WWE3 bacterium RBG_16_52_45
MAKLYLPPDLAGLYERQENRRIERVDSLKRHHEKSSRFRRARNFSRRYRVQKIARLSGVNVWLVDGTGIRRDVDVDFTMGGHAYRYLYIPPNEIWVESSLAEGDLWPTIWHEFTERRMMRAGLNYDRAHTYASRLEIVLRDGRTFVLPVGTFRQTEGLCGPAALKIVAEYHGYNFSEPHLARLCHTTKEKGTDPADIIRAARKLRFTAWQREHFTVEMVKETLKRGLPIIANFQLTPDFGEGHYAVIIGFTAKEFILSDPQEDAGYRKVPIKKFMGLWYELEDKTAAQGIIVSVP

SEQ ID NO: 30 - protein, 153 aa, unidentified
  source 1..153: note = Parcubacteria group bacterium Gr01-1014_13
MLRVLPFRQSSGFCGPASLKMVLEYFGIKKSEKELAKLSNAHPHHGTSAKNLVLTAKKLGLKGFYKDFSSIKEIKKYLDKKIPVIVDWFSHDDGHYSVVIGIDKKFIYLQDPELGKINKIDLVTFQRIWFDFEEKMLNKKQDIFIRRLIVIYR

SEQ ID NO: 31 - protein, 141 aa, unidentified
  source 1..141: note = Candidatus Azambacteria bacterium GW2011_GWE1_42_9
MCGPASLKMVLEYFGMEKSEEELGQLTHCNPDTGTKAEDIVLAAKQLGFNALIKDMADFDDIKKYLDQEIPVIVDWFSENDGHYSVAVNMDKENIYLQDPEIGSIRELPLETFKRVWFDFPDKFINSKDDLILRRIIVIQK

SEQ ID NO: 32 - protein, 157 aa, unidentified
  source 1..157: note = Candidatus Pacearchaeota archaeon
MFLLKISGDKMKIKLTKQSEGYCGPASLKMVLSIYGINKSENELAKLTKTSRKKGCDEKDIVKVAKKLGLKGYVKKNSSIFEIKKLVNKGIPVIVDWFSPEEAGHYSVVVGFEKDKILIADPHFGKVKKYRIDWFEERWFDMPFKKIIKKEIIVITK

SEQ ID NO: 33 - protein, 157 aa, unidentified
  source 1..157: note = Candidatus Uhrbacteria bacterium
MAITLNPFRQSPGYCGPASLKILLEYYGKSFSEDELARICGSTQEKGTDHAELVAALETLGEKPVAKERATIEDVRSYIRKGTPVIVGWWSTDEDHYSVVYDMRDETISLMDPEVEGGSVVMPITDFEKVWHDFDGAQNIRVERWMLAIERPAPERP

SEQ ID NO: 34 - protein, 148 aa, unidentified
  source 1..148: note = Candidatus Woesearchaeota archaeon
MLKLKPYKQSRGYCGPACLKMVLGYYGVVKSERTLAKMTKTSRTKGCEEKNIVAAAKKLGFKAYVKQKSSIAELRRLVRKGIPVIVDWFSPEEAGHYSVVVGFEKDNIILADPHFGRIKKHKIDWFSDRWFDRPFKKTLIREIIVIQG

SEQ ID NO: 35 - protein, 164 aa, unidentified
  source 1..164: note = Candidatus Brennerbacteria bacterium
MTRKGVKLLNVKPFRETSGFCGPASLKMVLAYYGVRKSERALARLAGATRSQGVEAPGLVKAARRLGFIARIKDRATLADIKKYIRKCIPVIVDWFSNDDGHYSVVVGITKSTIYLQDPEIGRVRAMDTKTFKRVWFDFPGDILRSKNAVVLQRMLVVRRSGKN

SEQ ID NO: 36 - protein, 163 aa, unidentified
  source 1..163: note = uncultured archaeon
MKLKIPFFKQRTGFYCGPASLEMVLAFFKKYKTQKELAKEARTKKDGTTHEEMIKVARREGFYCYVHNNSTIEEIKHFIKQGLPVIINYKEPSSEEGHYAVISGYKKSRLILNDPWNGNNFKIKEKEFLRRWHDYHKKHEYKNWILVLSRKNLNLGKLFLPTK

SEQ ID NO: 37 - protein, 165 aa, unidentified
  source 1..165: note = Candidatus Harrisonbacteria bacterium CG10_big_fil_rev_8_21_14_0_10_44_23
MLPVKPFRQRTGYCGPASLKMVLDYFGIRKTEKELAVLSGATKKQGVQDGEGLVKAARSLGLKAALKDKATLADIRKYIKQEIPVIVNWFSVNEGHYSVVVDIDKENLYIKDPEWGYTRAMRLDTFMEVWFSIRGNVPSSKEDFNIRRLIAVEPHKPRRGRAKKK

SEQ ID NO: 38 - protein, 222 aa, unidentified
  source 1..222: note = Methanobacterium virus PhiF1
MDIKLKEFKDMIRRVKHFLSTKKRPPRYVNTSMGRITYNQYIDALKRYTDFVKKNKREPKYVRIYSEAKPTHKPGGPNQELFEKTFNVQIESAQDLYAWLRDNVKYSYYYNDKYDNREVIEKIKRGEGVNCTDLAQFCRPILRDLGYECDFIRAEVKCSNNKWYGHVFLRIRGKEYNDWVYFDGTAVTHENFDRPLGKLICTGGTREITINPKWLLNAEQNL

SEQ ID NO: 39 - protein, 220 aa, unidentified
  source 1..220: note = Methanobacteriales archaeon HGW-Methanobacteriales-1
MIISYKEYQDMSVRIMQFQMERGRKPKYVTLNGSKIGQRQYEDMMRRVDLFIKSKGRNPKSVRLDEYIEIVKPSLGRKKSASWIKLEEALDKKFNDAKDLYKAIADQGEYKYYYNDKFDNKMALTRLRKGLGINCTDYAQLIRPVLEDMGYDVRYAHGRVKCGDKQWYGHVWLQIKGRDYGNWVNYDVVAVTHHGIKRPIGSLVCVNGVKDVEYNPKWLI

SEQ ID NO: 40 - protein, 221 aa, Methanobacterium sp.
MIISYKQYQDMSIRIMQFQMERGRKPKYVTLNGTKIGQKQYEDMMKRVDVFIKAKGRNPKSVRLDEDLEIIKPSLGKKKSASWIKLEEALKNKFNNAEELYNALSDQGQYKYYYNDKFDNKMALTRLRKGLGINCTDYSQLLRPVLEDMGYDVRYAHGRVKCGDKQWYGHVWLQIKGRAYDNWINYDVVAVTHHGIKRPIGSLVCVAGVKDVEYDPNWLAQ

SEQ ID NO: 41 - protein, 232 aa, Methanobacterium sp.
MTEKIKRDNYQDVIERTLLYVFKNGKLPSYASINGKKILKKDIQDALTRSNNYFKKNGKCAGNVNMVLQDSTPVSTNPIKTELLKTIEKAVNGTFKTATQFYNLVKANEKYDHYSNDIYPQGKALTRLINNQGLNCADFAQIGHASIFELNKVYRTKYQVDYVHVACKSSSGQYNIGHIVLRVKGEEFKDWTVFDVAEAASGGLPIGRTMCSLGYKVLSYNDPWLLSDDGKT

SEQ ID NO: 42 - protein, 165 aa, unidentified
  source 1..165: note = Methanosphaera sp. WGK6
MDGLDQKHAKKMNQENTSTSKDEVIKTIEKYGGITVTGPVSLYNNFKKFKYDYYYNDIYPLSTELKRIANNQGLNCTDLAQLYYTAYKEMGFTNEIQIVRGTVTCKSGKTFGHVWCRVKDDGKWINVDPSAAAAHGYSYGTLICTNGYTITNINPNWALSDDGKT

SEQ ID NO: 43 - protein, 454 aa, Methanobacterium bryantii
MLFAVLLISSLSLTGITGASATDVGTAQTQAVNNSTVQEKANIQINNTCNTNKSVATQNSTDASVKTVQNTSTAISNVSTNQTTVKTQAVAQNTVSTKDNQTAVTSTATSINSTSKNSTNTTSTQTQEAAAGETKVSKNFTVSQITDAAARVKAYVETNHRLPNYVTIGTTQVEMPEFLKLLTAGLLQLNNGTTTSITLKDINSAAKPSESVKSGNINKAGYLDLAKRVNAFIDANGALPNYATSSLGKLNYQSLIYTFSKIMAFYKTNERLPSYVTVKSWSTVGTSSTSTSNSSNTSTSVPASLQQYLKSTTNCQVTNSQIQALAKSITSGKTSTYDKAVAIFNWVRDNIGYSFYYNTKYGAVGTLSKKTGNCVDTAHLLIALERAAGIPARYEHVKAKFTSGNWYGHVIAQVYVNGKWYNADATSSKNTFGVIKNWNTGTATYKGTYATLPF

SEQ ID NO: 44 - protein, 441 aa, Methanobacterium sp.
MGIAGASAADAGTAQTQAVNNSTVQEKANIQINNTCNTNKSVAAQNSTDASVKTVQNTSTATSNVSTNQTTVKTQAVAQNTVSTKDNQTAVTSTATSINSTSKNSTNTTSTQTQAAAAGETKVSKNFTVSQITDAAARVKAYVETNHKLPNYVTIGTTQVEMPEFLKLLTAGLLQLNNGTTTSITLKDINSAAKPSESVKSGNINKAGYLDLAKRVNAFIDANGALPNYATSSLGKLNYQSLIYTFSKIMAFYKTNERLPSYVTVKSWSTVGTSSTSTSNSSNTSTSVPASLQQYLKSTTNCQVTNSQIQALAKSITSGKTSTYDKAVAIFNWVRDNIGYSFYYNTKYGAVGTLSKKTGNCVDTAHLLIALERAAGIPARYEHVKAKFTSGNWYGHVIAQVYVNGKWYNADATSSKNTFGVIKNWNTGTATYKGTYATLPF

SEQ ID NO: 45 - protein, 502 aa, Methanobacterium sp.
MGIGGVIIKKKLPFAVLLILGLFLASIAGASAADVGTAQSQAVNNSTVQENAGIHTNDTYNENESVATQNSTDENITEGTSTATSNTSTNQTTVKTEPVAQDIANTSADQTATTLTVTSTNSTSKNSTVSETNTKNAVSTALNNSQNSTSTKTNTWNNTTSTQTQSTGYDTIKNNDSYQKAAAGETKVTSKSFTAKQINNAAAKVKAYVETNHRLPNYVTIGTIQVQMSDFLKLLTANILQLYNKKTSSITLKSINSAAKPSESVKSGTMTKSCYLDLAKRVNVFINTNGALPNYATSSLGKLNYKSLIYTFSKVLAFYNTNSRLPNYVTVKPWSKVGTSDTSNSSTPVPASLQQYLKSTTNCQVNNAQIQALAKSITSGKSSTYAKAAAIFNWVRDNIGYSFYYNTKYGAVGTLSSKTANCVDTSHLLIALERAAGIPARYEHVYAKFSSGTWYGHVIAQVWVNGKWYYADASSSRNTFGVINNWNTTTATVYGTYTSISF

SEQ ID NO: 46 - protein, 351 aa, Methanobacterium veterum
NVLTITLNNNLTKATQYAIILNPGSITDLAGNPINAYGIRFTTNGTTFTTTQITTAAASVKSYIETNNKLPNYVTIGTNQITMPQFLQLLVTSLLQINSGTNTAISIGNVSSPTNSTGDYIYGNINQTEFLKIAQSIKSFIDSNGTAPSYASSSLGKVQYETLIYMFSKVVNYYGTNKVLPNYVSVDSAVANPGSYSIPSDLQQYLQATTNCQVTNSQIHALAKSITSGKSSTYDKAVAIFNWVRDNIGYSFYYNTKYGAIGVLSSKTANCVDTAHLLIALERAAGIPARYEHVYAKFSSGNWYGHVIAQVWINGKWYNADATSSSNTFGVINSWNTATATVYGTYATLPF

SEQ ID NO: 47 - protein, 328 aa, Methanobacterium sp.
MVVNANTTTAASVNQTNGTNSVYKVGNNNTVSSTTLTDNNKSTNPISKVAVNSTTKPKINSTMAAGEPVKVNGLTLTQMKDGISRAEAFYKKNGRLPNYISFGNRNISITTFQKNIATQGLKINTTTTTKINGLTIAQIKEGISRAEAFYKKNGRLPNYVSYGSRNISITTFQKNVATAGLKINTSSSNKIDTSSVSALAASLKKGSSSQYETATKIFNWVRDHISYSFYYNSKYGASGTLQKRIGNCCDTANLMVALARAAGIQARYLHGTCKFSSGTWYGHVWAQLYVNGKWLNADGTSYKNSLGVVKNWNTATYTLHGIYQKLPF

SEQ ID NO: 48 - protein, 283 aa, unidentified
  source 1..283: note = Clostridia bacterium
MTKIATINKIAKKVKSNVDKNQKLPTFDNYTQPQYAYLLAQSVITPNKDIKSIKVAKAPNASGTSISRSIDKADYIKLAKYFVKFIKDNGRLPNYVQFKTYKIKPKVFIYAMAKIVVFYEENKKLPSYCNFNSKVFSTAPKVTGNEVFDYFVKIFGNVKTIDEAFAKVKDRGYSYYYDDKYANKTVIDRIKNKWGVNCTDSCQMFWHIAKALGYEVKCMHVQCSTGGHVRLKLKHSKHTEGNWINRDPAAILSDNGQPLTYIWCSNAPLNATNPKWFLDNLYR

SEQ ID NO: 49 - protein, 631 aa, unidentified
  source 1..631: note = Methanobacterium sp. A39
NNGSSGYALFRYGLINSSFGMDSTIPTVKSIDPANNSVNVPINKVIKVTFSESIKRGTLSIYVKNSSTGKYVPITTSISGNVLTITLNSNLTKGTTYNIILNAGSITDLAGNALKAYTTKFTTDATAPTVKSIDPSNNSANIPINKTIKITFSEPVKAGTLSIYLKNLKTGKYVTVTKKISSNILTITPAISLTKATLYYVILNAGSITDLAGNAIRSYTTKFTTDSTAPTVKSIDPSNNSVNVPLGKVINVTFSESIKAGTLGVGVKDRTGKYIPITTSISGNVLTITPKSNLTNGTLYYIVLNASSVTDLAGNALKAYTTQFTTNGTIFTITQINAAAASVKSYVESNNRLPNYVTIGTSQVKMPQFLQLLVTSLLQINNKTNTFMIPGSINAPESPTGSYMYGNIVLTEYLSIAQNIRNYINSNNCAPNYMTSSLGNIQYESLVYIYSKILNYYKTNGNLPNYVLVDSSVTNTAIPSELQQYLQATTNCQVTNSQIQTLAKSLTSGKISAYDKAVAIFNWVRDKLGYSFYYNTKYGAVGTLNAMTGNCVDTAHLLIALERAAGIPARYEHVYAKFSSGNWYGHVIAQVWVNGKWYNVDATSYSNTFGVIKNWNASTATYKGTYATLPF

SEQ ID NO: 50 - protein, 1146 aa, Methanobacterium bryantii
MLSINCISAATETGNTTNTSEQNITSNVINSSNTTIQKSMDTANTTNSSNTTIQKSMEAAGGTSYSKVKAVWIPSDYVSSLNVTALQNLGITDVFVKSNLISTPSYSSVLTSILQKLKGTNIRVHAWITCFKDANGDWIDPANKTQQDFLINSITSIVKNYNVSGINLDYIRYSGSGSNTAGESGTAAITAFVQRVYNTVKSINSSVAVSADLMPEGSANAYYYGQNYTQLAQYLDFLVPMIYKGNYGYNSSTGTSSSGKNGTSWIASTVAYIVSQANGTPVIAGLQTYRSDKNVTAIPASELQNDIDAAINNGSSGYALFRYGLINSSFGMDSTIPTVKSIDPANNSVNVPINKVIKVTFSESIKRGTLSIYVKNSSTGKYVPITTSISGNVLTITLNSNLTKGTTYNIILNAGSITDLAGNALKAYTTKFTTDATAPTVKSIDPSNNSANIPINKTIKITFSEPVKAGTLSIYLKNLKTGKYVTVTKKISSNILTITPAISLTKATLYYVILNAGSITDLAGNSLKSYTTKFTTDKTAPTIKSIDPSNNSANIAINKVIKVTFSEPVKAGTLSIRVKNLKTGSYISIAKKISGNTLTITPTSNLIKATQYAVVLNAGSITDLAGNSLKSYTTKFTTDKTAPTIKSIDPSNNSANIAINKVIKVTFNEPVKTGTLSIKLKNLKTGTYVSITKTISGNTLTIKPTSTLTKATQYAVVLNAGSITDLAGNAIRSYTTKFTTDSTAPTVKSIDPSNNSVNVPLGKVINVTFSESIKAGTLGVGVKDRTGKYIPITTSISGNVLTITPKSNLTNGTLYYIVLNASSVTDLAGNALKAYTTQFTTNGTIFTITQINAAAASVKSYVESNNRLPNYVTIGTSQVKMPQFLQLLVTSLLQINNKTNTFMIPGSINAPESPTGSYMYGNIVLTEYLSIAQNIRNYINSNNCAPNYMTSSLGNIQYESLVYIYSKILNYYKTNGNLPNYVLVDSSVTNTAIPSELQQYLQATTNCQVTNSQIQTLAKSLTSGKISAYDKAVAIFNWVRDKLGYSFYYNTKYGAVGTLNAMTGNCVDTAHLLIALERAAGIPARYEHVYAKFSSGNWYGHVIAQVWVNGKWYNVDATSYSNTFGVIKNWNASTATYKGTYATLPF

SEQ ID NO: 51 - protein, 329 aa, Methanobacterium bryantii
MVVNVNTTTAASVNQTNGSSSVYKVGNNNTVSNITLTDTSKSTTPISKVTVNSTNKSKINSTMAAGEPVKVNGLTLTQMKDGISRAEAFYKKNGRLPNYLSFGTRNISITTFQKNIATQGLKINTTTTTKINGLTIAQIKDGISRAEAFYKKNGRLPNYLSFGTRNISITTFQKNVSTAGLKINTSGSGNKIDTSSVSALAASLKKGSSSQYQTAVNIFNWVRDHISYSFYYNSKYGASGTLKNRIGNCCDTANLMVALARAAGIQARYLHGTCKFSSGTWYGHVWAQLYVNGKWLNADGTSYKNSLGVVKNWNTATYTLHGIYQTLPF

SEQ ID NO: 52 - protein, 242 aa, unidentified
  source 1..242: note = Methanobacterium virus PhiF3
MKDRFDIDEFLELKRRWEAFRFTHGRKPKYIRTKDGEEIPQCIFDDMILRYENFVKKYNRRPRVVSILSDKWIRTGFWKQDKQDTGYTCGPSSLQMALSALGCNVSEDVLAKAGGTGTSGTGHEGMLRAVEYASKKCKMKITGRFQSFNGYGWSKVVDGIKAGAEYVIHLKPLPALKYDYKGSQVWKSFTGGHYVYLVGVNMVDRLVLIADPTKGLRIFGMKQVEDAMAGISKESLLILRKG

SEQ ID NO: 53 - protein, 292 aa, unidentified
  source 1..292: note = Clostridia bacterium
MTKKENVAKIAKEVKTSVEKKYKIPSKTDAGIGYTYPEYAYILAKAVITPGKDVAHKKNIGEAPKPSGSTISRTVKKGDYVKLAKNLISFVDNNKRLPNYLTYGGYKIRTRVYIDAFARIVNYYYTNKRMPAYVNVNSKAFTKPSTAAQSKDEVFNYFVKKFGNVKTIDEAFNKVKERGYGYYYDDVYSNKTSIDRMYSKSGINCTDSCQVFWHIGKALGYSVRCLHVRCSGGDGHVRLQFKHPKNTEGNWINRDPAAILSANGRPLTYNWCTSNYTLLATNPSWFTANLNR

SEQ ID NO: 54 - protein, 874 aa, Methanobacterium sp.
METIGGGIIKRHSLLVMLIICIMAISTIGSSYATVDNQQSAETNADSALNNNTTVNQNPVETVKNNQTGIQNRVQNNSSTGTTGNSTTKTLQAQNTTNSTSNISSNSQNSINALTNTQNAVQTAETNNQNSANTSLTNSANNTTIPQLAAAGTTYTNVHGIWLRAEDAGNITVSELKKANITDIFVKANLISAPTYSSVLSSIITKFKNSGIRVHAWITCFQDANGKWINPANTTQQTTLLNAIKKIVTNYSVDGIFLDYVRYSGVGSNTAGTSGTKTITSFVQKVYNAVKSIKPKAAVSATVMPEGATNANTYGQNYTQLSKYLDFLVPMIYKGNYGQNTTWIGTTTKYIVNNAGGKPVIVGLQSYISDYDTTPLSAGELNADIKSALSNGASGYALYRYGLISKDFIAPPSFNTSDIQSAAAKVKAYIETNHKLPNYVTIGTTQVTMPAFLKLMVKGLIQINSKITTPIILKTVNNPQNSTGSFTKGNINKTSYMDIAQRINSFIDSNGLAPNYATTGLGKVQYEQLVYMFSKILNYYNTNKALPSYVSMDPGVKVSPSSGSDIQTTQVFTLDQIKSAATSVKSYIESNHDLPNYVTIGSVQVKMTDFLRLLLIGTIEVNDGSNAQISLKTVSAAATPSESIKSGNITKANYMDLANRVKAFIDANGALPNHASTTLGEIGYSSLIYMYSKILAYDSTNKALPNYVSVSPWSIVSTVPIPSELQQYLKETKNCQVTNSQIQALAKSITSGKSSTYDKAVAIFNWVRDNIGYSFYYNTKYGAVGTLNAKTGNCVDTAHLLIALERAAGIPARYEHVKAQFTSGTWYGHVIAQVWVNGKWYNADGTSSKNTFGVINNWNTATATYYGTYAELPF

SEQ ID NO: 55 - protein, 435 aa, unidentified
  source 1..435: note = Methanobacterium spitsbergense
MLLLFSITLLSTDYIYATTPNQQNSDIIANTNIVGEITVDVQQDVKSTANIQQTTVGTLNDVQAVSNTVTSQKATKNAVKTVSSSVTSKDSSKNSVKIQSASEAAGDVNNNKAAGTQTTVKSTTVKYTLSDIKDAASRVKIFIESKKKLPKYVQMGTRQITMPQFLQLLTAGLTQVNKGSTGSIALKSVETSLKPTQNLKSGTIKKSEYLVMAGRITSFINSNGKTPNFASSSLGKIQYESLVYMYSRIMKYQSVNKDLPNYATMQPWQKSADTPVTPVTPIPAELQKYLLPTNHCQSTDSRIIALASSITQGSSSAKDRATKLFNWVRDNIGYSFYYDTNYGAVGTLNSRTANCVDTSHLLVALSRASGIPARYVHGVCKFSSGTWYGHVWAQLYVDGTWYNADAISYRNSLGVINNWNTASYTLKGIYAALPF

SEQ ID NO: 56 - protein, 856 aa, unidentified
  source 1..856: note = Methanobacterium sp. A39
MLIICIMAMSTLGSSYAAADNQQSAETTADSALNNNTTVNQNPVETVKNNQTGIQNRVQNTSLTGTTGNRTTQDLQAQNTANSTLNISSNSQNSINTLTNTQNALQTAGTNNQNSANASLTNSANNTTIQPAAGTTYTNVHGIWLRAEDAGNITVSELKKANITDIFVKANLISAPTYSSVLSGIITKFKNSGIRIHAWITCFQDANGKWINPANTTQQTTLLNAIKKIVTNYSVDGIFLDYVRYSGAGSNTAGTSGTKTITSFVQKVYNAVKSIKPKVAVSATVMPEGATNANTYGQNYTQLSKYLDFIVPMIYKGNYGQNTTWIGTTTKYIVNNAGGKPVIVGLQSYISDYDTTPLSAGELNADIKSALSNGASGYALYRYGLISKDFIAPPSFNTSDIQSAAAKVKAYIETNHKLPNYVTIGTTQVTMSAFLKLMVKGLLQINSKITTPITLKTVNNPQNSTGSFTKGNINKTSYIDIAQRINSFIDSNGLAPNYATTGLGKVQYEQLVYMFSKILNYYNTNKALPNYVSMDPGVKVSPPSDSDIQTTQVFTLSQIKSAATSVKSYIESNHDLPNYVTIGLVQVKMTDFLRLLIIGTIEVNDGSNAQISLKTVNAAAKPSESIKGGNITKANYVDLANRVKAFIDANGALPNHATTTLGEIGYPSLIYMYSKILAYDSTNKALPNYVSVSPWSAVSTVPIPSELQQYLKETKNCQVTNSQIQALAKSITSGKSSTYDKAVAIFNWVRDNIGYSFYYNTKYGAVGTLNAKTGNCVDTAHLLIALERAAGIPARYEHVKAQFTSGTWYGHVIAQVWVNGKWYNADGTSSKNTFGVINNWNTATATYYGTYAELPF

SEQ ID NO: 57 - protein, 829 aa, unidentified
  source 1..829: note = Methanobacterium aggregans
MSRNFFQTIISEMYISVHGSEFQGRSGHGGGIIKRNLLLAMLLVMSLALSNVGSIYAYSLSETVSDNNTSNAQSLLENTTNYTNTTTNYTNTQTTANLTVNTTSSVNATNTTNDSGISNTATSNTASNSSTNATAAAGGDYSKIQGIWVSSTDALKVNVTQLLSMGITDIYVKCNIYSDPTYKTLLSQVVQMFNGTGIRVNAWVTCFKDANGNWIDPQGTYNYTVKTPVITSTQVAVKNYYKSWYKSWYKSWYKSWYKSWYKSWYKYRGHWKYKWKSTWKSTWKYTWKYVWKYTLKYTTSYKTVTTTTYTTKTVTSYNTTHNDEVISAISDMVKNYGVDGVNLDYVRYPGTAYKYANGTDAVTSFVEKVYTTVKNINQNAAVSADLMPEGAANAYYYGQDYAQLSQYLDYLVPMIYNGNYNEDTDWIGDMTDYIVKHSGGKPVVVGIQTYESDSNVTPLPASELNQDIKAAIDNGASGYVLFRYGLIDDGFSGYTGNMSSGTTTSDNSTEENNTSTSNNTTISLADIEDAATRVADYVEANYRLPTYVTIGSQNINMSNFLNLLVNALRNIYNNDTDSVVLGNFTSATSSSENVTAGNIYTTEYLSMANSIASYMALNGKAPNYASSTLGNMGFESIVYMYSKILRFEDTNARLPSYVGVKAWSVVTTNSSSTTTVPASLQQYLQATTNCQSTNAQIVALSKSIIASAGATTTYAKAVAIFNWVRDNIGYSFYYNTKYGAVGTLNAKTGNCVDTTHLLIALERAAGIPARYVHGYCKFSSGSWYGHVWAEIYVDGKWYSADAISYRNTFGVINNWNTATIYGRYASISF

SEQ ID NO: 58 - protein, 245 aa, Methanosphaera sp.
YYFAKAIKKPNTSIQKLSFEEAKNPTGDYISRDVPKADYLDMAKRLITYVEKNKKMPNYVTYKGIKVLPRLVIYAFAKILIYYEDHNKTAPKYVTISSKAYTKPTETGNVVYDYWVKKFGFKPTSLDEILEYVADHFEYQYYYDDQKSNREVIDSQAGNCTDLLQMLYNLTKQLGYEIEVLHVLCSGGDGHVRGRFKHPKNTDGEWVYRDIACAASSGNPTCNWCTSGYTLLAVNPSWFFESINR

SEQ ID NO: 59 - protein, 373 aa, unidentified
  source 1..373: note = Methanobacterium sp. SMA-27
MFDLDQIFILFDFVKQGGGIINRKLLLSVLLFFSLALALNASTSSAASMDQKNISISNTSPDNLGNNICSSNYITGNANSTTKTNIKNTTTKKAKINSTMAAGAPVKVNGLTLAQLKGGTSRVQSFYNKNNRLPKYVTFGTRKISISTFKKNIATQGLKIKTKTISTKTVTTKKVNGLTVLQLKEGTYRVQSFYNKNGRLPKYVKFGTRKITMATFQENITTQGLKINTTRKTVKIDTSSVASLAKSLKAGSTSTYNTAVAIFNWVRDNINYSFYYNTRYGASGTLKNRSGNCVDTSHLLISLARSAGITARYVHGTCKFSSGTWYGHVWAQLYLNGKWVIADGTSYRNSLGVVKNWDTSTYKLNGIYTTLPF

SEQ ID NO: 60 - protein, 260 aa, Methanobrevibacter sp.
MGLLTDSNMCIDKTTFKDMLKRKINFVNINKKDPSIVYLSPNAQKDWIEIKQFNDMERRWNSWIDIKKSEPNCVFIGNTFNSPTLQTGNVYVNKYINDYNIRQDTSYYCALNIIQNIWYTLFGQIIKESEIAKVAGTTNPNGTDHKGIEKALNYLAEKYKVTISVEWKHFSDIGWNGVEKIYKSQNQAIGFHVLYKLKFGHYEVGYFINKSTNTIGIVNSLGNKDKNGTFLGYFEKRSFKEMLSYINGINQPSCLIVTKK

SEQ ID NO: 61 - protein, 231 aa, unidentified
  source 1..231: note = Methanothermobacter sp. THM-1
MELKKNKNTPEYVSITQFKDMLSRYNRFKEFNGREPRVVFIYSGGGPSVSLETFKDMCKRYNQFLEENRREPRIVYVTPPEPPVPEEVREMRRVLGEFKTATQLYTLVSRRCKYKFYYNDQTPNREALKKMVTDGINCTDACQLFKPVIEGLGYSVRIEHVKVKCNDNKWYGHYFLRVAGKELASVSLPPERWTVWDYVSATKTGRPLGAPCCSRGIQHLGWGIVGPVYDK

SEQ ID NO: 62 - protein, 259 aa, Methanothermobacter sp.
MPLKNGKIEIKKFREMLRRYEDFVKNEGHEPNIIYIEQGKSDHVSLETFKDMLRRYDRFKKETGREPALIYLDPLGRSGDYVNLATFKDMLRRYDRFKADKGREPSYVMIESMRPLPRLDGKWTRRVIQEIGLFLDASELYRLVSEKCKYNYYYNDQFSSEEALRRMTTTGINCTDACQLFEKVFLEMGYETRIEHCKVKCRDGKWYGHYLLNVKGFELKDWVIWDYVSATKTGRPLGKPCCLNGLKHLGWGIVSPKND

SEQ ID NO: 63 - protein, 204 aa, unidentified
  source 1..204: note = Methanothermobacter sp. K4
DMLRRYEDFVRINGREPNTIYIEQGKSDHVPLGTFKDMLRRYEDFVRINGREPNYISIQPQPSLKGPWTTKVIEKIGTFHDATSLYEQVKKTCKYKYYYNDQVPNHVAVMRMTTSGINCTDACQLFQRVMEEMGYMVKIEHVQVKCSDGEWYGHYLLNVKGYEFKDWVIWDYVSATKTGRPLGKPCCLNGLKHLGWGIVSPKHD

SEQ ID NO: 64 - protein, 221 aa, Methanobacterium sp.; identical to SEQ ID NO: 40

SEQ ID NO: 65 - protein, 220 aa, unidentified; identical to SEQ ID NO: 39
  source 1..220: note = Methanobacteriales archaeon HGW-Methanobacteriales-1

SEQ ID NO: 66 - protein, 222 aa, unidentified; identical to SEQ ID NO: 38
  source 1..222: note = Methanobacterium virus PhiF1

SEQ ID NO: 67 - protein, 120 aa, unidentified
  source 1..120: note = Methanomassiliicoccales archaeon
MEQQRAGIGDKKMIDISNTTICLKKENFKDMQNRYQNFLKTKGREPNIIYIKKNGNTYITLQQFKNMKQRWEEFIKTNNREPNCVYIRKIDPNEPATNFDLYEVVYYQVEKQDTGYTCGP

SEQ ID NO: 68 - protein, 260 aa, unidentified
  source 1..260: note = Thermoplasmatales archaeon
MVDISNITICLPKETFKDMKQRWEEFIQKNKREPNIIYTKKGGDTFVTLKRFKDMKQRWEEFIQKNNREPNCVYIRKIDPNEPATNFDLYEVVYYQVEKQDTGYTCGPSSLLMMLRELAQSALPQYSEAYVAKLAGTSTGGTTHDGMINAAKKIAADLGWKLTAKRVNFSDTSWEQLGKWIADPKVGVVAHVMTAGLPYWGNSDYGHYIFPIGLDLKDKTITIADPARGVPDVYKATFDQFARACRRVTWAPSLLLFIKG

SEQ ID NO: 69 - protein, 328 aa, Methanobacterium sp.; identical to SEQ ID NO: 47

SEQ ID NO: 70 - protein, 329 aa, unidentified; identical to SEQ ID NO: 51
  source 1..329: note = Methanobacterium lacus

SEQ ID NO: 71 - protein, 228 aa, synthetic construct
MVRFSRDMLQDGAKRMFKWLRKGEGLPNYLIMYDMDRNKEYKLVPKEYAGLYESRNIFWIKNGREPNYVTLTSVARNPLVMDYQNTNYTCCPTSLSLASQMLYHYKSESECAKALGTSKGSGTSPAQLIANAPKLGFKIIPIKRDSKEVKKYLKKGFPVICHWQVNQSRNCKGDYTGNFGHYGLIWDMTSTHYVVADPAKGVNRKYKFSCLDNANKGYRQNYYVVCPA

SEQ ID NO: 72 - protein, 153 aa, synthetic construct
RNPLVMDYQNTNYTCCPTSLSLASQMLYHYKSESECAKALGTSKGSGTSPAQLIANAPKLGFKIIPIKRDSKEVKKYLKKGFPVICHWQVNQSRNCKGDYTGNFGHYGLIWDMTSTHYVVADPAKGVNRKYKFSCLDNANKGYRQNYYVVCPA

SEQ ID NO: 73 - protein, 143 aa, synthetic construct
TNYTCCPTSLSLASQMLYHYKSESECAKALGTSKGSGTSPAQLIANAPKLGFKIIPIKRDSKEVKKYLKKGFPVICHWQVNQSRNCKGDYTGNFGHYGLIWDMTSTHYVVADPAKGVNRKYKFSCLDNANKGYRQNYYVVCPA

SEQ ID NO: 74 - protein, 143 aa, synthetic construct
TNYTCCPTSLSNASQMLYHYKRESECAKALGTSKGSGTSPAQLIANAPKLGFKIIPIKRDSKEVKKYLKKGFPVICHWQVNQSRNCKGDYTGNFGHYGLIWDMTSTHYVVADPAKGVNRKYKFSCLDNANKGYRQNYYVVCPA

SEQ ID NO: 75 - protein, 311 aa, synthetic construct; identical to SEQ ID NO: 10

SEQ ID NO: 76 - protein, 228 aa, synthetic construct
ASNQKFLKDEIIRAGKVFRQHIKNNNNYPNYIEMKNSSGKTYQVSKALYMGLFEGMNMFAIKNGRLPNFVVASSTANNPLCIDYQNNGVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIV

SEQ ID NO: 77 - protein, 141 aa, synthetic construct
GVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIV

SEQ ID NO: 78 - protein, 318 aa, Methanosphaera cuniculi
MGGLNCKTINLKKGSKGAEVKELQTILQNKGYYDGKIDGDYGDITVQAVKLLQRKQGNDPDGNFGPKTCSKLDQKTGTTPTGNINASNQKFLKDEIIRAGKVFRQHIKNNNNYPNYIEMKNSSGKTYQVSKALYMGLFEGMNMFAIKNGRLPNFVVASSTANNPLCIDYQNNGVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIVSSGGS

SEQ ID NO: 79 - protein, 165 aa, unidentified
  source 1..165: note = Methanobacterium paludis
MGSLSSTTLSTFNSDKTAYLKLFSESVGVKFNTFTQGFNLIANRVYGHYANDVYSQLNALNRLKKKLGLNCSDITQLLYQMGKDLGYNVAYEHVMCKVSGGHIRLKISGGEFGNLGTQIKNWKTVDGAAQLKSGQGINNIWCPDGAIVSYNDKWLQSDDGKTGSG

SEQ ID NO: 80 - protein, 227 aa, unidentified
  source 1..227: note = Candidatus Methanovirga australis
MGLISYDIYKDMQIRFDEYVEKNNTDPERIFILPGKSESTILTHDLKALAYLNPDKFPIEYNTGENPIESKPDTVKSDLDKLRDKFGYFDTICNWYDNVVRKCDYAYYYNQSHSVRQIIDTLLNDGFIALNCTDWSLITSWIAKELGYPFRVYNVVCSSGAGHVRPAIQDRDNLGDWMSTDPAAIADKSEIYYACLFDVWCDTKNGATIVDTSDENQLLGWMHWGSG

SEQ ID NO: 81 - protein, 228 aa, unidentified
  source 1..228: note = Methanobrevibacter sp. V14
MGTKFSKKEMKKAGKTIRENIKKGKGLIRTINMTDMSGKTHRLNKADYAGLFEAQNVFILKNNRYPNYVTLNSEANNPLVLNYQDDKYSCCVASFNMAVQMLYGWINEDKIKKTFKTDINGTDPSNMIIGAKNLGYKVSRISRNFAGVKQSLNKGYPVIAHIQTKSAKCLGYQNDYGHYVLIYGTTPSGAYKVADPTKGLKVCKANELDKATNGRSINYYSVGILGSG

SEQ ID NO: 82 - protein, 229 aa, Methanobrevibacter sp.
MGSSFSKSEVLRGAKIFRNSIVKGKGVPKEVTLKVRETGKNKTLTRKQFAGIMEATNVFRLKQGRYPNYTTLNSTANNPLVMNYQDDSVSCGCASFNMCVQLTYGEWIDEKAIKKTFGTGSSGTAPSQIIAGAKKYGYAVEKIARNSSAVKKALEKGYAILAHIDTKPATCLGYQNNYGHWTVIYGLDDGKYKIADPTKGIKTCSTKIYDKAMLGRTIYYYQVRPLGSG

SEQ ID NO: 83 - protein, 230 aa, unidentified
  source 1..230: note = uncultured Methanobrevibacter sp.
MGTRFMNDVIVKASKTIQKSIREGKGLPDKISMKDSNSKTHNLSKKEYAGLFESRNVFILKHGRVPNYVTKNSTANNPLVMDYQDSSVTCGPTSLSMAAQMLYGNTSESAFKKACNTDSNGTSPDELVAGAKKLGYKVTKIGRNSSAVKNSLKLGKPVVAHIETGGGTKPKCLGFLNNYGHYILIYAVSGDYYLVADPTKGIKKCAFSQINKATNGRSIHYYSVSPLGSG

SEQ ID NO: 84 - protein, 232 aa, Methanobrevibacter sp.
MGTRFSKNEMNKASKTIVKSIRNGKGLPRIVEMKEMDHNKTIRVDKSHYNGLFEAQNVFIRGHGRLPNYVTMNSTANNPLVMDYQDNGYTCGPTSLSMAIQMLYGWKSEKRCAEACHTGSSGTSPDDLIAGAKSLGYKVTPIKRNFAGVKQSLNKGYPVIAHIETGGRTKPKCLGYVNNYGHYILIYGTTASGGYKVADPTKGLRVCSASELDKATNGRSINYYSVKPLGSG

SEQ ID NO: 85 - protein, 232 aa, Methanosphaera cuniculi
MGLDCKTINLKKGSTGTKVKELQIILQKKGYYDGVIDAIYGDLTVQAVKLLQREQGNSPDGWFGTKTCSKINQNTTLETPQGNTNASGQKFMKDEIIKAGKVFRQHIKNNNNYPNYLEMKNSNGKTYQVSRALYMGIFEGMNMFSIKKGRLPNYVVANSTANNPLCIDYQNNGYNCGPTSLSMCIQMYGEYISESTLANVCGTTRNGTSPASLNSGAKKIWIQIIRNSTGSG

SEQ ID NO: 86 - protein, 234 aa, Methanobrevibacter gottschalkii
MGMVRFSKKEMQKASKIIRESIKKGKGQPLSVKMVEMDKGKTIKVSKAYYMGLFEAQNIFIRNKGRKPNYVTLNSKANNPLVMDYQDNAYTCCPTSFSMATQLLFDNISEKECAKALNTVLGSGTDPSDLIKNSGKLGYKVTPIKRDYKSVKSSLDKGFAVIAHIQTRPATCLGYSGDYGHYVLLYGAYRKLLTKYYKVADPTKGLKTCKASILDKATNGRNIKYYSVGILGSG

SEQ ID NO: 87 - protein, 234 aa, Methanobrevibacter sp.
MGSNPIRFSKAEMKKAAKTIQNNVKKGKGQPKTIKMTDLNGKLHSLKKKEYCGLFDNQAGYFNEHGRLPNYTTLLYETPTAFRGQKQPTSVECGDTGLHNAMTQLMIYHTVAECKKACGTNQNGTTPSQLIAGGKKLGVEVKKIKRTFQAVKEAIDKGYSVIAHIQTAGSTRPKCLGYVYDYGHWISIYNISGKDKFKVYDPTKLYKVCTAIEIIKATGGRSINFYSVSPLGSG

SEQ ID NO: 88 - protein, 273 aa, unidentified
  source 1..273: note = Caudoviricetes sp.
MGDRDHISFTEYTYLMCRIAYWLINNAKKFNTRNVYGYMNRSADYGTIIRVIKERSTNYQPDSLIAEFVECAIYDNTDLSFLPNYVTGRNGTKYFKQTYVNMAQRVSAYEVLNGVSPAIVYLKKEELTSNNKSSILNEARKIFGDFNTCDGWLGKIQGRGYSFYYNNKYSNRNTLQRIKNKQGANCTDSTELSYQILVGLGYTVQVIHVMCKSGTGHVRLRCKHPKYTDNQWIYRDPAAVLKGNGIRSNWCANGRVVAYDPKWIMDTINWGSG

SEQ ID NO: 89 - protein, 320 aa, Methanosphaera stadtmanae
MGLHCNTINLTKGSKGETVKELQTILKNKGYYTGKIDGYYGDLTIQAVKLLQRQQGNSEDGQFGPKTCRKLNQNINNNTTHTTPTNNTNPVGKKFLLDDLITARKTFRKHIKNNNNYPNYITIPDNTGKKYDVLKADYMGIFEAMHMFAIRNGRLPNWVIHNTTANNPLVIDYQNNGYNCGPTSLSMCLQMFADYISEPTLAKYAGTNRNGTGPADLKRTAKHYGYGMTEIGRNSKAVQDSLSKCRPVLMHIHTSYAGGRSCLGYRGAYGHYIMCYGMRNGRYLIADPTKGFKVCNPVAIDNARSSSNMKYYSISILGSG

SEQ ID NO: 90 - protein, 320 aa, Methanosphaera sp.
MGLNCNTINLTKGSKGETVKELQTILKNKGYYTGKIDGYYGDLTIQAVKLLQRQQGNSEDGQFGPKTCRKLNQNINNNTTHTTPTNNTNPVGKKFLLDDLITARKTFRKHIKNNNNYPNYITIPDNTGKKYDVLKADYMGIFEAMHMFAIRNGRLPNWVIHNTTANNPLVIDYQNNGYNCGPASLSMCLQMFADYISEPTLAKYAGTNRNGTGPADLKRTAKHYGYGMTEIGRNSKAVQDSLSKCRPVLMHIHTSYAGGRSCLGYRGAYGHYIMCYGMRNGRYLIADPTKGFKVCNPVAIDNARSSSNMKYYSISILGSG

SEQ ID NO: 91 - protein, 332 aa, unidentified
  source 1..332: note = Methanobacterium sp. ERen5
MGVVNANTTTAASVNQTNGSISANKVGNNNTVSNITLTDTSKSTTPISKVAVNSTTKSKINSTMAAGEPVKVNGLTLTQMKDGISRAEAFYKKNGRLPNYLSFGNRNISITTFQKNIATQGLKINTSTATKINGLTIAQIKEGISRAEAFYKKNGRLPNYLSFGNRNISITTFQKNVSTAGLKINTSSSNKVDTSSVSALAASLKKGSSSQYETAVKIFNWVRDHINYSFYYNSKYGASGTLRNRTGNCCDTSNLMVALARAAGIQARYLHGTCKFSSGTWYGHVWAQLYVNGKWLNADGTSYKNSLGVVKNWNTATYTLHGIYQTLPFGSG

SEQ ID NO: 92 - protein, 333 aa, unidentified
  source 1..333: note = Methanobacterium lacus
MGVVNVNTTTAASVNQTNGSSSVYKVGNNNTVSNITLTDTSKSTTPISKVTVNSTNKSKINSTMAAGEPVKVNGLTLTQMKDGISRAEAFYKKNGRLPNYLSFGTRNISITTFQKNIATQGLKINTTTTTKINGLTIAQIKDGISRAEAFYKKNGRLPNYLSFGTRNISITTFQKNVSTAGLKINTSGSGNKIDTSSVSALAASLKKGSSSQYQTAVNIFNWVRDHISYSFYYNSKYGASGTLKNRIGNCCDTANLMVALARAAGIQARYLHGTCKFSSGTWYGHVWAQLYVNGKWLNADGTSYKNSLGVVKNWNTATYTLHGIYQTLPFGSG

SEQ ID NO: 93 - protein, 473 aa, Methanobacterium sp.
MGVGTAQSQAVNNSTVQENAGIHTNDTYNENESVATQNSTDENITEGTSTATSNTSTNQTTVKTEPVAQDIANTSADQTATTLTVTSTNSTSKNSTVSETNTKNAVSTALNNSQNSTSTKTNTWNNTTSTQTQSTGYDTIKNNDSYQKAAAGETKVTSKSFTAKQINNAAAKVKAYVETNHRLPNYVTIGTIQVQMSDFLKLLTANILQLYNKKTSSITLKSINSAAKPSESVKSGTMTKSCYLDLAKRVNVFINTNGALPNYATSSLGKLNYKSLIYTFSKVLAFYNTNSRLPNYVTVKPWSKVGTSDTSNSSTPVPASLQQYLKSTTNCQVNNAQIQALAKSITSGKSSTYAKAAAIFNWVRDNIGYSFYYNTKYGAVGTLSSKTANCVDTSHLLIALERAAGIPARYEHVYAKFSSGTWYGHVIAQVWVNGKWYYADASSSRNTFGVINNWNTTTATVYGTYTSISFGSG

SEQ ID NO: 94 - protein, 312 aa, unidentified
  source 1..312: note = Caudoviricetes sp.
MGDKESINYTNYSNICNRIAYWLVNNNKKFNTRNVYGYMNRSADYGTIIRVIKERGTNYQSDSLITEFVECAIHDNKDLSFLPNYVIDKNGKKYMKDTYVDMCRRVSAYEVLNGVSPAIVYLTGNTPVQNTTNNNKLHNYLTNKGCSGMGQCTPYNCACNSLQQGFYRLTGIHVSESTIASVAGTTTAGTGHQGINTTVVWFNRKYGKNIKIAWKNFNDLGSSDSARWNKLNEYASNGAVFCHILYRNKYGHYEVLKSVNGNNVTVLNSLGSRCSKPAYCGYIETRSKSNQLSYMRGISQPSVAILTKGGSG

SEQ ID NO: 95 - protein, 276 aa, Methanobrevibacter smithii
MGVNEHISFTKYTYLMNRIAYWLVNNNKKFNTRQVYSYMNRVADYGTIIKAIKERGTNYQQDSLIAEFVECAIFDNKDLSFLPNYVSDTDGTKYYKNCYVSMANRVSAYEVLNGVSPAIVYLEDPHGNGTTSDTTDITLKRFTDKFGGVTDIDSCLNKIRGRGYGYYYNSKYNTQETINKIYNKQGVNCTDSSQLFYRLGLALGYNVQFIHVRCRSGTGHVRLRLKHSKHTGGSWIYRDPAAVLNGNSVSSNWCMNGTILAYDPAWIFSDLYQGSG

SEQ ID NO: 96 - protein, 264 aa, unidentified
  source 1..264: note = Caudoviricetes sp.
MGVNEHISFTKYSYLMNRIAYWLINNNKKFNTRPVYGYMNRTADYGTIIKVIKERGTNYQSDGLITEFVECAIHDNKDLSFLPNYVSDENGVKYYKNCFVSMANRVSAYEVLNGVSPAIVYIEDPHGNGTTSDTTDTTLKRFTDKFGGVTDIDSCLNKIRGRGYGYYYNSKYNTQETINKIYNKQGVNCTDSSQLFYRLGLALGYNVQFIHVRCRSGTGHVRLRLKHSKHTGGSWIYRDPAAVLNGNSVSSNWCMNGTILAGSG

SEQ ID NO: 97 - protein, 278 aa, Methanobrevibacter smithii
MGSKERRMYELYGNDMNRIAYWLVNNNKKFNTRPKYGYQGREADYATIIRVIKERGTNYQSDGLITEFVECAIVDNKDHSFLPNYVTGKDGKRFYKSTYVDMAKRCSLWEVEHNGQSPNFIWYETSTNTSASSSVVGNVTKGISNATNTKISDYKSLYNAFYKGRYGLYYNDVYTQSQSLSRLKSRLPLNCTDLNQLAYYSLKEQGYNVRIVRGLIYCNKTYGHVWCQVFLNGKWVNFDASAAAKGKGLGSLICGSRFSVTNINPAWAVSDDGRTGSG

SEQ ID NO: 98 - protein, 273 aa, Methanobrevibacter sp.
MGSSNSISWTRFCQGMNSICYWLIQNKKKYKKRDYYQILKLKGSCADVEKKAKKLGNNKLVAMYTMAVIKDNKSLDFLPNYVTLKNGLQIDKAEYIDMAIRTEAYITANKRLPAIVYRMSKLPDYKDTTMNFFIKTFNYKGNTIDEALAIIANKKLYSKYFDSQKTDKQTINDASKGKGSNCVDWGQVYYRIAKSLGYDVQFIHVKCRVSGTGHIRLRLRHKKHTSGNWINRDPAAVADTTSGNVRSLWCEDGYVIAYDPSWIFTDLYSSGSG

SEQ ID NO: 99 - protein, 312 aa, unidentified
  source 1..312: note = Caudoviricetes sp.
MGTREYINYTNYSYMCNRIAYWLVNNNKKFNTRNVYGYMNRSADYGTIIRVIKERGTNYPSDSLITEFVECAIHDNKDLSFLPNYVVDKNGKKYMKDTYVDMCRRVSAYEVLHGVSPAIVYLTGNTSVQNTTNNNKLYNYLTTSGCAGMGQCTPYNCACNSLQQCFYRLTGIQVSESTIASVAGTTTSGTGHPGINTAVAWFNRKYGQNIKITWKNFKDLGGSDSARWNKLNQYASNGAVFCHILYRNKWGHYEVLKSVNGNNVTVLNSLGNRCHKPAYCGYIETRSKSNQLSYMRGISQPSVAILTKGGSG

SEQ ID NO: 100 - protein, 227 aa, Methanobrevibacter sp.
MGKYSKTEMKTAAKTIRKNIKKGNGRPEKIKMTDTNGDKHKLKLKQYLGLFKQWNIFRLKNGRYPNYVTYVGKSKEPTVINYQDNGYQCACASFNMALQSLFDWEDETTIAQAFNTGKYGTSPSDMINGAKKFGYKIQPINRNQKALQKAFDKGYGVIAHIDTIKAPCLGYKNNYGHYINIQRITKAGNYRVFDPTKGVLSVKPSCIDNAMLNRTLNYYQVVIEGSG

SEQ ID NO: 101 - protein, 324 aa, synthetic construct
MGHHHHHHGSGGLNCKTINLKKGSKGAEVKELQTILQNKGYYDGKIDGDYGDITVQAVKLLQRKQGNDPDGNFGPKTCSKLDQKTGTTPTGNINASNQKFLKDEIIRAGKVFRQHIKNNNNYPNYIEMKNSSGKTYQVSKALYMGLFEGMNMFAIKNGRLPNFVVASSTANNPLCIDYQNNGVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIVSS

SEQ ID NO: 102 - protein, 324 aa, synthetic construct
MGGLNCKTINLKKGSKGAEVKELQTILQNKGYYDGKIDGDYGDITVQAVKLLQRKQGNDPDGNFGPKTCSKLDQKTGTTPTGNINASNQKFLKDEIIRAGKVFRQHIKNNNNYPNYIEMKNSSGKTYQVSKALYMGLFEGMNMFAIKNGRLPNFVVASSTANNPLCIDYQNNGVNCGPTSLSMCIQMYGEYISEPILARACDTGSNGTGPSELKMGAKKYGYKLVEIPRNINAVKESLAKCRPVIMHIHTSYSGGRSCLGYRGAYGHYIMCYGTNGNYYKLADPTKGFKTCSSTSIDNARSGEFMKYYSVCIVSSGGSHHHHHH

SEQ ID NO: 103 - DNA, 1004 bp, Mycobacterium tuberculosis
  source 1..1004: mol_type = genomic DNA
tatgggggaggattttatgatcggatgttgagtcagtctgagtggtctaaaattcctacggtgggaattgtgtttgcggccgccagggtggatactctccctagggatagttgggaccagcctttggcggcggtttgtacagaggagggtatatgggagtttccaaaataacttctggtgggggggcaaattatcgggcgatcgccacttatatggtattaattgctatagcgatcgctatgctatttcccctattttggttggtgggaactgcctttaaatcccctaccgaaaacatctttcaggttcccccccagtttattccaagggaacctactttcactaattttgtcagagtttggcaaaccaatccctttggtcgctatttatttaatagtaccctcgtcgcccttttaaccgtcggattaaatctgctattttgttccttggcggcctatcctttggcgcgcttaaacttttggggtcgcgattttctcctaagtgcgatcgtttccaccatcatgataccctttcagattgtcatgattcccctgtatattctagcagtacagttaggattaagaaatacttatttaggcattatttttccatctttagcctccgcttttggcatctttctattgcgacaggcatttatgggagttcccaaagagttagaagaagccgccagaattgatggctgttcagaattagggatttggtggcatattatgatcccctcagtccgtcccgccttaatgaccttagccatttttgtatttatcgggtcttggagtgattttctctggcccttaattgtagtcgatcgcccagaatattataccatccctttaggcgtggcaaatctagccggaactttttctctcgattggcggatagtcgccgccggttccgtcatttctatcgctcctgtgttactactatttttaatcgtacaacggtatattgtgcctacagatacagctacgggggttaaaggttga

SEQ ID NO: 104 - DNA, 1020 bp, Mycobacterium tuberculosis
  source 1..1020: mol_type = genomic DNA
ggaatcatgaatcatcccacaactaagcatcaaaaaattacatcctgttactggggaatttctgaactacctgggttagatacaggtaatattaaactcctgaatcaatgtggtattgaaaacacccagcaactgctcacgcgcgggagtaataatgctaataaaatcgccttatctaaccaactaggtatcaacatcagagatatcagtaaatgggtcgcaatgtccgacttagccagggttcccagtattggctatcaatattgtggtgtccttttgcatagtggcgtaagttcagttaaccaactatctcagatgtctattcaacaactgcataaacagattttacggctctatgtaggaaccttgcaatctcgccaactctgtccatctgttgatgtcatccaaacctggattaaacaagctaaaattttgcataaaattaattaataattactagatgaaatcatgaatttttccgaagatttgcgacaaaatgcaccagccaccgaaagaaaccgcgaacctattttagaggtcttaaaaagggtgttgcccgccacgggaaccgttttagaagtagccagcggcacaggacaacacgcggttttttttgctccccgtctctcccctaggcggtggctaccttccgacccgcaacctttgttacaacagagtattaaagcatggcaaattcattcaccttcagataatctttaccctcccctacagttaaatgtagaagctaatccttggccagtggaagggaatgatttaccctgggaattatcggaatttcccatcactgctattgtagcaattaacctgattcatatttcgccttggtcggcttgtgaagggttaatggcgggaagcgatcgcattttgaaaccggggggaattttatatctctatggaccctataaaatagctggaaaacacaccgcacctagtaacgcttcctttgatgaatatttgcgaacttctaaccctaagtggggtgtcagaaaccttgatgat

SEQ ID NO: 105 - DNA, 36 bp, synthetic construct
  source 1..36: mol_type = other DNA
atgacggatcagccgcaagcggaattggcgacataa

SEQ ID NO: 106 - DNA, 36 bp, synthetic construct
  source 1..36: mol_type = other DNA
ttatgtcgccaattccgcttgcggctgatccgtcat

SEQ ID NO: 107 - protein, 180 aa, synthetic construct
SVCVTGASGFIGSWLVMRLLFHGYTVRATVRDPTNQKKVKHLLDLPKAETHLTLWKADLADFGSFDEAIQGCSGVFHVATPMDFESKDPENEVIKPTINGLLDILKACQKAKTVKKLVFTSSAGTVNVEEHDKPVYQESNWSDVEFCKSVKMTGWMYFVSKTLAEQAAWKYAKENNIDFI

SEQ ID NO: 108 - protein, 180 aa, synthetic construct
SVCVTGASGFIGSWLVMRLLDRGYTVRATVRDPANKKKVNHLLDLPKAATHLTLWKADLAEEGSFDEAIKGCTGVFHVATPMDFESKDPENEVIKPTINGVLDIMQACLKAKTVKKLVFTSSAGSVNVEETQKPVYNESNWSDVEFCRRVKMTGWMYFASKTLAEQEAWKFAKENNIDFI

SEQ ID NO: 109 - protein, 180 aa, synthetic construct
SVCVTGASGFVGSWLVMRLLEHGYTVRATVRDPANLKKVKHLLDLPQAATHLTLWKADLDVEGSFDEAIKGCTGVFHVATPMDFESFDPENEVIKPTINGMLDIMKACLKAKTVKKLVFTSSAGAVAIEEHPKEVYSENNWSDVVFCKKVKMTGWMYFVSKTLAEQAAWKFAKENNIDFI

SEQ ID NO: 110 - protein, 180 aa, synthetic construct
TVCVTGASGFIGSWLVMRLLERGYTVRATVRDPTNVKKVKHLLDLPKAETHLTLWKADLADEGSFDEAIKGCTGVFHVATPMDFESKDPENEVIKPTIEGMLGIMKSCAAAKTVKKLVFTSSAGTVNIGEHDLPVYQESCWSDMEFCKAKKMTAWMYFVSKTLAEQAAWKYAKENNIDFI

SEQ ID NO: 111 - protein, 180 aa, synthetic construct
TVCVTGASGFIGSWLVMRLLERGYFVRATVRDPGNLKKVGHLLDLPNAKTGLTLWKADLSEDGSFDDAINGCDGVFHVATPMDFESKDPCNEVIKPTVNGMLGIMKACVKAKTVKKFVFTSSAGTVNVEEHDKNVYQENDWSDLEFIMSKKMTGWMYFVSKSLAEKAAWDFAEEKGLDFI

SEQ ID NO: 112 - protein, 180 aa, synthetic construct
TVCVTGASGFIGSWLAMRLLERGYTVRATVRDPGNLKKVCHLLELPKASTNLTLLKADLTEEGSFDEAIHGCHGVFHVATPMDFESKDPKNEVIKPTIDGFLSIIRSCVKAKTVKKLVFTSSAGTVDVGECQKPVYQENDWSDLDFINSTKMTGWMYFVSKILAEKAAWEVTKANDIGFI

SEQ ID NO: 113 - protein, 180 aa, synthetic construct
TVCVTGASGFIGSWLVMRLLERGYVVHATVRDPGDLKKVKHLLELPKAGTNLKLWKADLTGEGSFDEAIQGCHGVFHLATPMDFESKDPENEIIKPTIEGVLSIIRSCVKAKTVKKLVFTSSAGTVNGGEKDLHVYQESHWSDLDFIYSKKMTAWMYFVSKTLAEKAAWDATKGNNISFI

SEQ ID NO: 114 - protein, 180 aa, synthetic construct
TVCVTGAAGFIGSWLVMRLLERGYNVHATVRDPENKKKVKHLFELPKADTNLTLWKADLSVEGSFDEAIQGCGGVFHVATPMDFESEDPENEVIKPTVRGMLSIIFSCAKAKTVKKLVFTSSAGTLDAGEKDKLFYQETSWSDLDFIYAKKMTGWMYFVSKILAEKAAMEAAKKKNFDFI

SEQ ID NO: 115 - protein, 180 aa, synthetic construct
PVVATGAAGFIGSWLVMKLLRAGYTVRATVRDPANTKKLKPLLDLPGAGSHLTIWKADLNDEGSFDDVIDGCTGVFHVATPMDFESKDPENEVIKPTVDGMLSILKSCKKAGTVKKVIFTSSAGTVNVEERQKPEYQENSWSDIEFCRRVKMTGWMYFVSKSLAERAAWDFARENSMDLI

SEQ ID NO: 116 - protein, 180 aa, synthetic construct
PVAVTGAGGYIGSWLVMKLLRGGYTVRATVRDPDNMKKVKPLLDLPGAASHLTIWKADLNDEGSFDDVINGCAGVFHVATPMDFESKDPENEVIKPTVDGMLSILKSCRKACTVKKVVFTSSAGTVNVEERQKPEYQESAWSDIEFCRRVKMTGWMYFVSKSLAERAAWDFARENNIDLI

SEQ ID NO: 117 - protein, 180 aa, synthetic construct
AVVVTGAGGYVGSWLVMKLLHYGYTVRATVRDSSDEAKTKPLLELPGADTHLSLWCADLLGDGSFDKVISGSIAVFHVATPMDFESIDPENEVIKPAVNGMLSIMKSCKKAGTVKKVIFTSSAGTVNVEEHQKPEYQENSWSDIDFCRRVKMTGWMYFVSKSLAEKAAWEFAKANGIDLV

SEQ ID NO: 118 - protein, 180 aa, synthetic construct
PVVVTGAGGYVGSWLVMKLLQYGYTVRATLRDPRDLKKTKPLLDLPGADEHLTIWKADLSEEGSFDEAINGCTGVFHVATPMDFESEDPENEVIKPTINGVLGIMKSCKKAGTVKKIIFTSSAGTVNVHEHQMPEYQESSWSDIDFIKRVKMTGWMYFVSKILAEKAAWDFAKENDIQFI

SEQ ID NO: 119 - protein, 180 aa, synthetic construct
TVVVTGASGYVGSWLVMKLLQNGYAVRATVRDPTNLKKTKPLLDLSGADRLLTIWKADLGEEGSFDEVIQGCTAVFHVATPMDFESGDPENEVIKPAINGMLSIMRSCKKAGSVEKVIFTSSAGTVNVEEHRRAEYQENCWSDVEFCRRVKMTGWMYFVSKTLAEKAAWDFALENGIHLI

SEQ ID NO: 120 - protein, 180 aa, synthetic construct
PVVVTGASGYVGSWLLMKLLQAGYTVRATVRDPENVGKTKPLLDLPGATFHLSIWKADMADEDSFDEAIKGCTGVFHVATPTDFESKDPENEVIKPTVEGMLKILRACKEAGTVKKIVFTSSAGTVGVEERKRPVYQHDNWSDVDFCRRVKMTGWMYFVSKSLAEKAAMAYAAEHGLDLV

SEQ ID NO: 121 - protein, 180 aa, synthetic construct
TVCVTGASGFIGSWLVMKLLEKGYTVRATVRDPXNLKKVKHLLDLPXADTHLTLWKADLAXEGSFDEAIQGCTGVFHVATPMDFESKDPENCVIKPTINGMLSIMKSCKKAKTVKKLVFTSSAGTVNVEENQKPVYQEXXWSDVCFCRRVKMTGWMYFVSKTLACKAAWQFAKENNIDFI

SEQ ID NO: 122 - DNA, 6905 bp, synthetic construct
  source 1..6905: mol_type = other DNA
ggaatcatgaatcatcccacaactaagcatcaaaaaattacatcctgttactggggaatttctgaactacctgggttagatacaggtaatattaaactcctgaatcaatgtggtattgaaaacacccagcaactgctcacgcgcgggagtaataatgctaataaaatcgccttatctaaccaactaggtatcaacatcagagatatcagtaaatgggtcgcaatgtccgacttagccagggttcccagtattggctatcaatattgtggtgtccttttgcatagtggcgtaagttcagttaaccaactatctcagatgtctattcaacaactgcataaacagattttacggctctatgtaggaaccttgcaatctcgccaactctgtccatctgttgatgtcatccaaacctggattaaacaagctaaaattttgcataaaattaattaataattactagatgaaatcatgaatttttccgaagatttgcgacaaaatgcaccagccaccgaaagaaaccgcgaacctattttagaggtcttaaaaagggtgttgcccgccacgggaaccgttttagaagtagccagcggcacaggacaacacgcggttttttttgctccccgtctctcccctaggcggtggctaccttccgacccgcaacctttgttacaacagagtattaaagcatggcaaattcattcaccttcagataatctttaccctcccctacagttaaatgtagaagctaatccttggccagtggaagggaatgatttaccctgggaattatcggaatttcccatcactgctattgtagcaattaacctgattcatatttcgccttggtcggcttgtgaagggttaatggcgggaagcgatcgcattttgaaaccggggggaattttatatctctatggaccctataaaatagctggaaaacacaccgcacctagtaacgcttcctttgatgaatatttgcgaacttctaaccctaagtggggtgtcagaaaccttgatgattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaatagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccattcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgcctatgggggaggattttatgatcggatgttgagtcagtctgagtggtctaaaattcctacggtgggaattgtgtttgcggccgccagggtggatactctccctagggatagttgggaccagcctttggcggcggtttgtacagaggagggtatatgggagtttccaaaataacttctggtgggggggcaaattatcgggcgatcgccacttatatggtattaattgctatagcgatcgctatgctatttcccctattttggttggtgggaactgcctttaaatcccctaccgaaaacatctttcaggttcccccccagtttattccaagggaacctactttcactaattttgtcagagtttggcaaaccaatccctttggtcgctatttatttaatagtaccctcgtcgcccttttaaccgtcggattaaatctgctattttgttccttggcggcctatcctttggcgcgcttaaacttttggggtcgcgattttctcctaagtgcgatcgtttccaccatcatgataccctttcagattgtcatgattcccctgtatattctagcagtacagttaggattaagaaatacttatttaggcattatttttccatctttagcctccgcttttggcatctttctattgcgacaggcatttatgggagttcccaaagagttagaagaagccgccagaattgatggctgttcagaattagggatttggtggcatattatgatcccctcagtccgtcccgccttaatgaccttagccatttttgtatttatcgggtcttggagtgattttctctggcccttaattgtagtcgatcgcccagaatattataccatccctttaggcgtggcaaatctagccggaactttttctctcgattggcggatagtcgccgccggttccgtcatttctatcgctcctgtgttactactatttttaatcgtacaacggtatattgtgcctacagatacagctacgggggttaaaggttgaatgaggattattaatctcagtcctaataatcaaaatcatatccaccaggcggcgactttattagtcgcagaatttcgggaaaattggccgaatgcttggccgacttatgaacgtggtttagccgaggttatggagtcttttggcgacgatagagttaatctagtcgccgtcgatgaaaatgataatcttctaggctggattggtggtattagccaatatcaaggccatgtttgggaattacatcctttggtagtcaaaagcgattatcaagggttaggaatagggcgaaaattggtcgccaatttagaggattatgtccgctcacaaggagggttaaccttatggttgggtaccgatgacgaaaataatttaacctccctatctggtgtcgaattatacccccattttttggaaaatattgctaacataaaaaaccatggtcgtcatccctatgaattttatcagaaatgcggttttgtgattatgggagtagtccccgatgctaacggtatcgggaaacctgatattttgatggctaaatctctgagaattgacaatcatcctaaatccaactgaatgttgtcagttaagtttatagggctgctatggtatgataaaaaaatccccccagcagttgggggggaatgattaattaggtattgaattggttaaattggaggtgtgttggggctgacttttgctgccttgttggggggggcgattttaactgaggtcactttttcttggccgggattggctaatcgcttgtatgaggctatccgtctgcgggattatcccacggttcaaggggtggtggtatttttggccgttattgtggtggcggctagtttggcgatcgatattttgaatgcttgtattgacccacggattcgctattaattccctgtcagaaaatatacttagttattaatacttagttatttttcttaataaattttaacaaaccaaagggcgttggcagtgtataaaggatatttgaaggggtgagaaggaaggcgaatgttgatttatgaagtttgattaacatttgtatcaaaatataaaattcttctcataaaccctgtacaatcttttaagatttcggaaagtgttctaggatactgaagaaatgaaccacggggcaattgttaaaagcctttgtcgatggttcgccccggaaggggtcttaggaggtgacaccgatggattgattgtcgtgatcattcatggtgtgtccaatcccaactcaactctaagcaagtcaacaagtaggagataaatctatgggacatcatcatcaccaccacggttctggtggattgaactgcaaaaccattaacttaaaaaagggaagtaagggcgcggaagtcaaggaactgcagaccattctccagaacaaagggtactatgatggtaaaatagacggtgattacggcgatatcaccgtacaagcagtgaaattacttcagcgcaaacaaggtaacgatccggacggtaacttcggaccgaaaacctgctccaaactggatcagaaaactggcacgaccccaactggcaatattaacgcgagcaatcagaagtttctgaaggatgagattattcgtgctggcaaagtatttcgtcaacacattaaaaacaataataactatcccaattacattgaaatgaaaaattccagcggcaaaacctaccaggtgtcaaaagcgctgtatatgggcttgtttgaaggcatgaacatgttcgccattaaaaacggtcgcctgccgaattttgtggttgcctcgagcacggcaaacaatccgctttgtattgactatcagaataacggtgtcaattgtggcccgacgagtttatcaatgtgcatccagatgtacggggaatacatctcagaaccgatcctcgcgcgtgcgtgcgacacaggaagcaatggtacaggccctagcgaactgaaaatgggcgccaagaaatatggctataaactggttgaaattccgcgaaacataaacgcggtgaaagagtcgctagccaaatgtcgccctgtcattatgcatatccatactagttatagcgggggtcggtcttgtctgggctaccgtggcgcctatggccactacatcatgtgctatggtaccaacggaaattactataagctggcagatccaaccaaggggtttaaaacctgttcgtcgacgtctatcgataatgctcgctctggtgagttcatgaaatactatagcgtttgcattgtgtcaagctaacagccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccggatccggccggcttgaagacaggtgggacaggagggaggtaaatgc

SEQ ID NO: 123 - DNA, 6905 bp, synthetic construct
  source 1..6905: mol_type = other DNA
ggaatcatgaatcatcccacaactaagcatcaaaaaattacatcctgttactggggaatttctgaactacctgggttagatacaggtaatattaaactcctgaatcaatgtggtattgaaaacacccagcaactgctcacgcgcgggagtaataatgctaataaaatcgccttatctaaccaactaggtatcaacatcagagatatcagtaaatgggtcgcaatgtccgacttagccagggttcccagtattggctatcaatattgtggtgtccttttgcatagtggcgtaagttcagttaaccaactatctcagatgtctattcaacaactgcataaacagattttacggctctatgtaggaaccttgcaatctcgccaactctgtccatctgttgatgtcatccaaacctggattaaacaagctaaaattttgcataaaattaattaataattactagatgaaatcatgaatttttccgaagatttgcgacaaaatgcaccagccaccgaaagaaaccgcgaacctattttagaggtcttaaaaagggtgttgcccgccacgggaaccgttttagaagtagccagcggcacaggacaacacgcggttttttttgctccccgtctctcccctaggcggtggctaccttccgacccgcaacctttgttacaacagagtattaaagcatggcaaattcattcaccttcagataatctttaccctcccctacagttaaatgtagaagctaatccttggccagtggaagggaatgatttaccctgggaattatcggaatttcccatcactgctattgtagcaattaacctgattcatatttcgccttggtcggcttgtgaagggttaatggcgggaagcgatcgcattttgaaaccggggggaattttatatctctatggaccctataaaatagctggaaaacacaccgcacctagtaacgcttcctttgatgaatatttgcgaacttctaaccctaagtggggtgtcagaaaccttgatgattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaatagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccattcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgcctatgggggaggattttatgatcggatgttgagtcagtctgagtggtctaaaattcctacggtgggaattgtgtttgcggccgccagggtggatactctccctagggatagttgggaccagcctttggcggcggtttgtacagaggagggtatatgggagtttccaaaataacttctggtgggggggcaaattatcgggcgatcgccacttatatggtattaattgctatagcgatcgctatgctatttcccctattttggttggtgggaactgcctttaaatcccctaccgaaaacatctttcaggttcccccccagtttattccaagggaacctactttcactaattttgtcagagtttggcaaaccaatccctttggtcgctatttatttaatagtaccctcgtcgcccttttaaccgtcggattaaatctgctattttgttccttggcggcctatcctttggcgcgcttaaacttttggggtcgcgattttctcctaagtgcgatcgtttccaccatcatgataccctttcagattgtcatgattcccctgtatattctagcagtacagttaggattaagaaatacttatttaggcattatttttccatctttagcctccgcttttggcatctttctattgcgacaggcatttatgggagttcccaaagagttagaagaagccgccagaattgatggctgttcagaattagggatttggtggcatattatgatcccctcagtccgtcccgccttaatgaccttagccatttttgtatttatcgggtcttggagtgattttctctggcccttaattgtagtcgatcgcccagaatattataccatccctttaggcgtggcaaatctagccggaactttttctctcgattggcggatagtcgccgccggttccgtcatttctatcgctcctgtgttactactatttttaatcgtacaacggtatattgtgcctacagatacagctacgggggttaaaggttgaatgaggattattaatctcagtcctaataatcaaaatcatatccaccaggcggcgactttattagtcgcagaatttcgggaaaattggccgaatgcttggccgacttatgaacgtggtttagccgaggttatggagtcttttggcgacgatagagttaatctagtcgccgtcgatgaaaatgataatcttctaggctggattggtggtattagccaatatcaaggccatgtttgggaattacatcctttggtagtcaaaagcgattatcaagggttaggaatagggcgaaaattggtcgccaatttagaggattatgtccgctcacaaggagggttaaccttatggttgggtaccgatgacgaaaataatttaacctccctatctggtgtcgaattatacccccattttttggaaaatattgctaacataaaaaaccatggtcgtcatccctatgaattttatcagaaatgcggttttgtgattatgggagtagtccccgatgctaacggtatcgggaaacctgatattttgatggctaaatctctgagaattgacaatcatcctaaatccaactgaatgttgtcagttaagtttatagggctgctatggtatgataaaaaaatccccccagcagttgggggggaatgattaattaggtattgaattggttaaattggaggtgtgttggggctgacttttgctgccttgttggggggggcgattttaactgaggtcactttttcttggccgggattggctaatcgcttgtatgaggctatccgtctgcgggattatcccacggttcaaggggtggtggtatttttggccgttattgtggtggcggctagtttggcgatcgatattttgaatgcttgtattgacccacggattcgctattaattccctgtcagaaaatatacttagttattaatacttagttatttttcttaataaattttaacaaaccaaagggcgttggcagtgtataaaggatatttgaaggggtgagaaggaaggcgaatgttgatttatgaagtttgattaacatttgtatcaaaatataaaattcttctcataaaccctgtacaatcttttaagatttcggaaagtgttctaggatactgaagaaatgaaccacggggcaattgttaaaagcctttgtcgatggttcgccccggaaggggtcttaggaggtgacaccgatggattgattgtcgtgatcattcatggtgtgtccaatcccaactcaactctaagcaagtcaacaagtaggagataaatctatgggtggattgaactgcaaaaccattaacttaaaaaagggaagtaagggcgcggaagtcaaggaactgcagaccattctccagaacaaagggtactatgatggtaaaatagacggtgattacggcgatatcaccgtacaagcagtgaaattacttcagcgcaaacaaggtaacgatccggacggtaacttcggaccgaaaacctgctccaaactggatcagaaaactggcacgaccccaactggcaatattaacgcgagcaatcagaagtttctgaaggatgagattattcgtgctggcaaagtatttcgtcaacacattaaaaacaataataactatcccaattacattgaaatgaaaaattccagcggcaaaacctaccaggtgtcaaaagcgctgtatatgggcttgtttgaaggcatgaacatgttcgccattaaaaacggtcgcctgccgaattttgtggttgcctcgagcacggcaaacaatccgctttgtattgactatcagaataacggtgtcaattgtggcccgacgagtttatcaatgtgcatccagatgtacggggaatacatctcagaaccgatcctcgcgcgtgcgtgcgacacaggaagcaatggtacaggccctagcgaactgaaaatgggcgccaagaaatatggctataaactggttgaaattccgcgaaacataaacgcggtgaaagagtcgctagccaaatgtcgccctgtcattatgcatatccatactagttatagcgggggtcggtcttgtctgggctaccgtggcgcctatggccactacatcatgtgctatggtaccaacggaaattactataagctggcagatccaaccaaggggtttaaaacctgttcgtcgacgtctatcgataatgctcgctctggtgagttcatgaaatactatagcgtttgcattgtgtcaagcggcggtagtcaccatcaccaccatcactaacagccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccggatccggccggcttgaagacaggtaggctaagggcccggctaatgc

SEQ ID NO: 124 - DNA, 5953 bp, synthetic construct
  source 1..5953: mol_type = other DNA
cgagcggtatcagctcactcaaaggcggtaatacggttatccacagaatcaggggataacgcaggaaagaacatgtgagcaaaaggccagcaaaaggccaggaaccgtaaaaatagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccattcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccaagcttgcatgcctgcaggtggcgcgcctatgggggaggattttatgatcggatgttgagtcagtctgagtggtctaaaattcctacggtgggaattgtgtttgcggccgccagggtggatactctccctagggatagttgggaccagcctttggcggcggtttgtacagaggagggtatatgggagtttccaaaataacttctggtgggggggcaaattatcgggcgatcgccacttatatggtattaattgctatagcgatcgctatgctatttcccctattttggttggtgggaactgcctttaaatcccctaccgaaaacatctttcaggttcccccccagtttattccaagggaacctactttcactaattttgtcagagtttggcaaaccaatccctttggtcgctatttatttaatagtaccctcgtcgcccttttaaccgtcggattaaatctgctattttgttccttggcggcctatcctttggcgcgcttaaacttttggggtcgcgattttctcctaagtgcgatcgtttccaccatcatgataccctttcagattgtcatgattcccctgtatattctagcagtacagttaggattaagaaatacttatttaggcattatttttccatctttagcctccgcttttggcatctttctattgcgacaggcatttatgggagttcccaaagagttagaagaagccgccagaattgatggctgttcagaattagggatttggtggcatattatgatcccctcagtccgtcccgccttaatgaccttagccatttttgtatttatcgggtcttggagtgattttctctggcccttaattgtagtcgatcgcccagaatattataccatccctttaggcgtggcaaatctagccggaactttttctctcgattggcggatagtcgccgccggttccgtcatttctatcgctcctgtgttactactatttttaatcgtacaacggtatattgtgcctacagatacagctacgggggttaaaggttgaatgaggattattaatctcagtcctaataatcaaaatcatatccaccaggcggcgactttattagtcgcagaatttcgggaaaattggccgaatgcttggccgacttatgaacgtggtttagccgaggttatggagtcttttggcgacgatagagttaatctagtcgccgtcgatgaaaatgataatcttctaggctggattggtggtattagccaatatcaaggccatgtttgggaattacatcctttggtagtcaaaagcgattatcaagggttaggaatagggcgaaaattggtcgccaatttagaggattatgtccgctcacaaggagggttaaccttatggttgggtaccgatgacgaaaataatttaacctccctatctggtgtcgaattatacccccattttttggaaaatattgctaacataaaaaaccatggtcgtcatccctatgaattttatcagaaatgcggttttgtgattatgggagtagtccccgatgctaacggtatcgggaaacctgatattttgatggctaaatctctgagaattgacaatcatcctaaatccaactgaatgttgtcagttaagtttatagggctgctatggtatgataaaaaaatccccccagcagttgggggggaatgattaattaggtattgaattggttaaattggaggtgtgttggggctgacttttgctgccttgttggggggggcgattttaactgaggtcactttttcttggccgggattggctaatcgcttgtatgaggctatccgtctgcgggattatcccacggttcaaggggtggtggtatttttggccgttattgtggtggcggctagtttggcgatcgatattttgaatgcttgtattgacccacggattcgctattaattccctgtcagaaaatatacttagttattaatacttagttatttttcttaataaattttaacaaaccaaagggcgttggcagtgtataaaggatatttgaaggggtgagaaggaaggcgaatgttgatttatgaagtttgattaacatttgtatcaaaatataaaattcttctcataaaccctgtacaatcttttaagatttcggaaagtgttctaggatactgaagaaatgaaccacggggcaattgttaaaagcctttgtcgatggttcgccccggaaggggtcttaggaggtgacaccgatggattgattgtcgtgatcattcatggtgtgtccaatcccaactcaactctaagcaagtcaacaagtaggagataaatctatgtgagaccactagtggtctctcagccaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactccggatccggccggcttgaagacagnnnnnnnnnnnnnnnnnnnaatgcggaatcatgaatcatcccacaactaagcatcaaaaaattacatcctgttactggggaatttctgaactacctgggttagatacaggtaatattaaactcctgaatcaatgtggtattgaaaacacccagcaactgctcacgcgcgggagtaataatgctaataaaatcgccttatctaaccaactaggtatcaacatcagagatatcagtaaatgggtcgcaatgtccgacttagccagggttcccagtattggctatcaatattgtggtgtccttttgcatagtggcgtaagttcagttaaccaactatctcagatgtctattcaacaactgcataaacagattttacggctctatgtaggaaccttgcaatctcgccaactctgtccatctgttgatgtcatccaaacctggattaaacaagctaaaattttgcataaaattaattaataattactagatgaaatcatgaatttttccgaagatttgcgacaaaatgcaccagccaccgaaagaaaccgcgaacctattttagaggtcttaaaaagggtgttgcccgccacgggaaccgttttagaagtagccagcggcacaggacaacacgcggttttttttgctccccgtctctcccctaggcggtggctaccttccgacccgcaacctttgttacaacagagtattaaagcatggcaaattcattcaccttcagataatctttaccctcccctacagttaaatgtagaagctaatccttggccagtggaagggaatgatttaccctgggaattatcggaatttcccatcactgctattgtagcaattaacctgattcatatttcgccttggtcggcttgtgaagggttaatggcgggaagcgatcgcattttgaaaccggggggaattttatatctctatggaccctataaaatagctggaaaacacaccgcacctagtaacgcttcctttgatgaatatttgcgaacttctaaccctaagtggggtgtcagaaaccttgatgattcacatacgcggccgcctgggccttgagctcgaatttcctgcattaatgaatcggccaacgcgcggggagaggcggtttgcgtattgggcgctcttccgcttcctcgctcactgactcgctgcgctcggtcgttcggctgcgg